SEQUENCE LISTING (59 sequences)

SEQ ID NO: 1 - DNA, 34 bp, synthetic construct
  source 1..34: mol_type = other DNA
tggagggttcgattctggctcaggatgaacgctg

SEQ ID NO: 2 - DNA, 18 bp, synthetic construct
  source 1..18: mol_type = other DNA
ccggatgctccatcacac

SEQ ID NO: 3 - DNA, 19 bp, synthetic construct
  source 1..19: mol_type = other DNA
acaaagtgccttgctccct

SEQ ID NO: 4 - DNA, 20 bp, synthetic construct
  source 1..20: mol_type = other DNA
ttccagttgatcgcatggtc

SEQ ID NO: 5 - DNA, 20 bp, synthetic construct
  source 1..20: mol_type = other DNA
ggaaaccccatctctgggat

SEQ ID NO: 6 - DNA, 20 bp, synthetic construct
  source 1..20: mol_type = other DNA
gctggcaatgccatcgtgct

SEQ ID NO: 7 - DNA, 20 bp, synthetic construct
  source 1..20: mol_type = other DNA
tctcaacggttgctgtatcg

SEQ ID NO: 8 - DNA, 22 bp, synthetic construct
  source 1..22: mol_type = other DNA
cctttctaaggaagcgaaggat

SEQ ID NO: 9 - DNA, 21 bp, synthetic construct
  source 1..21: mol_type = other DNA
acgcttggtattccaaatcgc

SEQ ID NO: 10 - DNA, 22 bp, synthetic construct
  source 1..22: mol_type = other DNA
gccgatcgttgacgttagttgg

SEQ ID NO: 11 - DNA, 21 bp, synthetic construct
  source 1..21: mol_type = other DNA
cagcggttatgcgatgcgaat

SEQ ID NO: 12 - DNA, 20 bp, synthetic construct
  source 1..20: mol_type = other DNA
caatgccgtggttgttggaa

SEQ ID NO: 13 - DNA, 20 bp, synthetic construct
  source 1..20: mol_type = other DNA
gccaatcaccgcattaatcg

SEQ ID NO: 14 - DNA, 1538 bp, Bifidobacterium animalis
  source 1..1538: mol_type = unassigned DNA
tttgtggagggttcgattctggctcaggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaaggtgagactcgtgattgggactaagtcgtaacaaggtagccgtaccggaaggtgcggctggatcacctccttt

SEQ ID NO: 15 - DNA, 3057 bp, Bifidobacterium animalis
  source 1..3057: mol_type = unassigned DNA
gcagacagtaccgatgaaggacgtgtggggccgcgatagtcctgggggagccgccgacatggctttgatcccagggtgtccgaatggggcaacccgccagccgtcatgggctggcaccgcacttgtgcggggggtacgcagggaagtgaaacatctcagtacctgcaggagaggatactccgtgagtagtggcgagcgaaagcggatgatggctaaaccgtgcgcgtgtgatacccgtcgggggttgcgcgcatggtgtcgtgggaacgcctgtccaacggccgacgccgttggctgtggtgataaatgcatgcgtgaggcgaaccgggttggataccgggccgtagagggtgatggccccgtagccgtatgcgcgtgcaccatggatggtgtctcccgagtagcgcgggactcgtggaatcccgtgtgaatccgcccggaccgtcgggtaagcctgagtatacctgtctgaccgatagcgtacgagtaccgtgagggaaaggtgaaaagcaccccgggaggggagtgaaagagttcctgaaaccgtgcgcccgcgaaccgtcggagcccccttggtggggtgacggcgtgcctatcgaaaaatgagtctgcgagtcagtggcatgtggcgagcataagccgtgtggtgtatgcgtagcgaaagcgagtcttaaaaggcgtcttttcagtcgcgtgtcctggacccgaagcgggatgatctagccctggtcaggttgaagcgcgggtaagaccgcgtggaggaccgcacccacttaggttgaaaactgaggggatgaactggggttaggggtgaaaggccaatcaaattccgtgatagctggttctcttcgaaatgcatttaggtgcagcgtcggttgattgcgtgtggggggtagagctactggatgcttgcgggcccgtgccgggtaccaacagcaaccaaactccgaatacccatcgcgtctattccggcagtgagtcggcgggggataagctccgtcgtcgagggggagacagccctgaccgtcgtctaaggtccccaagcgcgtgctaagtgggaaaggatgtggagtcgcatagacagccaggaggttggctcagaagcagccatccttgaaagagtgcgtaacagctcactggtctagtggttccgcgccgacaatgtagcggggctcaagcacgccaccgaagacgcggatgcggccgtatggccgtgtggtaggagagcgtcccacaccggggcgaagcggcgggggaacccggccgtggactgtgtgggagtgagaatgcagacatgagtagcgagagcagggtgaggatccctgccgctggaagaccaagggttccagggccacgttcgtcgtccctgggtgagtcgggtcctaaggcgaggccgacaggcgtagtcgaatggatgaacgggtcgatattcccgtaccggctttcggaccgaccaacagcccgtgtgaacaagggccatccgatacggttgatgggggcttcggcctcctgattccgttgctgtttggcctggacgcgcatgggttgagcgtttcaggagtgacgcggacgggtagccggccggggaggtggttttccctggtcaagcgtgtggccccgcccccaggcaaatccgggggcttcatggggtgaggcgtgatgatgggcgctttcatggcgtgatatccggtgatcccgtcggccgagaaaagcttcggcgcgaggtccgttgccgcccgtaccccaaaccgacacaggtggtctggtagagtataccgaagcgatcgagcgaatcctggtcaaggaactcggcaaattgctcccgtgccttcggcataagggagacccccggccgtgccggccccttgcgggtcgtgggcggctgggggtggcacaggccagggggtagcgactgtttaccaaaaacacaggtgcatgccaagacgcaagtcgccgtatatgcactgacgcctgcccggtgccggaaggttaagaggatccatcagcgtcccctcgcgggatgcgaagtggtgaattcaagccccggtaaacggcggtggtaactataaccatcctaaggtagcgaaattccttgtcgggtaagttccgacctgcacgaatggcgtaacgacttccccgctgtctcgaccaggagctcggcgaaattgcagtacgagtaaagatgctcgttaagcgcagaaggacgaaaagaccccgggacctttactataccttggtattggcatcaggtgcggattgtgtagcataggcgggaggcttcgaagccgcggcgccagccgtggtggagccggccagtgaaataccgctctgtccccatctgatgcctaacccggacaagtcagccttgtcggggagagtgcctggcgggtagtttaactggggcggttgcctcccaaagagtaacggaggcgctcaaaggtccgctcggcccggttggcaatcgggtgtcgagtgtaatcgcacaagcgggcttgactgcgagatcgacggatcgagcagggacgaaagtcggagatagtgatccggtgccggcgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatcctgaggaggcctgcccatagtacgagaggacctgggtggacgaacctctggtatgccagttgtcgcgccagcggcacggctggttggccacgttcgggagggataaccgctgaaagcatctaagcgggaagcccgctccaagataaggattcctgacaccccccccgggggtgtgtgagaccccatagagaacatgtggttgatagaccggacgtggaagccccgcgaggggtggagccgaccggcactaacggtcgaagacaaccaacacc

SEQ ID NO: 16 - DNA, 3127 bp, Bifidobacterium animalis
  source 1..3127: mol_type = unassigned DNA
gtggttgtcgtgatgggcgtatggtggatgccttggcagacagtaccgatgaaggacgtgtggggccgcgatagtcctgggggagccgccgacatggctttgatcccagggtgtccgaatggggcaacccgccagccgtcatgggctggcaccgcacttgtgcggggggtacgcagggaagtgaaacatctcagtacctgcaggagaggatactccgtgagtagtggcgagcgaaagcggatgatggctaaaccgtgcgcgtgtgatacccgtcgggggttgcgcgcatggtgtcgtgggaacgcctgtccaacggccgacgccgttggctgtggtgataaatgcatgcgtgaggcgaaccgggttggataccgggccgtagagggtgatggccccgtagccgtatgcgcgtgcaccatggatggtgtctcccgagtagcgcgggactcgtggaatcccgtgtgaatccgcccggaccgtcgggtaagcctgagtatacctgtctgaccgatagcgtacgagtaccgtgagggaaaggtgaaaagcaccccgggaggggagtgaaagagttcctgaaaccgtgcgcccgcgaaccgtcggagcccccttggtggggtgacggcgtgcctatcgaaaaatgagtctgcgagtcagtggcatgtggcgagcataagccgtgtggtgtatgcgtagcgaaagcgagtcttaaaaggcgtcttttcagtcgcgtgtcctggacccgaagcgggatgatctagccctggtcaggttgaagcgcgggtaagaccgcgtggaggaccgcacccacttaggttgaaaactgaggggatgaactggggttaggggtgaaaggccaatcaaattccgtgatagctggttctcttcgaaatgcatttaggtgcagcgtcggttgattgcgtgtggggggtagagctactggatgcttgcgggcccgtgccgggtaccaacagcaaccaaactccgaatacccatcgcgtctattccggcagtgagtcggcgggggataagctccgtcgtcgagggggagacagccctgaccgtcgtctaaggtccccaagcgcgtgctaagtgggaaaggatgtggagtcgcatagacagccaggaggttggctcagaagcagccatccttgaaagagtgcgtaacagctcactggtctagtggttccgcgccgacaatgtagcggggctcaagcacgccaccgaagacgcggatgcggccgtatggccgtgtggtaggagagcgtcccacaccggggcgaagcggcgggggaacccggccgtggactgtgtgggagtgagaatgcagacatgagtagcgagagcagggtgaggatccctgccgctggaagaccaagggttccagggccacgttcgtcgtccctgggtgagtcgggtcctaaggcgaggccgacaggcgtagtcgaatggatgaacgggtcgatattcccgtaccggctttcggaccgaccaacagcccgtgtgaacaagggccatccgatacggttgatgggggcttcggcctcctgattccgttgctgtttggcctggacgcgcatgggttgagcgtttcaggagtgacgcggacgggtagccggccggggaggtggttttccctggtcaagcgtgtggccccgcccccaggcaaatccgggggcttcatggggtgaggcgtgatgatgggcgctttcatggcgtgatatccggtgatcccgtcggccgagaaaagcttcggcgcgaggtccgttgccgcccgtaccccaaaccgacacaggtggtctggtagagtataccgaagcgatcgagcgaatcctggtcaaggaactcggcaaattgctcccgtgccttcggcataagggagacccccggccgtgccggccccttgcgggtcgtgggcggctgggggtggcacaggccagggggtagcgactgtttaccaaaaacacaggtgcatgccaagacgcaagtcgccgtatatgcactgacgcctgcccggtgccggaaggttaagaggatccatcagcgtcccctcgcgggatgcgaagtggtgaattcaagccccggtaaacggcggtggtaactataaccatcctaaggtagcgaaattccttgtcgggtaagttccgacctgcacgaatggcgtaacgacttccccgctgtctcgaccaggagctcggcgaaattgcagtacgagtaaagatgctcgttaagcgcagaaggacgaaaagaccccgggacctttactataccttggtattggcatcaggtgcggattgtgtagcataggcgggaggcttcgaagccgcggcgccagccgtggtggagccggccagtgaaataccgctctgtccccatctgatgcctaacccggacaagtcagccttgtcggggagagtgcctggcgggtagtttaactggggcggttgcctcccaaagagtaacggaggcgctcaaaggtccgctcggcccggttggcaatcgggtgtcgagtgtaatcgcacaagcgggcttgactgcgagatcgacggatcgagcagggacgaaagtcggagatagtgatccggtgccggcgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatcctgaggaggcctgcccatagtacgagaggacctgggtggacgaacctctggtatgccagttgtcgcgccagcggcacggctggttggccacgttcgggagggataaccgctgaaagcatctaagcgggaagcccgctccaagataaggattcctgacaccccccccgggggtgtgtgagaccccatagagaacatgtggttgatagaccggacgtggaagccccgcgaggggtggagccgaccggcactaacggtcgaagacaaccaacaccaccatgacgaagccggaaaaccccggcggcatcac

SEQ ID NO: 17 - DNA, 1531 bp, Bifidobacterium breve
  source 1..1531: mol_type = unassigned DNA
tttgtggagggttcgattctggctcaggatgaacgctggcggcgtgcttaacacatgcaagtcgaacgggatccatcgggctttgcttggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcattttttgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgcgggagggagccgtctaaggtgaggctcgtgattgggactaagtcgtaacaaggtagccgtaccggaaggtgcggctggatcacctccttt

SEQ ID NO: 18 - DNA, 3064 bp, Bifidobacterium breve
  source 1..3064: mol_type = unassigned DNA
tgttgcctgcaagggcgtatggtggatgccttggcagacaggaccgatgaaggacgtttgaggctgcgatatgcctcggggagccgccaacagggctttgatccgaggatttccgaatgggggaacccaccggccgtcatgggccggtaccgccgtgtgcggggggtacgcagggaagtgaaacatctcagtacctgcaggaaaggatattccgtgagtagtggcgagcgaaagcggatcaggccaaaccttgcgcgtgtgatacccgtcgggggttgcgcgcggggtgtagagggatcgcgtttgccggctccgacgggccggccgtcagttgcataaaaccatgtgccaggggaaccgggttgaataccgggccgcagagggtgagggccccgtacccgaacgcgcatggtctggcgatcgcgtctcccaagtagcacgggccccgtggaatcccgtgcgaatctgtcccgaccgtgggataagcctaaatattcctgtctgaccgatagcgaacgagtaccgtgagggaaaggtgaaaagtaccccgggaggggagtgaaacagtctctgaaaccatgcgcctacgaaccgtcggggcccccttgtggggtgacggcgtgcctatcgaaaaatgagtctgcgagtcagtggcatgtggcgagcataacccgtgtggggtatgcgtagcgaaggcgagtcttaaaaggcgtttaaagtcgcgtgtcctggacccgaagcgggatgatctagccctgagcaggttgaagcgcgggtaagaccgtgtggaggaccgaacccacctgggttgaaaaccggggggatgacttggggctaggggtgaaaggccaatcaaattccgtgatagctggttctctccgaaatgcatttaggtgcagcgtcggatcgattacatccggggggtagagcgactggatgcttgcgggcccatatcgggtaccaacagcaaccaaactccgaatacccgtgatgcgtatcccggcagtgagtcggcgggggataagctccgtcgtcgaaagggaaacagcccagatcgtcgtctaaggtccccaagcgtgtgctaagtgggaaaggatgtggagtcgcatagacagccaggaggttggctcagaagcagccacccttgaaagagtgcgtaacagctcactggtctagtggttccgcgccgacaatgtagcggggctcaagcacaccaccgaagacgcggcagtagctttgctactgggtaggagagcgtcccgtgcggggcgaagcggcggcgcaagccggccgtggaccgcatgggagtgagaatgcagacatgagtagcgaaagacgggtgaagatcccgtccgctggacgaccaagggttccagggccacgttcatcgtccctgggtgagtcgggtcctaaggcgaggccgacaggcgtagtcgaatggatgaacgggtcgatattcccgtaccggcatgcaaccgacaaaaccgaatccgcgagtactaacctccgggtccgggctcacgtctccttcgggagccggatgccctggcctgttgggaccgtaacggtagtaggtcagcgcgggagtgacgcagaagggtagccggccgcggaggtggtcttccgtggtcaagcacgcagcccgtcccacaggcaaatccgtggggcgtgtgggcgaggtgcgatgatgggcgccgttgggcgcgatatccggtgatcccggctgccgagaaaagcttcggcgtgaggcggcgtgccgcccgtaccccaaaccgacactggtggtcaggtagagaataccaaagcgatcgagcgaatcctggtcaaggaactcggcaaatcactcccgttccttcggtttaagggagacccccgatggtgaagcggcttgcccgcggagcttttgggggtggcacagaccagggggtagcgactgtttaccaaaaacacaggagcgtgcgaaggcgcaagccgctgtatacgctctgacgcctgcccggtgccggaaggttaagaggatccgtcaggcctcggccgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcggaaggcttcgaagccacggcgccagccgcggtggagccgcaaggtgaaataccgctctgttcgcattggatgtctaacctcgaccagtcatcctggtcagggacagtgcctggcgggtagtttaactggggcggttgcctcccaaagagtaacggaggcgctcaaaggttccctcagcccggttggcaatcgggtggcgagtgtaagtgcacaagggagcttgactgcgagactgacgggtcgagcagggacgaaagtcggaactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgtcggaatgttgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccataggactacgggtccttgaagtccccatgcagaacacgtggttgataggccggacgtggaagccccgcgaggggtggagccgaccggtactaacggacgaaaggcaacacaac

SEQ ID NO: 19 - DNA, 3062 bp, Bifidobacterium breve
  source 1..3062: mol_type = unassigned DNA
gtgttgcctgcaagggcgtatggtggatgccttggcagacaggaccgatgaaggacgtttgaggctgcgatatgcctcggggagccgccaacagggctttgatccgaggatttccgaatgggggaacccaccggccgtcatgggccggtaccgccgtgtgcggggggtacgcagggaagtgaaacatctcagtacctgcaggaaaggatattccgtgagtagtggcgagcgaaagcggatcaggccaaaccttgcgcgtgtgatacccgtcgggggttgcgcgcggggtgtagagggatcgcgtttgccggctccgacgggccggccgtcagttgcataaaaccatgtgccaggggaaccgggttgaataccgggccgcagagggtgagggccccgtacccgaacgcgcatggtctggcgatcgcgtctcccaagtagcacgggccccgtggaatcccgtgcgaatctgtcccgaccgtgggataagcctaaatattcctgtctgaccgatagcgaacgagtaccgtgagggaaaggtgaaaagtaccccgggaggggagtgaaacagtctctgaaaccatgcgcctacgaaccgtcggggccctcttgtggggtgacggcgtgcctatcgaaaaatgagtctgcgagtcagtggcatgtggcgagcataacccgtgtggggtatgcgtagcgaaggcgagtcttaaaaggcgtttaaagtcgcgtgtcctggacccgaagcgggatgatctagccctgagcaggttgaagcgcgggtaagaccgtgtggaggaccgaacccacctgggttgaaaaccggggggatgacttggggctaggggtgaaaggccaatcaaattccgtgatagctggttctctccgaaatgcatttaggtgcagcgtcggatcgattacatccggggggtagagcgactggatgcttgcgggcccatatcgggtaccaacagcaaccaaactccgaatacccgtgatgcgtatcccggcagtgagtcggcgggggataagctccgtcgtcgaaagggaaacagcccagatcgtcgtctaaggtccccaagcgtgtgctaagtgggaaaggatgtggagtcgcatagacagccaggaggttggctcagaagcagccacccttgaaagagtgcgtaacagctcactggtctagtggttccgcgccgacaatgtagcggggctcaagcacaccaccgaagacgcggcagtagctttgctactgggtaggagagcgtcccatgcggggcgaagcggcggcgcaagcccgccgtggaccgcatgggagtgagaatgcagacatgagtagcgaaagacgggtgaagatcccgtccgctggacgaccaagggttccagggccacgttcatcgtccctgggtgagtcgggtcctaaggcgaggccgacaggcgtagtcgaatggatgaacgggtcgatattcccgtaccggcatgcaaccggcaaaaccgaatccgcgagtactaacctccgggtccgggcttacgtctccttcgggagccggatgccctggcctgttgggaccgtaacggtagtaggtcagcgcgggagtgacgcagaagggtagccggccgcggaggtggtcttccgtggtcaagcacgcagcccgtcccacaggcaaatccgtggggcgcgtgggcgaggtgcgatgatgggcgccgcagggcgcgatatccggtgatcccggctgccgagaaaagcttcggcgtgaggcggcgtgccgcccgtaccccaaaccgacactggtggtcaggtagagaataccaaagcgatcgagcgaatcctggtcaaggaactcggcaaatcactcccgttccttcggtttaagggagacccccgatggtgaagcggcttgcccgcggagcttttgggggtggcacagaccagggggtagcgactgtttaccaaaaacacaggagcgtgcgaaggcgcaagccgctgtatacgctctgacgcctgcccggtgccggaaggttaagaggatccgtcaggcctcggccgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcggaaggcttcgaagccacggcgccagccgcggtggagccgcaaggtgaaataccgctctgttcgcattggatgtctaacctcgaccagtcatcctggtcagggacagtgcctggcgggtagtttaactggggcggttgcctcccaaagagtaacggaggcgctcaaaggttccctcagcccggttggcaatcgggtggcgagtgtaagtgcacaagggagcttgactgcgagactgacgggtcgagcagggacgaaagtcggaactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgtcggaatgttgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccataggactacgggtccttgaagtccccatgcagaacacgtggttgataggccggacgtggaagccccgcgaggggtggagccgaccggtactaacggacgaaaggcaacac

SEQ ID NO: 20 - DNA, 3061 bp, Bifidobacterium breve
  source 1..3061: mol_type = unassigned DNA
gtgttgcctgcaagggcgtatggtggatgccttggcagacaggaccgatgaaggacgtttgaggctgcgatatgcctcggggagccgccaacagggctttgatccgaggatttccgaatgggggaacccaccggccgtcatgggccggtaccgccgtgtgcggggggtacgcagggaagtgaaacatctcagtacctgcaggaaaggatattccgtgagtagtggcgagcgaaagcggatcaggccaaaccttgcgcgtgtgatacccgtcgggggttgcgcgcggggtgtagagggatcgcgtttgccggctccgacgggccggccgtcagttgcataaaaccatgtgccaggggaaccgggttgaataccgggccgcagagggtgagggccccgtacccgaacgcgcatggtctggcgatcgcgtctcccaagtagcacgggccccgtggaatcccgtgcgaatctgtcccgaccgtgggataagcctaaatattcctgtctgaccgatagcgaacgagtaccgtgagggaaaggtgaaaagtaccccgggaggggagtgaaacagtctctgaaaccatgcgcctacgaaccgtcggggccctcttgtggggtgacggcgtgcctatcgaaaaatgagtctgcgagtcagtggcatgtggcgagcataacccgtgtggggtatgcgtagcgaaggcgagtcttaaaaggcgtttgagtcgcgtgtcctggacccgaagcgggatgatctagccctgagcaggttgaagcgcgggtaagaccgtgtggaggaccgaacccacctgggttgaaaaccggggggatgacttggggctaggggtgaaaggccaatcaaattccgtgatagctggttctctccgaaatgcatttaggtgcagcgtcggatcgattacatccggggggtagagcgactggatgcttgcgggcccatatcgggtaccaacagcaaccaaactccgaatacccgtgatgcgtatcccggcagtgagtcggcgggggataagctccgtcgtcgaaagggaaacagcccagatcgtcgtctaaggtccccaagcgtgtgctaagtgggaaaggatgtggagtcgcatagacagccaggaggttggctcagaagcagccacccttgaaagagtgcgtaacagctcactggtctagtggttccgcgccgacaatgtagcggggctcaagcacaccaccgaagacgcggcagtagctttgctactgggtaggagagcgtcccatgcggggcgaagcggcggcgcaagcccgccgtggaccgcatgggagtgagaatgcagacatgagtagcgaaagacgggtgaagatcccgtccgctggacgaccaagggttccagggccacgttcatcgtccctgggtgagtcgggtcctaaggcgaggccgacaggcgtagtcgaatggacgaacgggtcgatattcccgtaccggcatgcaaccggcaaaaccgaatccgcgagtactaacctccgggtccgggcttacgtctccttcgggagccggatgccctggcctgttgggaccgtaacggtagtaggtcagcgcgggagtgacgcagaagggtagccggccgcggaggtggtcttccgtggtcaagcacgcagcccgtcccacaggcaaatccgtggggcgcgtgggcgaggtgcgatgatgggcgccgcagggcgcgatatccggtgatcccggctgccgagaaaagcttcggcgtgaggcggcgtgccgcccgtaccccaaaccgacactggtggtcaggtagagaataccaaagcgatcgagcgaatcctggtcaaggaactcggcaaatcactcccgttccttcggtttaagggagacccccgatggtgaacacacttgctgtgggagcttttgggggtggcacagaccagggggtagcgactgtttaccaaaaacacaggagcgtgcgaaggcgcaagccgctgtatacgctctgacgcctgcccggtgccggaaggttaagaggatccgtcaggcttcggccgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcggaaggcttcgaagccacggcgccagccgcggtggagccgcaaggtgaaataccgctctgttcgcattggatgtctaacctcgaccagtcatcctggtcagggacagtgcctggcgggtagtttaactggggcggttgcctcccaaagagtaacggaggcgctcaaaggttccctcagcccggttggcaatcgggtggcgagtgtaagtgcacaagggagcttgactgcgagactgacgggtcgagcagggacgaaagtcggaactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgtcggaatgttgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccataggactacgggtccttgaagtccccatgcagaacacgtggttgataggccggacgtggaagccccgcgaggggtggagccgaccggtactaacggacgaaaggcaacac

SEQ ID NO: 21 - DNA, 1526 bp, Bifidobacterium longum
  source 1..1526: mol_type = unassigned DNA
tttgtggagggttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 22 - DNA, 3065 bp, Bifidobacterium longum
  source 1..3065: mol_type = unassigned DNA
tgtgttgcttgcaagggcgtatggtggatgccttggcagacaggaccgatgaaggacgtttgaggctgcgataagcctcggggagccgcctacagggctttgatccgaggatttccgaatgggggaacccaccggccgtcatgggtcggtaccggcttcggccggggggtacgcagggaagtgaaacatctcagtacctgcaggaaaggatattccgtgagtagtggcgagcgaaagcggatgatggccaaaccttgcgcgtgtgatacccgtcgggggttgcgtgtggggtgttgcgggatcgcgtgtgccggctccgacgggccggccggcagttgtagaaaaccatgtgtcaggggaaccgggttgaataccgggccgcagagggtgagggccccgtacctgaacgcgcatggtctgccgatcgcgtctcccaagtagcacgggtcccgtggaaccccgtgcgaatccgcccggaccgtcgggtaagcctgaatattcctgtctgaccgatagcgaacgagtaccgtgagggaaaggtgaaaagtaccccgggaggggagtgaaacagtctctgaaaccgtgcgcctacaatccgtcggagcctctttgtggggtgacggcgtgcctatcgaaaaatgagtctgcgagtcagtggcatgtggcgagcataacccgtgtggggtatgcgtagcgaaggcgagtcttaaaaggcgtttaaagtcgcgtgtcctggacccgaagcgggatgatctagccctgagcaggttgaagcgcgggtaagaccgtgtggaggaccgaacccacctgggttgaaaaccggggggatgacttggggttaggggtgaaaggccaatcaaattccgtgatagctggttctctccgaaatgcatttgggtgcagcgtcgggtcattgcgtcccgggggtagagctactggatgcttgcgggcccgtatcgggtaccaacagcaaccaaactccgaataccggtgacgtgtatcccggcagtgagtcggcgggggataagcttcgtcgtcgaaagggaaacagcccagaccgtcgtctaaggtcccgaagcgtgtgctaagtgggaaaggatgtggagtcgcatagacagccaggaggttggctcagaagcagccatccttgaaagagtgcgtaacagctcactggtctagtggttccgcgccgacaatgtagcggggctcaagcacaccaccgaagacgcggcagtattttgtactgggtaggagagcgtcccatgaggggcgaagcggccgtgtgaacgcgccgtggacttcatgggagtgagaatgcagacatgagtagcgaaagacgggtgaagatcccgtccgctggatgaccaagggttccagggccacgttcatcgtccctgggtgagtcgggtcctaaggcgaggccgacaggcgtagtcgaatggatgaacgggtcgatattcccgtaccggctttcagccgccagaaccgaggcttggagtactaacctccgggtccgggcttgcttccccttcgggggtgggatgcctgggcctgttgggaccgatccttgtagtaggtcagcgcaggagtgacgcagaagggtagccggccgcggaggtggttttccgtggtcaagcacgcagcccgtcccgtaggcaaatccgcggggcgtgtgggcgaggtgcgatggtgggggccttgtggcccgatatccggtgatcccggctgccgagaaaagcttcggcgtgaggctcgaagccgcccgtaccccaaaccgacactggtggtcaggtagagaataccaaagcgatcgagcgaatcctggtcaaggaactcggcaaatcactcccgttccttcggtttaagggagacccccgatggtgagccgcctcgcgcggggagcttttgggggtggcacagaccagggggtagcgactgtttaccaaaaacacaggtgcatgcgaaggcgcaagccgctgtatatgcactgacgcctgcccggtgccggaaggttaagaggatccgtcagccttcgggtgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcgggaggccgagaggcatggtcgccagatcgtgcggagccgcaaggtgaaataccgctctgttcgcattggatgtctaacctcgaacagtgatcctgttcagggacagtgcctggcgggtagtttaactggggcggttgcctcccaaagagtaacggaggcgcccaaaggtccgctcagcccggttggcaatcgggtggcgagtgcaagtgcacaagcgggcttgactgcgaggacgacggtccgagcagggacgaaagtcgggactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatgttgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccgtgcagcctcgggctgctgtagtccccatgcagaacacgtggtcgataggccggacgtggaagccccgcgaggggtggagccgaccggtactaacggacgaaaggcaacacta

SEQ ID NO: 23 - DNA, 3055 bp, Bifidobacterium longum
  source 1..3055: mol_type = unassigned DNA
gcttgcaagggcgtatggtggatgccttggcagacaggaccgatgaaggacgtttgaggctgcgatatgcctcggggagccgccaacagggctttgatccgaggatttccgaatgggggaacccaccggccgtcatgggtcggtaccggcttcggccggggggtacgcagggaagtgaaacatctcagtacctgcaggaaaggatattccgtgagtagtggcgagcgaaagcggatgatggccaaaccttgcgcgtgtgatacccgtcgggggttgcgcgcggggtgttgcgggatcgcgtgtgccggctccgacgggccggccggcagttgcagaaaaccatgtgtgaggggaaccgggttgaataccgggccgcagagggtgagggccccgtacccgaacgcgcatggtctgccgatcgcgtctcccaagtagcacgggtcccgtggaaccccgtgcgaatccgcccagaccgttgggtaagcctgaatattcctgtctgaccgatagcgaacgagtaccgtgagggaaaggtgaaaagtaccccgggaggggagtgaaacagtctctgaaaccgtgcgcctacaatccgtcggagccttctttgtggggtgacggcgtgcctatcgaaaaatgagtctgcgagtcagtggcatgtggcgagcataacccgtgtggggtatgcgtagcgaaggcgagtcttaaaaggcgtttgagtcgcgtgtcctggacccgaagcgggatgatctagccctgagcaggttgaagcgcgggtaagaccgcgtggaggaccgaacccacctgggttgaaaaccggggggatgacttggggctaggggtgaaaggccaatcaaattccgtgatagctggttctctccgaaatgcatttgggtgcagcgtcgggtcattacgtcccgggggtagagctactggatgcttgcgggcccgtatcgggtaccaacagcaaccaaactccgaataccggtgacgcgtatcccggcagtgagtcggcgggggataagcttcgtcgtcgaaagggaaacagcccagaccgtcgtctaaggtcccgaagcgtgtgctaagtgggaaaggatgtggagtcgcatagacagccaggaggttggctcagaagcagccatccttgaaagagtgcgtaacagctcactggtctagtggttccgcgccgacaatgtagcggggctcaagcacaccaccgaagacgcggcagtatcgtttggtactgggtaggagagcgtcccatgaggggcgaagcgggcgtggaagcgtccgtggacttcatgggagtgagaatgcagacatgagtagcgaaagacgggtgaagatcccgtccgctggatgaccaagggttccagggccacgttcatcgtccctgggtgagtcgggtcctaaggcgaggccgacaggcgtagtcgaatggatgaacgggtcgatattcccgtaccggctttcagccgccagaaccgaggcttggagtactaacctccgggtccgggcttaccgctccttcgggagggggatgccctggcctgttgggaccgatccttgtagtaggtcagcgcaggagtgacgcagaagggtagccggccgcggaggtggttttccgtggtcaagcacgcagcccgtcccgtaggcaaatccgcggggcgtgtgggcgaggtgcgatggtgggggccttatggcccgatatccggtgatcccggctgccgagaaaagcttcggcgtcaggctcgaagccgcccgtaccccaaaccgacactggtggtcaggtagagaataccaaagcgatcgagcgaatcctggtcaaggaactcggcaaatcactcccgttccttcggtttaagggagacccccgatggtgagccgcctcgcgcggggagcttttgggggtggcacagaccagggggtagcgactgtttaccaaaaacacaggtgcatgcgaaggcgcaagccgctgtatatgcactgacgcctgcccggtgccggaaggttaagaggatccgtcagccttcgggtgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcgggaggccgagaggcatggtcgccagatcgtgcggagccgcaaggtgaaataccgctctgttcgcattggatgtctaacctcgaacagtcatcctgttcagggacagtgcctggcgggtagtttaactggggcggttgcctcccaaagagtaacggaggcgcccaaaggtccgctcagcccggttggcaatcgggtggcgagtgcaagtgcacaagcgggcttgactgcgaggacgacggtccgagcagggacgaaagtcgggactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatgctgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccgtgcagcctcgagctgctgtagtccccatgcagaacacgtggtcgataggccggacgtggaagccccgcgaggggtggagccgaccggtactaacggacgaaaggca

SEQ ID NO: 24 - DNA, 3066 bp, Bifidobacterium longum
  source 1..3066: mol_type = unassigned DNA
tgtgttgcttgcaagggcgtatggtggatgccttggcagacaggaccgatgaaggacgtttgaggctgcgatatgcctcggggagccgccaacagggctttgatccgaggatttccgaatgggggaacccaccgaccgtcatgggtcggtaccggcttcggccggggggtacgcagggaagtgaaacatctcagtacctgcaggaaaggatattccgtgagtagtggcgagcgaaagcggatgatggccaaaccttgcgcgtgtgatacccgtcgggggttgcgcgcggggtgttgcgggatcgcgtgcgccggctccgacgggccggccggcagttgcagaaaaccatgtgtcaggggaaccgggttgaataccgggccgcagagggtgagggccccgtacctgaacgcgcatggtctgccgatcgcgtctcccaagtagcacgggtcccgtggaaccccgtgcgaatccgcccagaccgttgggtaagcctgaatattcctgtctgaccgatagcgaacgagtaccgtgagggaaaggtgaaaagtaccccgggaggggagtgaaacagtctctgaaaccgtgcgcctacaatccgtcggagcccttttgtggggtgacggcgtgcctatcgaaaaatgagtctgcgagtcagtggcatgtggcgagcataacccgtgtggggtatgcgtagcgaaggcgagtcttaaaaggcgtttgagtcgcgtgtcctggacccgaagcgggatgatctagccctgagcaggttgaagcgcgggtaagaccgcgtggaggaccgaacccacctgggttgaaaaccggggggatgacttggggctaggggtgaaaggccaatcaaattccgtgatagctggttctctccgaaatgcatttgggtgcagcgtcgggtcattacgtcccgggggtagagctactggatgcttgcgggcccgtatcgggtaccaacagcaaccaaactccgaataccggtgacgcgtatcccggcagtgagtcggcgggggataagcttcgtcgtcgaaagggaaacagcccagaccgtcgtctaaggtcccgaagcgtgtgctaagtgggaaaggatgtggagtcgcatagacagccaggaggttggctcagaagcagccacccttgaaagagtgcgtaacagctcactggtctagtggttccgcgccgacaatgtagcggggctcaagcacaccaccgaagacgcggcagtattttgtactgggtaggagagcgtcccatgaggggcgaagcggccgtgtgaacgcgccgtggacttcatgggagtgagaatgcagacatgagtagcgagagacgggtgaagatcccgtccgctggatgaccaagggttccagggccacgttcatcgtccctgggtgagtcgggtcctaaggcgaggccgacaggcgtagtcgaatggatgaacgggtcgatattcccgtaccggctttcagccgccaaaaccgaggcttggagtactaacctccgggtccgggcttactgctccttcgggggcgggatgccctggcctgttgggaccgatccttgtagtaggtcagcgcaggagtgacgcagaagggtagccggccgcggaggtggtcttccgtggtcaagcacgcagggcgtgggacaggcaaatccgtcccgcatgggtccgaggtgcgatgatgggggccttatggcccgatatccggtgatcccggctgccgagaaaagcttcggcgtcaggctcaaagccgcccgtaccccaaaccgacactggtggtcaggtagagaataccaaagcgatcgagcgaatcctggtcaaggaactcggcaaatcactcccgttccttcggtttaagggagacccccgatggtgaacacacttgctgtgggagcttttgggggtggcacagaccagggggtagcgactgtttaccaaaaacacaggtgcatgcgaaggcgcaagccgctgtatatgcactgacgcctgcccggtgccggaaggttaagaggatccgtcagccttcgggtgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcgggaggccgagaggcgcggtcgccagatcgcgcggagccgtaaggtgaaataccgctctgttcgcattggatgtctaacctcgaacagtcatcctgttcagggacagtgcctggcgggtagtttaactggggcggttgcctcccaaagagtaacggaggcgcccaaaggtccgctcagcccggttggcaatcgggtggcgagtgcaagtgcacaagcgggcttgactgcgaggacgacggtccgagcagggacgaaagtcgggactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatgctgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccgtggaaccttcgagttcctgtagtccccatgcagaacacgtggtcgataggccggacgtggaagccccgcgaggggtggagccgaccggtactaacggacgaagggcaacactaa

SEQ ID NO: 25 - DNA, 1181 bp, Bifidobacterium longum
  source 1..1181: mol_type = unassigned DNA
ccgcctcgcgcggggagcttttgggggtggcacagaccagggggtagcgactgtttaccaaaaacacaggtgcatgcgaaggcgcaagccgctgtatatgcactgacgcctgcccggtgccggaaggttaagaggatccgtcagccttcgggtgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcgggaggcttcgaagcgttggcgccagccagtgcggagccgtaaggtgaaataccgctctgttcgcattggatgtctaacctcgaacagtcatcctgttcagggacagtgcctggcgggtagtttaactggggcggttgcctcccaaagagtaacggaggcgcccaaaggtccgctcagcccggttggcaatcgggtggcgagtgcaagtgcacaagcgggcttgactgcgaggacgacggtccgagcagggacgaaagtcgggactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatgttgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccgtgcagcctcgggctgctgtagtccccatgcagaacacgtggtcgataggccggacgtggaagccccgcgaggggtggagccgaccggtactaacggacgaaaggcaacactca

SEQ ID NO: 26 - DNA, 3064 bp, Bifidobacterium longum
  source 1..3064: mol_type = unassigned DNA
tgttgcttgcaagggcgtatggtggatgccttggcagacaggaccgatgaaggacgtttgaggctgcgatatgcctcggggagccgccaacagggctttgatccgaggatttccgaatgggggaacccaccgaccgtcatgggtcggtaccggcttcggccggggggtacgcagggaagtgaaacatctcagtacctgcaggaaaggatattccgtgagtagtggcgagcgaaagcggatgatggccaaaccttgcgcgtgtgatacccgtcgggggttgcgcgcggggtgttgcgggatcgcgtgcgccggctccgacgggccggccggcagttgcagaaaaccatgtgtcaggggaaccgggttgaataccgggccgcagagggtgagggccccgtacctgaacgcgcatggtctgccgatcgcgtctcccaagtagcacgggtcccgtggaaccccgtgcgaatccgcccagaccgttgggtaagcctgaatattcctgtctgaccgatagcgaacgagtaccgtgagggaaaggtgaaaagtaccccgggaggggagtgaaacagtctctgaaaccgtgcgcctacaatccgtcggagcccttttgtggggtgacggcgtgcctatcgaaaaatgagtctgcgagtcagtggcatgtggcgagcataacccgtgtggggtatgcgtagcgaaggcgagtcttaaaaggcgtttgagtcgcgtgtcctggacccgaagcgggatgatctagccctgagcaggttgaagcgcgggtaagaccgcgtggaggaccgaacccacctgggttgaaaaccggggggatgacttggggttaggggtgaaaggccaatcaaattccgtgatagctggttctctccgaaatgcatttgggtgcagcgtcgggtcattacgtcccgggggtagagctactggatgcttgcgggcccgtatcgggtaccaacagcaaccaaactccgaataccggtgacgcgtatcccggcagtgagtcggcgggggataagcttcgtcgtcgaaagggaaacagcccagaccgtcgtctaaggtcccgaagcgtgtgctaagtgggaaaggatgtggagtcgcatagacagccaggaggttggctcagaagcagccatccttgaaagagtgcgtaacagctcactggtctagtggttccgcgccgacaatgtagcggggctcaagcacaccaccgaagacgcggcagtattttgtactgggtaggagagcgtcccatgaggggcgaagcggccgtgtgaacgcgccgtggacttcatgggagtgagaatgcagacatgagtagcgagagacgggtgaagatcccgtccgctggatgaccaagggttccagggccacgttcatcgtccctgggtgagtcgggtcctaaggcgaggccgacaggcgtagtcgaatggatgaacgggtcgatattcccgtaccggctttcagccgccaaaaccgaggcttggagtactaacctccgggtccgggcttactgctccttcgggggcgggatgccctggcctgttgggaccgatccttgtagtaggtcagcgcaggagtgacgcagaagggtagccggccgcggaggtggtcttccgtggtcaagcacgcagggcgtgggacaggcaaatccgtcccgcatgggtccgaggtgcgatgatgggggccttatggcccgatatccggtgatcccggctgccgagaaaagcttcggcgtcaggctcaaagccgcccgtaccccaaaccgacactggtggtcaggtagagaataccaaagcgatcgagcgaatcctggtcaaggaactcggcaaatcactcccgttccttcggtttaagggagacccccgatggtgaaccgcctcgcgcggggagcttttgggggtggcacagaccagggggtagcgactgtttaccaaaaacacaggtgcatgcgaaggcgcaagccgctgtatatgcactgacgcctgcccggtgccggaaggttaagaggatccgtcagccttcgggtgaagcggtgaattcaagccccggtaaacggcggtggtaactataaccatcctaaggtagcgaaattccttgtcgggtaagttccgacctgcacgaatggcgtaacgacttccccactgtctcgaccaggagctcggcgaaattgcagtacgagtaaagatgctcgttaagcgcagaaggacgaaaagaccccgggacctttactataccttggtattggcattcggtgtggattgtgtagcataggcgggaggccgagaggcgcggtcgccagatcgcgcggagccgtaaggtgaaataccgctctgttcgcattggatgtctaacctcgaacagtcatcctgttcagggacagtgcctggcgggtagtttaactggggcggttgcctcccaaagagtaacggaggcgcccaaaggtccgctcagcccggttggcaatcgggtggcgagtgcaagtgcacaagcgggcttgactgcgaggacgacggtccgagcagggacgaaagtcgggactagtgatccggtgccggtgtacggacgcggcatcgctcaacggataaaaggtaccccggggataacaggctgatcattcccaagagtccatatcgacgggatggtttggcacctcgatgtcggctcgtcgcatcctggggctggagcaggtcccaagggttcggctgttcgccgattaaagcggcacgcgagctgggttcagaacgtcgtgagacagtttggtctctatcctctgcgctcgttggaatgctgaggaggcctgcccatagtacgagaggacctgggtggacgaacctctggtatgccggttgtcacgccagtggcacggccggttggctacgttcggaagggataaccgctgaaagcatctaagcgggaagcctgctccaagataagcattccgtggaaccttcgagttcctgtagtccccatgcagaacacgtggttgataggccggacgtggaagccccgcgaggggtggagccgaccggtactaacggacgaaaggcaacactga

SEQ ID NO: 27 - DNA, 1474 bp, Lactobacillus plantarum
  source 1..1474: mol_type = unassigned DNA
gctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtggacagatgat

SEQ ID NO: 28 - DNA, 2923 bp, Lactobacillus plantarum
  source 1..2923: mol_type = unassigned DNA
aggttaagttaacaagggcgcatggtgaatgccttggcactaggagccgatgaaggacgggactaacaccgatatgcttcggggagctgtacgtaagctatgatccggagatttccgaatggggcaacccagcagttttaatcaactgttaccactagatgaattcatagtctagttggaggtaaacgctgtgaactgaaacatctcattagcagcaggaatataaagaaatttcgattccctaagtagcggcgagcgaacggggaacagcccaaaccaaagtgcttgcactttggggttgtaggactgaacatttgagttaccaaagaacttgatagtcgaaggatttgggaaaatccgccatagatggtgatagcccagtagattaaatcaaattctctcagttcaggatcctgagtacggcggaacacgtgaaattccgtcggaatccgggaggaccatctcccaaggctaaatactacctagtgaccgatagtgaaccagtaccgtgagggaaaggtgaaaagcaccccgggaggggagtgaaatagttcctgaaaccatgtgcctacaataagtcagagcgcgttaatgcgtgatggcgtgccttttgtagaatgaaccggcgagttatgatcccgtgcaaggttaagactaaaaagtcggagccgtagcgaaagcgagtctgaaatgggcgttttgagtacgaggttatagacccgaaaccaggtgacctatccatgtccaggttgaaggtgcggtaaaacgcactggaggaccgaacccgtgtaagttgaaaattgctgggatgaggtgtggatagcggtgaaattccaaacgaacttggagatagctggttctctccgaaatagctttagggctagcctcggaattaggatcatggaggtagagcactatttggactaggggcccgtcttgggttactgaattcagataaactccgaatgccattgattcatatccgggagtcagacgatgagtgataagatccaccgtcgaaaggggaacagcccagaccatcagttaaggtccctaaatgtatgctaagtggaaaaggatgtggagttgcatagacaactaggatgttggctcagaagcagccaccatttaaagagtgcgtaatagctcactagtcgagtgatcctgcgccgaaaatgtaccggggctaagcatactaccgaaaccatggatacgaccattaggtcgcgtgataggagagcgttctaagggcggtgaagcaagatcgtgaggacttgtggagcgcttagaagtgagaatgccggtatgagtagcgaaagataggtgagaatcctatccaccgaatgactaaggtttcctggggaaggctcgtcctcccagggttagtcgggacctaagtcgaggccgagaggcgtagacgatggataacaggttgagattcctgtactagttaagtgcgtttgagcaatggagggacgcaggaggctaagatgtgcattctgttggattagaatgtccaagcagtaagtctggtgaagagtcaaatgcttttcactttaaggacaagctgtgatggggagcgaaatttagtagcgaagcgtctgatgtcacactgccgagaaaagcttctagtgagtacttaactacccgtaccgcaaaccgacacaggtagtcgaggagagaatcctaaggtgagcgagtgaactctcgttaaggaactcggcaaaatgaccccgtaacttcgggagaaggggtgctgatcgcaagatcagccgcagtgaataggcccaggcgactgtttatcaaaaacacaggtctctgcaaaatcgtaagatgacgtataggggctgacgcctgcccggtgctggaaggttaaaaggatgggttagcttcggcgaagctcagaattgaagccccagtaaacggcggccgtaactataacggtcctaaggtagcgaaattccttgtcgggtaagttccgacccgcacgaaaggcgtaacgatctgggcactgtctcaacgagagactcggtgaaattatattgtccgtgaagatgcggactacccgcgacaggacggaaagaccccatggagctttactgtagtttgatattgagtgtttgtacagcttgtacaggataggtaggagccatagaaaccggaacgctagtttcggtggaggcgttggtgggatactaccctcgctgtatgaccactctaacccgcaccactaatcgtggtgggagacagtgtcaggtgggcagtttgactggggcggtcgcctcctaaaaagtaacggaggcgcccaaaggttccctcagaatggttggaaatcattcgcagagtgtaaaggcacaagggagcttgactgcgagacagacaggtcgagcagggacgaaagtcgggcttagtgatccggtggtaccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgtaggaaatttgagaggacctgtccttagtacgagaggaccgggatggacatacctctggtgtaccagttgtgccgccaggcgcatcgctgggtagctacgtatggatgtgataaacgctgaaagcatctaagtgtgaaacacacctcgagatgagatttcccattcctttatggaagtaagacccctgaaagatgatcaggtagataggttagaagtggcagtgcggtgacgcatgaagcggactaatactaatcggtcgaggacttaaccaag

SEQ ID NO: 29 - DNA, 2919 bp, Lactobacillus plantarum
  source 1..2919: mol_type = unassigned DNA
ttaagttaacaagggcgcatggtgaatgccttggcactaggagccgatgaaggacgggactaacaccgatatgcttcggggagctgtacgtaagctatgatccggagatttccgaatggggcaacccagcagttttaatcaactgttaccactagatgaattcatagtctagttggaggtaaacgctgtgaactgaaacatctcattagcagcaggaatataaagaaatttcgattccctaagtagcggcgagcgaacggggaacagcccaaaccaaagtgcttgcactttggggttgtaggactgaacatttgagttaccaaagaacttgatagtcgaaggatttgggaaaatccgccatagatggtgatagcccagtagattaaatcaaattctctcagttcaggatcctgagtacggcggaacacgtgaaattccgtcggaatccgggaggaccatctcccaaggctaaatactacctagtgaccgatagtgaaccagtaccgtgagggaaaggtgaaaagcaccccgggaggggagtgaaatagttcctgaaaccatgtgcctacaataagtcagagcgcgttaatgcgtgatggcgtgccttttgtagaatgaaccggcgagttatgatcccgtgcaaggttaagactaaaaagtcggagccgtagcgaaagcgagtctgaaatgggcgttttgagtacgaggttatagacccgaaaccaggtgacctatccatgtccaggttgaaggtgcggtaaaacgcactggaggaccgaacccgtgtaagttgaaaattgctgggatgaggtgtggatagcggtgaaattccaaacgaacttggagatagctggttctctccgaaatagctttagggctagcctcggaattaggatcatggaggtagagcactatttggactaggggcccgtcttgggttactgaattcagataaactccgaatgccattgattcatatccgggagtcagacgatgagtgataagatccaccgtcgaaaggggaacagcccagaccatcagttaaggtccctaaatgtatgctaagtggaaaaggatgtggagttgcatagacaactaggatgttggctcagaagcagccaccatttaaagagtgcgtaatagctcactagtcgagtgatcctgcgccgaaaatgtaccggggctaagcatactaccgaaaccatggatgcgaccattaggtcgcgtgataggagagcgttctaagggcggtgaagcaagatcgtgaggacttgtggagcgcttagaagtgagaatgccggtatgagtagcgaaagataggtgagaatcctatccaccgaatgactaaggtttcctggggaaggctcgtcctcccagggttagtcgggacctaagtcgaggccgagaggcgtagacgatggataacaggttgagattcctgtactagttaagtgcgtttgagcaatggagggacgcaggaggctaagatgtgcattctgttggattagaatgtccaagcagtaagtcttgtgaagagtcaaatgcttttcactttaaggacaagctgtgatggggagcgaaatttagtagcgaagcgtctgatgtcacactgccgagaaaagcttctagtgagtacttaactacccgtaccgcaaaccgacacaggtagtcgaggagagaatcctaaggtgagcgagtgaactctcgttaaggaactcggcaaaatgaccccgtaacttcgggagaaggggtgctgatcgcaagatcagccgcagtgaataggcccaggcgactgtttatcaaaaacacaggtctctgcaaaatcgtaagatgacgtataggggctgacgcctgcccggtgctggaaggttaaaaggatgggttagcttcggcgaagctcagaattgaagccccagtaaacggcggccgtaactataacggtcctaaggtagcgaaattccttgtcgggtaagttccgacccgcacgaaaggcgtaacgatctgggcactgtctcaacgagagactcggtgaaattatattgtccgtgaagatgcggactacccgcgacaggacggaaagaccccatggagctttactgtagcttgatattgagtgtttgtacagcttgtacaggataggtaggagccatagaaaccggaacgctagtttcggtggaggcgttggtgggatactaccctcgctgtatgaccactctaacccgcaccactaatcgtggtgggagacagtgtcaggtgggcagtttgactggggcggtcgcctcctaaaaagtaacggaggcgcccaaaggttccctcagaatggttggaaatcattcgcagagtgtaaaggcacaagggagcttgactgcgagacagacaggtcgagcagggacgaaagtcgggcttagtgatccggtggtaccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgtaggaaatttgagaggacctgtccttagtacgagaggaccgggatggacatacctctggtgtaccagttgtgccgccaggcgcatcgctgggtagctacgtatggatgtgataaacgctgaaagcatctaagtgtgaaacacacctcgagatgagatttcccattcctttatggaagtaagacccctgaaagatgatcaggtagataggttagaagtggcagtgcggtgacgcatgaagcggactaatactaatcggtcgaggacttaaccaa

SEQ ID NO: 30 - DNA, 2923 bp, Lactobacillus plantarum
  source 1..2923: mol_type = unassigned DNA
aggttaagttaacaagggcgcatggtgaatgccttggcactaggagccgatgaaggacgggactaacaccgatatgcttcggggagctgtacgtaagctatgatccggagatttccgaatggggcaacccagcagttttaatcaactgttaccactagatgaattcatagtctagttggaggtaaacgctgtgaactgaaacatctcattagcagcaggaatataaagaaatttcgattccctaagtagcggcgagcgaacggggaacagcccaaaccaaagtgcttgcactttggggttgtaggactgaacatttgagttaccaaagaacttgatagtcgaaggatttgggaaaatccgccatagatggtgatagcccagtagattaaatcaaattctctcagttcaggatcctgagtacggcggaacacgtgaaattccgtcggaatccgggaggaccatctcccaaggctaaatactacctagtgaccgatagtgaaccagtaccgtgagggaaaggtgaaaagcaccccgggaggggagtgaaatagttcctgaaaccatgtgcctacaataagtcagagcgcgttaatgcgtgatggcgtgccttttgtagaatgaaccggcgagttatgatcccgtgcaaggttaagactaaaaagtcggagccgtagcgaaagcgagtctgaaatgggcgttttgagtacgaggttatagacccgaaaccaggtgacctatccatgtccaggttgaaggtgcggtaaaacgcactggaggaccgaacccgtgtaagttgaaaattgctgggatgaggtgtggatagcggtgaaattccaaacgaacttggagatagctggttctctccgaaatagctttagggctagcctcggaattaggatcatggaggtagagcactatttggactaggggcccgtcttgggttactgaattcagataaactccgaatgccattgattcatatccgggagtcagacgatgagtgataagatccaccgtcgaaaggggaacagcccagaccatcagttaaggtccctaaatgtatgctaagtggaaaaggatgtggagttgcatagacaactaggatgttggctcagaagcagccaccatttaaagagtgcgtaatagctcactagtcgagtgatcctgcgccgaaaatgtaccggggctaagcatactaccgaaaccatggatgcgaccattaggtcgcgtgataggagagcgttctaagggcggtgaagcaagatcgtgaggacttgtggagcgcttagaagtgagaatgccggtatgagtagcgaaagataggtgagaatcctatccaccgaatgactaaggtttcctggggaaggctcgtcctcccagggttagtcgggacctaagtcgaggccgagaggcgtagacgatggataacaggttgagattcctgtactagttaagtgcgtttgagcaatggagggacgcaggaggctaagatgtgcattctgttggattagaatgtccaagcagtaagtcttgtgaagagtcaaatgcttttcactttaaggacaagctgtgatggggagcgaaatttagtagcgaagcgtctgatgtcacactgccgagaaaagcttctagtgagtacttaactacccgtaccgcaaaccgacacaggtagtcgaggagagaatcctaaggtgagcgagtgaactctcgttaaggaactcggcaaaatgaccccgtaacttcgggagaaggggtgctgatcgcaagatcagccgcagtgaataggcccaggcgactgtttatcaaaaacacaggtctctgcaaaatcgtaagatgacgtataggggctgacgcctgcccggtgctggaaggttaaaaggatgggttagcttcggcgaagctcagaattgaagccccagtaaacggcggccgtaactataacggtcctaaggtagcgaaattccttgtcgggtaagttccgacccgcacgaaaggcgtaacgatctgggcactgtctcaacgagagactcggtgaaattatattgtccgtgaagatgcggactacccgcgacaggacggaaagaccccatggagctttactgtagcttgatattgagtgtttgtacagcttgtacaggataggtaggagccatagaaaccggaacgctagtttcggtggaggcgttggtgggatactaccctcgctgtatgaccactctaacccgcaccactaatcgtggtgggagacagtgtcaggtgggcagtttgactggggcggtcgcctcctaaaaagtaacggaggcgcccaaaggttccctcagaatggttggaaatcattcgcagagtgtaaaggcacaagggagcttgactgcgagacagacaggtcgagcagggacgaaagtcgggcttagtgatccggtggtaccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgtaggaaatttgagaggacctgtccttagtacgagaggaccgggatggacatacctctggtgtaccagttgtgccgccaggcgcatcgctgggtagctacgtatggatgtgataaacgctgaaagcatctaagtgtgaaacacacctcgagatgagatttcccattcctttatggaagtaagacccctgaaagatgatcaggtagataggttagaagtggcagtgcggtgacgcatgaagcggactaatactaatcggtcgaggacttaaccaag

SEQ ID NO: 31 - DNA, 2923 bp, Lactobacillus plantarum; identical to SEQ ID NO: 30
  source 1..2923: mol_type = unassigned DNA

SEQ ID NO: 32 - DNA, 2921 bp, Lactobacillus plantarum
  source 1..2921: mol_type = unassigned DNA
ggttaagttaacaagggcgcatggtgaatgccttggcactaggagccgatgaaggacgggactaacaccgatatgcttcggggagctgtacgtaagctatgatccggagatttccgaatggggcaacccagcagttttaatcaactgttaccactagatgaattcatagtctagttggaggtaaacgctgtgaactgaaacatctcattagcagcaggaatataaagaaatttcgattccctaagtagcggcgagcgaacggggaacagcccaaaccaaagtgcttgcactttggggttgtaggactgaacatttgagttaccaaagaacttgatagtcgaaggatttgggaaaatccgccatagatggtgatagcccagtagattaaatcaaattctctcagttcaggatcctgagtacggcggaacacgtgaaattccgtcggaatccgggaggaccatctcccaaggctaaatactacctagtgaccgatagtgaaccagtaccgtgagggaaaggtgaaaagcaccccgggaggggagtgaaatagttcctgaaaccatgtgcctacaataagtcagagcgcgttaatgcgtgatggcgtgccttttgtagaatgaaccggcgagttatgatcccgtgcaaggttaagactaaaaagtcggagccgtagcgaaagcgagtctgaaatgggcgttttgagtacgaggttatagacccgaaaccaggtgacctatccatgtccaggttgaaggtgcggtaaaacgcactggaggaccgaacccgtgtaagttgaaaattgctgggatgaggtgtggatagcggtgaaattccaaacgaacttggagatagctggttctctccgaaatagctttagggctagcctcggaattaggatcatggaggtagagcactatttggactaggggcccgtcttgggttactgaattcagataaactccgaatgccattgattcatatccgggagtcagacgatgagtgataagatccaccgtcgaaaggggaacagcccagaccatcagttaaggtccctaaatgtatgctaagtggaaaaggatgtggagttgcatagacaactaggatgttggctcagaagcagccaccatttaaagagtgcgtaatagctcactagtcgagtgatcctgcgccgaaaatgtaccggggctaagcatactaccgaaaccatggatgcgaccattaggtcgcgtgataggagagcgttctaagggcggtgaagcaagatcgtgaggacttgtggagcgcttagaagtgagaatgccggtatgagtagcgaaagataggtgagaatcctatccaccgaatgactaaggtttcctggggaaggctcgtcctcccagggttagtcgggacctaagtcgaggccgagaggcgtagacgatggataacaggttgagattcctgtactagttaagtgcgtttgagcaatggagggacgcaggaggctaagatgtgcattctgttggattagaatgtccaagcagtaagtcttgtgaagagtcaaatgcttttcactttaaggacaagctgtgatggggagcgaaatttagtagcgaagcgtctgatgtcacactgccgagaaaagcttctagtgagtacttaactacccgtaccgcaaaccgacacaggtagtcgaggagagaatcctaaggtgagcgagtgaactctcgttaaggaactcggcaaaatgaccccgtaacttcgggagaaggggtgctgatcgcaagatcagccgcagtgaataggcccaggcgactgtttatcaaaaacacaggtctctgcaaaatcgtaagatgacgtataggggctgacgcctgcccggtgctggaaggttaaaaggatgggttagcttcggcgaagctcagaattgaagccccagtaaacggcggccgtaactataacggtcctaaggtagcgaaattccttgtcgggtaagttccgacccgcacgaaaggcgtaacgatctgggcactgtctcaacgagagactcggtgaaattatattgtccgtgaagatgcggactacccgcgacaggacggaaagaccccatggagctttactgtagcttgatattgagtgtttgtacagcttgtacaggataggtaggagccatagaaaccggaacgctagtttcggtggaggcgttggtgggatactaccctcgctgtatgaccactctaacccgcaccactaatcgtggtgggagacagtgtcaggtgggcagtttgactggggcggtcgcctcctaaaaagtaacggaggcgcccaaaggttccctcagaatggttggaaatcattcgcagagtgtaaaggcacaagggagcttgactgcgagacagacaggtcgagcagggacgaaagtcgggcttagtgatccggtggtaccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgtaggaaatttgagaggacctgtccttagtacgagaggaccgggatggacatacctctggtgtaccagttgtgccgccaggcgcatcgctgggtagctacgtatggatgtgataaacgctgaaagcatctaagtgtgaaacacacctcgagatgagatttcccattcctttatggaagtaagacccctgaaagatgatcaggtagataggttagaagtggcagtgcggtgacgcatgaagcggactaatactaatcggtcgaggacttaaccaa

SEQ ID NO: 33 - DNA, 1564 bp, Lactobacillus acidophilus
  source 1..1564: mol_type = unassigned DNA
aaaaacgagagtttgatcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggcagggcagatgactggggtgaagtcgtaacaaggtagccgtaggagaacctgcggctggatcacctccttt

SEQ ID NO: 34 - DNA, 3051 bp, Lactobacillus acidophilus
  source 1..3051: mol_type = unassigned DNA
gcaaaaaaccgagacaatcaaagagaacagattgtagagcgaccgagaagagaattcttgggtaaggtcaagtagaaaagggcgcacggtgaatgcctaggcactaacagccgatgaaggacgtgacgaactacgaaaagcttcggggagcggtaagtacgcagtgatccggagatgtccgaatgggggaacccaatgcagcgatgcattattggttgatgaatagatagtcaatcaaaggaagacgcagtgaactgaaacatctaagtagctgcaggaagagaaagaaaaatcgatttccttagtagcggcgagcgaagaggaaagagcccaaaccaagtgatttatcatttggggttgtaggactgcaaagtggtagcgtaagcgatagttgaattatctgggaaggtaagccagagagggtgagagccccgtaagcgaaattgcgagcgcgcctagcagaatcctgagtaggccgggacacgaggaatcccggttgaagccgcgaggaccatctcgcaaggctaaatactagttagtgaccgatagtgaaccagtaccgtgagggaaaggtgaaaagaaccccggaaggggagtgaaagagaacctgaaaccgtgtgtctacaagtagtcaaagcacattaaagtgcgatggcgtgccttttgtagaatgaaccggcgagttacgttatctagcgaggttaagtcagaaaagacggagccggagcgaaagcgagtctgaatagggcgaagagttaggtgacgtagacccgaaaccaagtgacctacccatggccaggctgaaggtgtggtaaaacgcactggagggccgaacccacgtaagttaaaaattgcggggatgagctgtgggtagcggtgaaattccaaacgaacttggagatagctggttctctccgaaatagctttagggctagcctcgtggagaggataatggaggtagagctctgtttggactaggggcccgtcaggggttactgaatccagataaactgcgaattccatatatccatacacgggagtcagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtggcgctgcgccgaaaatttaccggggctaagcatagtaccgaaactgtggatgcatcgaaagatgcgtggtaggagagcgttctaagtgcggcgaaggttaaccgagaggataattggagcgcttagaagtgagaatgccggtatgagtagcgaaagacaggtgagaatcctgtccgccgaaagactaaggtttcctggggcaggctcgtccgcccagggtaagtcgggacctaaggcaaggccgagaggcgtagtcgatggataacaggtagaaattcctgtactgtgtttaatcgttatgagcgatggagggacgcaggaggtgaaacacgcatcgagctggatcgatgttcaagcaacaagtgcggttaagagtcaaatgcttctaaccagcaacacgagttgtgatgagtagcgaagtaatagtagcgaaggtgatgtaatcacactgccaagaaaagcttctagccagagaggacagacccgtaccgcaaaccgacacaggtagtcgagtggagaacactaaggtgagcgagagaactctcgttaaggaactcggcaaaatgaccccgtaacttcggaagaaggggtgctggccacgagagtggttagccgcagtgaataggcccaaacaactgtttatcaaaaacacaggtctctgcaaaatcgtaagatgacgtatagaggctgacacctgcccggtgctggaaggttaagaggagagcttagcgaaagcggaggttcgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagcttcactgtagcttgatattgagtatcttttaaacatgtacaggataggtaggagccaaggaaggcaggacgctagttttgctggaggcaatgttgggatactacccttgtttgaaggatgctctaacctcgacctgtaagccaggtcagggacagtgtcaggtgggcagtttgactggggcggtcgcctcctaaagtgtaacggaggcgctcaaaggttccctcagaatggttggaaatcattcgcagagtgtaaaggtataagggagcttgactgcgagagaaacagctcgagcagggacgaaagtcggacttagtgatctggtggtaccgtatggaatggccatcactcaacggataaaagctaccctggggataacaggcttatctcccccaagagttcacatcgacggggaggtttggcacctcgatgtcggctcgtcgcatcctggggctgaagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcaatagcagtaagacacctcaaagacgatgaggtagataggctgggagtggaagttctgtgaagaatggagcggaccagtactaatcagtcgaggacttgaccaaaagcgaagcaaactgtaaggttttttgcgagagattatgtttagttttgagcgtagtagctcaaaagagtacggtggcga

SEQ ID NO: 35 - DNA, 2906 bp, Lactobacillus acidophilus
  source 1..2906: mol_type = unassigned DNA
ggtcaagtagaaaagggcgcacggtgaatgcctaggcactaacagccgatgaaggacgtgacgaactacgaaaagcttcggggagcggtaagtacgcagtgatccggagatgtccgaatgggggaacccaatgcagcgatgcattattggttgatgaatagatagtcaatcaaaggaagacgcagtgaactgaaacatctaagtagctgcaggaagagaaagaaaaatcgatttccttagtagcggcgagcgaagaggaaagagcccaaaccaagtgatttatcatttggggttgtaggactgcaaagtggtagcgtaagcgatagttgaattatctgggaaggtaagccagagagggtgagagccccgtaagcgaaattgcgagcgcgcctagcagaatcctgagtaggccgggacacgaggaatcccggttgaagccgcgaggaccatctcgcaaggctaaatactagttagtgaccgatagtgaaccagtaccgtgagggaaaggtgaaaagaaccccggaaggggagtgaaagagaacctgaaaccgtgtgtctacaagtagtcaaagcacattaaagtgcgatggcgtgccttttgtagaatgaaccggcgagttacgttatctagcgaggttaagtcagaaaagacggagccggagcgaaagcgagtctgaatagggcgaagagttaggtgacgtagacccgaaaccaagtgacctacccatggccaggctgaaggtgtggtaaaacgcactggagggccgaacccacgtaagttaaaaattgcggggatgagctgtgggtagcggtgaaattccaaacgaacttggagatagctggttctctccgaaatagctttagggctagcctcgtggagaggataatggaggtagagctctgtttggactaggggcccgtcaggggttactgaatccagataaactgcgaattccatatatccatacacgggagtcagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtggcgctgcgccgaaaatttaccggggctaagcatagtaccgaaactgtggatgcatcgaaagatgcgtggtaggagagcgttctaagtgcggcgaaggttaaccgagaggataattggagcgcttagaagtgagaatgccggtatgagtagcgaaagacaggtgagaatcctgtccgccgaaagactaaggtttcctggggcaggctcgtccgcccagggtaagtcgggacctaaggcaaggccgagaggcgtagtcgatggataacaggtagaaattcctgtactgtgtttaatcgttatgagcgatggagggacgcaggaggtgaaacacgcatcgagctggatcgatgttcaagcaacaagtgcggttaagagtcaaatgcttctaaccagcaacacgagttgtgatgagtagcgaagtaatagtagcgaaggtgatgtaatcacactgccaagaaaagcttctagccagagaggacacacccgtaccgcaaaccgacacaggtagtcgagtggagaacactaaggtgagcgagagaactctcgttaaggaactcggcaaaatgaccccgtaacttcggaagaaggggtgctggccacgagagtggttagccgcagtgaataggcccaaacaactgtttatcaaaaacacaggtctctgcaaaatcgtaagatgacgtatagaggctgacacctgcccggtgctggaaggttaagaggagagcttagcgaaagcgaaggttcgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagcttcactgtagcttgatattgagtatcttttaaacatgtacaggataggtaggagccaaggaaggcaggacgctagttttgctggaggcaatgttgggatactacccttgtttgaaggatgctctaacctcgacctgtaagccaggtcagggacagtgtcaggtgggcagtttgactggggcggtcgcctcctaaagtgtaacggaggcgctcaaaggttccctcagaatggttggaaatcattcgcagagtgtaaaggtataagggagcttgactgcgagagaaacagctcgagcagggacgaaagtcggacttagtgatctggtggtaccgtatggaagggccatcactcaacggataaaagctaccctggggataacaggcttatctcccccaagagttcacatcgacggggaggtttggcacctcgatgtcggctcgtcgcatcctggggctgaagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcaatagcagtaagacacctcaaagacgatgaggtagataggctgggagtggaagttctgtgaagaatggagcggaccagtactaatcagtcgaggacttgaccaa

SEQ ID NO: 36 - DNA, 1873 bp, Lactobacillus acidophilus
  source 1..1873: mol_type = unassigned DNA
cagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtggcgctgcgccgaaaatttaccggggctaagcatagtaccgaaactgtggatgcatcgaaagatgcgtggtaggagagcgttctaagtgcggcgaaggttaaccgagaggataattggagcgcttagaagtgagaatgccggtatgagtagcgaaagacaggtgagaatcctgtccgccgaaagactaaggtttcctggggcaggctcgtccgcccagggtaagtcgggacctaaggcaaggccgagaggcgtagtcgatggataacaggtagaaattcctgtactgtgtttaatcgttatgagcgatggagggacgcaggaggtgaaacacgcatcgagctggatcgatgttcaagcaacaagtgcggttaagagtcaaatgcttctaaccagcaacacgagttgtgatgagtagcgaagtaatagtagcgaaggtgatgtaatcacactgccaagaaaagcttctagccagagaggacacacccgtaccgcaaaccgacacaggtagtcgagtggagaacactaaggtgagcgagagaactctcgttaaggaactcggcaaaatgaccccgtaacttcggaagaaggggtgctggccacgagagtggttagccgcagtgaataggcccaaacaactgtttatcaaaaacacaggtctctgcaaaatcgtaagatgacgtatagaggctgacacctgcccggtgctggaaggttaagaggagagcttagcgaaagcgaaggttcgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagcttcactgtagcttgatattgagtatcttttaaacatgtacaggataggtaggagccaaggaaggcaggacgctagttttgctggaggcaatgttgggatactacccttgtttgaaggatgctctaacctcgacctgtaagccaggtcagggacagtgtcaggtgggcagtttgactggggcggtcgcctcctaaagtgtaacggaggcgctcaaaggttccctcagaatggttggaaatcattcgcagagtgtaaaggtataagggagcttgactgcgagagaaacagctcgagcagggacgaaagtcggacttagtgatctggtggtaccgtatggaagggccatcactcaacggataaaagctaccctggggataacaggcttatctcccccaagagttcacatcgacggggaggtttggcacctcgatgtcggctcgtcgcatcctggggctgaagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcaatagcagtaagacacctcaaagacgatgaggtagataggctgggag

SEQ ID NO: 37 - DNA, 2904 bp, Lactobacillus acidophilus
  source 1..2904: mol_type = unassigned DNA
tcaagtagaaaagggcgcacggtgaatgcctaggcactaacagccgatgaaggacgtgacgaactacgaaaagcttcggggagcggtaagtacgcagtgatccggagatgtccgaatgggggaacccaatgcagcgatgcattattggttgatgaatagatagtcaatcaaaggaagacgcagtgaactgaaacatctaagtagctgcaggaagagaaagaaaaatcgatttccttagtagcggcgagcgaagaggaaagagcccaaaccaagtgatttatcatttggggttgtaggactgcaaagtggtagcgtaagcgatagttgaattatctgggaaggtaagccagagagggtgagagccccgtaagcgaaattgcgagcgcgcctagcagaatcctgagtaggccgggacacgaggaatcccggttgaagccgcgaggaccatctcgcaaggctaaatactagttagtgaccgatagtgaaccagtaccgtgagggaaaggtgaaaagaaccccggaaggggagtgaaagagaacctgaaaccgtgtgtctacaagtagtcaaagcacattaaagtgcgatggcgtgccttttgtagaatgaaccggcgagttacgttatctagcgaggttaagtcagaaaagacggagccggagcgaaagcgagtctgaatagggcgaagagttaggtgacgtagacccgaaaccaagtgacctacccatggccaggctgaaggtgtggtaaaacgcactggagggccgaacccacgtaagttaaaaattgcggggatgagctgtgggtagcggtgaaattccaaacgaacttggagatagctggttctctccgaaatagctttagggctagcctcgtggagaggataatggaggtagagctctgtttggactaggggcccgtcaggggttactgaatccagataaactgcgaattccatatatccatacacgggagtcagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtggcgctgcgccgaaaatttaccggggctaagcatagtaccgaaactgtggatgcatcgaaagatgcgtggtaggagagcgttctaagtgcggcgaaggttaaccgagaggataattggagcgcttagaagtgagaatgccggtatgagtagcgaaagacaggtgagaatcctgtccgccgaaagactaaggtttcctggggcaggctcgtccgcccagggtaagtcgggacctaaggcaaggccgagaggcgtagtcgatggataacaggtagaaattcctgtactgtgtttaatcgttatgagcgatggagggacgcaggaggtgaaacacgcatcgagctggatcgatgttcaagcaacaagtgcggttaagagtcaaatgcttctaaccagcaacacgagttgtgatgagtagcgaagtaatagtagcgaaggtgatgtaatcacactgccaagaaaagcttctagccagagaggacacacccgtaccgcaaaccgacacaggtagtcgagtggagaacactaaggtgagcgagagaactctcgttaaggaactcggcaaaatgaccccgtaacttcggaagaaggggtgctggccacgagagtggttagccgcagtgaataggcccaaacaactgtttatcaaaaacacaggtctctgcaaaatcgtaagatgacgtatagaggctgacacctgcccggtgctggaaggttaagaggagagcttagcgaaagcgaaggttcgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagcttcactgtagcttgatattgagtatcttttaaacatgtacaggataggtaggagccaaggaaggcaggacgctagttttgctggaggcaatgttgggatactacccttgtttgaaggatgctctaacctcgacctgtaagccaggtcagggacagtgtcaggtgggcagtttgactggggcggtcgcctcctaaagtgtaacggaggcgctcaaaggttccctcagaatggttggaaatcattcgcagagtgtaaaggtataagggagcttgactgcgagagaaacagctcgagcagggacgaaagtcggacttagtgatctggtggtaccgtatggaagggccatcactcaacggataaaagctaccctggggataacaggcttatctcccccaagagttcacatcgacggggaggtttggcacctcgatgtcggctcgtcgcatcctggggctgaagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcaatagcagtaagacacctcaaagacgatgaggtagataggctgggagtggaagttctgtgaagaatggagcggaccagtactaatcagtcgaggacttgaccaa

SEQ ID NO: 38 - DNA, 671 bp, Lactobacillus acidophilus
  source 1..671: mol_type = unassigned DNA
cccgtcacaccatgggagtctgcaatgcccaaagccggcgttctaaccttcgggaaggagccgtctaaggcagggcagatgactggggtgaagtcgtaacaaggtagccgtaggagaacctgcggctggatcacctcctttctaaggaagcgaaggatatggagagtagaaatactaagagaagtatccagagcaagcggaagcacactaagaaactttgtttagttttgagggtagtacctcaaaagagttagtacattgaaaactgaatataatccaagcaaaaaaccgagacaatcaaagagaacagattgtagagcgaccgagaagagaattcttgggtaaggtcaagtagaaaagggcgcacggtgaatgcctaggcactaacagccgatgaaggacgtgacgaactacgaaaagcttcggggagcggtaagtacgcagtgatccggagatgtccgaatgggggaacccaatgcagcgatgcattattggttgatgaatagatagtcaatcaaaggaagacgcagtgaactgaaacatctaagtagctgcaggaagagaaagaaaaatcgatttccttagtagcggcgagcgaagaggaaagagcccaaaccaagtgatttatcatttggggttgtaggactgcaaagtggtagcgtaagcgatag

SEQ ID NO: 39 - DNA, 1330 bp, Lactobacillus acidophilus
  source 1..1330: mol_type = unassigned DNA
gggttgtgaggttaagcgactaagcgtacacggtggatgccctggcagtcagaggcgatgaagggcgtgctaatctgcgataagcgccggtaaggtgatatgaaccgttataaccggcgatacccgaatggggaaacccagtgtgactcgtcacactatcattaactgaatccataggttaatgaggcgaaccgggggaactgaaacatctaagtaccccgaggaaaagaaatcaaccgagattcccccagtagcggcgagcgaacggggaggagcccagagcctgaatcagcatgtgtgttagtggaagcgtctggaaaggcgcgcgatacagggtgacagccccgtacacaaaagcgcatgtgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcaccctttgggtgtgactgcgtaccttttgtataatgggtcagcgacttatattctgtagcaaggttaaccgaataggggagccggagggaaaccgagtcttaaccgggcgttaagttgcagggtatagacccgaaacccggtgatctagccatgggcaggttgaaggttgggtaacactaactggaggaccgaaccgactaatgttgaaaaattagcggatgacctgtggctgggggtgaaaggccaatcaaaccgggagatagctggttctccccgaaagctatttaggtagcgcctcgtgaattcatctccgggggtagagcactgtttcggctagggggccatcccggcttaccaacccgatgcaaactgcgaataccggagaatgttatcacgggagacacacgkcgggtgctaacgtccggygtsaagagggaaacaacccagaccgccagctaaggtccctaaatattgctaagtgggaaacgaagtgggaaggctaaaacagtcaggaggttggcttagaagcagccaccctttaaagaaagcgtaatagctcactgatcgagtcgtcctgcgcggaagatgtaacggggctaagcaatataccgaagctgcggatgcacgtaagtgcatggtaggagagcgttctgtaagcctgtgaaggtgtcttgtaaaggatgctggaggtatcagaagtgcgaatgctgacatgagtagcgataaagggggtgaaaggccccctcgccgtaagcccaaggtttcctacgcaacgttcat

SEQ ID NO: 40 - DNA, 1521 bp, Lactobacillus rhamnosus
  source 1..1521: mol_type = unassigned DNA
grtsaacgctsgcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactctacgggaggcagcagtagggaatcttccacaatggacgcaagtctgatggagcaacgccgcgtgagtnaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtncagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggncaaatgattagggtgaagtcgtaacaaggtagccgtaggagaacc

SEQ ID NO: 41 - DNA, 2800 bp, Lactobacillus rhamnosus
  source 1..2800: mol_type = unassigned DNA
agggcgcacggtggatgccttggcactaggagccgatgaaggacggaactaataccgatatgcttcggggagctataagtaagctttgatccggagatttccgaatgggggaacccagtacacatcagtgtattgcctgcaagtgaatacatagcttgttggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagttcgacgtagtcgaagtcagctggaaagctgcgccatagaaggtgaaagccctgtaaacgaaacggcggactctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaacgggtaatggcgtgccttttgtagaatgaaccggcgagttacgtttgcctgcgaggttaagatgaaaagtcggagccggagcgaaagcgagtctgaacagggcgcttcagtaggtagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccgtcaagggttactgaattcagataaactccgaataccattgatcttactccgggagtcagacagtgagcgataaggtccattgtcgaaaggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtcagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgcaaggcgtagtcgatgacaagcaggttgagattcctgcactagtttattttgtttaagcgatggagggacgcaggaggctaaggaaagcgcacggctggaaaagtgcgtccaagcagtaagtccggtagcgagtgaaatgcttgccgccttaaggacaagctgtgatggggagcgaaattaaagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgttggtaccgcttgtacaggataggtaggagccgtagagatcggaacgctagtttcgatggaggcattggtgggatactaccctagctgtatgaacactctaacccgcgccactgatcgtggcgggagacagtgtcaggtaggcagtttgactggggcggtcgcctcctaaaatgtaacggaggcgcccaaaggttccctcagaatggttggaaatcattcgcagagtgtaaaggtagaagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggaagggataaacgctgaaagcatctaagtgtgaagcccccctcgagatgagatttcccattcct

SEQ ID NO: 42 - DNA, 2920 bp, Lactobacillus rhamnosus
  source 1..2920: mol_type = unassigned DNA
aggttaagttacaaagggcgcacggtggatgccttggcactaggagccgatgaaggacggaactaataccgatatgcttcggggagctataagtaagctttgatccggagatttccgaatgggggaacccagtacacatcagtgtattgcctgcaagtgaatacatagcttgttggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagttcgacgtagtcgaagtcagctggaaagctgcgccatagaaggtgaaagccctgtaaacgaaacggcggactctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaacgggtaatggcgtgccttttgtagaatgaaccggcgagttacgtttgcctgcgaggttaagatgaaaagtcggagccggagcgaaagcgagtctgaacagggcgcttcagtaggtagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccgtcaagggttactgaattcagataaactccgaataccattgatcttactccgggagtcagacagtgagcgataaggtccattgtcgaaaggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtcagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgcaaggcgtagtcgatgacaagcaggttgagattcctgcactagtttattttgtttaagcgatggagggacgcaggaggctaaggaaagcgcacggctggaaaagtgcgtccaagcagtaagtccggtagcgagtgaaatgcttgccgccttaaggacaagctgtgatggggagcgaaattaaagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgttggtaccgcttgtacaggataggtaggagccgtagagatcggaacgctagtttcgatggaggcattggtgggatactaccctagctgtatgaacactctaacccgcgccactaatcgtggcgggagacagtgtcaggtaggcagtttgactggggcggtcgcctcctaaaatgtaacggaggcgcccaaaggttccctcagaatggttggaaatcattcgcagagtgtaaaggtagaagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggaagggataaacgctgaaagcatctaagtgtgaagcccccctcgagatgagatttcccattcctatatggaagtaagacccctgaaagatgatcaggtagataggctggaagtggaagtgcagcgatgcatggagcggaccagtactaatcggtcgaggacttaaccaag

SEQ ID NO: 43 - DNA, 2920 bp, Lactobacillus rhamnosus
  source 1..2920: mol_type = unassigned DNA
aggttaagttacaaagggcgcacggtggatgccttggcactaggagccgatgaaggacggaactaataccgatatgcttcggggagctataagtaagctttgatccggagatttccgaatgggggaacccagtacacatcagtgtattgcctgcaagtgaatacatagcttgttggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagttcgacgtagtcgaagtcagctggaaagctgcgccatagaaggtgaaagccctgtaaacgaaacggcggactctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaacgggtaatggcgtgccttttgtagaatgaaccggcgagttacgtttgcctgcgaggttaagatgaaaagtcggagccggagcgaaagcgagtctgaacagggcgcttcagtaggtagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccgtcaagggttactgaattcagataaactccgaataccattgatcttactccgggagtcagacagtgagcgataaggtccattgtcgaagggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtcagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgcaaggcgtagtcgatgacaagcaggttgagattcctgcactagtttattttgtttaagcgatggagggacgcaggaggctaaggaaagcgcacggctggaaaagtgcgtccaagcagtaagtccggtagcgagtgaaatgcttgccgccttaaggacaagctgtgatggggagcgaaattaaagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgttggtaccgcttgtacaggataggtaggagccgtagagatcggaacgctagtttcgatggaggcattggtgggatactaccctagctgtatgaacactctaacccgcgccactgatcgtggcgggagacagtgtcaggtaggcagtttgactggggcggtcgcctcctaaaatgtaacggaggcgcccaaaggttccctcagaatggttggaaatcattcgcagagtgtaaaggtagaagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggaagggataaacgctgaaagcatctaagtgtgaagcccccctcgagatgagatttcccattcctatatggaagtaagacccctgaaagatgatcaggtagataggctggaagtggaagtgcagcgatgcatggagcggaccagtactaatcggtcgaggacttaaccaag

SEQ ID NO: 44 - DNA, 1530 bp, Lactobacillus rhamnosus
  source 1..1530: mol_type = unassigned DNA
gaagtgagaatgccggcatgagtagcgaaagatcagtgagaatctgatccaccgtatgactaaggtttcctggggaaggctcgtcctcccagggttagtcgggatctaaggcgaggccgcaaggcgtagtcgatgacaagcaggttgagattcctgcactagtttattttgtttaagcgatggagggacgcaggaggctaaggaaagcgcacggctggaaaagtgcgtccaagcagtaagtccggtagcgagtgaaatgcttgccgccttaaggacaagctgtgatggggagcgaaattaaagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgttggtaccgcttgtacaggataggtaggagccgtagagatcggaacgctagtttcgatggaggcattggtgggatactaccctagctgtatgaacactctaacccgcgccactaatcgtggcgggagacagtgtcaggtaggcagtttgactggggcggtcgcctcctaaaatgtaacggaggcgcccaaaggttccctcagaatggttggaaatcattcgcagagtgtaaaggtagaagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggaagggataaacgctgaaagcatctaagtgtgaagcccccctcgagatgagatttcccattcct

SEQ ID NO: 45 - DNA, 1522 bp, Lactobacillus paracasei
  source 1..1522: mol_type = unassigned DNA
gatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacc

SEQ ID NO: 46 - DNA, 2916 bp, Lactobacillus paracasei
  source 1..2916: mol_type = unassigned DNA
ttaagttacaaagggcgcacggtggatgccttggcactaggagccgatgaaggacggaactaataccgatatgcttcggggagctataagtaagctttgatccggagatttccgaatgggggaacccagtacacatcagtgtattgcttgtcagtgaatacatagctggccggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagtccgacgtagtcgaagtcagctggaaagctgcgccacagaaggtgaaagccctgtaagcgaaacgtcgaaccctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaatgggtaatggcgtgccttttgtagaatgaaccggcgagttacgtttgcttgcgaggttaagatgaaaagtcggagccgtagcgaaagcgagtctgaacagggcgaatgagtaagcagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccatcaagggttactgaattcagataaactccgaataccatcgatcttactccgggagtcagacagtgagcgataaggtccattgtcgaaaggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtaagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgagaggcgtagtcgatgacaagcaggttgagattcctgcactagtttattttgtttaagcgatggagggacgcaggaggctaaggaaagcgcacggctggaaatgtgcgcccaagcagcaagtctgacagcgagtgaaatgcttgcagtcttaaggacaagctgtgatggggagcgaaattatagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgtttgtaccgcttgtacaggataggtaggagccgtagagatcggaacgctagtttcgatggaggcgttggtgggatactaccctagctgtatgaacactctaacccgcgccactaatcgtggcgggagacagtgtcaggtaggcagtttgactggggcggtcgcctcctaaaatgtaacggaggcgcccaaaggttccctcagaatggttggaaatcattcgcagagtgtaaaggtataagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggcagggataaacgctgaaagcatctaagtgtgaagcccccctcgagatgagatttcccattcctatatggaagtaagacccctgagagatgatcaggtagataggctggaagtggaagtgcagcgatgcatggagcggaccagtactaatcggtcgaggacttaaccaa

SEQ ID NO: 47 - DNA, 2919 bp, Lactobacillus paracasei
  source 1..2919: mol_type = unassigned DNA
aggttaagttacaaagggcgcacggtggatgccttggcactaggagccgatgaaggacggaactaataccgatatgcttcggggagctataagtaagctttgatccggagatttccgaatgggggaacccagtacacatcagtgtattgcttgtcagtgaatacatagctggccggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagtccgacgtagtcgaagtcagctggaaagctgcgccacagaaggtgaaagccctgtaagcgaaacgtcgaaccctccgttcaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaatgggtaatggcgtgccttttgtagaatgaaccggcgagttacgtttgcttgcgaggttaagatgaaaagtcggagccgtagcgaaagcgagtctgaacagggcgaatgagtaagcagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccatcaagggttactgaattcagataaactccgaataccatcgatcttactccgggagtcagacagtgagcgataaggtccattgtcgaaaggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtaagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgagaggcgtagtcgatgacaagcaggttgagattcctgcactagtttattttgtttaagcgatggagggacgcaggaggctaaggaaagcgcacggctggaaatgtgcgcccaagcagcaagtctgacagcgagtgaaatgcttgcagtcttaaggacaagctgtgatggggagcgaaattatagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgtttgtaccgcttgtacaggataggtaggagccgtagagatcggaacgctagtttcgatggaggcgttggtgggatactaccctagctgtatgaacactctaacccgcgccactaatcgtggcgggagacagtgtcaggtaggcagtttgactggggcggtcgcctcctaaaatgtaacggaggcgcccaaaggttccctcagaatggttggaaatcattcgcagagtgtaaaggtataagggagcttgactgcgagactgacaagtcgagcagggacgaaagtcgggcttagtgatccggtggttccgtatggaagggccatcgctcaacggataaaagctaccctggggataacaggcttatctccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggcagggataaacgctgaaagcatctaagtgtgaagcccccctcgagatgagatttcccattcctatatggaagtaagacccctgagagatgatcaggtagataggctggaagtggaagtgcagcgatgcatggagcggaccagtactaatcggtcgaggacttaaccaag

SEQ ID NO: 48 - DNA, 2920 bp, Lactobacillus paracasei
  source 1..2920: mol_type = unassigned DNA
aggttaagttacaaagggcgcacggtggatgccttggcactaggagccgatgaaggacggaactaataccgatatgcttcggggagctataagtaagctttgatccggagatttccgaatgggggaacccagtacacatcagtgtattgcttgtcagtgaatacatagctggccggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagtccgacgtagtcgaagtcagctggaaagctgcgccacagaaggtgaaagccctgtaagcgaaacgtcgaaccctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaatgggtaatggcgtgccttttgtagaatgaaccggcgagttacgtttgcttgcgaggttaagatgaaaagtcggagccgtagcgaaagcgagtctgaacagggcgaatgagtaagcagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccatcaagggttactgaattcagataaactccgaataccatcgatcttactccgggagtcagacagtgagcgataaggtccattgtcgaaaggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtaagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgagaggcgtagtcgatgacaagcaggttgagattcctgcactagtttattttgtttaagcgatggagggacgcaggaggctaaggaaagcgcacggctggaaatgtgcgcccaagcagcaagtctgacagcgagtgaaatgcttgcagtcttaaggacaagctgtgatggggagcgaaattatagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgtttgtaccgcttgtacaggataggtaggagccgtagagatcggaacgctagtttcgatggaggcgttggtgggatactaccctagctgtatgaacactctaacccgcgccactaatcgtggcgggagacagtgtcaggtaggcagtttgactggggcggtcgcctcctaaaatgtaacggaggcgcccaaaggttccctcagaatggttggaaatcattcgcagagtgtaaaggtataagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggcagggataaacgctgaaagcatctaagtgtgaagcccccctcgagatgagatttcccattcctatatggaagtaagacccctgagagatgatcaggtagataggctggaagtggaagtgcagcgatgcatggagcggaccagtactaatcggtcgaggacttaaccaag

SEQ ID NO: 49 - DNA, 1517 bp, Lactobacillus casei
  source 1..1517: mol_type = unassigned DNA
tcctggctcaggatgaacgctggcggcgtgcctaatacatgcaagtcgaacgagttttggtcgatgaacggtgcttgcactgagattcgacttaaaacgagtggcggacgggtgagtaacacgtgggtaacctgcccttaagtgggggataacatttggaaacagatgctaataccgcataaatccaagaaccgcatggttcttggctgaaagatggcgtcaagctatcgcttttggatggacccgcggcgtattagctagttggtgaggtaacggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcattgagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaag

SEQ ID NO: 50 - DNA, 483 bp, Lactobacillus casei
  source 1..483: mol_type = unassigned DNA
ggttaagttacaaagggcgcacggtggatgccttggcactaggagccgatgaaggacggaactaataccgatatgcttcggggagctataagtaagctttgatccggagatttccgaatgggggaacccagtacacatcagtgtattgcctgcaagtgaatacatagcttgttggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagttcgacgtagtcgaagtcagctggaaagctgcgccatagaaggtgaaagccctgtaaacgaaacggcggactctccgtccaggatcctgagtacggcggaacacgtgaaattccgtcggaatccgggaggaccatctcccaaggctaaatactccctag

SEQ ID NO: 51 - DNA, 556 bp, Lactobacillus casei
  source 1..556: mol_type = unassigned DNA
taggagcgatgaaggacggaactaataccgatatgcttcggggagctataagtaagctttgatccggagatttccgaatgggggaacccagtacacatcagtgtattgcttgtcagtgaatacatagctggccggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagtccgacgtagtcgaagtcagctggaaagctgcgccacagaaggtgaaagccctgtaagcgaaacgtcgaaccctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaatggtaat

SEQ ID NO: 52 - DNA, 2916 bp, Lactobacillus casei
  source 1..2916: mol_type = unassigned DNA
ttaagttacaaagggcgcacggtggatgccttggcactaggagccgatgaaggacggaactaataccgatatgcttcggggagctataagtaagctttgatccggagatttccgaatgggggaacccagtacacatcagtgtattgcttgtcagtgaatacatagctggccggcggcagacgcggggaactgaaacatctcagtacccgcaggaagagaaagaaaactcgattcccatagtagcggcgagcgaagtgggaagagcccaaaccgagaagcttgcttctcggggttgtaggactggacattggagttaccaaagtccgacgtagtcgaagtcagctggaaagctgcgccacagaaggtgaaagccctgtaagcgaaacgtcgaaccctccgtccaggatcctgagtacggcggaacacgtgaaattccgtcggaatccgggaggaccatctcccaaggctaaatactccctagtgaccgatagtgaaccagtaccgtgagggaaaggtgaaaagcaccccggaaggggagtgaaacagttcctgaaaccgtgtgcctacaattagtcaaagcccgttaatgggtaatggcgtgccttttgtagaatgaaccggcgagttacgtttgcttgcgaggttaagatgaaaagtcggagccgtagcgaaagcgagtctgaacagggcgaatgagtaagcagatgtagacccgaaaccaagtgacctacccatgaccaggttgaaggtgtggtgaaacacactggaggaccgaacccatgtatgttgaaaaatgctgggatgagttgtgggtagcggtgaaattccaaacgaacttggagatagctggttctctccgaaatagctttagggttagcctcggaggatggatcatggaggtagagcactgtttgaactaggggcccatcaagggttactgaattcagataaactccgaataccatcgatcttactccgggagtcagacagtgagcgataaggtccattgtcgaaaggggaacagcccagatcaccagttaaggtccctaaatttatgctaagtggaaaaggatgtggcgttgcacagacaactaggatgttggctcagaagcagccaccatttaaagagtgcgtaatagctcactagtcgagtggcactgcgccgaaaatataccggggctaagcataataccgaaactgtgggtgcacccgtaagggtgcgcggtaggagagcgttctaagggcgttgaaggtcgatcgtgaggacggctggagcgcttagaagtgagaatgccggcatgagtagcgaaagatcagtgagaatctgatccaccgtatgactaaggtttcctggggaaggctcgtcctcccagggttagtcgggatctaaggcgaggccgagaggcgtagtcgatgacaagcaggttgagattcctgcactagtttattttgtttaagcgatggagggacgcaggaggctaaggaaagcgcacggctggaaatgtgcgcccaagcagcaagtctgacagcgagtgaaatgcttgcagtcttaaggacaagctgtgatggggaacgaaattatagtagcgaagttcctgatgtcacactgccaagaaaagcttctagtgagaaataaactacccgtaccgcaaaccgacacaggtagtcgaggagagtatcctcaggtgagcgagcgaactctcgttaaggaactcggcaaaatgaccccgtaacttcggaagaaggggtgctgaccgcaaggtcagccgcagtgaataggcccaaacaactgtttatcaaaaacacaggtctctgctaaatcgtaagatgatgtataggggctgacgcctgcccggtgctggaaggttaagaggatgagttagcgcaagcgaagcccagaattgaagccccagtaaacggcggccgtaactataacggtcctaaggtagcgaaattccttgtcgggtaagttccgacccgcacgaaaggcgtaatgatttgggcactgtctcaacgagagactcggtgaaattatagtacccgtgaagatgcgggttacccgcgacaggacggaaagaccccatggagctttactgtagcttgatattgagtgtttgtaccgcttgtacaggataggtaggagccgtagagatcggaacgctagtttcgatggaggcgttggtgggatactaccctagctgtatgaacactctaacccgcgccactaatcgtggcgggagacagtgtcaggtaggcagtttgactggggcggtcgcctcctaaaatgtaacggaggcgcccaaaggttccctcagaatggttggaaatcattcgcagagtgtaaaggtataagggagcttgactgcgagactgacaagtcgagcagggacgaaagtcgggcttagtgatccggtggttccgtatggaagggccatcgctcaacggataaaagctaccctggggataacaggcttatctcccccaagagtccacatcgacggggaggtttggcacctcgatgtcggctcatcgcatcctggggctgtagtcggtcccaagggttgggctgttcgcccattaaagcggtacgcgagctgggttcagaacgtcgtgagacagttcggtccctatccgtcgcgggcgcaggaaatttgagaggagctgtccttagtacgagaggaccgggatggacgttccgctggtgtaccagttgtgccgccaggcgcatcgctgggtagctatgaacggcagggataaacgctgaaagcatctaagtgtgaagcccccctcgagatgagatttcccattcctatatggaagtaagacccctgagagatgatcaggtagataggctggaagtggaagtgcagcgatgcatggagcggaccagtactaatcggtcgaggacttaaccaa

SEQ ID NO: 53 - DNA, 1836 bp, Astragalus complanatus
  source 1..1836: mol_type = unassigned DNA
atcatactcaaaagaagagtttgatcctggctcagaaggaacgctagctatatgcttaacacatgcaagtcgaacgttgttttcggggagctgggcagaaggaaaagaggctcctagcgtgaaggtagcttgtctcgcccaggaggtgggaacagttgaaaacaaagtggcgaacgggtgcgtaatgcgtgggaatctgccgaacagttcgggccaaatcctgaagaaagctaaaaagcgctgtttgatgagcctgcgtagtattaggtagttggtcaggtaaaggctgaccaagccaatgatgcttagctggtcttttcggatgatcagccacactgggactgagacacggcccggactcccacggggggcagcagtggggaatcttggacaatgggcgaaagcccgatccagcaatatcgcgtgagtgaagaagggcaacgccgcttgtaaagctctttcgtcgagtgcgcgatcatgacaggactcgaggaagaagccccggctaactccgtgccagcagccgcggtaagacggggggggcaagtgttcttcggaatgactgggcgtaaagggcacgtaggcggtgaatcgggttgaaagtgaaagtcgccaaaaactggtggaatgctctcgaaaccaattcacttgagtgagacagaggagagtggaatttcgtgtgtaggggtgaaatccgcagatatacgaaggaacgccaaaagcgaaggcagctctctgggtccctaccgacgctggagtgcgaaagcatggggagcgaacgggattagataccctggtagtccatgccgtaaacgatgagtgttcgcccttggtctacgtggatcaggggcccagctaacgcgtgaaacactccgcctggggagtacggtcgcaagaccgaaactcaaaggaattgacgggggcctgcacaagcggtggagcatgtggtttaattcgatacaacgcgcaaaaccttaccagcccttgacatatgaacaagaaaacctgtccttaacgggatggtactgactttcatacaggtgctgcatggctgtcgtcagctcgtgtcgtgagatgtttggtcaagtcctataacgagcgaaaccctcgttttgtgttgctgagacatgcgcctaaggagaaagtctttgcaaccgaagtgagccgaggagccgagtgacgcgccagcgctactaattgagtgccagcacgtagctgtgctgtcagtaagaagggagccggcgcctttcgaattcgaagcactctctagtgtgcgctgttttttgattgcagctagagagcaagactcggcattcaggcgagccgcccggtggtgtggtccgtagtgggtttagtacgccccgccaaaacggctccgaaacaaactaaaaggtgcatgccgcactcacgagggactgccagtgatatactggaggaaggtggggatgacgtcaagtccgcatggcccttatgggctgggccacacacgtgctacaatggcaattacaatgggaagcaaggctgtaaggcggagcgaatccggaaagattgcctcagttcggattgttctctgcaactcgggaacatgaagttggaatcgctagtaatcgcggatcagcatgccgcggtgaatatgtacccgggccctgtacacaccgcccgtcacaccctgggaattggtttcgcccgaagcatcggaccaatgatcacccatgacttctgtgtaccactagtgccacaaaggcttttggtggtcttattggcgcataccacggtggggtcttcgactggggtgaagtcgtaacaaggtagccgtaggggaacctgtggctggat

SEQ ID NO: 54 - DNA, 716 bp, Astragalus membranaceus
  source 1..716: mol_type = unassigned DNA
ggaagtaaaagtcgtaacaaggtttccgtaggtgaacctgcggaaggatcattgtcgatgccttacatgcagaccaactagtgaatctgtttgaatacttagggatggctggggtgttttgcaccacgacctccctttgggtggggggtggtgcgcaatgcgttccccctcctgcccgaacacaaaccccggcgctcaatgcgccaaggaactaaaattcgatcaatgtgccccgtcggcccggagacggtgcttcggcggtggtgccttgtcacatgatacagaatgactctcggcaacggatatctaggctcttgcatcgatgaagaacgtagcgaaatgcgatacttggtgtgaattgcagaatcccgtgaaccatcgagtctttgaacgcaagttgcgcccgaagccattaggttgagggcacgcctgcctgggcgtcacatatcgttgcccgatgcctattgcagtgtgataggaatttttagggcgaatgatggcttcccgtgagcgttgttgcctcgcggctggttgaaaattgagtccttggtggggtgtgccatgatagatggtggtcgagttagcacgagacccatcatgtgtacgctccccataatatggcttcgatgacccacatgcgtcttttgactctcatgacgagacctcaggtcaggcggggctacccgctgaatttaagcatatcaataagcggagga

SEQ ID NO: 55 - DNA, 1491 bp, Withania somnifera
  source 1..1491: mol_type = unassigned DNA
atggcgatacatttatacaaaacttctaccccgagcacacgcaatggaaccgtagacagtcaagtgaaatccaatccacgaaataatttgatctatggacagcgtcgttgtggtaaaggtcgtaatgccagaggaatcattaccgcaaggcatagagggggaggtcataagcgtctataccgtaaaatcgattttcgacggaatgaaaaagacatatatggtagaatcgtaaccatagaatacgaccctaatcgaaatgcatacatttgtctcatacactatggggatggtgagaagagatatattttacatcccagaggggctataattggagataccattgtttctggtacagaagttcctataaaaatgggaaatgccctacctttgagtgcggtttgaactattgatttacgtaattggaaataaccaattaggtttacgacgaaacctataaatcgatcactgatccaatttgagtacctctgcaggatagacctcaacagaaaactgaagagtaacggcagcaagtgattgagttcagtagttcctcatataaaattattgactctagagatatagtaatatggagaagacaaaattgtttcaagcaccgacagaaccggaagcgccccttctttcaaagataggaggacgggttattcacatttcatttgatggtcagaggcgaattgaaagttaagcagtgggaattctaaagattccccgggggaaaaatagagatgtctcctacgttacccataatatgtggaagtatcgacgtaatttcatagagtcattcggtctgaatgctacatgaagaacataagccagatgacggaacgggaagacccaggatgtagaagatcataacatgagtgattcggcagatttggattcatatatatatccacccatgtggtacttcattctacgatatatataagatccatatgtatagatatcatcatctacatccagaaagccgtatgctttggaagaagcttgtacagtttgggaaggggttttgattgatcaaaagaagaatctacttcaaccgatatgcccttaggcacggccatacataacatagaaatcacacttggaaagggtggacaattagctagagcagcgggtgctgtagcgaaactgattgcaaaagaggggaaatcggccacattaaaattaccttctggggaggtccgtttgatatccaaaaactgctcagcaacagtcggacaagtggggaatgttggggtgaaccagaaaagtttgggtagagccggatctaagcgttggctaggtaagcgtcctgtagtaagaggagtagttatgaaccctgtagaccatccccatgggggtggtgaagggagagccccaattggtagaaaaaaacccacaaccccttggggttatcctgcacttggaagaagaagtagaaaaaggaataaatatagtgataattttattcttcgtcgccgtagtaaatag

SEQ ID NO: 56 - DNA, 1491 bp, Withania somnifera; identical to SEQ ID NO: 55
  source 1..1491: mol_type = unassigned DNA

SEQ ID NO: 57 - DNA, 103 bp, Withania somnifera
  source 1..103: mol_type = unassigned DNA
gaaggtcacggcgagacgagccgtttatcattacgataggtgtcaagtggaagtgcagtgatgtatgcagctgaggcatcctaacagaccggtagacttgaac

SEQ ID NO: 58 - DNA, 121 bp, Withania somnifera
  source 1..121: mol_type = unassigned DNA
tattctggtgtcctaggcgtagaggaaccacaccaatccatcccgaacttggtggttaaactctactgcggtgacgatactgtaggggaggtcctgcggaaaaatagctcgacgccaggat

SEQ ID NO: 59 - DNA, 1490 bp, Withania somnifera
  source 1..1490: mol_type = unassigned DNA
tctcatggagagttcgatcctggctcaggatgaacgctggcggcatgcttaacacatgcaagtcggacgggaaacacgggaaaccgtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttcccggagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagctggagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccatcgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggat

(19) United States
(12) Patent Application Publication
(10) Pub. No.: US 2025/0064882 A1
(43) Pub. Date: Feb. 27, 2025

(54) POSTBIOTIC COMPOSITIONS AND METHODS

(71) Applicant: POSTBIOTICS PLUS RESEARCH LLC, Houston, TX (US)

(72) Inventors: Aubrey Elizabeth LEVITT, Houston, TX (US); Jonas SCHLÜTER, Houston, TX (US)

(73) Assignee: POSTBIOTICS PLUS RESEARCH LLC, Houston, TX (US)

(21) Appl. No.: 18/728,273

(22) PCT Filed: Jan. 13, 2023
(86) PCT No.: PCT/US2023/060616

§ 371 Date: Jul. 11, 2024

Related U.S. Application Data
(60) Provisional application No. 63/299,607, filed on Jan. 14, 2022.

(51) Int. Cl. A61K 36/481; A61K 31/122; A61K 31/19; A61K 31/192; A61K 31/194; A61K 31/198; A61K 31/20; A61K 31/352; A61K 31/366; A61K 31/375; A61K 31/60; A61K 35/745; A61K 35/747; A61K 36/185; A61K 36/48; A61K 36/81; A61K 45/06; A61K 47/36; A61P 1/00

(52) U.S. Cl. CPC A61K 36/481; A61K 31/122; A61K 31/19; A61K 31/192; A61K 31/194; A61K 31/198; A61K 31/20; A61K 31/352; A61K 31/366; A61K 31/375; A61K 31/60; A61K 35/745; A61K 35/747; A61K 36/185; A61K 36/48; A61K 36/81; A61K 45/06; A61K 47/36; A61P 1/00; A61K 2236/19

(57) ABSTRACT

Provided herein are postbiotic compositions prepared using fermentation and unique herbal substrate compositions. Also provided herein are methods for the treatment or prevention of the disruption of gut microbiota, or dysbiosis, associated with an antibiotic treatment, chemotherapy treatment, or administration of a dysbiosis-causing medications or medical treatments, using said postbiotic compositions.

### CROSS-REFERENCE TO RELATED APPLICATIONS

This application claims benefit under 35 U.S.C. § 119(e) of U.S. Provisional Application No. 63/299,607 filed Jan. 14, 2022, the contents of which are incorporated herein by reference in their entirety.

### SEQUENCE LISTING

The instant application contains a Sequence Listing which has been submitted in XML format via Patent Center and is hereby incorporated by reference in its entirety. Said XML copy, created on Jan. 12, 2023, is named 084487-191310WOPT_SL.xml and is 147 kilobytes in size.

### FIELD

The present disclosure is broadly concerned with postbiotic compositions and methods of using them. The disclosure is also concerned with herbal/botanical/plant-based substrate compositions for use in preparing fermented or postbiotic compositions as well as postbiotic compositions and methods for the treatment or reconstitution or prevention of the disruption of gut microbiota associated with an antibiotic treatment, chemotherapy treatment, or administration of dysbiosis-causing medications or medical treatments or environmental or lifestyle factors in a subject. The present disclosure is also related to the treatment or prevention of dysbiosis or dysbacteriosis in a subject receiving an antibiotic treatment, a chemotherapy treatment, or administration of a dysbiosis-causing medication or medical treatment. The present disclosure is also related to the adjuvant treatment of cancer patients and other patients receiving immunotherapy, in some instances such as hematopoietic cell therapies, or chimeric antigen receptor T cell therapies or immune checkpoint inhibitor therapies. The present disclosure is also related to the prevention or treatment of antimicrobial resistance gene selection.

### BACKGROUND

Antibiotics are indispensable tools to treat and cure microbial infections, especially in immunocompromised patients, or populations with otherwise suboptimal immune function such as the elderly and young children. Most antibiotics are derivatives of molecules that microbes secrete to kill each other, and as a defense, microbes are readily able to evolve antimicrobial resistance (AMR). Treating multi-resistant strain infections is one of the grand challenges of modern medicine, and therefore, it is pivotal to limit the emergence of resistant strains. Oral antibiotics can collaterally destroy the vast majority of commensal microbes, dramatically reducing microbial diversity in the gut and eventually leading to invasion and domination of the gut microbiota by resistant strains, few surviving strains that can include opportunistic or obligate pathogens.

Chemotherapy treatments are also known to disrupt gut microbiota. In the case of some cancer treatments, antibiotics are administered in association with chemotherapy treatments. For example, patients with blood cancer who receive hematopoietic cell transplants (HCT) are treated with high doses of chemotherapy and/or radiation therapy. These medications lower the patient's white blood cell count and destroy the diversity and balance in their gut microbiome, leaving patients prone to infections and complications. Many patients also receive a high dose of prophylactic or empirical antibiotics which further destroy the gut microbiome. It has also been shown that exposure to antibiotic therapy influences the probability of response to immune checkpoint inhibitor (ICPI) therapy and is predictive of shorter patient survival across malignancies. It has also been shown that antibiotics influence the probability of response and positive treatment outcome of chimeric antigen receptor T cell (CAR T) therapy. It has been shown that loss of anaerobe bacteria is associated with loss of bacterial diversity, and it has been shown that loss of anaerobe bacteria is associated with decreased probability of response to CAR T therapy.

By disrupting the gut ecosystem, antibiotics and other therapies including chemotherapies and CAR-T therapies themselves can instigate downstream metabolic alterations within the microenvironment with complex repercussions to the tumour-host-microbe interface.

Additionally, other therapeutic treatments that can be administered in association with chemotherapy such as HCT cause large shifts in microbiota populations. It has been shown that the resulting low microbiota diversity at the time of neutrophil engraftment in patients undergoing HCT is associated with 5-fold increased transplant-related mortality, indicating that microbiota diversity is critical for clinical outcomes.

Furthermore, the administration of many human-targeted drugs has been linked to unintentional dysbiosis or dysbacteriosis in subjects. Such dysbiosis-causing drugs can include acid-blocking medications such as proton-pump inhibitors (PPIs) and H2 blockers, birth control, steroids, antipsychotics, opioids, metformin, selective serotonin reuptake inhibitors (SSRIs), and nonsteroidal anti-inflammatory drugs (NSAIDs).

Furthermore, it has been shown that antibiotics directly kill and/or interfere with the natural growth of most gut microbiota bacteria commonly found in healthy subjects.

The disruption of gut microbiota can have severe health consequences including deleterious effects on the immune system, inflammation, secondary infections, and other complications. Accordingly, additional compositions and methods for preventing or treating the disruption of gut microbiota is desirable. In particular, additional compositions and methods for preventing or treating the disruption of gut microbiota as a result of a subject receiving an antibiotic treatment, a chemotherapy treatment, and/or administration of a dysbiosis-causing medication or medical treatments are desirable. Additionally, compositions and methods to reconstitute a microbiome damaged by one or many of the afore mentioned factors, or other factors are desirable.

### SUMMARY

Numerous examples are provided herein to enhance understanding of the present disclosure.

In one aspect, described herein is a fermentation substrate composition for use in a fermentation process to prepare a postbiotic composition. In some embodiments of any of the aspects, the fermentation substrate is or comprises an herbal substrate composition or herbal material. In some embodiments of any of the aspects, the substrate composition or herbal material comprises at least one of an herb of the Astragalus family, an herb of the Solanaceae or nightshade family, a berry of the Sambucus L. genus, and a legume of the Lens orientalis or Lens culinaris family. The substrate composition also comprises liquid water, e.g., sufficient to suspend or submerge the plant substrate material.

In one aspect, described herein is a fermentation substrate composition for use in a fermentation process to prepare a postbiotic composition, the fermentation substrate comprising herbal material comprising at least one of an herb of the Astragalus family, an herb of the Solanaceae or nightshade family, a berry of the Sambucus L. genus, and a legume of the Lens orientalis or Lens culinaris family, in combination with liquid water sufficient to suspend or submerge the herbal material.

In one aspect, described herein is a fermentation substrate composition for use in a fermentation process to prepare a postbiotic composition, the fermentation substrate comprising herbal material comprising an herb of the Solanaceae or nightshade family and a berry of the Sambucus L. genus, in combination with liquid water sufficient to suspend or submerge the herbal material.

In one aspect, described herein is a fermentation substrate composition for use in a fermentation process to prepare a postbiotic composition, the fermentation substrate comprising herbal material comprising ashwagandha root and elderberry, in combination with liquid water sufficient to suspend or submerge the herbal material.

In one aspect, described herein is a fermentation substrate composition for use in a fermentation process to prepare a postbiotic composition, the fermentation substrate comprising herbal material comprising an herb of the Solanaceae or nightshade family and a berry of the Sambucus L. genus, in combination with at least one Bifidobacterium species and at least one Lactobacillus species, and liquid water sufficient to suspend or submerge the herbal material.

In one aspect, described herein is a fermentation substrate composition for use in a fermentation process to prepare a postbiotic composition, the fermentation substrate comprising herbal material comprising ashwagandha root and elderberry, in combination with at least one Bifidobacterium species and at least one Lactobacillus species, and liquid water sufficient to suspend or submerge the herbal material.

In one aspect, described herein is a fermentation substrate composition for use in a fermentation process to prepare a postbiotic composition, the fermentation substrate comprising herbal material comprising an herb of the Solanaceae or nightshade family and a berry of the Sambucus L. genus, in combination with at least two of the following: B. lactis, B. infantis, B. breve, L. paracasei, L. rhamnosus, and/or L. casei, and liquid water sufficient to suspend or submerge the herbal material.

In one aspect, described herein is a fermentation substrate composition for use in a fermentation process to prepare a postbiotic composition, the fermentation substrate comprising herbal material comprising ashwagandha root and elderberry, in combination with at least two of the following: B. lactis, B. infantis, B. breve, L. paracasei, L. rhamnosus, and/or L. casei, and liquid water sufficient to suspend or submerge the herbal material.

In one aspect, described herein is a fermentation substrate composition for use in a fermentation process to prepare a postbiotic composition, the fermentation substrate comprising herbal material comprising an herb of the Solanaceae or nightshade family and a berry of the Sambucus L. genus, in combination with B. lactis, B. infantis, B. breve, L. paracasei, L. rhamnosus, and L. casei, and liquid water sufficient to suspend or submerge the herbal material.

In one aspect, described herein is a fermentation substrate composition for use in a fermentation process to prepare a postbiotic composition, the fermentation substrate comprising herbal material comprising ashwagandha root and elderberry, in combination with B. lactis, B. infantis, B. breve, L. paracasei, L. rhamnosus, and L. casei, and liquid water sufficient to suspend or submerge the herbal material.

In some embodiments of any of the aspects, the herbal material is provided as a dried powder prior to combination with water. In some embodiments of any of the aspects, the dried powder comprises dried powder of a juice.

In some embodiments of any of the aspects, the herb of the Astragalus family is Astragalus membranaceus. In some embodiments of any of the aspects, the herb of the Astragalus family is Astragalus membranaceus root. In some embodiments of any of the aspects, the herb is provided as a dried powder.

In some embodiments of any of the aspects, the herb of the Solanaceae or nightshade family is ashwagandha root. In some embodiments of any of the aspects, the herb is provided as a dried powder.

In some embodiments of any of the aspects, the berry of the Sambucus L. genus is elderberry. In some embodiments of any of the aspects, the berry is provided as a dried powder.

In some embodiment of any of the aspects s, the legume of the Lens orientalis or Lens culinaris family is a lentil. In some embodiments of any of the aspects, the legume is red lentil. In some embodiments of any of the aspects, the legume is provided as a dried powder.

In some embodiments of any of the aspects, the fermentation substrate comprises at least an herb of the Astragalus family and an herb of the Solanaceae or nightshade family. In some embodiments of any of the aspects, the fermentation substrate comprises an herb of the Astragalus family and a berry of the Sambucus L. genus. In some embodiments of any of the aspects, the fermentation substrate comprises an herb of the Astragalus family and a legume of the Lens orientalis or Lens culinaris family. In some embodiments of any of the aspects, the fermentation substrate comprises an herb of the Astragalus family, an herb of the Solanaceae or nightshade family and a berry of the Sambucus L. genus. In some embodiments of any of the aspects, the fermentation substrate comprises an herb of the Astragalus family, an herb of the Solanaceae or nightshade family and a legume of the Lens orientalis or Lens culinaris family. In some embodiments of any of the aspects, the fermentation substrate comprises an herb of the Astragalus family, a berry of the Sambucus L. genus and a legume of the Lens orientalis or Lens culinaris family. In some embodiments of any of the aspects, the fermentation substrate comprises an herb of the Solanaceae or nightshade family and a berry of the Sambucus L. genus. In some embodiments of any of the aspects, the fermentation substrate comprises ashwagandha root and elderberry (e.g., elderberry juice). In some embodiments of any of the aspects, the fermentation substrate comprises an herb of the Solanaceae or nightshade family and a legume of the Lens orientalis or Lens culinaris family. In some embodiments of any of the aspects, the fermentation substrate comprises an herb of the Solanaceae or nightshade family, a berry of the Sambucus L. genus and a legume of the Lens orientalis or Lens culinaris family. In some embodiments of any of the aspects, the fermentation substrate comprises a berry of the Sambucus L. genus and a legume of the Lens orientalis or Lens culinaris family. In some embodiments of any of the aspects, an herb of the Astragalus family, an herb of the Solanaceae or nightshade family, a berry of the Sambucus L. genus and a legume of the Lens orientalis or Lens culinaris family.

In some embodiments of any of the aspects, the fermentation substrate comprises Astragalus and Ashwagandha. In some embodiments of any of the aspects, the fermentation substrate comprises Astragalus and elderberry. In some embodiments of any of the aspects, the fermentation substrate comprises Astragalus and red lentil. In some embodiments of any of the aspects, the fermentation substrate comprises ashwagandha and elderberry. In some embodiments of any of the aspects, the fermentation substrate comprises elderberry and red lentil. In some embodiments of any of the aspects, the fermentation substrate comprises Astragalus, ashwagandha and elderberry. In some embodiments of any of the aspects, the fermentation substrate comprises Astragalus, ashwagandha and red lentil. In some embodiments of any of the aspects, the fermentation substrate comprises Astragalus, elderberry and red lentil.

In some embodiments of any of the aspects, the fermentation substrate further comprises one or more of glucose, sucrose, fructose, honey, and molasses. In some embodiments of any of the aspects, the fermentation substrate further comprises glucose, sucrose, fructose, honey, or molasses.

The pH of the fermentation substrate can vary, and can change over the course of fermentation. In some embodiments of any of the aspects, the pH of the fermentation substrate is from about 5.0 to about 8.0. In some embodiments of any of the aspects, the pH of the fermentation is from about 5.0 to about 7.5. In some embodiments of any of the aspects, the pH of the fermentation is from about 5.0 to about 7.5, about 5.0 to about 7.0, about 5.0 to about 6.8, about 5.0 to about 6.6, about 5.0 to about 6.4, about 5.0 to about 6.2, about 5.0 to about 6.0, about 5.5 to about 8.0, about 5.5 to about 7.5, about 5.5 to about 7.0, about 5.5 to about 6.8. about 5.5 to about 6.6, about 5.5 to about 6.4, about 5.5 to about 6.2, about 5.5 to about 6.0, about 6.0 to about 8.0, about 6.0 to about 7.5, about 6.0 to about 7.0, about 6.0 to about 6.8, about 6.0 to about 6.6, about 6.0 to about 6.4, about 6.0 to about 6.2, about 6.2 to about 8.0, about 6.2 to about 7.5, about 6.2 to about 7.0, about 6.2 to about 6.8, about 6.2 to about 6.6, about 6.2 to about 6.4, about 6.4 to about 8.0, about 6.4 to about 7.5, about 6.4 to about 7.0, about 6.4 to about 6.8, about 6.4 to about 6.6, about 6.6 to about 8.0, about 6.6 to about 7.5, about 6.6 to about 7.0, about 6.6 to about 6.8, about 6.8 to about 8.0, about 6.8 to about 7.5, about 6.8 to about 7.0, about 7.0 to about 8.0 or about 7.0 to about 7.5. In some embodiments of any of the aspects, the pH of the fermentation is from about 3.5 to 6.7 (e.g., during the fermentation). In some embodiments of any of the aspects, the pH of the fermentation is from about 3.5 to 4.2 (e.g., end of fermentation). In some embodiments of any of the aspects, the pH of the end product of the fermentation process (e.g., a postbiotic composition) is from 5.0 to 8.0.

In one aspect, described herein is a fermentation substrate composition for use in a fermentation process to prepare a postbiotic composition, the fermentation substrate comprising at least one herbal material selected from Astragalus membranaceus root, ashwagandha, elderberry and red lentil, in combination with liquid water sufficient to suspend or submerge the herbal material.

In some embodiments of any of the aspects, the fermentation substrate composition further comprises glucose, sucrose, fructose, honey, or molasses. In some embodiments of any of the aspects, the fermentation substrate composition further comprises a source of fermentable sugar, e.g., glucose, sucrose, fructose, malt extract, molasses, honey, or other fermentable sugar.

In some embodiments of any of the aspects, the herbal material is provided as a dried powder prior to combination with water.

In some embodiments of any of the aspects, the fermentation substrate comprises Astragalus membranaceus root and Ashwagandha.

In some embodiments of any of the aspects, the fermentation substrate comprises Astragalus membranaceus root and elderberry.

In some embodiments of any of the aspects, the fermentation substrate comprises Astragalus membranaceus root and red lentil.

In some embodiments of any of the aspects, the fermentation substrate comprises ashwagandha and elderberry.

In some embodiments of any of the aspects, the fermentation substrate comprises elderberry and red lentil.

In some embodiments of any of the aspects, the fermentation substrate comprises Astragalus membranaceus root, ashwagandha and elderberry.

In some embodiments of any of the aspects, the fermentation substrate comprises Astragalus membranaceus root, ashwagandha and red lentil.

In some embodiments of any of the aspects, the fermentation substrate comprises Astragalus membranaceus root, elderberry and red lentil.

In some embodiments of any of the aspects, the fermentation substrate comprises Astragalus membranaceus root, ashwagandha, elderberry and red lentil.

In some embodiments of any of the aspects, the fermentation substrate composition comprises from about 2% by weight to about 10% by weight herb of the Astragalus family. In some embodiments of any of the aspects, the substrate composition comprises from about 2.5% by weight to about 5% by weight herb of the Astragalus family.

In some embodiments of any of the aspects, the fermentation substrate composition comprises from about 2% by weight to about 10% by weight Astragalus. In some embodiments of any of the aspects, the fermentation substrate composition comprises from about 2.5% by weight to about 5% by weight Astragalus.

In some embodiments of any of the aspects, the fermentation substrate composition comprises from about 2% by weight to about 10% by weight herb of the Solanaceae or nightshade family. In some embodiments of any of the aspects, the fermentation substrate composition comprises from about 2.5% by weight to about 5% by weight herb of the Solanaceae or nightshade family.

In some embodiments of any of the aspects, the fermentation substrate composition comprises from about 2% by weight to about 10% by weight ashwagandha. In some embodiments of any of the aspects, the fermentation substrate composition comprises from about 2.5% by weight to about 5% by weight ashwagandha.

In some embodiments of any of the aspects, the fermentation substrate composition comprises from about 2% by weight to about 10% by weight berry of the Sambucus L. genus. In some embodiments of any of the aspects, the fermentation substrate composition comprises from about 2% by weight to about 10% by weight berry of the Sambucus L. genus.

In some embodiments of any of the aspects, the fermentation substrate composition comprises from about 2% by weight to about 10% by weight elderberry.

In some embodiments of any of the aspects, the fermentation substrate composition comprises from about 2.5% by weight to about 5% by weight elderberry.

In some embodiments of any of the aspects, the fermentation substrate composition comprises from about 2% by weight to about 10% by weight glucose, sucrose, fructose, honey, or molasses.

In some embodiments of any of the aspects, the fermentation substrate composition comprises from about 2.5% by weight to about 5% by weight glucose, sucrose, fructose, honey, or molasses.

In some embodiments of any of the aspects, the fermentation substrate composition comprises from about 0.5% by weight to about 3% by weight legume of the Lens orientalis or Lens culinaris family. In some embodiments of any of the aspects, the fermentation substrate composition comprises from about 0.5% by weight to about 1.5% by weight legume of the Lens orientalis or Lens culinaris family.

In some embodiments of any of the aspects, the fermentation substrate composition comprises from about 0.5% by weight to about 3% by weight red lentil. In some embodiments of any of the aspects, the fermentation substrate composition comprises from about 0.5% by weight to about 1.5% by weight red lentil.

In some embodiments of any of the aspects, the fermentation substrate composition comprises from about 70% by weight to about 95% by weight water.

In some embodiments of any of the aspects, the fermentation substrate composition comprises from about 80% by weight to about 90% by weight water.

In some embodiments of any of the aspects, the fermentation substrate composition comprises: from about 2.5% by weight to about 5% by weight Astragalus; from about 2.5% by weight to about 5% by weight ashwagandha; from about 2.5% by weight to about 5% by weight elderberry; from about 2.5% by weight to about 5% by weight sucrose or molasses; from about 0.5% by weight to about 1.5% by weight red lentil; and from about 80% by weight to about 90% by weight water.

In some embodiments of any of the aspects, the fermentation substrate composition comprises: from about 2.5% by weight to about 5% by weight ashwagandha; from about 2.5% by weight to about 5% by weight elderberry; from about 2.5% by weight to about 5% by weight sucrose or molasses; from about 0.5% by weight to about 1.5% by weight red lentil; and from about 80% by weight to about 90% by weight water.

In some embodiments of any of the aspects, the substrate composition comprises: from about 2.5% by weight to about 5% by weight ashwagandha; from about 2.5% by weight to about 5% by weight elderberry; from about 2.5% by weight to about 5% by weight sucrose or molasses; and from about 80% by weight to about 90% by weight water.

In some embodiments of any of the aspects, the substrate composition comprises: from about 2.5% by weight to about 5% by weight Astragalus; from about 2.5% by weight to about 5% by weight ashwagandha; from about 2.5% by weight to about 5% by weight elderberry; from about 2.5% by weight to about 5% by weight sucrose or molasses; and from about 80% by weight to about 90% by weight water.

In some embodiments of any of the aspects, the fermentation substrate further comprises at least one Bifidobacterium species and at least one Lactobacillus species.

In some embodiments of any of the aspects, the Bifidobacterium is selected from the group consisting of B. lactis, B. breve, B. infantis, and any combination thereof.

In some embodiments of any of the aspects, the Lactobacillus is selected from the group consisting of L. plantarum, L. acidophilus, L. rhamnosus, L. paracasei, L. casei, and any combination thereof.

In some embodiments of any of the aspects, the fermentation substrates comprise at least 2 (e.g., at least 3, at least 4, at least 5, or 6) of the following: B. lactis, B. infantis, B. breve, L. paracasei, L. rhamnosus, and/or L. casei.

In one aspect, described herein is a postbiotic composition, wherein the postbiotic composition is prepared according to a process comprising: (a) preparing a culture of microorganisms; (b) preparing a fermentation substrate composition as described herein, e.g., an herbal fermentation substrate composition as described herein; (c) inoculating the fermentation substrate composition with the culture of microorganisms to generate an inoculate composition; (d) fermenting the inoculate composition for a predetermined amount of time to generate a fermented inoculate composition and; (e) lyophilizing or spray-drying the fermented inoculate composition to obtain the postbiotic composition.

In some embodiments of any of the aspects, the postbiotic composition further comprises a carrier. In some embodiments of any of the aspects, the carrier is resistant starch or maltodextrin.

In some embodiments of any of the aspects described herein, the predetermined amount of time for fermentation is from about 24 hours to about 10 days. In some embodiments of any of the aspects, the predetermined amount of time is from about 24 hours to about 8 days, about 24 hours to about 7 days, about 24 hours to about 6 days, about 24 hours to about 5 days, about 24 hours to about 4 days, about 24 hours to about 72 hours, about 24 hours to about 48 hours, about 24 hours to about 36 hours, about 36 hours to about 8 days, about 36 hours to about 7 days, about 36 hours to about 6 days, about 36 hours to about 5 days, about 36 hours to about 4 days, about 36 hours to about 72 hours, about 36 hours to about 48 hours, about 48 hours to about 8 days, about 48 hours to about 7 days, about 48 hours to about 6 days, about 48 hours to about 5 days, about 48 hours to about 4 days, about 48 hours to about 72 hours, about 72 hours to about 8 days, about 72 hours to about 7 days, about 72 hours to about 6 days, about 72 hours to about 5 days, or about 72 hours to about 4 days. In some embodiments of any of the aspects, the culture of microorganisms comprises at least one Bifidobacterium species and at least one Lactobacillus species.

In some embodiments of any of the aspects, the Bifidobacterium is selected from the group consisting of B. lactis, B. breve, B. infantis, and any combination thereof.

In some embodiments of any of the aspects, the Lactobacillus is selected from the group consisting of L. plantarum, L. acidophilus, L. rhamnosus, L. paracasei, L. casei, and any combination thereof.

It is contemplated that in some embodiments of any of the aspects, other immune-supportive bacteria can be used, alone or in combination with species described herein, to ferment substrates as described herein to provide postbiotic compositions as described herein. Examples are provided, for example, in Schluter et al., Nature 588: 303-307 (2020), the contents of which are incorporated herein by reference in their entirety.

In some embodiments of any of the aspects, the culture of microorganisms comprises a microorganism concentration from about 1.0×10⁸ CFU/mL to about 1×10¹² CFU/mL. In some embodiments of any of the aspects, the culture of microorganisms comprises a microorganism concentration from about 1.0×10⁹ CFU/mL to about 1×10¹¹ CFU/mL.

In some embodiments of any of the aspects, the culture of microorganisms further comprises de Man, Rogosa & Sharpe (MRS) broth. In some embodiments or any of the aspects, the fermentation substrate or culture of microorganisms does not include MRS broth. In some embodiments or any of the aspects, the fermentation substrate or culture of microorganisms does not include any animal product.

In some embodiments of any of the aspects, fermenting the inoculate composition comprises sealing the inoculate composition in a fermentation vat under substantially anaerobic conditions.

In some embodiments of any of the aspects, fermenting the inoculate composition further comprises incubating the inoculate composition at a temperature from about 33° C. to about 40° C. In some embodiments of any of the aspects, fermenting the inoculate composition further comprises incubating the inoculate composition at a temperature from about 33° C. to about 37° C.

In some embodiments of any of the aspects, fermenting the inoculate composition further comprises purging the fermentation vat with nitrogen gas such that the percentage of oxygen in the fermentation vat is maintained <1.5%.

In some embodiments of any of the aspects, the inoculate composition is maintained at a pH from about 5.0 to about 8.0. In some embodiments, the pH of the fermentation is maintained from about 5.0 to about 7.5. In some embodiments, the pH of the fermentation is maintained from about 5.0 to about 7.5, about 5.0 to about 7.0, about 5.0 to about 6.8, about 5.0 to about 6.6, about 5.0 to about 6.4, about 5.0 to about 6.2, about 5.0 to about 6.0, about 5.5 to about 8.0, about 5.5 to about 7.5, about 5.5 to about 7.0, about 5.5 to about 6.8. about 5.5 to about 6.6, about 5.5 to about 6.4, about 5.5 to about 6.2, about 5.5 to about 6.0, about 6.0 to about 8.0, about 6.0 to about 7.5, about 6.0 to about 7.0, about 6.0 to about 6.8, about 6.0 to about 6.6, about 6.0 to about 6.4, about 6.0 to about 6.2, about 6.2 to about 8.0, about 6.2 to about 7.5, about 6.2 to about 7.0, about 6.2 to about 6.8, about 6.2 to about 6.6, about 6.2 to about 6.4, about 6.4 to about 8.0, about 6.4 to about 7.5, about 6.4 to about 7.0, about 6.4 to about 6.8, about 6.4 to about 6.6, about 6.6 to about 8.0, about 6.6 to about 7.5, about 6.6 to about 7.0, about 6.6 to about 6.8, about 6.8 to about 8.0, about 6.8 to about 7.5, about 6.8 to about 7.0, about 7.0 to about 8.0 or about 7.0 to about 7.5. In some embodiments of any of the aspects, the pH of the fermentation is from about 3.5 to 6.7 (e.g., during the fermentation). In some embodiments of any of the aspects, the pH of the fermentation is from about 3.5 to 4.2 (e.g., end of fermentation). In some embodiments of any of the aspects, the pH of the end product of the fermentation process (e.g., a postbiotic composition) is from 5.0 to 8.0.

In some embodiments of any of the aspects, the final bacteria content post fermentation is about 1.0×10⁸ to about 1×10¹¹ colony-forming units per milliliter (cfu/ml) of living bacteria. In some embodiments of any of the aspects, the final bacteria content post fermentation is about 1.0×10⁹ to about 1×10¹⁰ cfu/ml. In some embodiments of any of the aspects, the final bacteria content post fermentation is about 1.0×10⁶ to about 1.5×10⁹ cfu/ml.

In some embodiments of any of the aspects, the bacterial content after drying (e.g., spray drying or freeze drying) is about 1×10⁸ active-fluorescent units per gram (afu/g; e.g., using flow cytometry) to about 2×10⁹ afu/g of bacterial cells, e.g., living or non-living. In some embodiments of any of the aspects, the bacterial content after drying is about 1×10⁸ afu/g to about 1.5×10⁹ afu/g. In some embodiments of any of the aspects, the bacterial content after drying is about 1×10⁸ afu/g to about 1×10⁹ afu/g. In some embodiments of any of the aspects, the bacterial content after drying is about 5×10⁸ afu/g to about 2×10⁹ afu/g. In some embodiments of any of the aspects, the bacterial content after drying is about 5×10⁸ afu/g to about 1.5×10⁹ afu/g. In some embodiments of any of the aspects, the bacterial content after drying is about 5×10⁸ afu/g to about 1×10⁹ afu/g. In some embodiments of any of the aspects, the bacterial content after drying is about 5×10⁸ afu/g to about 8.8×10⁸ afu/g. In some embodiments of any of the aspects, the bacterial content after drying is about 5×10⁸ afu/g to about 8.5×10⁸ afu/g. In some embodiments of any of the aspects, the bacterial content after drying is about 5×10⁸ afu/g to about 8.2×10⁸ afu/g. In some embodiments of any of the aspects, the bacterial content after drying is about 5×10⁸ afu/g to about 8.0×10⁸ afu/g.

In some embodiments of any of the aspects, the postbiotic composition after drying (e.g., spray drying or freeze drying) comprises non-viable or non-living bacteria. In some embodiments of any of the aspects, the postbiotic composition after drying (e.g., spray drying or freeze drying) comprises a low level of viable or living bacteria. In embodiments where the postbiotic composition is administered to an immunocompromised subject, it is beneficial for the composition to comprise a low level of viable or living bacteria. In embodiments where the postbiotic composition is administered to a non-immunocompromised subject, the composition can comprise viable or non-viable bacteria.

In some embodiments of any of the aspects, the bacterial content after spray drying is about 0 cfu/g of living or viable bacteria. In some embodiments of any of the aspects, the bacterial content after spray drying is greater than 0 cfu/g of living or viable bacteria. In some embodiments of any of the aspects, the bacterial content after spray drying is less than 100,000 cfu/g of living or viable bacteria. In some embodiments of any of the aspects, the bacterial content after spray drying is less than 10⁷ cfu/g of living or viable bacteria. In some embodiments of any of the aspects, the bacterial content after spray drying is less than 10¹ cfu/g, less than 10² cfu/g, less than 10³ cfu/g, less than 10⁴ cfu/g, less than 10⁵ cfu/g, less than 10⁶ cfu/g, or less than 10⁷ cfu/g of living or viable bacteria.

In some embodiments of any of the aspects, the bacterial content after freeze drying is at most about 10¹⁰ cfu/g. In some embodiments of any of the aspects, the bacterial content after freeze drying is less than 10¹ cfu/g, less than 10² cfu/g, less than 10³ cfu/g, less than 10⁴ cfu/g, less than 10⁵ cfu/g, less than 10⁶ cfu/g, or less than 10⁷ cfu/g.

In some embodiments of any of the aspects, the viability of the bacteria in the postbiotic composition after drying (e.g., spray drying or freeze drying) is at most 10%. In some embodiments of any of the aspects, the viability of the bacteria in the postbiotic composition after drying (e.g., spray drying or freeze drying) is at most 0.01%, at most 0.02%, at most 0.03%, at most 0.04%, at most 0.05%, at most 0.06%, at most 0.07%, at most 0.08%, at most 0.09%, at most 0.1%, at most 0.2%, at most 0.3%, at most 0.4%, at most 0.5%, at most 0.6%, at most 0.7%, at most 0.8%, at most 0.9%, at most 1%, at most 2%, at most 3%, at most 4%, at most 5%, at most 6%, at most 7%, at most 8%, at most 9%, or at most 10%.

In some embodiments of any of the aspects, the postbiotic composition comprises at least one metabolite selected from Table 2.

In some embodiments of any of the aspects, the postbiotic composition comprises at least one metabolite selected from the group consisting of 3-hydroxybutyric acid, quercetin, phloionolic acid, wedelolactone, luteolin, N-[(2S)-2-hydroxypropanoyl]-L-leucine, an indole organic acid or any combination thereof.

In some embodiments of any of the aspects, the postbiotic composition comprises at least one metabolite selected from the group consisting of 3-hydroxybutyric acid, quercetin, phloionolic acid, wedelolactone, luteolin, N-[(2S)-2-hydroxypropanoyl]-L-leucine, and any combination thereof. The postbiotic composition can also include organic acids produced by the fermentation, including, for example, citric acid, succinic acid, lactic acid, glycerol and acetic acid.

In some embodiments of any of the aspects, the postbiotic composition comprises each of 3-hydroxybutyric acid, quercetin, phloionolic acid, wedelolactone, luteolin and N-[(2S)-2-hydroxypropanoyl]-L-leucine and an indole organic acid.

In some embodiments of any of the aspects, the postbiotic composition comprises one or more organic acids produced by the fermentation, selected from citric acid, succinic acid, lactic acid, glycerol and acetic acid.

In some embodiments of any of the aspects, the postbiotic composition comprises each of citric acid, succinic acid, lactic acid, glycerol and acetic acid.

Exemplary amounts in the final fermentation broth include 0.5 to 3.0 gL citric acid, 0.02 to 0.9 g/L succinic acid, 2.0 to 20 g/L lactic acid, 0.1 to 2.5 g/L glycerol, and 1.0 to 20 g/L acetic acid. An exemplary organic acid profile for the final fermentation broth includes 1.18 g/L citric acid, 0.1 g/L succinic acid, 14.69 g/L lactic acid, 0.55 g/L glycerol, and 7.73 g/L acetic acid. See also an example of organic acid content over fermentation time in Table 1.

TABLE 1
Organic Acid Content
Concentration (g/L)

| Time | Citric | Succinic | Lactic | | Acetic |
|---|---|---|---|---|---|
| (hr) | Acid | Acid | Acid | Glycerol | Acid |
| 0 | 2.839 | 0.236 | 0.769 | 0.119 | 0.029 |
| 49 | 2.55 | 0.601 | 6.207 | n.d. | 3.801 |
| 96 | 2.493 | 0.73 | 12.611 | n.d. | 5.716 |

In some embodiments ofany of the aspects, the postbiotic composition comprises one or more components selected from 3-hydroxybutyric acid, quercetin, phloionolic acid, wedelolactone, luteolin, N-[(2S)-2-hydroxypropanoyl]-L-leucine, and indole organic acids. In some embodiments of any of the aspects, the indole organic acids comprise indole-3-acetate and/or indole-3-lactate.

In some embodiments of any of the aspects, the postbiotic composition comprises at least one metabolite selected from Table 2. Hundreds of metabolites exhibited a significant increase in concentration in the postbiotic product. In these increased metabolite categories, the average increase was calculated at 5.6 fold. In some embodiments of any of the aspects, the postbiotic composition comprises at least one metabolite selected from: 3-Hydroxybutyric acid, 6-Methoxysalicylic acid, trans-caffeic acid, Phloroglucinol carboxylic acid, 1, 6, 8-trimethyl-allantoate, Vitamin C, wedelolactone, 9, 10-Dihydroxystearic acid, Isorhamnetin, Pseudopurpurin, Quercetin, Luteolin, 2-Ethylglutaric acid, and Phloionolic acid (see e.g., Table 2).

TABLE 2
Non-limiting examples of metabolites in the postbiotic composition.

| | Enriched in | %-age | |
|---|---|---|---|
| Metabolite | fermentation | increase | Description |
| 3- | 4.0 | 68% | increases brain-derived neurotrophic factor (BDNF) |
| Hydroxybutyric | | | clinical relevance in the treatment of depression, |
| acid | | | anxiety, and cognitive impairment |
| 6- | 4.0 | 68% | role as a bacterial metabolite |
| Methoxysalicylic | | | discovered to have highest cox-1 inhibitory |
| acid | | | compounds, thus anti-inflammatory |
| | | | See e.g., Periwal et al 2022, PLoS Comp Biol |
| | | | (PMID: 35468126) |
| trans-caffeic acid | 5.0 | 85% | bioavailable plant organic compound |
| Phloroglucinol | 5.0 | 85% | metabolite of the microbiota from quercetin |
| carboxylic acid | | | conversion |
| | | | See e.g., Kawabata et al. 2019 Molecules (PMID: |
| | | | 30669635) |
| 1,6,8-trimethyl- | 10.0 | 170% | involved in direct interactions of gut microbiome and host |
| allantoate | | | See e.g., Gao et al. 2014 Biomed Res Int (PMID: 25126572) |
| Vitamin C | 15.0 | 254% | |
| wedelolactone | 18.0 | 305% | a plant-derived natural product |
| | | | increased bioavailability |
| | | | anti-inflammatory |
| | | | antioxidant |
| | | | anticancer |
| | | | antiosteoporosis |
| 9,10- | 21.0 | 356% | natural product |
| Dihydroxystearic | | | decreased in intensity over time during high fat diet |
| acid | | | See e.g., Curtasu et al 2020 Metabolites |
| | | | (PMC7697781) |
| Isorhamnetin | 22.0 | 373% | major active substance of Puhuang, a traditional herb |
| | | | medicine widely used in clinical practice |
| | | | quercetin is a katabolic product of Isorhamnetin |
| Pseudopurpurin | 25.0 | 424% | improves bone geometry |
| | | | selectively exhibits tumor inhibitory potential |
| | | | Humbare 2022 Antioxidants (PMID: 35624869) |
| Quercetin | 32.0 | 542% | suppresses pro-inflammatory cytokines, such as |
| | | | interleukin (IL)-17, tumor necrosis factor alpha, and |
| | | | IL-6 |
| | | | promotes the production of IL-10 |
| | | | alleviates Citrobacter rodentium-induced colitis |
| | | | See e.g., Lin et al 2019 Frontiers Microbiology |
| | | | (PMID: 31156598) |
| Luteolin | 34.0 | 576% | Anti-inflammatory |
| | | | benefits on nonalcoholic fatty liver disease |
| | | | reduces blood lipids |
| | | | reduce intestinal permeability |
| | | | See e.g., Sun et al. 2019 Bioorg Chem (PMID: |
| | | | 33991837) |
| 2-Ethylglutaric | 49.0 | 830% | fatty acid |
| acid | | | |
| Phloionolic acid | 63.0 | 1067% | natural product |
| | | | increased bioavailability |

In some embodiments of any of the aspects, the postbiotic composition comprises a metabolite profile exhibiting elevated levels or one or more metabolites selected from the group consisting of 3-hydroxybutyric acid, quercetin, phloionolic acid, wedelolactone, luteolin, N-[(2S)-2-hydroxypropanoyl]-L-leucine, indole organic acid(s), and any combination thereof.

In some embodiments of any of the aspects, the postbiotic composition comprises nucleic acid including sequences selected from: B. lactis, B. breve, B. infantis, L. plantarum, L. acidophilus, L. rhamnosus, L. casei, and/or L. paracasei.

In some embodiments of any of the aspects, the postbiotic composition comprises at least a portion of the 16S rRNA gene sequence from at least one of the following bacterial species: B. lactis, B. breve, B. infantis, L. plantarum, L. acidophilus, L. rhamnosus, L. casei, and/or L. paracasei. In some embodiments of any of the aspects, the postbiotic composition comprises the V4 and/or V5 region of the 16S rRNA gene sequence from at least one of the following bacterial species: B. lactis, B. breve, B. infantis, L. plantarum, L. acidophilus, L. rhamnosus, L. casei, and/or L. paracasei. In some embodiments, the V4 and/or V5 region is about 250 base pairs long. In some embodiments of any of the aspects, the postbiotic composition comprises nucleic acid, including nucleic acid molecules that can hybridize with primer sequences (e.g., 16S primer) sequences selected from SEQ ID NOs: 1-13 or nucleic acid complementary to at least one of SEQ ID NOs: 1-13: B. lactis:

(SEQ ID NO: 1)
TGGAGGGTTCGATTCTGGCTCAGGATGAACGCTG,
B. breve:
(SEQ ID NO: 2)
CCGGATGCTCCATCACAC,
B. breve:
(SEQ ID NO: 3)
ACAAAGTGCCTTGCTCCCT,
B. infantis:
(SEQ ID NO: 4)
TTCCAGTTGATCGCATGGTC,
B. infantis:
(SEQ ID NO: 5)
GGAAACCCCATCTCTGGGAT,
L. plantarum:
(SEQ ID NO: 6)
GCTGGCAATGCCATCGTGCT,
L. plantarum:
(SEQ ID NO: 7)
TCTCAACGGTTGCTGTATCG,
L. acidophilus:
(SEQ ID NO: 8)
CCTTTCTAAGGAAGCGAAGGAT,
L. acidophilus:
(SEQ ID NO: 9)
ACGCTTGGTATTCCAAATCGC,
L. rhamnosus:
(SEQ ID NO: 10)
GCCGATCGTTGACGTTAGTTGG,
L. rhamnosus:
(SEQ ID NO: 11)
CAGCGGTTATGCGATGCGAAT,
L. paracasei:
(SEQ ID NO: 12)
CAATGCCGTGGTTGTTGGAA,
or
L. paracasei:

GCCAATCACCGCATTAATCG (SEQ ID NO: 13). In some embodiments, the primer(s) hybridizes specifically under stringent conditions to a DNA fragment having the nucleotide sequence (e.g., at least a portion of the 16S rRNA gene sequence). As herein used, the term “stringent conditions” means hybridization will occur only if there is at least 95% identity in nucleotide sequences. In another embodiment, hybridization under “stringent conditions” occurs when there is at least 97% identity between the sequences.

In one aspect, described herein is a postbiotic composition comprising 3-hydroxybutyric acid, quercetin, phloionolic acid, wedelolactone, luteolin, N-[(2S)-2-hydroxypropanoyl]-L-leucine and an indole organic acid.

In some embodiments of any of the aspects, the postbiotic composition further comprises bacteria of the genera Bifidobacterium and Lactobacillus.

In some embodiments of any of the aspects, the postbiotic composition further comprises a 16S RNA having a nucleic acid sequence at least 90% identical to one of SEQ ID NO: 14-16 (B. lactis), SEQ ID NO: 17-20 (B. breve), SEQ ID NO: 21-26 (B. infantis), SEQ ID NO: 27-32 (L. plantarum), SEQ ID NO: 33-39 (L. acidophilus), SEQ ID NO: 40-44 (L. rhamnosus), SEQ ID NO: 45-48 (L. paracasei), or SEQ ID NO: 49-52 (L. casei).

In some embodiments of any of the aspects, the postbiotic composition further comprises one or more organic acids selected from citric acid, succinic acid, lactic acid, glycerol and acetic acid.

In some embodiments of any of the aspects, the postbiotic composition comprises each of citric acid, succinic acid, lactic acid, glycerol and acetic acid.

In one aspect, described herein is an oral postbiotic formulation, the formulation comprising a composition as described herein (e.g., a postbiotic composition). In one aspect, described herein is a composition for oral delivery, the composition comprising a postbiotic composition as described herein, formulated for oral delivery

In some embodiments of any of the aspects, the oral postbiotic formulation is formulated as a tablet, pill, capsule, or microcapsule.

In some embodiments of any of the aspects, the oral postbiotic formulation is formulated for buccal, sublabial, or sublingual administration.

In some embodiments of any of the aspects, the oral postbiotic formulation is a liquid suspension. In some embodiments of any of the aspects, the formulation comprises a liquid suspension.

In one aspect, described herein is a pharmaceutical composition comprising a composition as described herein (e.g., a postbiotic composition) and a pharmaceutically acceptable carrier.

In some embodiments of any of the aspects, the oral postbiotic formulation is for the treatment or prevention of disruption of gut microbiota associated with an antibiotic treatment, chemotherapy treatment, or administration of a dysbiosis-causing medication or medical treatment in a subject.

In some embodiments of any of the aspects, the oral postbiotic formulation is for the treatment or prevention of dysbiosis or dysbacteriosis associated with an antibiotic treatment, chemotherapy treatment, or administration of a dysbiosis-causing medication or medical treatment in a subject.

In some embodiments of any of the aspects, the oral postbiotic formulation is for the treatment or prevention of disruption of gut microbiota associated with a chemotherapy treatment in a subject.

In some embodiments of any of the aspects, the oral postbiotic formulation is for the treatment or prevention of dysbiosis or dysbacteriosis associated with a chemotherapy treatment in a subject. In some embodiments of any of the aspects, the oral postbiotic formulation is for the treatment or prevention of dysbiosis or dysbacteriosis associated with cancer immunotherapy, including but not limited to immune checkpoint modulator/inhibitor therapy, hematopoietic cell transplantation therapy and CAR-T therapy, vaccination (e.g., a dendritic cell vaccine), or any other approach that facilitates or activates an immune cell response against a cancer.

In some embodiments of any of the aspects, the oral postbiotic formulation is for the treatment or prevention of disruption of gut microbiota associated with administration of a dysbiosis-causing drug in a subject.

In some embodiments of any of the aspects, the oral postbiotic formulation is for the treatment or prevention of dysbiosis or dysbacteriosis associated with administration of a dysbiosis-causing drug in a subject.

In one aspect, described herein is a method of treating or preventing disruption of gut microbiota associated with an antibiotic treatment, chemotherapy treatment, or administration of a dysbiosis-causing medication or medical treatment in a subject, the method comprising administering to the subject an amount of a composition as described herein (e.g., a postbiotic composition) effective to treat or prevent the disruption.

In some embodiments of any of the aspects, the medical treatment comprises a cancer immunotherapy.

In some embodiments of any of the aspects, the cancer immunotherapy comprises immune checkpoint modulator/inhibitor therapy, hematopoietic cell transplantation therapy, CAR-T therapy, a dendritic cell vaccine, or any other approach that facilitates or activates an immune cell response against a cancer.

In some embodiments of any of the aspects, the medical treatment comprises vaccination.

In some embodiments of any of the aspects, the medical treatment comprises treatment with a dysbiosis-causing drug.

In some embodiments of any of the aspects, the dysbiosis-causing drug is selected from the group consisting of acid-blocking medications, proton-pump inhibitors (PPIs), H2 blockers, birth control, steroids, antipsychotics, opioids, metformin, SSRIs, nonsteroidal anti-inflammatory drugs (NSAIDs), and any combination thereof. Metabolic diseases, such as diabetes, can also cause or be associated with dysbiosis, as can poor management of blood sugar in such conditions. As such, insulin can also be considered a drug that influences dysbiosis, and the compositions described herein are specifically contemplated for use in treating or preventing dysbiosis related to diabetes.

In one aspect, described herein is a method of treating cancer, the method comprising administering a cancer immunotherapy and administering a composition as described herein to a subject in need thereof, wherein the administering is effective to treat the cancer. In some embodiments of any of the aspects, such a combination therapy increases the efficacy of the cancer immunotherapy.

In one aspect, described herein is a method of treating cancer, the method comprising administering a CAR-T therapy and administering a composition as described herein to a subject in need thereof, wherein the administering is effective to treat the cancer. In some embodiments of any of the aspects, such a combination therapy increases the efficacy of the CAR-T therapy.

In one aspect, described herein is a method of treating cancer, the method comprising administering chemotherapy and administering a composition as described herein to a subject in need thereof, wherein the administering is effective to treat the cancer. In some embodiments of any of the aspects, such a combination therapy increases the efficacy of the chemotherapy.

In one aspect, described herein is a method of treating an infection, the method comprising administering at least one antibiotic and administering a composition as described herein to a subject in need thereof, wherein the administering is effective to treat the infection. In some embodiments of any of the aspects, such a combination therapy increases the efficacy of the at least one antibiotic.

In one aspect, described herein is a method of increasing neutrophil engraftment, the method comprising administering an effective amount of a composition as described herein to a subject in need thereof.

In one aspect, described herein is a method of treating or preventing intestinal mucositis associated with chemotherapy, the method comprising administering chemotherapy and administering a composition as described herein to a subject in need thereof, wherein the administering is effective to treat the intestinal mucositis.

Mucositis occurs when cancer treatments break down the rapidly dividing epithelial cells lining the gastro-intestinal tract. Non-limiting examples of measurements for mucositis include: microscopic inspection of intestinal biopsy; a patient-reported assessment scale based on symptoms such as vomiting, diarrhea, pain, abdominal complaints, and/or nutritional support (e.g., the National Cancer Institute Common Terminology Criteria for Adverse Events (NCI-CTCAE) scale and the Daily Gut Score (DGS)); and/or a biomarker in a blood, fecal, breath, or urine sample. Non-limiting examples of such mucositis biomarkers or tests include: citrulline; pro-inflammatory cytokines such as TNF-α, IL1-beta, or IL-6; C-reactive protein (CRP); intestinal fatty acid binding protein (I-FABP); ileal bile acid binding protein (I-BABP); calprotectin; calgranulin (S100A12); ratio of fecal human DNA/total DNA; sugar permeability test; hydrogen breath test; 13Clactose test; 3C-sucrose breath test; see e.g., Kuiken et al., “Biomarkers and non-invasive tests for gastrointestinal mucositis,” Support Care Cancer. 2017; 25(9): 2933-2941, the contents of which are incorporated by reference herein in its entirety.

In some embodiments of any of the aspects, administration of a composition as described herein (e.g., a postbiotic composition; e.g., an oral postbiotic formulation; e.g., in combination with a cancer treatment) is associated with at least one of the following outcomes, as compared to a negative control such as a subject not receiving the composition or the treated subject prior to being administered the composition: decreased incidence of cancer relapse (e.g., relapse-free); increased cancer survival; decreased time to neutrophil engraftment; increased peripheral blood mononuclear cell recovery trajectories (e.g., higher peripheral blood mononuclear cell counts); decreased incidence of febrile neutropenia; decreased blood stream infection incidence; and/or decreased 30-day readmission events (see e.g., Example 6).

In some embodiments of any of the aspects, administration of a composition as described herein (e.g., a postbiotic composition; e.g., an oral postbiotic formulation; e.g., in combination with chemotherapy) is associated with an improvement in intestinal mucositis associated with chemotherapy (e.g., decreased intestinal mucositis), as compared to a negative control such as a subject not receiving the composition or the treated subject prior to being administered the composition (see e.g., Example 6).

In one aspect, described herein is a method of preparing a postbiotic composition, the method comprising: (a) preparing a culture of microorganisms; (b) preparing a fermentation substrate composition as described herein, e.g., an herbal fermentation substrate; (c) inoculating the fermentation substrate composition with the culture of microorganisms to generate an inoculate composition; and (d) fermenting the inoculate composition for a predetermined amount of time to generate a fermented inoculate composition.

In some embodiments of any of the aspects, the predetermined amount of time is from about 24 hours to about 10 days, or a period therebetween as described herein above.

In some embodiments of any of the aspects, the culture of microorganisms comprises at least one Bifidobacterium species and at least one Lactobacillus species.

In some embodiments of any of the aspects, the Bifidobacterium is selected from the group consisting of B. lactis, B. breve, B. infantis, and any combination thereof.

In some embodiments of any of the aspects, the Lactobacillus is selected from the group consisting of L. plantarum, L. acidophilus, L. rhamnosus, L. paracasei, L. casei, and any combination thereof.

In some embodiments of any of the aspects, the culture of microorganisms comprises a microorganism concentration from about 1.0×10⁸ CFU/mL to about 1×10¹² CFU/mL.

In some embodiments of any of the aspects, the culture of microorganisms comprises a microorganism concentration from about 1.0×10⁹ CFU/mL to about 1×10¹¹ CFU/mL.

In some embodiments of any of the aspects, fermenting the inoculate composition comprises sealing the inoculate composition in a fermentation vat under substantially anaerobic conditions.

In some embodiments of any of the aspects, fermenting the inoculate composition further comprises incubating the inoculate composition at a temperature from about 33° C. to about 40° C.

In some embodiments of any of the aspects, fermenting the inoculate composition further comprises incubating the inoculate composition at a temperature from about 33° C. to about 37° C.

In some embodiments of any of the aspects, fermenting the inoculate composition further comprises purging the fermentation vat with nitrogen gas such that the percentage of oxygen in the fermentation vat is maintained ≤1.5%.

In some embodiments of any of the aspects, the pH of the fermentation is maintained in the range of about 5.0 to about 7.5, about 5.0 to about 7.0, about 5.0 to about 6.8, about 5.0 to about 6.6, about 5.0 to about 6.4, about 5.0 to about 6.2, about 5.0 to about 6.0, about 5.5 to about 7.5, about 5.5 to about 7.0, about 5.5 to about 6.8. about 5.5 to about 6.6, about 5.5 to about 6.4, about 5.5 to about 6.2, about 5.5 to about 6.0, about 6.0 to about 7.5, about 6.0 to about 7.0, about 6.0 to about 6.8, about 6.0 to about 6.6, about 6.0 to about 6.4, about 6.0 to about 6.2, about 6.2 to about 7.5, about 6.2 to about 7.0, about 6.2 to about 6.8, about 6.2 to about 6.6, about 6.2 to about 6.4, about 6.4 to about 7.5, about 6.4 to about 7.0, about 6.4 to about 6.8, about 6.4 to about 6.6, about 6.6 to about 7.5, about 6.6 to about 7.0, about 6.6 to about 6.8, about 6.8 to about 7.5, about 6.8 to about 7.0, or about 7.0 to about 7.5. In some embodiments of any of the aspects, the inoculate composition is maintained at a pH from about 6.0 to about 6.8. In some embodiments of any of the aspects, the pH of the fermentation is from about 3.5 to 6.7 (e.g., during the fermentation). In some embodiments of any of the aspects, the pH of the fermentation is from about 3.5 to 4.2 (e.g., end of fermentation). In some embodiments of any of the aspects, the pH of the end product of the fermentation process (e.g., a postbiotic composition) is from 5.0 to 8.0.

In some embodiments of any of the aspects, the method further comprises lyophilizing the fermented inoculate composition to obtain the postbiotic composition. In some embodiments of any of the aspects, the method further comprises spray-drying the fermented inoculate composition to obtain the postbiotic composition.

In some embodiments of any of the aspects, the method further comprises formulating the postbiotic composition for oral delivery.

In some embodiments of any of the aspects, the method further comprises formulating the postbiotic composition as a tablet, pill, capsule, or microcapsule.

In some embodiments of any of the aspects, the method further comprises formulating the postbiotic composition in a liquid suspension.

In one aspect, described herein is a pharmaceutical composition comprising a composition (e.g., a postbiotic composition) prepared by a method as described herein, and a pharmaceutically acceptable carrier.

In one aspect, described herein is a method of treating or preventing the disruption of gut microbiota associated with an antibiotic treatment, chemotherapy treatment, or administration of a dysbiosis-causing medication or medical treatment in a subject, the method comprising administering to the subject a pharmaceutically effective amount of a composition as described herein (e.g., a postbiotic composition).

In one aspect, described herein is a method of treating or preventing dysbiosis or dysbacteriosis associated with an antibiotic treatment, chemotherapy treatment, or administration of a dysbiosis-causing medication or medical treatment in a subject, the method comprising administering to the subject a pharmaceutically effective amount of a composition as described herein (e.g., a postbiotic composition).

In one aspect, described herein is a method of treating or preventing the disruption of gut microbiota associated with an antibiotic treatment, chemotherapy treatment, or administration of a dysbiosis-causing medication or medical treatment in a subject, the method comprising administering to the subject a pharmaceutically effective amount of an oral postbiotic formulation as described herein.

In one aspect, described herein is a method of treating or preventing dysbiosis or dysbacteriosis associated with a radiation therapy treatment in a subject, the method comprising administering to the subject a pharmaceutically effective amount of an oral postbiotic formulation as described herein.

In one aspect, described herein is a method of mitigating or preventing antibiotic resistance in a subject receiving an antibiotic treatment, the method comprising administering to the subject a pharmaceutically effective amount of a composition as described herein (e.g., a postbiotic composition) or a pharmaceutically effective amount of an oral postbiotic formulation as described herein.

In some embodiments of any of the aspects, administering to the subject comprises oral administration in the form of a tablet, pill, capsule, or microcapsule.

In some embodiments of any of the aspects, administering to the subject comprises buccal, sublabial, or sublingual administration.

In some embodiments of any of the aspects, administering to the subject comprises oral administration in the form of a liquid suspension.

In one aspect, described herein is a method of treating or preventing dysbiosis or dysbacteriosis associated with a chemotherapy treatment in a subject, the method comprising administering to the subject a pharmaceutically effective amount of a composition as described herein (e.g., a postbiotic composition).

In one aspect, described herein is a method of treating or preventing the disruption of gut microbiota associated with a chemotherapy treatment in a subject, the method comprising administering to the subject a pharmaceutically effective amount of an oral postbiotic formulation as described herein.

In one aspect, described herein is a method of treating or preventing dysbiosis or dysbacteriosis associated with a chemotherapy treatment in a subject, the method comprising administering to the subject a pharmaceutically effective amount of an oral postbiotic formulation as described herein.

In one aspect, described herein is a method of treating or preventing the disruption of gut microbiota associated with administration of a dysbiosis-causing drug in a subject, the method comprising administering to the subject a pharmaceutically effective amount of a composition as described herein (e.g., a postbiotic composition).

In one aspect, described herein is a method of treating or preventing dysbiosis or dysbacteriosis associated with administration of a dysbiosis-causing drug in a subject, the method comprising administering to the subject a pharmaceutically effective amount of a composition as described herein (e.g., a postbiotic composition).

In one aspect, described herein is a method of treating or preventing the disruption of gut microbiota associated with administration of a dysbiosis-causing drug in a subject, the method comprising administering to the subject a pharmaceutically effective amount of an oral postbiotic formulation as described herein.

In one aspect, described herein is a method of treating or preventing dysbiosis or dysbacteriosis associated with administration of a dysbiosis-causing drug in a subject, the method comprising administering to the subject a pharmaceutically effective amount of an oral postbiotic formulation as described herein.

In some embodiments of any of the aspects, the dysbiosis-causing drug is selected from the group consisting of acid-blocking medications, proton-pump inhibitors (PPIs), SSRIs, H2 blockers, birth control, steroids, antipsychotics, opioids, metformin, nonsteroidal anti-inflammatory drugs (NSAIDs), and any combination thereof.

In some embodiments of any of the aspects, administering to the subject comprises oral administration in the form of a tablet, pill, capsule, or microcapsule.

In some embodiments of any of the aspects, administering to the subject comprises buccal, sublabial, or sublingual administration.

In some embodiments of any of the aspects, administering to the subject comprises oral administration in the form of a liquid suspension.

In one aspect, described herein is a combination therapy for administering an antibiotic to a subject in need thereof while reducing or preventing antimicrobial resistance in the subject, the combination therapy comprising: administration of a therapeutically effective amount of an antibiotic to the subject; and administration of a therapeutically effective amount of a composition as described herein (e.g., a postbiotic composition) or a therapeutically effective amount of an oral postbiotic formulation as described herein.

In one aspect, described herein is a combination therapy comprising: administration of a therapeutically effective amount of an antibiotic to the subject; administration of a chemotherapy treatment to the subject; and administration of a therapeutically effective amount of a composition as described herein (e.g., a postbiotic composition) or a therapeutically effective amount of an oral postbiotic formulation as described herein.

In one aspect, described herein is a combination therapy comprising: administration of an immune checkpoint inhibitor (ICPI) therapy to a subject; and administration of a therapeutically effective amount of a composition as described herein (e.g., a postbiotic composition) or a therapeutically effective amount of an oral postbiotic formulation as described herein.

In one aspect, described herein is a combination therapy comprising: administration of a bone marrow transplant to a subject; and administration of a therapeutically effective amount of a composition as described herein (e.g., a postbiotic composition) or a therapeutically effective amount of an oral postbiotic formulation as described herein.

In one aspect, described herein is a combination therapy comprising: administration of a hematopoietic cell transplantation (HCT) or stem cell engraftment to a subject; and administration of a therapeutically effective amount of a composition as described herein (e.g., a postbiotic composition) or a therapeutically effective amount of an oral postbiotic formulation as described herein.

In one aspect, described herein is a combination therapy comprising: administration of a chimeric antigen receptor T cell therapy to a subject; and administration of a therapeutically effective amount of a composition as described herein (e.g., a postbiotic composition) or a therapeutically effective amount of an oral postbiotic formulation as described herein.

In some embodiments of any of the aspects, the combination therapy further comprising administering a chemotherapy treatment to the subject.

In one aspect, described herein is a method of treating cancer, the method comprising administering at least one cancer treatment and administering a postbiotic composition to a subject in need thereof, wherein the administering is effective to treat the cancer, wherein the postbiotic composition is prepared according to a process comprising: (a) preparing a culture of microorganisms comprising B. lactis, B. infantis, B. breve, L. paracasei, L. rhamnosus, and/or L. casei; (b) preparing a fermentation substrate composition comprising herbal material comprising ashwagandha root and elderberry in combination with liquid water sufficient to suspend or submerge the herbal material; (c) inoculating the fermentation substrate composition with the culture of microorganisms to generate an inoculate composition; (d) fermenting the inoculate composition for a predetermined amount of time to generate a fermented inoculate composition and; (e) lyophilizing or spray-drying the fermented inoculate composition to obtain the postbiotic composition.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered limiting of its scope, the principles herein are described and explained with additional specificity and detail through the use of the accompanying drawings.

FIG. 1 illustrates diversity score data for stool samples collected from patients treated with the presently disclosed postbiotic compositions as compared to conventional probiotic control only without the disclosed postbiotic composition as described in Example 5, according to an exemplary embodiment of the present disclosure.

FIG. 2 illustrates microbial family abundance data for patients treated with the presently disclosed postbiotic compositions as compared to conventional probiotic control without the disclosed postbiotic composition as described in Example 5, according to an exemplary embodiment of the present disclosure.

FIG. 3 illustrates diversity data for stool samples collected from patients treated additionally with the presently disclosed postbiotic compositions as compared to only with conventional probiotic control without the disclosed postbiotic composition as described in Example 5, according to an exemplary embodiment of the present disclosure.

FIG. 4 illustrates relative microbial phylum abundance data for beneficial (left) and harmful (right) microbial phyla for stool samples collected from patients treated with the presently disclosed postbiotic compositions as compared to conventional probiotic control without the disclosed postbiotic composition as described in Example 5, according to an exemplary embodiment of the present disclosure.

FIG. 5 is a schematic showing experimental design. Postbiotic-001 (PB001) is a postbiotic prepared using strains of Bifidobacteria and Lactobacillus bacteria to ferment red lentils, ashwaganda, Astragalus, Elderberry, and Molasses. See e.g., exemplary data in FIGS. 6A-6C, 7A-7B, and 8B.

FIG. 6A-6C show the increased microbial diversity of subjects after consuming the PB001 postbiotic. FIG. 6A is a bar graph showing the difference in diversity between placebo and PB001. FIG. 6B is a line graph of a receiver operating characteristic curve that indicates the performance of a classification model at all classification thresholds showing that the microbiome diversity in subjects that consumed the postbiotic was significantly improved compared with control. FIG. 6C is a forest plot of association coefficients between bacterial families and treatment modality showing that subjects who consumed the postbiotic PB001 had more health-associated bacterial families in their microbiome, while the abundance of dysbiotic bacteria were reduced.

FIG. 7A-7B compare the microbiomes of the postbiotic and control groups. FIG. 7A is a bar graph showing that health-associated gut bacteria were significantly enriched because they were supported by the postbiotic product, PB001. FIG. 7B is a bar graph showing that high blood levels of C-reactive protein as an inflammation marker was observed less when the postbiotic, PB001, was consumed.

FIG. 8A-8B illustrates that the postbiotic significantly increased immune system supporting genera. FIG. 8A shows that Faecalibacterium, Akkermansia, and Ruminococcus 2 were higher in recipients of the postbiotic PB001 compared to placebo; thus, PB001 administration can support better immunotherapy results and faster immune recovery. Scardovia, Escherichia-Shigella, and Streptococcus were higher in the placebo, compared to PB001. FIG. 8B shows that specific members of the microbiome (e.g., Faecalibacterium, Akkermansia, and Ruminococcus 2) support immune system recovery; FIG. 8B is adapted from Schluter et al. “The gut microbiota is associated with immune cell dynamics in humans.” Nature 588: 303-307 (2020), the contents of which are incorporated herein by reference in their entirety.

FIG. 9A-9B are heat maps showing a comparison of metabolite profiles in different in vitro fermentation processes. Untargeted metabolomics (HPLC-MS Analysis) on naïve fermentation strategies (1-3) was compared with unfermented raw materials (RAW), unincubated raw materials (CTRL), and the fermentation process used to produce PB001. Shading indicates metabolite concentrations enrichment (light grey: high, dark grey/black: low); two rows per sample category represent replicated measurements.

### DETAILED DESCRIPTION

The present disclosure provides methods for preparing postbiotic compositions with unique and advantageous metabolite and secondary metabolite and organic acid levels and profiles as well as methods for the treatment or prevention of the disruption of gut microbiota associated with an antibiotic treatment, a chemotherapy treatment, or administration of a dysbiosis-causing medication or medical treatment in a subject or the treatment or prevention of dysbiosis or dysbacteriosis in a subject receiving an antibiotic treatment, chemotherapy treatment, or administration of a dysbiosis-causing medication or medical treatment. Postbiotics are primary and secondary metabolic products, molecular cues membrane-bound or dissolved, and secreted products created by probiotic microorganisms that influence the gut microbiome and its host.

According to at least one aspect of the present disclosure, the presently disclosed methods and compositions are effective in protecting the gut microbiota from collateral destruction by oral antibiotics. As a result, the opportunity of gut invasion by resistant microbes or pathogens is prevented, antimicrobial resistance gene domination reduced, and healthy diversity and levels of commensal bacteria are protected. The presently disclosed gut microbiome protecting postbiotic compositions can be prepared according to a process that uses fermentation. In particular, the presently disclosed compositions provide probiotics combined with fermented herbal substrates, and compounds from microbial activity that protects, stimulates and stabilizes a healthy gut ecosystem, thereby protecting the normal microbiome population during and after antibiotic assault as well as supporting the gut's natural ability to rebuild a normal microbiome population after antibiotic assault. Higher gut microbiota diversity, protecting that diversity, and a faster rate of return to a healthy microbiome population is a marker of overall health and can help reduce negative side effects of antibiotic use and other drug use such as yeast infections, acne, diarrhea, bad mood, and inflammation. It has been surprisingly discovered that the fermentation provides postbiotic compositions with unique metabolite profiles. In particular, the fermentation results in postbiotic compositions with elevated advantageous metabolites, such as 3-hydroxybutyric acid, quercetin, phloionolic acid, wedelolactone, luteolin, N-[(2S)-2-hydroxypropanoyl]-L-leucine, organic acids such as citric acid, succinic acid, lactic acid, glycerol and acetic acid, as well as indole organic acids, and vitamin C.

As used herein, the term “postbiotic” or “postbiotics” is defined as the metabolites in, the secondary metabolites of, the secretions, membrane proteins, intra- and/or extra-cellular components of a microbial community.

According to at least one aspect of the present disclosure, a method of preparing a postbiotic composition is provided. The method can include preparing a culture of microorganisms and herbal fermentation substrate composition as described herein, e.g., an herbal fermentation substrate as described herein, followed by inoculating the herbal substrate composition with the culture of microorganisms to generate an inoculate composition. The method can also include fermenting the inoculate composition for a predetermined amount of time to generate a fermented inoculate composition. The fermented inoculate composition can be lyophilized to obtain the postbiotic composition.

In some embodiments, the postbiotic composition can further comprise a carrier, e.g., a carrier suitable for spray drying and/or freeze drying (also referred to as lyophilization). In some embodiments, the carrier is resistant starch (e.g., digestion resistant starch). In some embodiments, the carrier comprises maltodextrin. In some embodiments, the carrier comprises resistant maltodextrin (e.g., FIBERSOL). In some embodiments, the carrier comprises at least one prebiotic fiber co-drying agent. The term “excipient” can be used interchangeably with “carrier.”

The predetermined amount of time can be as described herein above. In at least some instances, the culture of microorganisms comprises at least one microorganism selected from the group consisting of Bifidobacterium and Lactobacillus. In some cases, the culture of microorganisms comprises Bifidobacterium. In some cases, the culture of microorganisms comprises Lactobacillus. The Bifidobacterium can be selected, for example, from the group consisting of B. lactis (also referred to as B. animalis subsp. lactis), B. breve, B. infantis, and any combination thereof. The Lactobacillus can be selected, for example, from the group consisting of L. plantarum, L. acidophilus, L. rhamnosus, L. paracasei, L. casei, and any combination thereof.

In at least some aspects, the culture of microorganisms can comprise a microorganism concentration from about 1.0×10⁸ CFU/mL to about 1×10¹² CFU/mL, or from about 1.0×10⁹ CFU/mL to about 1×10¹ CFU/mL, or from about 1.0×10⁹ CFU/mL to about 1×10¹⁰ CFU/mL, or from about 1.0×10⁸ CFU/mL to about 1×10¹ CFU/mL. In some embodiments, the postbiotic compositions described herein are prepared by spray-drying. In some embodiments, the bacterial content after spray drying is about 0 cfu/g. In some embodiments, the postbiotic compositions described herein are prepared by freeze-drying. In some embodiments, the bacterial content after freeze drying is at most about 10¹⁰ cfu/g. The fermentation substrate composition can be as described herein above.

In some embodiments, the postbiotic composition comprises nucleic acid including sequences from the plant and/or bacterial species described herein. In some embodiments, the postbiotic composition comprises nucleic acid including sequences selected from any of the following bacterial species: B. lactis, B. breve, B. infantis, L. plantarum, L. acidophilus, L. rhamnosus, L. casei, and/or L. paracasei. Such nucleic acids can include DNA or RNA indicative of any one of these bacterial species. In some embodiments, the postbiotic composition comprises nucleic acid including sequences selected from any of the following plant genera or species: the Astragalus family, the Solanaceae or nightshade family, the Sambucus L. genus, and/or the Lens orientalis or Lens culinaris family. In some embodiments, the postbiotic composition comprises nucleic acid including sequences selected from any of the following plant genera or species: Astragalus membranaceus, Astragalus complanatus, ashwagandha (species Withania somnifera, family Solanaceae), elderberry (species Sambucus nigra) and/or red lentil (species Lens culinaris or L. culinaris subsp. orientalis). Such nucleic acids can include DNA or RNA indicative of any one of these plant genera or species.

In some embodiments, the plant is an Astragalus species, e.g., Astragalus membranaceus or Astragalus complanatus (see e.g., Accession number NC_065024.1). In some embodiments, the plant comprises a nucleic acid sequence (e.g., an 18S sequence) comprising one of SEQ ID NOs: 53-54 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NOs: 53-54 or a fragment thereof.

SEQ ID NO: 53 Astragalus complanatus 18S Accession number NC_065024.1:
ATCATACTCAAAAGAAGAGTTTGATCCTGGCTCAGAAGGAACGCTAGCTATATGCTTAAC
ACATGCAAGTCGAACGTTGTTTTCGGGGAGCTGGGCAGAAGGAAAAGAGGCTCCTAGCG
TGAAGGTAGCTTGTCTCGCCCAGGAGGTGGGAACAGTTGAAAACAAAGTGGCGAACGGG
TGCGTAATGCGTGGGAATCTGCCGAACAGTTCGGGCCAAATCCTGAAGAAAGCTAAAAA
GCGCTGTTTGATGAGCCTGCGTAGTATTAGGTAGTTGGTCAGGTAAAGGCTGACCAAGCC
AATGATGCTTAGCTGGTCTTTTCGGATGATCAGCCACACTGGGACTGAGACACGGCCCGG
ACTCCCACGGGGGGCAGCAGTGGGGAATCTTGGACAATGGGCGAAAGCCCGATCCAGCA
ATATCGCGTGAGTGAAGAAGGGCAACGCCGCTTGTAAAGCTCTTTCGTCGAGTGCGCGA
TCATGACAGGACTCGAGGAAGAAGCCCCGGCTAACTCCGTGCCAGCAGCCGCGGTAAGA
CGGGGGGGGCAAGTGTTCTTCGGAATGACTGGGCGTAAAGGGCACGTAGGCGGTGAATC
GGGTTGAAAGTGAAAGTCGCCAAAAACTGGTGGAATGCTCTCGAAACCAATTCACTTGA
GTGAGACAGAGGAGAGTGGAATTTCGTGTGTAGGGGTGAAATCCGCAGATATACGAAGG
AACGCCAAAAGCGAAGGCAGCTCTCTGGGTCCCTACCGACGCTGGAGTGCGAAAGCATG
GGGAGCGAACGGGATTAGATACCCTGGTAGTCCATGCCGTAAACGATGAGTGTTCGCCC
TTGGTCTACGTGGATCAGGGGCCCAGCTAACGCGTGAAACACTCCGCCTGGGGAGTACG
GTCGCAAGACCGAAACTCAAAGGAATTGACGGGGGCCTGCACAAGCGGTGGAGCATGTG
GTTTAATTCGATACAACGCGCAAAACCTTACCAGCCCTTGACATATGAACAAGAAAACCT
GTCCTTAACGGGATGGTACTGACTTTCATACAGGTGCTGCATGGCTGTCGTCAGCTCGTG
TCGTGAGATGTTTGGTCAAGTCCTATAACGAGCGAAACCCTCGTTTTGTGTTGCTGAGAC
ATGCGCCTAAGGAGAAAGTCTTTGCAACCGAAGTGAGCCGAGGAGCCGAGTGACGCGCC
AGCGCTACTAATTGAGTGCCAGCACGTAGCTGTGCTGTCAGTAAGAAGGGAGCCGGCGC
CTTTCGAATTCGAAGCACTCTCTAGTGTGCGCTGTTTTTTGATTGCAGCTAGAGAGCAAG
ACTCGGCATTCAGGCGAGCCGCCCGGTGGTGTGGTCCGTAGTGGGTTTAGTACGCCCCGC
CAAAACGGCTCCGAAACAAACTAAAAGGTGCATGCCGCACTCACGAGGGACTGCCAGTG
ATATACTGGAGGAAGGTGGGGATGACGTCAAGTCCGCATGGCCCTTATGGGCTGGGCCA
CACACGTGCTACAATGGCAATTACAATGGGAAGCAAGGCTGTAAGGCGGAGCGAATCCG
GAAAGATTGCCTCAGTTCGGATTGTTCTCTGCAACTCGGGAACATGAAGTTGGAATCGCT
AGTAATCGCGGATCAGCATGCCGCGGTGAATATGTACCCGGGCCCTGTACACACCGCCC
GTCACACCCTGGGAATTGGTTTCGCCCGAAGCATCGGACCAATGATCACCCATGACTTCT
GTGTACCACTAGTGCCACAAAGGCTTTTGGTGGTCTTATTGGCGCATACCACGGTGGGGT
CTTCGACTGGGGTGAAGTCGTAACAAGGTAGCCGTAGGGGAACCTGTGGCTGGAT
KY316029.1 Astragalus membranaceus
SEQ ID NO: 54
GGAAGTAAAAGTCGTAACAAGGTTTCCGTAGGTGAACCTGCGGAAGGATCATTGTCGAT
GCCTTACATGCAGACCAACTAGTGAATCTGTTTGAATACTTAGGGATGGCTGGGGTGTTT
TGCACCACGACCTCCCTTTGGGTGGGGGGTGGTGCGCAATGCGTTCCCCCTCCTGCCCGA
ACACAAACCCCGGCGCTCAATGCGCCAAGGAACTAAAATTCGATCAATGTGCCCCGTCG
GCCCGGAGACGGTGCTTCGGCGGTGGTGCCTTGTCACATGATACAGAATGACTCTCGGCA
ACGGATATCTAGGCTCTTGCATCGATGAAGAACGTAGCGAAATGCGATACTTGGTGTGA
ATTGCAGAATCCCGTGAACCATCGAGTCTTTGAACGCAAGTTGCGCCCGAAGCCATTAGG
TTGAGGGCACGCCTGCCTGGGCGTCACATATCGTTGCCCGATGCCTATTGCAGTGTGATA
GGAATTTTTAGGGCGAATGATGGCTTCCCGTGAGCGTTGTTGCCTCGCGGCTGGTTGAAA
ATTGAGTCCTTGGTGGGGTGTGCCATGATAGATGGTGGTCGAGTTAGCACGAGACCCATC
ATGTGTACGCTCCCCATAATATGGCTTCGATGACCCACATGCGTCTTTTGACTCTCATGAC
GAGACCTCAGGTCAGGCGGGGCTACCCGCTGAATTTAAGCATATCAATAAGCGGAGGA

In some embodiments, the plant is an Withania species, e.g., Withania somnifera (see e.g., Accession number NC_047245.1). In some embodiments, the plant comprises a nucleic acid sequence (e.g., an 18S sequence) comprising one of SEQ ID NO: 54-58 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical one of SEQ ID NO: 54-58 or a fragment thereof.

SEQ ID NO: 55 Withania 18S, Accession number NC_047245.1:
ATGGCGATACATTTATACAAAACTTCTACCCCGAGCACACGCAATGGAACCGTAGACAG
TCAAGTGAAATCCAATCCACGAAATAATTTGATCTATGGACAGCGTCGTTGTGGTAAAGG
TCGTAATGCCAGAGGAATCATTACCGCAAGGCATAGAGGGGGAGGTCATAAGCGTCTAT
ACCGTAAAATCGATTTTCGACGGAATGAAAAAGACATATATGGTAGAATCGTAACCATA
GAATACGACCCTAATCGAAATGCATACATTTGTCTCATACACTATGGGGATGGTGAGAA
GAGATATATTTTACATCCCAGAGGGGCTATAATTGGAGATACCATTGTTTCTGGTACAGA
AGTTCCTATAAAAATGGGAAATGCCCTACCTTTGAGTGCGGTTTGAACTATTGATTTACG
TAATTGGAAATAACCAATTAGGTTTACGACGAAACCTATAAATCGATCACTGATCCAATT
TGAGTACCTCTGCAGGATAGACCTCAACAGAAAACTGAAGAGTAACGGCAGCAAGTGAT
TGAGTTCAGTAGTTCCTCATATAAAATTATTGACTCTAGAGATATAGTAATATGGAGAAG
ACAAAATTGTTTCAAGCACCGACAGAACCGGAAGCGCCCCTTCTTTCAAAGATAGGAGG
ACGGGTTATTCACATTTCATTTGATGGTCAGAGGCGAATTGAAAGTTAAGCAGTGGGAAT
TCTAAAGATTCCCCGGGGGAAAAATAGAGATGTCTCCTACGTTACCCATAATATGTGGAA
GTATCGACGTAATTTCATAGAGTCATTCGGTCTGAATGCTACATGAAGAACATAAGCCAG
ATGACGGAACGGGAAGACCCAGGATGTAGAAGATCATAACATGAGTGATTCGGCAGATT
TGGATTCATATATATATCCACCCATGTGGTACTTCATTCTACGATATATATAAGATCCATA
TGTATAGATATCATCATCTACATCCAGAAAGCCGTATGCTTTGGAAGAAGCTTGTACAGT
TTGGGAAGGGGTTTTGATTGATCAAAAGAAGAATCTACTTCAACCGATATGCCCTTAGGC
ACGGCCATACATAACATAGAAATCACACTTGGAAAGGGTGGACAATTAGCTAGAGCAGC
GGGTGCTGTAGCGAAACTGATTGCAAAAGAGGGGAAATCGGCCACATTAAAATTACCTT
CTGGGGAGGTCCGTTTGATATCCAAAAACTGCTCAGCAACAGTCGGACAAGTGGGGAAT
GTTGGGGTGAACCAGAAAAGTTTGGGTAGAGCCGGATCTAAGCGTTGGCTAGGTAAGCG
TCCTGTAGTAAGAGGAGTAGTTATGAACCCTGTAGACCATCCCCATGGGGGTGGTGAAG
GGAGAGCCCCAATTGGTAGAAAAAAACCCACAACCCCTTGGGGTTATCCTGCACTTGGA
AGAAGAAGTAGAAAAAGGAATAAATATAGTGATAATTTTATTCTTCGTCGCCGTAGTAA
ATAG
SEQ ID NO: 56 Withania 18S, Accession number NC_047245.1:
ATGGCGATACATTTATACAAAACTTCTACCCCGAGCACACGCAATGGAACCGTAGACAG
TCAAGTGAAATCCAATCCACGAAATAATTTGATCTATGGACAGCGTCGTTGTGGTAAAGG
TCGTAATGCCAGAGGAATCATTACCGCAAGGCATAGAGGGGGAGGTCATAAGCGTCTAT
ACCGTAAAATCGATTTTCGACGGAATGAAAAAGACATATATGGTAGAATCGTAACCATA
GAATACGACCCTAATCGAAATGCATACATTTGTCTCATACACTATGGGGATGGTGAGAA
GAGATATATTTTACATCCCAGAGGGGCTATAATTGGAGATACCATTGTTTCTGGTACAGA
AGTTCCTATAAAAATGGGAAATGCCCTACCTTTGAGTGCGGTTTGAACTATTGATTTACG
TAATTGGAAATAACCAATTAGGTTTACGACGAAACCTATAAATCGATCACTGATCCAATT
TGAGTACCTCTGCAGGATAGACCTCAACAGAAAACTGAAGAGTAACGGCAGCAAGTGAT
TGAGTTCAGTAGTTCCTCATATAAAATTATTGACTCTAGAGATATAGTAATATGGAGAAG
ACAAAATTGTTTCAAGCACCGACAGAACCGGAAGCGCCCCTTCTTTCAAAGATAGGAGG
ACGGGTTATTCACATTTCATTTGATGGTCAGAGGCGAATTGAAAGTTAAGCAGTGGGAAT
TCTAAAGATTCCCCGGGGGAAAAATAGAGATGTCTCCTACGTTACCCATAATATGTGGAA
GTATCGACGTAATTTCATAGAGTCATTCGGTCTGAATGCTACATGAAGAACATAAGCCAG
ATGACGGAACGGGAAGACCCAGGATGTAGAAGATCATAACATGAGTGATTCGGCAGATT
TGGATTCATATATATATCCACCCATGTGGTACTTCATTCTACGATATATATAAGATCCATA
TGTATAGATATCATCATCTACATCCAGAAAGCCGTATGCTTTGGAAGAAGCTTGTACAGT
TTGGGAAGGGGTTTTGATTGATCAAAAGAAGAATCTACTTCAACCGATATGCCCTTAGGC
ACGGCCATACATAACATAGAAATCACACTTGGAAAGGGTGGACAATTAGCTAGAGCAGC
GGGTGCTGTAGCGAAACTGATTGCAAAAGAGGGGAAATCGGCCACATTAAAATTACCTT
CTGGGGAGGTCCGTTTGATATCCAAAAACTGCTCAGCAACAGTCGGACAAGTGGGGAAT
GTTGGGGTGAACCAGAAAAGTTTGGGTAGAGCCGGATCTAAGCGTTGGCTAGGTAAGCG
TCCTGTAGTAAGAGGAGTAGTTATGAACCCTGTAGACCATCCCCATGGGGGTGGTGAAG
GGAGAGCCCCAATTGGTAGAAAAAAACCCACAACCCCTTGGGGTTATCCTGCACTTGGA
AGAAGAAGTAGAAAAAGGAATAAATATAGTGATAATTTTATTCTTCGTCGCCGTAGTAA
ATAG
Withania 18S, Accession number NC_047245.1
SEQ ID NO: 57
GAAGGTCACGGCGAGACGAGCCGTTTATCATTACGATAGGTGTCAAGTGGAAGTGCAGT
GATGTATGCAGCTGAGGCATCCTAACAGACCGGTAGACTTGAAC
Withania 18S, Accession number NC_047245.1
SEQ ID NO: 58
TATTCTGGTGTCCTAGGCGTAGAGGAACCACACCAATCCATCCCGAACTTGGTGGTTAAA
CTCTACTGCGGTGACGATACTGTAGGGGAGGTCCTGCGGAAAAATAGCTCGACGCCAGG
AT
Withania 18S, Accession number NC_047245.1
SEQ ID NO: 59
TCTCATGGAGAGTTCGATCCTGGCTCAGGATGAACGCTGGCGGCATGCTTAACACATGCA
AGTCGGACGGGAAACACGGGAAACCGTGTTTCCAGTGGCGGACGGGTGAGTAACGCGTA
AGAACCTGCCCTTGGGAGGGGAACAACAGCTGGAAACGGCTGCTAATACCCCGTAGGCT
GAGGAGCAAAAGGAGGAATCCGCCCGAGGAGGGGCTCGCGTCTGATTAGCTAGTTGGTG
AGGCAATAGCTTACCAAGGCGATGATCAGTAGCTGGTCCGAGAGGATGATCAGCCACAC
TGGGACTGAGACACGGCCCAGACTCCTACGGGAGGCAGCAGTGGGGAATTTTCCGCAAT
GGGCGAAAGCCTGACGGAGCAATGCCGCGTGGAGGTAGAAGGCCCACGGGTCGTGAAC
TTCTTTTCCCGGAGAAGAAGCAATGACGGTATCTGGGGAATAAGCATCGGCTAACTCTGT
GCCAGCAGCCGCGGTAATACAGAGGATGCAAGCGTTATCCGGAATGATTGGGCGTAAAG
CGTCTGTAGGTGGCTTTTTAAGTCCGCCGTCAAATCCCAGGGCTCAACCCTGGACAGGCG
GTGGAAACTACCAAGCTGGAGTACGGTAGGGGCAGAGGGAATTTCCGGTGGAGCGGTGA
AATGCGTAGAGATCGGAAAGAACACCAACGGCGAAAGCACTCTGCTGGGCCGACACTGA
CACTGAGAGACGAAAGCTAGGGGAGCGAATGGGATTAGATACCCCAGTAGTCCTAGCCG
TAAACGATGGATACTAGGCGCTGTGCGTATCGACCCGTGCAGTGCTGTAGCTAACGCGTT
AAGTATCCCGCCTGGGGAGTACGTTCGCAAGAATGAAACTCAAAGGAATTGACGGGGGC
CCGCACAAGCGGTGGAGCATGTGGTTTAATTCGATGCAAAGCGAAGAACCTTACCAGGG
CTTGACATGCCGCGAATCCTCTTGAAAGAGAGGGGTGCCTTCGGGAACGCGGACACAGG
TGGTGCATGGCTGTCGTCAGCTCGTGCCGTAAGGTGTTGGGTTAAGTCCCGCAACGAGCG
CAACCCTCGTGTTTAGTTGCCATCGTTGAGTTTGGAACCCTGAACAGACTGCCGGTGATA
AGCCGGAGGAAGGTGAGGATGACGTCAAGTCATCATGCCCCTTATGCCCTGGGCGACAC
ACGTGCTACAATGGCCGGGACAAAGGGTCGCGATCCCGCGAGGGTGAGCTAACCCCAAA
AACCCGTCCTCAGTTCGGATTGCAGGCTGCAACTCGCCTGCATGAAGCCGGAATCGCTAG
TAATCGCCGGTCAGCCATACGGCGGTGAATTCGTTCCCGGGCCTTGTACACACCGCCCGT
CACACTATGGGAGCTGGCCATGCCCGAAGTCGTTACCTTAACCGCAAGGAGGGGGATGC
CGAAGGCAGGGCTAGTGACTGGAGTGAAGTCGTAACAAGGTAGCCGTACTGGAAGGTGC
GGCTGGAT

In some embodiments, the postbiotic composition comprises at least one compound produced by any of the following plant genera or species: the Astragalus family, the Solanaceae or nightshade family, the Sambucus L. genus, and/or the Lens orientalis or Lens culinaris family. In some embodiments, the postbiotic composition comprises Withanolides (e.g., Withaferin A); the Withanolides can be produced by ashwaganda root. In some embodiments, the postbiotic composition comprises at least one of the following compounds, that can be produced by elderberry: Anthocyanins (e.g., cyanidin-3-glucoside; e.g., measured using a pH-Differential method); Anthocyanins (e.g., cyanidin-3-glucoside; e.g., measured using HPLC); Polyphenols (e.g., catechin; e.g., measured using Folin-Ciocalteu reagent); and/or Polyphenols (e.g., expressed as gallic acid equivalent; e.g., measured using Folin-Ciocalteu reagent).

In some embodiments, the microorganism is Bifidobacterium lactis (also referred to as Bifidobacterium animalis subsp. lactis), e.g., strain DSM 10140 (see e.g., NCBI Reference Sequence: NC_012815.1 for an exemplary B. lactis genome sequence). In some embodiments, the microorganism is B. lactis strain BLC1− (Centro sperimentale del Latte (CSL)/SACCO). In some embodiments, the microorganism is B. lactis strain PBP1418518. In some embodiments, a nucleic acid primer for 16S sequencing of B. lactis comprises TGGAGGGTTCGATTCTGGCTCAGGATGAACGCTG (SEQ ID NO: 1). In some embodiments, the microorganism comprises a nucleic acid sequence (e.g., 16S sequence) comprising one of SEQ ID NO: 14-16 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 14-16 or a fragment thereof.

In some embodiments, the microorganism is Bifidobacterium breve, e.g., strain ATCC 15700 (see e.g., RefSeq: NZ_CP006712.1 for an exemplary B. breve genome sequence). In some embodiments, the microorganism is B. breve strain Bbr8 (CSL/SACCO). In some embodiments, the microorganism is B. breve strain PBP2741300. In some embodiments, a nucleic acid primer for 16S sequencing of B. breve comprises CCGGATGCTCCATCACAC (SEQ ID NO: 2) or ACAAAGTGCCTTGCTCCCT (SEQ ID NO: 3). In some embodiments, the microorganism comprises a nucleic acid sequence (e.g., 16S sequence) comprising one of SEQ ID NO: 17-20 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 17-20 or a fragment thereof. For further non-limiting examples of 16S rRNA-gene-targeted Bifidobacteria-specific primers, see e.g., Matsuki et al., Appl Environ Microbiol. 2004 January; 70(1): 167-173 (see e.g., Table 1 of Matsuki 2004), the contents of which are incorporated herein by reference in their entirety.

In some embodiments, the microorganism is Bifidobacterium infantis (also referred to as Bifidobacterium longum subsp. infantis) e.g., strain ATCC 15697 (see e.g., RefSeq: NC_015052.1 for an exemplary B. infantis genome sequence). In some embodiments, the microorganism is B. infantis strain SP 37 (CSL/SACCO). In some embodiments, the microorganism is B. infantis strain PBP234451. In some embodiments, a nucleic acid primer for 16S sequencing of B. infantis comprises TTCCAGTTGATCGCATGGTC (SEQ ID NO: 4) or GGAAACCCCATCTCTGGGAT (SEQ ID NO: 5). In some embodiments, the microorganism comprises a nucleic acid sequence (e.g., 16S sequence) comprising one of SEQ ID NO: 21-26 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 21-26 or a fragment thereof.

In some embodiments, the microorganism is Lactobacillus plantarum (also referred to as Lactiplantibacillus plantarum), e.g., strain Korean Agricultural Culture Collection (KACC) 11451 (see e.g., RefSeq: NZ_CP030105.1 for an exemplary L. plantarum genome sequence). In some embodiments, a nucleic acid primer for sequencing of L. plantarum comprises GCTGGCAATGCCATCGTGCT (SEQ ID NO: 6) or TCTCAACGGTTGCTGTATCG (SEQ ID NO: 7). In some embodiments, the microorganism comprises a nucleic acid sequence (e.g., 16S sequence) comprising one of SEQ ID NO: 27-32 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 27-32 or a fragment thereof. For further non-limiting examples of 16S rRNA-gene-targeted Lactobacillus-specific primers, see e.g., Kim et al., BMC Microbiology volume 20, Article number: 96 (2020) (see e.g., Table 1 of Kim 2020), the contents of which are incorporated herein by reference in their entirety.

In some embodiments, the microorganism is Lactobacillus acidophilus, e.g., strain KACC 12419 (see e.g., RefSeq: NC_021181.2 for an exemplary L. acidophilus genome sequence). In some embodiments, a nucleic acid primer for 16S-23S region sequencing of L. acidophilus comprises CCTTTCTAAGGAAGCGAAGGAT (SEQ ID NO: 8) or ACGCTTGGTATTCCAAATCGC (SEQ ID NO: 9). In some embodiments, the microorganism comprises a nucleic acid sequence (e.g., 16S sequence) comprising one of SEQ ID NO: 33-39 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 33-39 or a fragment thereof.

In some embodiments, the microorganism is Lactobacillus rhamnosus (also referred to as Lacticaseibacillus rhamnosus), e.g., strain Korean Collection for Type Cultures (KCTC) 3237 (see e.g., RefSeq: NZ_CP086326.1, NZ_LR134331.1, or ASM284801v1 for an exemplary L. rhamnosus genome sequence). In some embodiments, the microorganism is L. rhamnosus strain CRL1505 (CSL/SACCO). In some embodiments, the microorganism is L. rhamnosus strain PBP4542118. In some embodiments, a nucleic acid primer for 16S-23S region sequencing of L. rhamnosus comprises GCCGATCGTTGACGTTAGTTGG (SEQ ID NO: 10) or CAGCGGTTATGCGATGCGAAT (SEQ ID NO: 11). In some embodiments, the microorganism comprises a nucleic acid sequence (e.g., 16S sequence) comprising one of SEQ ID NO: 40-43 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 40-43 or a fragment thereof.

In some embodiments, the microorganism is Lactobacillus paracasei (also referred to as Lacticaseibacillus paracasei), e.g., strain KACC 12361 (see e.g., RefSeq: NC_014334.2 for an exemplary L. paracasei genome sequence). In some embodiments, the microorganism is L. paracasei strain IMC502 (CSL/SACCO). In some embodiments, the microorganism is L. paracasei strain PBP5197148. In some embodiments, a nucleic acid primer for sequencing of L. paracasei comprises CAATGCCGTGGTTGTTGGAA (SEQ ID NO: 12) or GCCAATCACCGCATTAATCG (SEQ ID NO: 13). In some embodiments, the microorganism comprises a nucleic acid sequence (e.g., 16S sequence) comprising one of SEQ ID NO: 45-48 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 45-48.

In some embodiments, the microorganism is Lactobacillus casei, e.g., strain BGP 93 (CSL/SACCO) (see e.g., RefSeq: NZ_AP012544.1 or CP017065 for exemplary L. casei genome sequences; see e.g., Kang et al., Front Immunol. 2017; 8: 413, the contents of which are incorporated herein by reference in their entirety). In some embodiments, the microorganism is L. casei strain PBP4157051. In some embodiments, the microorganism is L. casei strain DSM 20011, JCM 1134, ATCC 393, or LC5.In some embodiments, the microorganism comprises a nucleic acid sequence (e.g., 16S sequence) comprising one of SEQ ID NO: 49-52 or a nucleic acid sequence that is at least 80%, at least 85%, at least 90%, at least 91%, at least 92%, at least 93%, at least 94%, at least 95%, at least 96%, at least 97%, at least 98%, at least 99%, or at least 99.5% or more identical to one of SEQ ID NO: 49-52.

In some embodiments of any of the aspects, the bacterial strain is selected from Table 3. While specific strains of bacteria are named herein, other strains of these species are contemplated to perform in a similar manner. As a non-limiting example, to determine whether any given strain of a species is effective, the strain can be substituted for another strain of the same species in the fermentation process of the fermentation substrate composition to prepare the postbiotic composition. The postbiotic composition prepared using the substituted strain can be tested for efficacy compared to the postbiotic composition prepared using the original strain. Non-limiting examples of tests for efficacy include cell or animal models or human clinical testing of diseases and disorders such as dysbiosis, a dysbiosis-causing medication or medical treatment, antibiotic treatment, chemotherapy treatment, cancer immunotherapy, or intestinal mucositis.

TABLE 3
Exemplary Bacterial Strains (sequence GENBANK accession
number and location of gene of interest, e.g., 16S)
Bifidobacterium animalis (Bifidobacterium animalis subsp. lactis)

| | | |
|---|---|---|
| NHMR02000002.447733-450831 | CP007522.1774581-1777619 | CP003039.1862400-1865487 |
| NHMR02000002.456838-459936 | CP007755.1854987-1858079 | CP002915.1770950-1774006 |
| NHMR02000002.357105-360203 | GQ340908.1-511 | CP003941.1894164-1897251 |
| JGYM01000004.99960-102996 | CP022724.426063-429157 | CP002915.1867126-1870182 |
| ABOT01000014.1-2831 | GQ340904.1-481 | CP003039.1871099-1874186 |
| PHUS01000023.178-932 | GQ340905.1-530 | CP009045.1862414-1865509 |
| AHGW01000005.465993-469070 | CP001853.463423-466479 | CP001892.1867944-1871070 |
| AHGW01000005.171115-174192 | CP015407.798614-801712 | CP003497.1471384-1474509 |
| PESQ01000015.402-3312 | CP003497.1871201-1874327 | CP003498.1471389-1474515 |
| PESQ01000044.176-943 | CP003498.1871423-1874549 | CP001892.1771715-1774841 |
| PHUS01000019.2351-4620 | CP031703.292508-295603 | CP003497.1766264-1769390 |
| PESQ01000016.2351-4620 | CP004053.1766069-1769153 | CP003498.1766484-1769610 |
| QDIR01000003.165853-168948 | CP001606.1471265-1474391 | CP002567.1484779-1487856 |
| QDIV01000030.239-3334 | CP001853.168853-171909 | CP001515.1871313-1874439 |
| QDIP01000008.4098-7193 | CP007522.1471327-1474424 | CP004053.1471267-1474351 |
| QDIK01000002.90881-93976 | CP015407.1251722-1254820 | CP001892.1476835-1479961 |
| CBUQ010000001.199-3287 | CP003039.1766112-1769199 | CP007522.1879511-1882543 |
| CBUQ010000003.88687-91877 | CP007522.1870805-1873900 | CP007522.1765921-1769016 |
| QDIU01000009.2351-5446 | CP007755.1863694-1866786 | CP031703.301218-304313 |
| CBWX010000074.246-3332 | CP003941.1794711-1797800 | CP017098.1876987-1880082 |
| QDIO01000050.2336-5431 | CP003497.1862491-1865617 | CP002915.1875836-1878891 |
| AWFQ01000007.2552-5646 | CP003498.1862713-1865839 | CP009045.1471213-1474308 |
| QDIN01000004.165858-168953 | CP022724.34874-37967 | CP017098.1868277-1871372 |
| MLZL01000021.2277-5365 | CP001853.559666-562722 | CP009045.1871124-1874219 |
| QDIZ01000011.2345-5440 | CP003941.1885457-1888543 | CP017098.1771957-1775051 |
| AWFM01000016.239-3334 | CP001515.1471333-1474459 | CP017098.1476864-1479959 |
| QDIZ01000052.1-359 | CP001606.1766146-1769272 | CP022724.434771-437864 |
| AWFP01000017.240-3334 | CP001213.902284-905348 | CP010433.1891255-1894380 |
| QDIL01000005.165858-168953 | CP002567.1856200-1859280 | CP022724.329746-332839 |
| AWFL01000014.239-3334 | CP015407.889242-892341 | CP010433.1476837-1479963 |
| QDIT01000009.239-3334 | CP001892.1876654-1879780 | CP028460.1764167-1766624 |
| AWFN01000009.252-3349 | CP001213.1080287-1083350 | CP010433.1882545-1885671 |
| AWFO01000013.3194-6288 | CP001606.1862373-1865501 | CP010433.1876767-1879938 |
| NIGR01000004.165695-168757 | CP002567.1764176-1767254 | CP028460.1856191-1858647 |
| CP001853.568367-571423 | CP001515.1766374-1769500 | CP010433.1868040-1871166 |
| AWFK01000023.2268-5362 | CP001606.1871087-1874213 | CP031154.1476721-1479816 |
| CP015407.789509-792607 | CP003039.1471229-1474316 | CP031703.692417-695511 |
| NIGQ01000005.165695-168757 | CP002915.1476216-1479272 | CP010433.1771720-1774845 |
| FTRP01000009.342-3322 | CP004053.1862387-1865471 | CP031154.1771603-1774697 |
| CP004053.1871094-1874178 | CP003941.1493276-1496365 | CP028460.1484769-1487229 |
| QSUF01000014.3937-7032 | CP002567.1864911-1867986 | CP028460.1864899-1867356 |
| FTRE01000009.342-3322 | CP001515.1862603-1865729 | CP031703.397539-400632 |
| QSSU01000013.5254-8349 | CP009045.1766093-1769188 | CP031154.1867923-1871018 |

Bifidobacterium breve

| | | |
|---|---|---|
| PKGQ01000008.2197-5277 | ACCG02000014.1-1799 | CP010413.2274068-2277141 |
| JGYR01000006.88316-91378 | BCXQ01000015.45340-48402 | CP006714.2222715-2225775 |
| NAPZ01000021.449-2485 | BCXO01000053.1982-5044 | CP021387.1477845-1480905 |
| MWVR01000023.1-1394 | BCXX01000043.1786-4849 | CP021388.2276729-2279790 |
| AVQE01000009.123432-126512 | BCXP01000010.8139-11201 | CP021393.2276773-2279833 |
| NAQG01000032.537-3600 | BCXN01000046.6481-9543 | CP021553.2369819-2372879 |
| AVQA01000016.2002-5083 | BCXS01000057.141-3098 | CP006711.1481840-1484903 |
| AVQB01000011.1889-4970 | QDIS01000011.267-3347 | CP006715.1558123-1561185 |
| NAQI01000022.2323-5385 | BCXX01000023.121613-124571 | CP006715.2247131-2250193 |
| BCXM01000027.261696-264759 | BCXP01000010.1891-4953 | CP006716.1553646-1556709 |
| BCXS01000055.149-3211 | BCXL01000101.2196-5259 | CP010413.552370-555443 |
| AVQD01000023.1896-4976 | FNFW01000004.174218-177270 | CP006716.2194146-2197209 |
| MWVR01000020.1-1747 | AWUG01000001.567561-570622 | CP006716.2200394-2203457 |
| MWVR01000021.1-1173 | AWFR01000017.262-3342 | CP019596.1613108-1616188 |
| AFVV01000031.2196-5256 | AWFT01000016.1989-5067 | CP023192.1533021-1536082 |
| AFXX01000047.2198-5261 | BCXL01000027.260918-263876 | CP023195.1533048-1536109 |
| MWVR01000024.442-1217 | AWFS01000022.81-3161 | CP023197.1533046-1536107 |
| AVQC01000028.1785-4865 | AWFU01000011.270-3350 | CP021385.2197977-2201037 |
| JDUD01000016.2023-5085 | AWSX01000035.280-3342 | CP021392.2298990-2302051 |
| BCXX01000044.124990-128053 | AWFV01000032.137-3217 | CP006713.2272548-2275608 |
| BCXL01000099.373-3331 | FNFW01000022.6643-9695 | CP021553.1696481-1699541 |
| BCXT01000047.1981-5043 | LFII01000018.29-1478 | CP021556.2310591-2313651 |
| BCXQ01000006.2096-5158 | CP006712.2192941-2196001 | CP021555.2195706-2198766 |
| BCXM01000015.23265-26223 | ATCB01000001.1-3062 | CP021559.1635063-1638123 |
| BCXN01000054.47799-50861 | NIGS01000008.116386-119425 | CP006713.1590347-1593407 |
| BCXW01000037.1900-4963 | NJOG01000035.1-1163 | CP021385.1533136-1536195 |
| BCXS01000053.1786-4848 | FTRL01000008.1892-4954 | CP021392.1609244-1612305 |
| BCXU01000019.382-3339 | PNHM01000012.2778-5858 | CP021387.2160069-2163129 |
| BCXM01000024.373-3331 | LRPP01000035.1-1152 | CP021388.2282979-2286039 |
| BCXL01000068.6717-9780 | BBAO01000074.2191-5245 | CP021393.2283024-2286085 |
| BCXS01000009.274-3336 | LFII01000019.1-1669 | CP021554.1588698-1591756 |
| BCXP01000019.12059-15016 | NJOA01000035.1-3074 | CP021384.1556548-1559608 |
| BCXM01000025.373-3331 | NJOG01000036.263-1157 | CP021389.2419632-2422693 |
| BCXR01000051.221-3283 | ATCB01000003.457-3519 | CP021389.1749967-1753028 |
| BCXT01000057.145412-148369 | CP021555.2201953-2205013 | CP021394.2317813-2320873 |
| BCXT01000059.2153-5215 | CP021559.2380434-2383492 | CP021386.1505976-1509035 |
| BCXV01000028.1895-4957 | FTRK01000007.382-3340 | CP021392.2305241-2308302 |
| BCXQ01000006.8346-11408 | CP000303.1610117-1613177 | CP010413.2267833-2270906 |
| BCXN01000037.1428-4385 | CP023198.2328111-2331171 | CP023198.1646572-1649632 |
| MWVR01000018.205053-205443 | GQ340900.1-906 | CP021386.2225223-2228283 |
| BCXM01000024.6618-9576 | NJOF01000022.13-1230 | CP021386.2231459-2234518 |
| BCXO01000043.127349-130306 | NJOB01000033.1-604 | CP023194.2197821-2200881 |
| BCXP01000024.135929-138886 | NJOD01000026.1-2142 | CP021390.2293765-2296825 |
| BCXU01000018.382-3339 | CP002743.683712-686759 | CP021557.2185059-2188120 |
| BCXV01000029.1895-4957 | AP012324.1556553-1559613 | CP021384.2174804-2177862 |
| BCXR01000050.377-3334 | ATCB01000002.688345-691407 | CP021389.2425880-2428941 |
| BCXO01000054.374-3331 | ATCB01000002.1-3062 | CP023192.2197852-2200913 |
| BCXL01000100.6614-9572 | AP012324.2174820-2177874 | CP021390.1610175-1613235 |
| BCXU01000041.48330-51392 | NJOB01000035.1-1819 | CP021557.1556372-1559430 |
| BCXW01000036.1900-4963 | CP000303.2315331-2318391 | CP021391.2314226-2317286 |
| BCXQ01000007.2096-5158 | CP002743.2232959-2236013 | CP021552.2328108-2331168 |
| BCXL01000100.373-3331 | CP021555.1592874-1595934 | CP021558.2507190-2510251 |
| BCXV01000028.8130-11192 | CP021556.1664886-1667946 | CP021391.2307975-2311036 |
| BCXO01000051.1982-5044 | CP023195.2197984-2201045 | CP021552.1646573-1649633 |
| BCXQ01000009.383-3340 | CP023197.2197883-2200944 | CP021558.2500114-2503175 |
| BCXO01000055.374-3331 | CP006714.1506536-1509596 | CP021391.1667860-1670921 |
| BCXP01000011.373-3330 | CP002743.2226712-2229765 | CP021558.1782121-1785182 |
| BCXU01000018.6626-9583 | CP019596.2323125-2326205 | CP023196.1533054-1536114 |
| BCXW01000038.383-3341 | CP019596.2329373-2332453 | CP023196.2197930-2200991 |
| BCXO01000053.8219-11281 | CP021387.2166302-2169362 | CP023199.2386695-2389754 |
| BCXS01000056.149-3211 | CP021394.1627964-1631024 | CP021390.2302560-2305620 |
| BCXT01000048.382-3339 | CP021554.2216991-2220051 | CP021557.2191312-2194373 |
| MNLA01000015.343-2254 | CP006712.1505194-1508255 | CP023199.1635080-1638139 |
| BCXR01000046.1634-4591 | CP006711.2148819-2151882 | CP023194.1532997-1536055 |
| BCXV01000039.127171-130233 | CP021388.1573876-1576936 | CP023199.2380446-2383504 |
| PNHM01000011.6787-9867 | CP021393.1566630-1569691 | CP023193.2197778-2200839 |
| BCXP01000013.373-3330 | CP021553.2363571-2366631 | CP023193.1533043-1536104 |
| AWUF01000002.3346-6408 | CP021556.2316841-2319901 | |

Bifidobacterium longum infantis

| | | |
|---|---|---|
| CCWT01000056.14-3060 | NAQJ01000023.2566-5629 | LN824140.1109875-1112929 |
| CCWS01000051.14-3060 | BCYF01000104.332-3393 | AP010890.2121545-2124610 |
| CCWN01000056.14-3060 | CCWP01000060.6461-9510 | AP010889.2757707-2760771 |
| BCYG01000058.1-1181 | BCYG01000043.56936-58116 | AP010889.962798-965862 |
| BCYF01000055.67-3051 | BCYG01000038.1-1181 | AP010890.2290976-2294041 |
| JNWB01000028.2168-5223 | BCYG01000026.1-1307 | LN824140.1661400-1664451 |
| CCWR01000050.14-3060 | BCYG01000031.1-1181 | LN824140.1858333-1861387 |
| CCWQ01000054.189-3238 | UAQL01000033.607-3670 | CP010411.1734200-1737284 |
| CCWR01000050.6175-9224 | BCYG01000032.375-3337 | LN824140.1852153-1855204 |
| ABQQ01000041.2175-5235 | BCYG01000030.1-1179 | AP010889.2539588-2542652 |
| CCWU01000050.6175-9224 | BCYF01000054.143-3206 | AP010890.2299614-2302679 |
| JDTT01000098.92-3053 | BCYF01000087.106436-109489 | AP010889.2751543-2754607 |
| CCWU01000050.14-3060 | UAQL01000014.719245-722308 | CP001095.2755591-2758448 |
| CCWP01000060.297-3346 | MOAF01000031.39089-42139 | CP001095.963165-966022 |
| UAQL01000032.205924-207042 | GQ340901.1-895 | CP001095.2761755-2764612 |
| CCWO01000067.6427-9476 | CP001095.2539820-2542677 | CP010411.657870-660934 |
| CCWO01000067.263-3312 | AP010890.1577985-1581050 | CP010411.454596-457660 |

Lactobacillus acidophilus

| | | |
|---|---|---|
| UGNR01000002.1-2298 | MNPO01000001.413502-416405 | AB092633.206-665 |
| UGNR01000002.916171-919076 | MNPO01000002.354985-357888 | AB092642.216-672 |
| UGNR01000002.924101-927006 | CBLR010000038.101-3004 | KC161273.1-427 |
| UGNR01000002.520355-523260 | CBLP010000007.101-3004 | CP000033.415728-418779 |
| UGNR01000001.62722-65627 | NXEY01000033.151-1480 | RBHY01000021.96-3006 |
| UGNR01000001.1-2299 | CBLP010000014.64525-67428 | CP000033.1627955-1631003 |
| UYIX01000011.1546621-1549539 | CBLT010000017.1006-3909 | JN368427.1-745 |
| UYIX01000009.1089868-1092786 | NXEY01000006.152872-155782 | AB092631.209-696 |
| UYIX01000009.346942-349860 | CBLT010000012.64463-67366 | AJ620360.1-2891 |
| UYIX01000011.725394-728312 | CBLS010000044.189-3092 | MG752953.1-1244 |
| UYIX01000011.238841-241759 | CBLT010000003.101-3004 | AYUB01000062.293-3081 |
| BDHM01000014.293-3081 | CBLQ010000055.1-2779 | CP005926.415727-418808 |
| LWSH01000065.1-1873 | NXEY01000032.1-1509 | CP000033.1630739-1632556 |
| LWSH01000069.1-836 | CBLT010000019.101-3004 | AB092630.209-696 |
| QAHL01000020.187-3097 | AF182726.210-661 | CP020620.1281087-1283997 |
| QAHQ01000047.1-843 | AZCS01000022.101-3004 | CP017062.1627228-1630138 |
| QAHQ01000051.1-353 | ACHN01000040.101-3004 | CP005926.1627499-1630579 |
| QAHR01000025.96-3006 | BALR01000023.292-3078 | CP017062.436000-438910 |
| QAHQ01000052.1-531 | CP005926.60960-64040 | CP017062.415780-418690 |
| QAHM01000023.96-3006 | JRUT01000009.96-3006 | CP005926.435948-439028 |
| QAHS01000020.187-3097 | NIGU01000015.195-3091 | CP017062.61020-63930 |
| QAHQ01000049.1-664 | AYUB01000055.880-3700 | CP020620.70406-73316 |
| QAZY01000020.187-3097 | AB092634.225-709 | CP020620.1706056-1708966 |
| QAHT01000020.187-3097 | CP000033.435949-438999 | CP010432.416090-418995 |
| QAHN01000020.187-3097 | CP020620.90625-93535 | CP010432.61327-64232 |
| QAHP01000018.187-3097 | AYUA01000140.1-1685 | CP010432.436310-439215 |
| QAHQ01000048.1-807 | CP000033.60957-64007 | CP022449.729894-732804 |
| QAHQ01000059.1-309 | AB092632.210-684 | CP010432.1628306-1631211 |
| BCVR01000014.293-3081 | AJ438156.210-689 | CP022449.375134-378044 |
| QAHO01000020.187-3097 | AB092620.209-697 | CP022449.1942103-1945013 |
| QAZX01000020.187-3097 | KC161284.1-671 | CP022449.750111-753021 |
| CBLP010000002.101-3004 | | |

Lactobacillus casei

| | | |
|---|---|---|
| LCUN01000026.1-2441 | MODS01000034.2128-5047 | MODT01000028.5-2924 |
| LDEJ01000075.1-702 | MODS01000025.157-3076 | AY221481.1-556 |
| JPKN02000019.29079-31998 | KC161274.1-786 | AJ542550.1-353 |
| LDEJ01000078.1-702 | FM177140.1986676-1989475 | AF182729.225-685 |
| LCUN01000018.1-382 | JXNV01000061.128-3043 | KC161276.1-856 |
| AFYS01000097.827-1232 | FM177140.260644-263559 | AY221474.1-556 |
| QAZD01000125.336-1720 | AJ542555.1-352 | AP012544.651131-654050 |
| LNQD01000056.793-3592 | KC161263.1-715 | AP012544.2478882-2481801 |
| JPKN02000021.33624-36543 | KC161264.1-718 | AJ542561.1-340 |
| AFYS01000105.580-931 | KC161265.1-688 | AP012544.711187-714106 |
| AFYK01000008.2720-5637 | AY221471.1-556 | HE970764.1984824-1987739 |
| LDEJ01000001.1-1400 | FM177140.2639820-2642619 | AF098107.1-1287 |
| QAZE01000108.20-2939 | LS991421.1772971-1775770 | CP017065.274780-277699 |
| AFYM01000133.1-424 | AY221480.1-556 | LS991421.749481-752396 |
| LDEJ01000068.1-851 | AP012544.211890-214809 | AP012544.1785700-1788619 |
| JPKN02000001.159860-162779 | AY221472.1-556 | HE970764.908184-911099 |
| QAZD01000113.41-2875 | AY221482.1-556 | AB092640.223-705 |
| AFYS01000021.20527-23444 | FM177140.909841-912756 | AY221478.1-556 |
| AFYK01000016.1-372 | BD107104.2-2751 | HE970764.260608-263523 |
| LTDP01000051.11022-13937 | AY112676.1-557 | HE970764.886510-889425 |
| AFYM01000079.20977-23894 | KC161277.1-735 | CP006690.780180-783097 |
| AFYS01000042.55792-56280 | AY221475.1-556 | CP017065.778359-781278 |
| FXZN01000024.423-3222 | AY221479.1-556 | LS991421.2481497-2484296 |
| MPOP01000089.2059-4978 | AJ542556.1-351 | AY112675.1-560 |
| FM177140.888163-891078 | AB109025.226-704 | CP017065.2602394-2605313 |
| JUPZ01000178.35-2950 | LS991421.298439-301354 | CP017065.1873810-1876729 |
| JUQW01000199.64-2863 | AY221476.1-556 | CP006690.278340-281257 |
| BALS01000124.22-2821 | KC161266.1-713 | CP006690.799547-802464 |
| AZOE01000162.18-1091 | LS991421.769905-772820 | CP006690.1836273-1839190 |
| AWZQ01000366.2-1315 | HE970764.2636323-2639238 | CP017065.801255-804174 |
| MODT01000024.157-3076 | AY221473.1-556 | CP006690.2469049-2471967 |
| AZCO01000094.128-3043 | AY221477.1-556 | |

Lactobacillus paracasei

| | | |
|---|---|---|
| JYBD01000071.128-3043 | ANLE01000030.125-3044 | CP013921.2761902-2764821 |
| QVHW01000051.5-2924 | ANKB01000105.125-3044 | CP001084.796507-799422 |
| AQPP01000001.260865-263770 | ANMI01000270.24-2941 | CP013921.515170-518089 |
| AQPP01000006.252371-255276 | ANJT01000005.1-1546 | CP017261.1492633-1495552 |
| PKQJ01000048.125-3044 | ANKI01000149.5-2924 | CP002616.260642-263557 |
| AQPP01000009.605644-608559 | ANKV01000065.111-2909 | CP014985.1600926-1603844 |
| AQPP01000009.600587-603502 | ANKY01000105.128-3044 | CP025582.1986706-1989608 |
| AQPP01000016.62953-65868 | ANLG01000694.230-3149 | CP002391.276730-279645 |
| RCFI01000003.1690584-1693503 | ANMN01000001.128-3043 | CP001084.1782359-1785274 |
| PKQJ01000093.1-396 | ANKH01000527.5-2924 | CP002618.2630785-2633584 |
| AFYO01000009.1-382 | ANJY01000202.5-2924 | CP016355.2612922-2615840 |
| AFYQ01000091.13157-13586 | ANKD01000024.5-2924 | CP025582.273208-276122 |
| MWVE01000057.125-3044 | BAYO01000006.111-2910 | CP002391.795807-798722 |
| AFYP01000020.875-3792 | ANMN01000130.126-3043 | CP001084.777140-780055 |
| AFYL01000005.8980-11897 | ANJV01000255.125-3044 | CP001084.2434661-2437576 |
| NOKL01000002.2119828-2122743 | QMJF01000007.337-3256 | CP002391.1862810-1865725 |
| AFYT01000013.7069-7489 | ANMK01000201.126-3043 | AP018392.1988835-1991740 |
| AFYL01000041.968-1425 | ANJX01000426.239-3158 | CP013921.2072431-2075350 |
| AFYT01000046.53948-54418 | MKFZ01000076.1572-4482 | CP002618.257941-260856 |
| AFYQ01000010.1-511 | ANJZ01000001.125-3043 | CP017261.2666438-2669357 |
| AFYU01000103.226-3143 | ANKJ01000077.5-2924 | CP005486.1966610-1969527 |
| NOKL01000002.2792646-2795561 | ANKF01000177.5-2924 | CP001084.278212-281127 |
| NOKL01000002.1414872-1417671 | JVDQ01000084.128-3043 | CP002616.2599898-2602697 |
| NOKL01000002.741022-743821 | CP014985.978915-981834 | CP002618.880145-883060 |
| NOKL01000002.2773205-2776120 | QMJV01000112.125-3044 | CP017716.799676-802595 |
| BAGT01000081.111-2910 | JVUB01000142.64-2863 | CP002616.1947764-1950563 |
| AFYO01000056.1-342 | ANKK01000016.125-3044 | AP018392.2671098-2673897 |
| AFYN01000039.173625-174028 | ANMH01000131.6-2923 | CP029686.647565-650484 |
| AFYP01000028.159237-159700 | ANKV01000091.6-2922 | CP017716.2497923-2500842 |
| AFYR01000129.582-954 | JVNI01000042.422-3221 | CP002391.2500295-2503210 |
| AFYP01000004.1-440 | JVUA01000226.344-3217 | CP013921.1586454-1589373 |
| AFYP01000018.168852-169235 | QMJW01000133.234-3153 | CP013921.1564702-1567621 |
| AFYN01000040.7582-10499 | CP005486.2687409-2690326 | CP032637.1657331-1660250 |
| ARNV02000013.382265-385064 | CP017716.819117-822036 | CP017261.403745-406664 |
| AFYT01000014.1-476 | AJ542563.1-370 | CP017261.426901-429820 |
| AFYR01000012.73045-75962 | JVNI01000043.420-3219 | CP016355.290838-293756 |
| RCFI01000003.2202534-2205453 | NCSO01000009.730-3645 | CP016355.953173-956092 |
| AFYO01000023.46683-49600 | JVUA01000259.1854-4727 | CP029686.2614715-2617632 |
| AFYQ01000064.46893-49810 | NCSN01000021.102454-105371 | CP016355.1970977-1973896 |
| ARNV02000003.606215-609130 | NCSP01000023.172796-175713 | CP012148.2629309-2632226 |
| ANKW01000115.123-2922 | JVDQ01000102.490-3289 | CP016355.972537-975456 |
| ANKY01000144.128-3029 | QMJP01000122.904-3823 | CP007122.112736-115655 |
| BDIT01000061.128-3041 | JVDQ01000020.2062-4977 | CP007122.2436073-2438992 |
| ANKM01000146.5-2924 | JVNI01000059.111-2910 | CP005486.935230-938147 |
| ARNV02000010.48774-51573 | AP012541.799669-802588 | CP005486.274272-277189 |
| MWVD01000103.124-3043 | AP012541.2608884-2611803 | CP007122.1978957-1981876 |
| AFYQ01000065.1-456 | CP000423.2501036-2503550 | CP012148.909974-912891 |
| AFYT01000008.879-3796 | MKFY01000031.329-3033 | CP007122.1957007-1959926 |
| NIGW01000035.14-2923 | AB109028.224-694 | CP007122.939212-942131 |
| ARNV02000001.261345-264260 | AP012541.258903-261822 | CP025582.2608203-2611002 |
| ARNV02000003.584537-587452 | CP000423.1825733-1828247 | CP026097.905657-908572 |
| LGIY01000002.246287-249206 | AB109026.225-712 | CP012148.1974186-1977103 |
| QXET01000145.1-779 | MKGA01000043.86-3001 | CP025582.905987-908902 |
| AUYM01000009.125-3044 | AP012541.819244-822163 | CP026097.1986397-1989301 |
| QXET01000132.1-1015 | AY221470.1-556 | CP029686.628199-631117 |
| NXET01000001.1486-4275 | CP025582.886623-889538 | CP012148.269317-272234 |
| NXES01000001.1009-3928 | MOAG01000022.1900-4819 | CP012148.890533-893450 |
| QXET01000102.5-2924 | CP002616.890255-893170 | CP026097.886295-889208 |
| ANLF01000280.5-2924 | CP014985.998357-1001276 | CP012187.245662-248581 |
| ANKL01000407.125-3044 | MBTZ01000129.5-2924 | CP032637.1051199-1054118 |
| AZGH01000016.20261-23176 | AF182724.225-685 | CP026097.273209-276124 |
| QRBH01000041.215-3130 | CP029686.1925852-1928771 | CP012187.2465034-2467953 |
| ANJS01000169.7-2926 | CP032637.3010302-3013221 | CP012187.1808296-1811214 |
| ANJU01000123.125-3044 | AYYJ01000181.135-2934 | CP012187.768777-771696 |
| BAYN01000045.111-2910 | CP017261.2119204-2122123 | CP029686.1209017-1211935 |
| NDXH01000075.1-1400 | CP005486.954596-957513 | CP026097.2607764-2610563 |
| ANML01000168.126-3043 | CP017716.285086-288005 | CP012187.744041-746960 |
| AP012541.1879414-1882333 | CP000423.261698-264212 | CP029546.2488232-2491151 |
| QXET01000222.1-329 | CP017716.1837722-1840641 | CP032637.2366308-2369227 |
| ANKC01000028.125-3044 | CP000423.825967-828481 | CP029536.598172-600969 |
| ANJW01000490.5-2924 | CP002391.776440-779355 | CP029536.1920349-1923263 |
| ANKW01000201.18-2935 | CP000423.847717-850231 | CP029546.743643-746562 |
| ANKA01000137.125-3044 | CP014985.2314829-2317748 | CP032637.1031834-1034753 |
| ANMM01000074.6-2923 | AP018392.948523-951438 | CP029536.2946091-2948890 |
| ANMJ01000090.126-3043 | CP002618.901823-904738 | CP029546.1822428-1825347 |
| ANJT01000004.1-590 | CP014985.2975376-2978295 | CP029536.1941358-1944271 |
| ANKE01000660.5-2924 | CP002618.1978650-1981449 | CP029546.763009-765928 |
| ACGY01000021.6-1348 | CP002616.911933-914848 | CP029536.1304492-1307407 |
| ANMH01000061.111-2910 | AP018392.929083-931997 | CP029546.249849-252768 |
| ANKG01000193.1-2871 | AP018392.289327-292242 | |

Lactobacillus plantarum

| | | |
|---|---|---|
| JSUW01000107.1916-4834 | MKDV01000039.140-3058 | CP017374.2849941-2852863 |
| JSUX01000107.203-3009 | QMJU01000098.188-3110 | CP017406.1550753-1553559 |
| QBKX01000050.290-3096 | QMJR01000080.100-3022 | CP018209.2494073-2496991 |
| QBKW01000066.290-3096 | AB092636.211-684 | CP017406.2263106-2266014 |
| JMEL01000116.1985-4901 | KC161272.1-681 | CP014780.2471956-2474878 |
| MJHI01000068.309-3115 | LKLZ01000013.1-1152 | CP017954.584052-586974 |
| MJAM01000014.49646-52452 | MKEB01000094.2021-4939 | CP021997.681976-684898 |
| MJHJ01000047.80-2998 | MKDZ01000034.329-3247 | CP019348.831360-834285 |
| MJHK01000031.202-3008 | CP018324.181357-184279 | CP018209.1662658-1665464 |
| MJHG01000067.1937-4855 | MKEG01000037.1916-4834 | CP019722.489693-492615 |
| MJHD01000007.284-3090 | LKHZ01000001.823379-826299 | CP016071.1529433-1532355 |
| MJHH01000017.1844-4762 | MKDY01000030.360-3166 | CP022294.2914570-2917492 |
| MCOL01000001.2093797-2096717 | QMJE01000030.17-2939 | CP023771.484016-486938 |
| LZXZ01000028.1-591 | MKDH01000022.1810-4728 | CP017954.1430807-1433729 |
| LZXZ01000020.332445-333232 | MKDK01000036.1925-4843 | CP004406.1983506-1986425 |
| MJHC01000023.290-3096 | FMBQ01000065.1925-4843 | CP017954.2027576-2030498 |
| LZXZ01000019.691030-691392 | AGRI01000003.162285-165204 | CP005942.2764119-2767037 |
| AVFJ02000024.24040-26961 | JHWA01000027.1404250-1407168 | CP011769.1835336-1838258 |
| AVFI01000045.1886-4808 | LUWG01000131.189-3109 | CP022373.995554-998476 |
| LZXZ01000014.1-1215 | MEGY01000029.77620-80426 | CP017066.2021423-2024345 |
| AVFI01000113.33084-33501 | MEGY01000065.140-3012 | CP004406.2408034-2410953 |
| LDEL01000022.1-1764 | LUWX01000124.189-3109 | CP004082.1359287-1362205 |
| NKCZ01000027.189-3109 | LUWY01000010.189-3109 | CP016071.683251-686173 |
| NQNQ01000042.2046-4968 | LUXJ01000104.189-3109 | CP020816.1134208-1137125 |
| LWKN01000138.137-3059 | LUHN01000063.1-579 | CP032460.1609394-1612316 |
| AVFJ02000001.133133-136055 | LUXG01000072.189-3109 | CP032648.2656491-2659413 |
| OKQT01000093.191-3107 | LUXB01000078.145-3065 | CP017363.1109529-1112451 |
| PEGW01000035.13-2935 | CP013149.1942992-1945914 | CP017374.478745-481667 |
| OKQP01000080.191-3107 | LUXF01000016.189-3109 | CP006033.1076904-1079823 |
| LMVD01000097.31-2951 | LUHN01000077.1-373 | CP012650.2306881-2309803 |
| OKQV01000099.191-3107 | MEGY01000071.103-2909 | CP018209.1275038-1277844 |
| MCOL01000001.511668-514588 | FJVL01000032.22170-24976 | CP017066.1153446-1156368 |
| QAHF01000047.1-538 | CP019348.1848495-1851417 | CP021086.1489538-1492343 |
| LDEL01000024.1-546 | AB092638.212-683 | CP018324.1034746-1037668 |
| LDEM01000002.712273-715070 | LKHZ01000002.1132871-1135751 | CP004082.733183-736101 |
| LNCP01000005.802632-805554 | AL935263.2015966-2018885 | CP011769.2322414-2325336 |
| AVAQ01000039.121-3043 | LKCO01000035.137-3059 | CP025586.1489914-1492832 |
| MCOL01000001.2575115-2578035 | LUWF01000065.189-3109 | CP025988.2500260-2503178 |
| LDEL01000006.153080-153991 | LUWU01000015.189-3109 | CP017363.503808-506730 |
| LNCP01000004.361-3283 | FMBS01000207.370-3176 | CP017066.501182-504103 |
| LNCP01000006.1-1923 | LKHZ01000002.693042-695962 | CP006033.515910-518829 |
| AVFJ02000055.9211-12133 | JXAX01000030.17-2939 | CP018209.811321-814127 |
| MCOL01000001.1218531-1221451 | AGRI01000001.299538-302457 | CP012650.691990-694912 |
| QAHH01000017.188-3110 | LUWN01000024.163-3083 | CP018324.1318125-1321047 |
| JPSU01000012.586575-589483 | LUXN01000006.3565-6485 | CP019722.1096963-1099885 |
| QAZQ01000017.188-3110 | LUXE01000014.138-3058 | CP021932.2526653-2529575 |
| JZSB01000005.47847-50765 | MKDX01000039.213-3135 | CP023728.2491914-2494836 |
| QAZS01000019.137-3059 | MKEE01000035.139-3057 | CP020816.2014188-2016994 |
| NQNI01000043.1-570 | MKED01000017.139-3057 | CP004406.2858596-2861515 |
| NKRH01000025.2095-5017 | JHWA01000003.78378-81183 | CP019722.1951442-1954364 |
| MCOL01000001.3037459-3040379 | LUWH01000063.73-2993 | CP021086.2105663-2108571 |
| JPSU01000004.1-2746 | MH624119.1-2922 | CP019722.2436175-2439097 |
| QOSF01000003.1913-4834 | MH624121.1-3706 | CP020861.1106044-1108966 |
| NQNE01000028.1-636 | CP013149.2887265-2890187 | CP012122.1453299-1456218 |
| LDEL01000025.1-547 | LUXI01000015.189-3109 | CP020861.481509-484431 |
| QAHG01000032.188-3110 | LUWQ01000054.189-3109 | CP012650.1181538-1184460 |
| FKLQ01000016.1-1699 | LUWL01000007.31-2951 | CP012343.1670520-1673442 |
| FKLQ01000030.313927-316733 | LUXK01000044.189-3109 | CP022294.1104008-1106930 |
| AXDQ01000020.32253-35171 | LUSM01000042.1-303 | CP018324.2530093-2533015 |
| NQNE01000011.420262-420613 | LUHN01000018.1-564 | CP021528.2697152-2700072 |
| NQNE01000022.1-2191 | AL935263.2941803-2944722 | CP010528.2511370-2514292 |
| LJDC01000068.1-401 | MEGY01000013.140-3058 | CP020816.489843-492761 |
| NQNI01000031.1-1892 | AGRI01000003.807476-810395 | CP010528.503295-506217 |
| AXDQ01000052.58-2976 | AGRI01000010.177928-180847 | CP025586.189392-192198 |
| JZSB01000011.16597-19403 | LUWP01000028.189-3109 | CP021501.499232-502152 |
| JZSB01000015.111505-114311 | LUXD01000007.189-3109 | CP021932.2061104-2064026 |
| QAHF01000044.135-2429 | PZPN01000092.188-3110 | CP022294.1961784-1964706 |
| QAZO01000030.188-3110 | CP013753.1412228-1415150 | CP022373.2575987-2578909 |
| QAHF01000052.1-346 | AB092637.212-701 | CP021086.644138-647056 |
| QAHE01000024.188-3110 | LUWW01000030.189-3109 | CP018324.1750277-1753199 |
| JPSU01000005.136384-139302 | LUXM01000017.1-1120 | CP020093.2911279-2914085 |
| NQNI01000042.188-571 | KC109451.1-680 | CP009236.491411-494329 |
| LJDC01000054.1-401 | CP010528.1171257-1174179 | CP021997.1145185-1148107 |
| LJDC01000069.1-401 | LUXL01000034.157471-160391 | CP020093.1976877-1979683 |
| QAZP01000028.188-3110 | JQAW01000005.102-3020 | CP020564.1058404-1061324 |
| LJDC01000047.1-583 | CP022373.3027547-3030469 | CP009236.2000194-2003112 |
| JZSB01000002.109373-112291 | LUXC01000022.189-3109 | CP021086.146212-149130 |
| QAZR01000025.188-3110 | NJPU01000011.189-3109 | CP022294.483653-486575 |
| PEGO01000039.462-3384 | LUXH01000090.189-3109 | CP025412.2415175-2418097 |
| PEBE01000031.13-2935 | LUWS01000007.189-3109 | CP025590.204603-207520 |
| JPSU01000011.22866-25672 | LRUO01000038.137-3059 | CP025988.1738374-1741292 |
| AXDQ01000131.12090-14998 | MEGY01000047.68816-71622 | CP023174.1290913-1293835 |
| AXDQ01000092.4233-7039 | JOJT01000024.163-3083 | CP021997.2635585-2638507 |
| PEGJ01000041.188-3110 | PZQK01000072.19-2941 | CP020861.1975117-1978039 |
| PEGS01000042.13-2935 | BD107240.10-2929 | CP020861.2901831-2904753 |
| AXDQ01000112.10152-12958 | CP023174.1837024-1839946 | CP023174.3135290-3138212 |
| AWTS01000185.360-3166 | LKLZ01000003.848448-851368 | CP021932.2992051-2994973 |
| FKLQ01000028.4324-5920 | MKDT01000006.1808-4726 | CP020093.2418751-2421557 |
| PEGI01000034.13-2935 | MKDW01000021.100-3022 | CP020564.472107-475027 |
| AZEJ01000033.2184-5102 | MKFX01000021.202-3008 | CP009236.1108333-1111251 |
| FWXC01000001.2016129-2018935 | MKDU01000042.1798-4716 | CP020861.2461072-2463994 |
| NQNI01000030.188-3110 | MKDS01000022.1782-4700 | CP020564.2812013-2814933 |
| PEGM01000040.13-2935 | KC109452.339-746 | CP020816.2494527-2497333 |
| JZSB01000012.280041-282847 | AF182722.211-673 | CP021501.2470413-2473333 |
| PEGP01000039.13-2935 | LUXA01000024.138-3058 | CP011769.1012672-1015594 |
| LJDC01000037.1-401 | LUWJ01000006.189-3109 | CP021501.1144241-1147161 |
| LJDC01000067.1-401 | CP001617.1156864-1159783 | CP020093.508572-511490 |
| LTCD01000061.188-3110 | KC161282.1-696 | CP020564.2358971-2361891 |
| FKLQ01000036.22437-25243 | ASJE01000130.134-3056 | CP009236.2916554-2919472 |
| LJDC01000046.1-401 | AGRI01000006.626817-629736 | CP021086.2878419-2881337 |
| AXZV01000022.141-3060 | CP013149.2425011-2427933 | CP020816.2944084-2946890 |
| LJDC01000041.1-401 | CP001617.2857559-2860478 | CP026743.575947-578869 |
| LGIM01000013.1-1425 | MKDQ01000018.352-3158 | CP023490.491639-494561 |
| AUTE01000080.1-2786 | MKDA01000064.193-3115 | CP023772.2911687-2914609 |
| PVNN01000013.1889-4811 | MKGF01000047.329-3011 | CP021501.1995703-1998623 |
| LYUK01000015.138-3058 | AL935263.504868-507787 | CP012122.2910937-2913859 |
| PEGK01000037.462-3384 | AL935263.1171166-1174085 | CP021528.2056693-2059613 |
| LSST01000060.188-3110 | LUWO01000069.158-3078 | CP023771.1922576-1925500 |
| PEGU01000018.13-2935 | LUWC01000011.158-3078 | CP012343.2953735-2956657 |
| PEGT01000015.39-2961 | LUWT01000036.158-3078 | CP011769.2706012-2708934 |
| LSND01000049.157-3079 | LUHN01000007.7531-8747 | CP029349.700760-703682 |
| PEKI01000076.1-1169 | LUWI01000010.475931-478851 | CP021528.1179917-1182837 |
| LJDC01000044.1-739 | LUXM01000033.144637-147557 | CP022373.185104-188026 |
| PEGQ01000039.188-3109 | LUWK01000023.158-3078 | CP021932.1159757-1162679 |
| LJDC01000045.1-518 | LUWE01000039.189-3109 | CP020093.1121054-1123972 |
| LTAU01000018.145-3065 | LUWA01000055.189-3109 | CP020564.1910968-1913888 |
| LGIM01000057.119246-119563 | LUWR01000004.189-3109 | CP009236.2475624-2478542 |
| PEGL01000040.462-3384 | LUWB01000047.189-3109 | CP024413.1156032-1158954 |
| PEGV01000039.13-2935 | CP015126.1987114-1990036 | CP021528.753056-755976 |
| LJDC01000040.1-401 | MH624120.1-2923 | CP023771.1061616-1064538 |
| PEGN01000039.188-3110 | KC109453.278-729 | CP021932.495932-498854 |
| LGIM01000097.29-1557 | CP015308.1444435-1447351 | CP031318.1088389-1091311 |
| LJDC01000038.1-647 | CP026743.2395587-2398509 | CP030105.2088451-2091257 |
| QPFU01000001.491374-494292 | AJ542548.1-372 | CP025991.3089010-3091933 |
| LEKW01000011.1-2530 | CP027349.2886932-2889854 | CP012650.1576752-1579674 |
| LEAY01000026.119-2925 | LUXL01000024.407613-408089 | CP021501.2905677-2908597 |
| LEBC01000047.292-3098 | CP013749.1037125-1040047 | CP021528.323436-326356 |
| OMOP01000095.191-3107 | CP015966.1370391-1373313 | CP025586.2419805-2422723 |
| FMBR01000251.1918-4836 | CP032460.1180052-1182974 | CP023728.1163334-1166256 |
| QXEU01000095.211-2586 | CP032648.2194216-2197138 | CP032464.1828348-1831270 |
| PEGR01000033.13-2935 | CP005942.1024850-1027768 | CP023490.2032035-2034957 |
| QPFU01000001.1130322-1133240 | CP017379.1061283-1064205 | CP023728.2014932-2017854 |
| QPQR01000030.130-3052 | LUWZ01000043.189-3109 | CP023490.978448-981370 |
| UASU01000001.1419270-1422188 | LUXO01000021.123964-126884 | CP025586.789343-792251 |
| FWXC01000001.2941972-2944778 | CP017354.1109529-1112451 | CP012122.486867-489785 |
| MPLC01000037.1821-4739 | CP017379.2850605-2853527 | CP032460.2769097-2772019 |
| JIBX01000032.1-1528 | CP013149.497314-500236 | CP030105.1656595-1659401 |
| RDEY01000027.1-1387 | CP001617.486614-489533 | CP026743.1941605-1944526 |
| LBDF01000013.69812-71946 | CP013753.1863827-1866749 | CP012343.350331-353253 |
| LEAX01000031.290-3096 | CP013753.2423757-2426679 | CP023771.2397631-2400553 |
| QPFU01000001.2521782-2524588 | CP002222.1985590-1988509 | CP012122.935301-938221 |
| LEBW01000018.290-3096 | CP019348.347087-349996 | CP025586.1941081-1943999 |
| AZFR01000081.140-3058 | CP015857.9170-12092 | CP025988.3106246-3109163 |
| UASU01000001.136855-139773 | CP015126.1175289-1178211 | CP023490.1352508-1355430 |
| QPFU01000001.2025563-2028369 | AP018405.2374309-2377227 | CP030881.13130-16048 |
| FWXC01000001.1171238-1174156 | CP015308.878893-881809 | CP025412.483014-485936 |
| LBHS01000001.1464239-1467161 | CP016270.1645484-1648403 | CP012343.1193796-1196718 |
| LUWD01000048.145-3065 | CP019348.1983926-1986848 | CP023728.499203-502125 |
| LUWM01000009.189-3109 | CP013749.1488688-1491610 | CP023771.2850950-2853872 |
| LEBG01000062.141-3059 | CP015966.2807600-2810522 | CP031702.1540052-1542974 |
| LEBI01000020.137-3055 | CP013749.2048618-2051540 | CP024413.1982715-1985637 |
| JIBX01000030.1-1390 | CP001617.1982443-1985362 | CP023772.2430472-2433394 |
| QPQS01000035.72-2994 | CP016270.296501-299421 | CP032464.489126-492048 |
| QPFU01000001.2960573-2963379 | CP022373.1727914-1730836 | CP023772.1534443-1537365 |
| LEBV01000020.290-3096 | CP004082.2718978-2721896 | CP023772.846859-849781 |
| QXEU01000111.1-626 | CP013749.2899745-2902667 | CP032648.298397-301319 |
| LEAW01000020.141-2947 | CP023174.440810-443732 | CP024413.2407246-2410168 |
| LEBR01000050.290-3096 | CP013753.565741-568663 | CP025412.1078359-1081281 |
| CBZW010000062.1-2686 | CP013753.3274883-3277805 | CP025988.806243-809161 |
| UASU01000001.2114123-2117040 | CP010528.2039478-2042400 | CP028977.1125250-1128172 |
| FZSL01000111.140-3012 | CP017354.2434261-2437183 | CP025590.1738648-1741454 |
| LEBS01000082.174-2980 | AP018405.1937444-1940362 | CP025412.1934213-1937135 |
| LEBJ01000078.290-3096 | CP023728.2946002-2948924 | CP025988.1277788-1280706 |
| LEBD01000049.116-2922 | AP018405.1164682-1167590 | CP023490.2623071-2625993 |
| FWXC01000001.2499027-2501833 | CP012122.2267052-2269973 | CP012650.2883876-2886798 |
| UASU01000001.590086-593004 | CP018209.3136103-3139021 | CP023772.17382-20304 |
| LEBT01000061.290-3096 | CP023174.2288594-2291516 | CP025590.2654460-2657266 |
| NDXC01000084.337-3255 | CP015126.2467241-2470163 | CP025590.2205535-2208341 |
| UASU01000006.211435-214353 | CP012343.2114963-2117885 | CP030881.2358764-2361672 |
| CP013149.1077123-1080045 | CP017354.2877400-2880322 | CP031318.2824376-2827298 |
| FWXC01000001.504932-507850 | AP018405.500919-503725 | CP025991.1073770-1076692 |
| LBHS01000001.824378-827300 | CP015308.2212592-2215508 | CP029349.2063132-2066054 |
| LEBA01000066.137-3055 | CP005942.466386-469304 | CP025991.2629573-2632495 |
| AWOY01000006.143-3065 | CP017379.483692-486614 | CP025991.1816263-1819185 |
| BALV01000030.141-3056 | CP017406.3105955-3108873 | CP028977.498885-501807 |
| LQHD01000032.1914-4834 | CP011769.453540-456462 | CP031771.497317-500235 |
| LEBH01000028.290-3096 | CP025412.2854281-2857203 | CP032642.1204573-1207495 |
| LEBB01000046.141-3059 | CP017954.2758565-2761487 | CP032751.2880765-2883687 |
| LEAZ01000050.182-2988 | CP015966.53197-56119 | CP031702.2822785-2825707 |
| RDEY01000026.135-1671 | CP002222.2465929-2468848 | CP032460.344843-347765 |
| LEAV01000040.290-3096 | CP015966.2039576-2042498 | CP032648.1735653-1738575 |
| LEBF01000021.290-3096 | CP026743.1475989-1478911 | CP032359.486897-489819 |
| LOMH01000102.188-3110 | CP002222.2915487-2918406 | CP032464.3190742-3193664 |
| LEBN01000034.140-3058 | CP002222.489648-492567 | CP030105.2534104-2536910 |
| LBHS01000002.465880-468802 | CP016270.1158827-1161743 | CP025590.896368-899286 |
| LEBE01000053.174-2980 | CP026743.3210296-3213218 | CP025991.214649-217571 |
| LEBL01000038.290-3096 | CP017066.2930737-2933659 | CP028977.2028846-2031768 |
| LEBU01000057.240-3046 | CP016071.2437340-2440262 | CP031318.2339638-2342560 |
| LQHB01000014.189-3109 | CP002222.1134012-1136931 | CP031771.1077126-1080044 |
| LQHC01000054.1914-4834 | CP017379.1922234-1925156 | CP032642.560683-563605 |
| LEBP01000039.141-3059 | CP015857.489172-492094 | CP032751.2440379-2443301 |
| LEBM01000047.290-3096 | CP015126.487442-490364 | CP029349.1308030-1310952 |
| LBDE01001655.1-1805 | CP017363.2388813-2391735 | CP028977.2453702-2456624 |
| LBDF01000010.75118-76053 | CP017374.1970620-1973542 | CP030105.766317-769235 |
| CP013749.190621-193543 | CP017354.1940712-1943634 | CP032460.2034535-2037457 |
| APHP01000043.1844-4766 | CP017406.312260-315178 | CP031318.481107-484029 |
| LEBK01000059.290-3096 | CP006033.2352904-2355823 | CP028977.2887998-2890920 |
| JHWA01000027.149669-152577 | CP019722.2932318-2935240 | CP030881.1726312-1729118 |
| NIGV01000012.52132-55044 | CP016270.2076717-2079637 | CP027349.1164485-1167407 |
| PYBS01000026.157-3079 | CP014780.494235-497157 | CP031702.19278-22200 |
| MKDF01000048.245-3051 | CP015308.15426-18342 | CP032464.973864-976786 |
| CP001617.2406988-2409907 | CP015857.1350320-1353242 | CP032642.2018330-2021252 |
| LEBQ01000040.141-3059 | AP018405.2860023-2862941 | CP032464.2435634-2438556 |
| MKEC01000050.1-2779 | CP004082.3178845-3181763 | CP027349.1980550-1983472 |
| LEBO01000047.141-3059 | CP016071.82305-85227 | CP027349.2451406-2454328 |
| QMJN01000080.113-3035 | CP016270.2834980-2837899 | CP030881.901800-904718 |
| QMJS01000121.137-3059 | CP017954.117313-120235 | CP032359.2267101-2270023 |
| CP015126.2914445-2917367 | CP014780.1182256-1185178 | CP032466.1809877-1812799 |
| CP015308.2682366-2685282 | CP021997.1955773-1958695 | CP029349.3044017-3046939 |
| MKEH01000004.141-2947 | CP017066.2483457-2486379 | CP029349.2559280-2562202 |
| MAXE01000020.42-1400 | CP014780.2038329-2041251 | CP030881.466256-469174 |
| NIGL01000004.510-1897 | CP031702.2369418-2372340 | CP031702.843836-846758 |
| JHWA01000027.908102-911020 | CP004406.486846-489765 | CP032642.2536334-2539256 |
| NIGH01000033.140-3058 | CP017363.1895262-1898184 | CP032642.2984777-2987699 |
| MKEF01000074.140-3058 | CP017374.1131181-1134103 | CP027349.481487-484409 |
| MKDB01000028.221-3143 | CP006033.1881510-1884429 | CP032359.1453344-1456266 |
| CP015966.515451-518373 | CP005942.1876469-1879387 | CP032466.1202604-1205526 |
| MQNK01000090.109232-112150 | CP015857.1940902-1943824 | CP032359.935339-938261 |
| JHWA01000015.24420-27230 | CP032648.967605-970526 | CP032466.348126-351048 |
| LUWV01000029.158-3078 | CP021997.250345-253267 | CP030105.144269-147187 |
| CP004082.2256665-2259583 | CP010528.2935198-2938120 | CP031771.2887268-2890176 |
| MKDP01000062.1992-4910 | CP004406.1156980-1159899 | CP032466.3061124-3064046 |
| OMOO01000100.191-3107 | CP017363.2831953-2834875 | CP032751.511351-514273 |
| LKLZ01000009.476405-476816 | CP017374.2420080-2423002 | CP031318.1843490-1846412 |
| MQNL01000032.18742-21660 | CP005942.2350602-2353520 | CP031771.1943094-1945900 |
| CP017354.503806-506728 | CP017406.731946-734864 | CP032751.1881129-1884051 |
| CP017379.2397287-2400209 | CP006033.2779990-2782909 | CP031771.2425113-2427919 |
| NIGG01000040.236-3042 | CP022294.2455107-2458029 | CP032751.1082317-1085239 |
| AJ542557.1-354 | CP015857.2712063-2714985 | CP032359.2910991-2913913 |
| AL935263.2498866-2501785 | CP014780.2918979-2921901 | CP032466.2564976-2567898 |
| MAMT01000045.73-1110 | CP016071.1966374-1969296 | |

Lactobacillus rhamnosus

| | | |
|---|---|---|
| NBVL01000096.1-359 | QAZK01000028.11-2930 | AY608417.359-956 |
| NBVL01000020.395947-396305 | MLKA01000027.125-3044 | AMQX01000036.126-3043 |
| NBVL01000038.107249-110168 | MLKC01000014.1-1442 | AP011548.1920458-1923378 |
| JYCS01000046.125-3044 | NXEW01000052.657-1051 | JQ952565.1-1064 |
| JMSI01000014.11-2731 | QFFR01000017.4-2923 | EF030190.1-2449 |
| MJHB01000049.1907-4822 | JVIZ01000304.304-3103 | AF182730.226-686 |
| LPNU01000052.112922-115720 | JUIS01000013.231-3150 | LP124489.309541-312456 |
| PKQF01000030.4-2923 | JUMO01000136.1-2498 | EU184020.1849-4770 |
| JNNV01000002.195931-198850 | NXEX01000013.10039-12705 | AP011548.2560406-2563325 |
| LPNW01000032.45107-47906 | MLKC01000050.1-1080 | KJ522771.175-600 |
| JTIT01000033.125-3044 | QFFQ01000030.4-2923 | LFNB01000054.125-3044 |
| JTDC01000070.125-3044 | JWHC01000055.125-3044 | JQ952566.1-1064 |
| LWBT01000053.4-2923 | NXEZ01000022.45151-48071 | FM179322.309541-312456 |
| AEYM01001955.1-336 | NXEV01000050.667-3586 | FM179323.1892336-1895251 |
| MSTB01000010.9-2926 | JUIL01000003.232-3151 | EF030191.1-2449 |
| JDFQ01000059.128-3043 | JUPA01000135.128-3043 | CP017063.780881-783800 |
| LPNW01000054.297-3212 | JUIP01000001.125-3044 | CP016823.26583-29501 |
| LPNW01000062.48132-50931 | NXEU01000011.44449-47667 | LT220504.1453029-1455828 |
| JTIL01000084.1-383 | LAZE01000069.42871-45790 | AYTR01000044.116-2915 |
| JTIX01000003.125-3044 | NXEZ01000009.1-457 | AYTQ01000062.167-2966 |
| JTIV01000001.233-3152 | JUII01000288.9-2928 | LP124490.308565-311480 |
| JTIH01000044.125-3044 | JUPX01000234.110-2909 | FM179322.825034-827949 |
| JNNV01000008.54289-57208 | MLKC01000062.1-396 | FM179323.2545004-2547919 |
| JTHY01000064.11-2930 | MLJZ01000022.1-2901 | FM179322.845075-847990 |
| PKJX01000001.973678-976597 | MLKB01000036.125-3044 | CP005484.752982-755899 |
| PKQF01000104.1-365 | AZCQ01000032.110-2909 | LP124489.1926158-1928958 |
| JDFR01000053.122-2921 | JUIN01000677.125-3044 | CP006804.1311020-1313939 |
| JDRW01000070.128-3043 | UWPA01000065.110-2909 | AY608413.572-1169 |
| PESP01000015.125-2349 | CBZU010000002.5-2920 | LP124489.825034-827949 |
| JTIA01000278.2-2921 | JUIR01000596.11-2930 | LP124490.2560513-2563312 |
| JTIM01000029.36124-36688 | JUKV01000156.235-3034 | KJ522769.279-800 |
| JTIG01000044.11-2930 | JUIO01000001.4-2923 | CP020464.2278682-2281601 |
| JTIU01000003.227-3146 | JUIJ01000273.9-2928 | KJ522768.236-686 |
| LPNT01000058.78246-79836 | JUON01000132.128-3043 | AY608412.572-1169 |
| LPNV01000046.509128-512042 | JUIH01000144.100-3019 | AY608416.359-956 |
| LPNT01000028.16075-17665 | OGTX02000322.7-2894 | FM179322.1926053-1928969 |
| AVFG02000084.81319-84238 | JUIM01000001.125-3044 | CP021426.1067790-1070709 |
| JTIC01000068.9-2928 | JUIK01000542.125-3044 | CP016823.490071-492987 |
| JTIP01000108.11-2930 | BALT01000056.14-2929 | FM179322.2565342-2568257 |
| LPNT01000027.1-1700 | PYRN01000027.227-3146 | CP016823.1567720-1570639 |
| JTIR01000061.125-3044 | JUTB01000139.394-3309 | CP005484.1799561-1802478 |
| JTHS01000058.232-3151 | JUIQ01000003.125-3044 | CP017063.760852-763771 |
| JPZB01000061.125-3044 | ACIZ01000149.128-3043 | CP006804.2596532-2599451 |
| QAHA01000029.11-2930 | JULF01000298.425-3340 | CP005484.2468527-2471444 |
| MWVF01000026.11-2930 | LT220504.1966955-1969754 | CP006804.2616571-2619490 |
| QAZJ01000027.1-2709 | JVPR01000263.122-2921 | LP124489.845075-847990 |
| JTHZ01000001.9-2928 | JUWG01000102.110-2909 | LT220504.331530-334445 |
| ABWJ01000062.125-3044 | JUTS01000152.466-3265 | CP003094.2464960-2467873 |
| JTIE01000095.11-2914 | JVIZ01000305.13985-16900 | CP016823.470038-472957 |
| JTHW01000071.125-3043 | MAGA01000079.1-1047 | CP017063.1800293-1803212 |
| JTHT01000106.1-2919 | MAGA01000089.1-436 | CP003094.1817448-1820361 |
| JTIW01000098.9-2928 | CP017063.2467680-2470599 | CP014201.1042874-1045793 |
| JNNV01000022.16023-16476 | CP005484.279215-282132 | CP014201.1702655-1705574 |
| JTHQ01000082.125-3044 | AMQW01000040.7-2924 | CP005484.773131-776048 |
| JUWQ01000124.110-2909 | CP006804.643619-646539 | CP016823.2207573-2210490 |
| QQWF01000027.2051-4970 | AGKC01000001.982077-983797 | CP014201.3038292-3041211 |
| LPNT01000036.112574-114165 | JVLT01000015.110-2909 | CP006804.2123604-2126523 |
| JUMP01000134.110-2909 | JVPR01000164.304-3103 | CP020464.1249824-1252743 |
| JTIL01000088.1-383 | MAGA01000092.1-434 | CP021426.2460816-2463736 |
| JTHX01000042.4-2923 | FM179323.819584-822499 | CP022109.2562767-2565566 |
| JTIM01000073.1-565 | JX665051.229-677 | CP014201.3018254-3021173 |
| QAZH01000027.125-3044 | MAGA01000077.1-2097 | CP005485.298950-301867 |
| QAGZ01000031.11-2239 | JVJA01000908.1-2515 | CP014201.2535397-2538316 |
| JTIK01000344.125-3044 | JVQV01000050.128-3043 | CP022109.815101-818016 |
| QAZI01000051.125-3044 | ATBI01000022.12-2927 | CP005485.813907-816824 |
| QAZJ01000036.1-338 | ATBI01000006.245094-247893 | CP014645.615844-618763 |
| QQWF01000763.1-527 | AP011548.822600-825519 | CP017063.270244-273163 |
| QAHB01000026.1685-4604 | JVIZ01000389.122-2921 | CP003094.822938-825853 |
| AFZY01000013.1-2623 | AGKC01000009.1-1444 | CP019305.835125-838040 |
| JTIL01000075.9-2545 | LP124490.842643-845558 | CP022109.835125-838040 |
| QAGY01000026.125-3044 | JVPR01000194.1927-4842 | CP003094.802914-805829 |
| JTIF01000010.125-3044 | JVLT01000016.128-3043 | CP005485.833931-836848 |
| MSTC01000071.125-3043 | AY608414.359-956 | CP005485.2563350-2566267 |
| JNNV01000004.1-770 | FM179323.291567-294482 | CP005485.1893930-1896847 |
| JTHU01000144.100-3019 | LT220504.1432902-1435807 | CP019305.815101-818016 |
| QAGZ01000041.1-376 | LT220504.2686837-2689752 | CP003094.291743-294658 |
| JTHV01000013.1-2918 | AP011548.842641-845560 | CP019305.1893891-1896690 |
| JTIB01000063.125-3044 | AP011548.308563-311482 | CP020016.346162-349081 |
| JTII01000049.9-2928 | LP124489.2565447-2568246 | CP019305.2562756-2565555 |
| QAGW01000012.11-2930 | CP021426.553754-556673 | CP021426.1820994-1823913 |
| PESP01000017.4-693 | AYTP01000043.128-3043 | CP021426.533713-536632 |
| JTIO01000001.64-2983 | LP124490.822602-825517 | CP020464.2298825-2301744 |
| JTIN01000086.137-3056 | JVDP01000077.2140-5055 | CP020464.2772592-2775511 |
| JTID01000072.10-2929 | AY608415.359-956 | CP022109.298952-301867 |
| QAGZ01000038.1-569 | FM179323.839608-842523 | CP020464.581711-584630 |
| JTHR01000069.124-3043 | AB092641.224-693 | CP019305.298952-301867 |
| JTIS01000001.9-2928 | LFNA01000055.125-3044 | CP022109.1893898-1896697 |
| JTIQ01000061.233-3152 | ATBI01000013.62458-65257 | CP031290.2565346-2568265 |
| JTIM01000061.125-2479 | PZQM01000035.277-3196 | CP031290.309538-312457 |
| JTIJ01000045.9-2928 | ATBI01000015.292-3098 | CP031290.825034-827953 |
| QAGX01000013.11-2930 | JVCX01000010.2140-5055 | CP031290.845075-847994 |
| AFYD01000005.110-1639 | LP124490.1920565-1923365 | CP031290.1926057-1928977 |
| NXEW01000004.73847-76759 | ATBI01000019.12-2927 | |

Exemplary B. lactis Sequences

SEQ ID NO: 14, B. lactis 16S, 1538 nucleotides (nt)
TTTGTGGAGGGTTCGATTCTGGCTCAGGATGAACGCTGGCGGCGTGCTTAACACATGCAA
GTCGAACGGGATCCCTGGCAGCTTGCTGTCGGGGTGAGAGTGGCGAACGGGTGAGTAAT
GCGTGACCAACCTGCCCTGTGCACCGGAATAGCTCCTGGAAACGGGTGGTAATACCGGA
TGCTCCGCTCCATCGCATGGTGGGGTGGGAAATGCTTTTGCGGCATGGGATGGGGTCGCG
TCCTATCAGCTTGTTGGCGGGGTGATGGCCCACCAAGGCGTTGACGGGTAGCCGGCCTGA
GAGGGTGACCGGCCACATTGGGACTGAGATACGGCCCAGACTCCTACGGGAGGCAGCAG
TGGGGAATATTGCACAATGGGCGCAAGCCTGATGCAGCGACGCCGCGTGCGGGATGGAG
GCCTTCGGGTTGTAAACCGCTTTTGTTCAAGGGCAAGGCACGGTTTCGGCCGTGTTGAGT
GGATTGTTCGAATAAGCACCGGCTAACTACGTGCCAGCAGCCGCGGTAATACGTAGGGT
GCGAGCGTTATCCGGATTTATTGGGCGTAAAGGGCTCGTAGGCGGTTCGTCGCGTCCGGT
GTGAAAGTCCATCGCCTAACGGTGGATCTGCGCCGGGTACGGGCGGGCTGGAGTGCGGT
AGGGGAGACTGGAATTCCCGGTGTAACGGTGGAATGTGTAGATATCGGGAAGAACACCA
ATGGCGAAGGCAGGTCTCTGGGCCGTCACTGACGCTGAGGAGCGAAAGCGTGGGGAGCG
AACAGGATTAGATACCCTGGTAGTCCACGCCGTAAACGGTGGATGCTGGATGTGGGGCC
CTTTCCACGGGTCCCGTGTCGGAGCCAACGCGTTAAGCATCCCGCCTGGGGAGTACGGCC
GCAAGGCTAAAACTCAAAGAAATTGACGGGGGCCCGCACAAGCGGCGGAGCATGCGGA
TTAATTCGATGCAACGCGAAGAACCTTACCTGGGCTTGACATGTGCCGGATCGCCGTGGA
GACACGGTTTCCCTTCGGGGCCGGTTCACAGGTGGTGCATGGTCGTCGTCAGCTCGTGTC
GTGAGATGTTGGGTTAAGTCCCGCAACGAGCGCAACCCTCGCCGCATGTTGCCAGCGGG
TGATGCCGGGAACTCATGTGGGACCGCCGGGGTCAACTCGGAGGAAGGTGGGGATGACG
TCAGATCATCATGCCCCTTACGTCCAGGGCTTCACGCATGCTACAATGGCCGGTACAACG
CGGTGCGACACGGTGACGTGGGGCGGATCGCTGAAAACCGGTCTCAGTTCGGATCGCAG
TCTGCAACTCGACTGCGTGAAGGCGGAGTCGCTAGTAATCGCGGATCAGCAACGCCGCG
GTGAATGCGTTCCCGGGCCTTGTACACACCGCCCGTCAAGTCATGAAAGTGGGTAGCACC
CGAAGCCGGTGGCCCGACCCTTGTGGGGGGAGCCGTCTAAGGTGAGACTCGTGATTGGG
ACTAAGTCGTAACAAGGTAGCCGTACCGGAAGGTGCGGCTGGATCACCTCCTTT
SEQ ID NO: 15, B. lactis, Accession Number: CP001853.168853.171909
Bifidobacterium; Bifidobacterium animalis subsp. lactis. Sequence:
GCAGACAGUACCGAUGAAGGACGUGUGGGGCCGCGAUAGUCCUGGGGGAGCCGCCGA
CAUGGCUUUGAUCCCAGGGUGUCCGAAUGGGGCAACCCGCCAGCCGUCAUGGGCUGGC
ACCGCACUUGUGCGGGGGGUACGCAGGGAAGUGAAACAUCUCAGUACCUGCAGGAGA
GGAUACUCCGUGAGUAGUGGCGAGCGAAAGCGGAUGAUGGCUAAACCGUGCGCGUGU
GAUACCCGUCGGGGGUUGCGCGCAUGGUGUCGUGGGAACGCCUGUCCAACGGCCGACG
CCGUUGGCUGUGGUGAUAAAUGCAUGCGUGAGGCGAACCGGGUUGGAUACCGGGCCG
UAGAGGGUGAUGGCCCCGUAGCCGUAUGCGCGUGCACCAUGGAUGGUGUCUCCCGAG
UAGCGCGGGACUCGUGGAAUCCCGUGUGAAUCCGCCCGGACCGUCGGGUAAGCCUGAG
UAUACCUGUCUGACCGAUAGCGUACGAGUACCGUGAGGGAAAGGUGAAAAGCACCCC
GGGAGGGGAGUGAAAGAGUUCCUGAAACCGUGCGCCCGCGAACCGUCGGAGCCCCCUU
GGUGGGGUGACGGCGUGCCUAUCGAAAAAUGAGUCUGCGAGUCAGUGGCAUGUGGCG
AGCAUAAGCCGUGUGGUGUAUGCGUAGCGAAAGCGAGUCUUAAAAGGCGUCUUUUCA
GUCGCGUGUCCUGGACCCGAAGCGGGAUGAUCUAGCCCUGGUCAGGUUGAAGCGCGG
GUAAGACCGCGUGGAGGACCGCACCCACUUAGGUUGAAAACUGAGGGGAUGAACUGG
GGUUAGGGGUGAAAGGCCAAUCAAAUUCCGUGAUAGCUGGUUCUCUUCGAAAUGCAU
UUAGGUGCAGCGUCGGUUGAUUGCGUGUGGGGGGUAGAGCUACUGGAUGCUUGCGGG
CCCGUGCCGGGUACCAACAGCAACCAAACUCCGAAUACCCAUCGCGUCUAUUCCGGCA
GUGAGUCGGCGGGGGAUAAGCUCCGUCGUCGAGGGGGAGACAGCCCUGACCGUCGUC
UAAGGUCCCCAAGCGCGUGCUAAGUGGGAAAGGAUGUGGAGUCGCAUAGACAGCCAG
GAGGUUGGCUCAGAAGCAGCCAUCCUUGAAAGAGUGCGUAACAGCUCACUGGUCUAG
UGGUUCCGCGCCGACAAUGUAGCGGGGCUCAAGCACGCCACCGAAGACGCGGAUGCGG
CCGUAUGGCCGUGUGGUAGGAGAGCGUCCCACACCGGGGCGAAGCGGCGGGGGAACCC
GGCCGUGGACUGUGUGGGAGUGAGAAUGCAGACAUGAGUAGCGAGAGCAGGGUGAGG
AUCCCUGCCGCUGGAAGACCAAGGGUUCCAGGGCCACGUUCGUCGUCCCUGGGUGAGU
CGGGUCCUAAGGCGAGGCCGACAGGCGUAGUCGAAUGGAUGAACGGGUCGAUAUUCC
CGUACCGGCUUUCGGACCGACCAACAGCCCGUGUGAACAAGGGCCAUCCGAUACGGUU
GAUGGGGGCUUCGGCCUCCUGAUUCCGUUGCUGUUUGGCCUGGACGCGCAUGGGUUG
AGCGUUUCAGGAGUGACGCGGACGGGUAGCCGGCCGGGGAGGUGGUUUUCCCUGGUC
AAGCGUGUGGCCCCGCCCCCAGGCAAAUCCGGGGGCUUCAUGGGGUGAGGCGUGAUGA
UGGGCGCUUUCAUGGCGUGAUAUCCGGUGAUCCCGUCGGCCGAGAAAAGCUUCGGCGC
GAGGUCCGUUGCCGCCCGUACCCCAAACCGACACAGGUGGUCUGGUAGAGUAUACCGA
AGCGAUCGAGCGAAUCCUGGUCAAGGAACUCGGCAAAUUGCUCCCGUGCCUUCGGCAU
AAGGGAGACCCCCGGCCGUGCCGGCCCCUUGCGGGUCGUGGGGGCUGGGGGUGGCAC
AGGCCAGGGGGUAGCGACUGUUUACCAAAAACACAGGUGCAUGCCAAGACGCAAGUC
GCCGUAUAUGCACUGACGCCUGCCCGGUGCCGGAAGGUUAAGAGGAUCCAUCAGCGUC
CCCUCGCGGGAUGCGAAGUGGUGAAUUCAAGCCCCGGUAAACGGCGGUGGUAACUAU
AACCAUCCUAAGGUAGCGAAAUUCCUUGUCGGGUAAGUUCCGACCUGCACGAAUGGC
GUAACGACUUCCCCGCUGUCUCGACCAGGAGCUCGGCGAAAUUGCAGUACGAGUAAAG
AUGCUCGUUAAGCGCAGAAGGACGAAAAGACCCCGGGACCUUUACUAUACCUUGGUA
UUGGCAUCAGGUGCGGAUUGUGUAGCAUAGGCGGGAGGCUUCGAAGCCGCGGCGCCA
GCCGUGGUGGAGCCGGCCAGUGAAAUACCGCUCUGUCCCCAUCUGAUGCCUAACCCGG
ACAAGUCAGCCUUGUCGGGGAGAGUGCCUGGCGGGUAGUUUAACUGGGGCGGUUGCC
UCCCAAAGAGUAACGGAGGCGCUCAAAGGUCCGCUCGGCCCGGUUGGCAAUCGGGUGU
CGAGUGUAAUCGCACAAGCGGGCUUGACUGCGAGAUCGACGGAUCGAGCAGGGACGA
AAGUCGGAGAUAGUGAUCCGGUGCCGGCGUACGGACGCGGCAUCGCUCAACGGAUAA
AAGGUACCCCGGGGAUAACAGGCUGAUCAUUCCCAAGAGUCCAUAUCGACGGGAUGG
UUUGGCACCUCGAUGUCGGCUCGUCGCAUCCUGGGGCUGGAGCAGGUCCCAAGGGUUC
GGCUGUUCGCCGAUUAAAGCGGCACGCGAGCUGGGUUCAGAACGUCGUGAGACAGUU
UGGUCUCUAUCCUCUGCGCUCGUUGGAAUCCUGAGGAGGCCUGCCCAUAGUACGAGAG
GACCUGGGUGGACGAACCUCUGGUAUGCCAGUUGUCGCGCCAGCGGCACGGCUGGUUG
GCCACGUUCGGGAGGGAUAACCGCUGAAAGCAUCUAAGCGGGAAGCCCGCUCCAAGAU
AAGGAUUCCUGACACCCCCCCCGGGGGUGUGUGAGACCCCAUAGAGAACAUGUGGUUG
AUAGACCGGACGUGGAAGCCCCGCGAGGGGUGGAGCCGACCGGCACUAACGGUCGAAG
ACAACCAACACC
SEQ ID NO: 16, B. lactis, Accession Number: CP001892.1476835.1479961
Bifidobacterium; Bifidobacterium animalis subsp. lactis. Sequence:
GUGGUUGUCGUGAUGGGCGUAUGGUGGAUGCCUUGGCAGACAGUACCGAUGAAGGAC
GUGUGGGGCCGCGAUAGUCCUGGGGGAGCCGCCGACAUGGCUUUGAUCCCAGGGUGU
CCGAAUGGGGCAACCCGCCAGCCGUCAUGGGCUGGCACCGCACUUGUGCGGGGGGUAC
GCAGGGAAGUGAAACAUCUCAGUACCUGCAGGAGAGGAUACUCCGUGAGUAGUGGCG
AGCGAAAGCGGAUGAUGGCUAAACCGUGCGCGUGUGAUACCCGUCGGGGGUUGCGCG
CAUGGUGUCGUGGGAACGCCUGUCCAACGGCCGACGCCGUUGGCUGUGGUGAUAAAU
GCAUGCGUGAGGCGAACCGGGUUGGAUACCGGGCCGUAGAGGGUGAUGGCCCCGUAG
CCGUAUGCGCGUGCACCAUGGAUGGUGUCUCCCGAGUAGCGCGGGACUCGUGGAAUCC
CGUGUGAAUCCGCCCGGACCGUCGGGUAAGCCUGAGUAUACCUGUCUGACCGAUAGCG
UACGAGUACCGUGAGGGAAAGGUGAAAAGCACCCCGGGAGGGGAGUGAAAGAGUUCC
UGAAACCGUGCGCCCGCGAACCGUCGGAGCCCCCUUGGUGGGGUGACGGCGUGCCUAU
CGAAAAAUGAGUCUGCGAGUCAGUGGCAUGUGGCGAGCAUAAGCCGUGUGGUGUAUG
CGUAGCGAAAGCGAGUCUUAAAAGGCGUCUUUUCAGUCGCGUGUCCUGGACCCGAAG
CGGGAUGAUCUAGCCCUGGUCAGGUUGAAGCGCGGGUAAGACCGCGUGGAGGACCGC
ACCCACUUAGGUUGAAAACUGAGGGGAUGAACUGGGGUUAGGGGUGAAAGGCCAAUC
AAAUUCCGUGAUAGCUGGUUCUCUUCGAAAUGCAUUUAGGUGCAGCGUCGGUUGAUU
GCGUGUGGGGGGUAGAGCUACUGGAUGCUUGCGGGCCCGUGCCGGGUACCAACAGCA
ACCAAACUCCGAAUACCCAUCGCGUCUAUUCCGGCAGUGAGUCGGCGGGGGAUAAGCU
CCGUCGUCGAGGGGGAGACAGCCCUGACCGUCGUCUAAGGUCCCCAAGCGCGUGCUAA
GUGGGAAAGGAUGUGGAGUCGCAUAGACAGCCAGGAGGUUGGCUCAGAAGCAGCCAU
CCUUGAAAGAGUGCGUAACAGCUCACUGGUCUAGUGGUUCCGCGCCGACAAUGUAGC
GGGGCUCAAGCACGCCACCGAAGACGCGGAUGCGGCCGUAUGGCCGUGUGGUAGGAG
AGCGUCCCACACCGGGGCGAAGCGGCGGGGGAACCCGGCCGUGGACUGUGUGGGAGUG
AGAAUGCAGACAUGAGUAGCGAGAGCAGGGUGAGGAUCCCUGCCGCUGGAAGACCAA
GGGUUCCAGGGCCACGUUCGUCGUCCCUGGGUGAGUCGGGUCCUAAGGCGAGGCCGAC
AGGCGUAGUCGAAUGGAUGAACGGGUCGAUAUUCCCGUACCGGCUUUCGGACCGACC
AACAGCCCGUGUGAACAAGGGCCAUCCGAUACGGUUGAUGGGGGCUUCGGCCUCCUGA
UUCCGUUGCUGUUUGGCCUGGACGCGCAUGGGUUGAGCGUUUCAGGAGUGACGCGGA
CGGGUAGCCGGCCGGGGAGGUGGUUUUCCCUGGUCAAGCGUGUGGCCCCGCCCCCAGG
CAAAUCCGGGGGCUUCAUGGGGUGAGGCGUGAUGAUGGGCGCUUUCAUGGCGUGAUA
UCCGGUGAUCCCGUCGGCCGAGAAAAGCUUCGGCGCGAGGUCCGUUGCCGCCCGUACC
CCAAACCGACACAGGUGGUCUGGUAGAGUAUACCGAAGCGAUCGAGCGAAUCCUGGU
CAAGGAACUCGGCAAAUUGCUCCCGUGCCUUCGGCAUAAGGGAGACCCCCGGCCGUGC
CGGCCCCUUGCGGGUCGUGGGCGGCUGGGGGUGGCACAGGCCAGGGGGUAGCGACUG
UUUACCAAAAACACAGGUGCAUGCCAAGACGCAAGUCGCCGUAUAUGCACUGACGCCU
GCCCGGUGCCGGAAGGUUAAGAGGAUCCAUCAGCGUCCCCUCGCGGGAUGCGAAGUGG
UGAAUUCAAGCCCCGGUAAACGGCGGUGGUAACUAUAACCAUCCUAAGGUAGCGAAA
UUCCUUGUCGGGUAAGUUCCGACCUGCACGAAUGGCGUAACGACUUCCCCGCUGUCUC
GACCAGGAGCUCGGCGAAAUUGCAGUACGAGUAAAGAUGCUCGUUAAGCGCAGAAGG
ACGAAAAGACCCCGGGACCUUUACUAUACCUUGGUAUUGGCAUCAGGUGCGGAUUGU
GUAGCAUAGGCGGGAGGCUUCGAAGCCGCGGCGCCAGCCGUGGUGGAGCCGGCCAGUG
AAAUACCGCUCUGUCCCCAUCUGAUGCCUAACCCGGACAAGUCAGCCUUGUCGGGGAG
AGUGCCUGGCGGGUAGUUUAACUGGGGCGGUUGCCUCCCAAAGAGUAACGGAGGCGC
UCAAAGGUCCGCUCGGCCCGGUUGGCAAUCGGGUGUCGAGUGUAAUCGCACAAGCGG
GCUUGACUGCGAGAUCGACGGAUCGAGCAGGGACGAAAGUCGGAGAUAGUGAUCCGG
UGCCGGCGUACGGACGCGGCAUCGCUCAACGGAUAAAAGGUACCCCGGGGAUAACAGG
CUGAUCAUUCCCAAGAGUCCAUAUCGACGGGAUGGUUUGGCACCUCGAUGUCGGCUCG
UCGCAUCCUGGGGCUGGAGCAGGUCCCAAGGGUUCGGCUGUUCGCCGAUUAAAGCGGC
ACGCGAGCUGGGUUCAGAACGUCGUGAGACAGUUUGGUCUCUAUCCUCUGCGCUCGU
UGGAAUCCUGAGGAGGCCUGCCCAUAGUACGAGAGGACCUGGGUGGACGAACCUCUG
GUAUGCCAGUUGUCGCGCCAGCGGCACGGCUGGUUGGCCACGUUCGGGAGGGAUAACC
GCUGAAAGCAUCUAAGCGGGAAGCCCGCUCCAAGAUAAGGAUUCCUGACACCCCCCCC
GGGGGUGUGUGAGACCCCAUAGAGAACAUGUGGUUGAUAGACCGGACGUGGAAGCCC
CGCGAGGGGUGGAGCCGACCGGCACUAACGGUCGAAGACAACCAACACCACCAUGACG
AAGCCGGAAAACCCCGGCGGCAUCAC
Exemplary B. breve sequences
SEQ ID NO: 17, B. breve 16S sequence, 1531 nt (see e.g., NCBI Reference Numbers
B7017_RS06345 or B7017_RS09250, 16S ribosomal RNA, Bifidobacterium breve JCM 7017,
NCBI Gene IDs: 56564913 or 56565465)
TTTGTGGAGGGTTCGATTCTGGCTCAGGATGAACGCTGGCGGCGTGCTTAACACATGCAA
GTCGAACGGGATCCATCGGGCTTTGCTTGGTGGTGAGAGTGGCGAACGGGTGAGTAATG
CGTGACCGACCTGCCCCATGCACCGGAATAGCTCCTGGAAACGGGTGGTAATGCCGGAT
GCTCCATCACACCGCATGGTGTGTTGGGAAAGCCTTTGCGGCATGGGATGGGGTCGCGTC
CTATCAGCTTGATGGCGGGGTAACGGCCCACCATGGCTTCGACGGGTAGCCGGCCTGAG
AGGGCGACCGGCCACATTGGGACTGAGATACGGCCCAGACTCCTACGGGAGGCAGCAGT
GGGGAATATTGCACAATGGGCGCAAGCCTGATGCAGCGACGCCGCGTGAGGGATGGAG
GCCTTCGGGTTGTAAACCTCTTTTGTTAGGGAGCAAGGCATTTTTTGTTGAGTGTACCTTT
CGAATAAGCACCGGCTAACTACGTGCCAGCAGCCGCGGTAATACGTAGGGTGCAAGCGT
TATCCGGAATTATTGGGCGTAAAGGGCTCGTAGGCGGTTCGTCGCGTCCGGTGTGAAAGT
CCATCGCTTAACGGTGGATCCGCGCCGGGTACGGGCGGGCTTGAGTGCGGTAGGGGAGA
CTGGAATTCCCGGTGTAACGGTGGAATGTGTAGATATCGGGAAGAACACCAATGGCGAA
GGCAGGTCTCTGGGCCGTTACTGACGCTGAGGAGCGAAAGCGTGGGGAGCGAACAGGAT
TAGATACCCTGGTAGTCCACGCCGTAAACGGTGGATGCTGGATGTGGGGCCCGTTCCACG
GGTTCCGTGTCGGAGCTAACGCGTTAAGCATCCCGCCTGGGGAGTACGGCCGCAAGGCT
AAAACTCAAAGAAATTGACGGGGGCCCGCACAAGCGGCGGAGCATGCGGATTAATTCGA
TGCAACGCGAAGAACCTTACCTGGGCTTGACATGTTCCCGACGATCCCAGAGATGGGGTT
TCCCTTCGGGGCGGGTTCACAGGTGGTGCATGGTCGTCGTCAGCTCGTGTCGTGAGATGT
TGGGTTAAGTCCCGCAACGAGCGCAACCCTCGCCCCGTGTTGCCAGCGGATTATGCCGGG
AACTCACGGGGGACCGCCGGGGTTAACTCGGAGGAAGGTGGGGATGACGTCAGATCATC
ATGCCCCTTACGTCCAGGGCTTCACGCATGCTACAATGGCCGGTACAACGGGATGCGAC
AGTGCGAGCTGGAGCGGATCCCTGAAAACCGGTCTCAGTTCGGATCGCAGTCTGCAACT
CGACTGCGTGAAGGCGGAGTCGCTAGTAATCGCGAATCAGCAACGTCGCGGTGAATGCG
TTCCCGGGCCTTGTACACACCGCCCGTCAAGTCATGAAAGTGGGCAGCACCCGAAGCCG
GTGGCCTAACCCCTTGCGGGAGGGAGCCGTCTAAGGTGAGGCTCGTGATTGGGACTAAG
TCGTAACAAGGTAGCCGTACCGGAAGGTGCGGCTGGATCACCTCCTTT
SEQ ID NO: 18, B. breve, Accession Number: BCXW01000037.1900.4963
Bifidobacterium; Bifidobacterium breve. Sequence:
UGUUGCCUGCAAGGGCGUAUGGUGGAUGCCUUGGCAGACAGGACCGAUGAAGGACGU
UUGAGGCUGCGAUAUGCCUCGGGGAGCCGCCAACAGGGCUUUGAUCCGAGGAUUUCC
GAAUGGGGGAACCCACCGGCCGUCAUGGGCCGGUACCGCCGUGUGCGGGGGGUACGCA
GGGAAGUGAAACAUCUCAGUACCUGCAGGAAAGGAUAUUCCGUGAGUAGUGGCGAGC
GAAAGCGGAUCAGGCCAAACCUUGCGCGUGUGAUACCCGUCGGGGGUUGCGCGCGGG
GUGUAGAGGGAUCGCGUUUGCCGGCUCCGACGGGCCGGCCGUCAGUUGCAUAAAACCA
UGUGCCAGGGGAACCGGGUUGAAUACCGGGCCGCAGAGGGUGAGGGCCCCGUACCCGA
ACGCGCAUGGUCUGGCGAUCGCGUCUCCCAAGUAGCACGGGCCCCGUGGAAUCCCGUG
CGAAUCUGUCCCGACCGUGGGAUAAGCCUAAAUAUUCCUGUCUGACCGAUAGCGAACG
AGUACCGUGAGGGAAAGGUGAAAAGUACCCCGGGAGGGGAGUGAAACAGUCUCUGAA
ACCAUGCGCCUACGAACCGUCGGGGCCCCCUUGUGGGGUGACGGCGUGCCUAUCGAAA
AAUGAGUCUGCGAGUCAGUGGCAUGUGGCGAGCAUAACCCGUGUGGGGUAUGCGUAG
CGAAGGCGAGUCUUAAAAGGCGUUUAAAGUCGCGUGUCCUGGACCCGAAGCGGGAUG
AUCUAGCCCUGAGCAGGUUGAAGCGCGGGUAAGACCGUGUGGAGGACCGAACCCACCU
GGGUUGAAAACCGGGGGGAUGACUUGGGGCUAGGGGUGAAAGGCCAAUCAAAUUCCG
UGAUAGCUGGUUCUCUCCGAAAUGCAUUUAGGUGCAGCGUCGGAUCGAUUACAUCCG
GGGGGUAGAGCGACUGGAUGCUUGCGGGCCCAUAUCGGGUACCAACAGCAACCAAAC
UCCGAAUACCCGUGAUGCGUAUCCCGGCAGUGAGUCGGCGGGGGAUAAGCUCCGUCGU
CGAAAGGGAAACAGCCCAGAUCGUCGUCUAAGGUCCCCAAGCGUGUGCUAAGUGGGA
AAGGAUGUGGAGUCGCAUAGACAGCCAGGAGGUUGGCUCAGAAGCAGCCACCCUUGA
AAGAGUGCGUAACAGCUCACUGGUCUAGUGGUUCCGCGCCGACAAUGUAGCGGGGCU
CAAGCACACCACCGAAGACGCGGCAGUAGCUUUGCUACUGGGUAGGAGAGCGUCCCGU
GCGGGGCGAAGCGGCGGCGCAAGCCGGCCGUGGACCGCAUGGGAGUGAGAAUGCAGA
CAUGAGUAGCGAAAGACGGGUGAAGAUCCCGUCCGCUGGACGACCAAGGGUUCCAGG
GCCACGUUCAUCGUCCCUGGGUGAGUCGGGUCCUAAGGCGAGGCCGACAGGCGUAGUC
GAAUGGAUGAACGGGUCGAUAUUCCCGUACCGGCAUGCAACCGACAAAACCGAAUCCG
CGAGUACUAACCUCCGGGUCCGGGCUCACGUCUCCUUCGGGAGCCGGAUGCCCUGGCC
UGUUGGGACCGUAACGGUAGUAGGUCAGCGCGGGAGUGACGCAGAAGGGUAGCCGGC
CGCGGAGGUGGUCUUCCGUGGUCAAGCACGCAGCCCGUCCCACAGGCAAAUCCGUGGG
GCGUGUGGGCGAGGUGCGAUGAUGGGCGCCGUUGGGCGCGAUAUCCGGUGAUCCCGG
CUGCCGAGAAAAGCUUCGGCGUGAGGCGGCGUGCCGCCCGUACCCCAAACCGACACUG
GUGGUCAGGUAGAGAAUACCAAAGCGAUCGAGCGAAUCCUGGUCAAGGAACUCGGCA
AAUCACUCCCGUUCCUUCGGUUUAAGGGAGACCCCCGAUGGUGAAGCGGCUUGCCCGC
GGAGCUUUUGGGGGUGGCACAGACCAGGGGGUAGCGACUGUUUACCAAAAACACAGG
AGCGUGCGAAGGCGCAAGCCGCUGUAUACGCUCUGACGCCUGCCCGGUGCCGGAAGGU
UAAGAGGAUCCGUCAGGCCUCGGCCGAAGCGGUGAAUUCAAGCCCCGGUAAACGGCGG
UGGUAACUAUAACCAUCCUAAGGUAGCGAAAUUCCUUGUCGGGUAAGUUCCGACCUG
CACGAAUGGCGUAACGACUUCCCCACUGUCUCGACCAGGAGCUCGGCGAAAUUGCAGU
ACGAGUAAAGAUGCUCGUUAAGCGCAGAAGGACGAAAAGACCCCGGGACCUUUACUA
UACCUUGGUAUUGGCAUUCGGUGUGGAUUGUGUAGCAUAGGCGGAAGGCUUCGAAGC
CACGGCGCCAGCCGCGGUGGAGCCGCAAGGUGAAAUACCGCUCUGUUCGCAUUGGAUG
UCUAACCUCGACCAGUCAUCCUGGUCAGGGACAGUGCCUGGCGGGUAGUUUAACUGG
GGCGGUUGCCUCCCAAAGAGUAACGGAGGCGCUCAAAGGUUCCCUCAGCCCGGUUGGC
AAUCGGGUGGCGAGUGUAAGUGCACAAGGGAGCUUGACUGCGAGACUGACGGGUCGA
GCAGGGACGAAAGUCGGAACUAGUGAUCCGGUGCCGGUGUACGGACGCGGCAUCGCU
CAACGGAUAAAAGGUACCCCGGGGAUAACAGGCUGAUCAUUCCCAAGAGUCCAUAUC
GACGGGAUGGUUUGGCACCUCGAUGUCGGCUCGUCGCAUCCUGGGGCUGGAGCAGGU
CCCAAGGGUUCGGCUGUUCGCCGAUUAAAGCGGCACGCGAGCUGGGUUCAGAACGUCG
UGAGACAGUUUGGUCUCUAUCCUCUGCGCUCGUCGGAAUGUUGAGGAGGCCUGCCCA
UAGUACGAGAGGACCUGGGUGGACGAACCUCUGGUAUGCCGGUUGUCACGCCAGUGG
CACGGCCGGUUGGCUACGUUCGGAAGGGAUAACCGCUGAAAGCAUCUAAGCGGGAAG
CCUGCUCCAAGAUAAGCAUUCCAUAGGACUACGGGUCCUUGAAGUCCCCAUGCAGAAC
ACGUGGUUGAUAGGCCGGACGUGGAAGCCCCGCGAGGGGUGGAGCCGACCGGUACUA
ACGGACGAAAGGCAACACAAC
SEQ ID NO: 19, B. breve, Accession Number: CP021558.1782121.1785182
Bifidobacterium; Bifidobacterium breve. Sequence:
GUGUUGCCUGCAAGGGCGUAUGGUGGAUGCCUUGGCAGACAGGACCGAUGAAGGACG
UUUGAGGCUGCGAUAUGCCUCGGGGAGCCGCCAACAGGGCUUUGAUCCGAGGAUUUC
CGAAUGGGGGAACCCACCGGCCGUCAUGGGCCGGUACCGCCGUGUGCGGGGGGUACGC
AGGGAAGUGAAACAUCUCAGUACCUGCAGGAAAGGAUAUUCCGUGAGUAGUGGCGAG
CGAAAGCGGAUCAGGCCAAACCUUGCGCGUGUGAUACCCGUCGGGGGUUGCGCGCGGG
GUGUAGAGGGAUCGCGUUUGCCGGCUCCGACGGGCCGGCCGUCAGUUGCAUAAAACCA
UGUGCCAGGGGAACCGGGUUGAAUACCGGGCCGCAGAGGGUGAGGGCCCCGUACCCGA
ACGCGCAUGGUCUGGCGAUCGCGUCUCCCAAGUAGCACGGGCCCCGUGGAAUCCCGUG
CGAAUCUGUCCCGACCGUGGGAUAAGCCUAAAUAUUCCUGUCUGACCGAUAGCGAACG
AGUACCGUGAGGGAAAGGUGAAAAGUACCCCGGGAGGGGAGUGAAACAGUCUCUGAA
ACCAUGCGCCUACGAACCGUCGGGGCCCUCUUGUGGGGUGACGGCGUGCCUAUCGAAA
AAUGAGUCUGCGAGUCAGUGGCAUGUGGCGAGCAUAACCCGUGUGGGGUAUGCGUAG
CGAAGGCGAGUCUUAAAAGGCGUUUAAAGUCGCGUGUCCUGGACCCGAAGCGGGAUG
AUCUAGCCCUGAGCAGGUUGAAGCGCGGGUAAGACCGUGUGGAGGACCGAACCCACCU
GGGUUGAAAACCGGGGGGAUGACUUGGGGCUAGGGGUGAAAGGCCAAUCAAAUUCCG
UGAUAGCUGGUUCUCUCCGAAAUGCAUUUAGGUGCAGCGUCGGAUCGAUUACAUCCG
GGGGGUAGAGCGACUGGAUGCUUGCGGGCCCAUAUCGGGUACCAACAGCAACCAAAC
UCCGAAUACCCGUGAUGCGUAUCCCGGCAGUGAGUCGGCGGGGGAUAAGCUCCGUCGU
CGAAAGGGAAACAGCCCAGAUCGUCGUCUAAGGUCCCCAAGCGUGUGCUAAGUGGGA
AAGGAUGUGGAGUCGCAUAGACAGCCAGGAGGUUGGCUCAGAAGCAGCCACCCUUGA
AAGAGUGCGUAACAGCUCACUGGUCUAGUGGUUCCGCGCCGACAAUGUAGCGGGGCU
CAAGCACACCACCGAAGACGCGGCAGUAGCUUUGCUACUGGGUAGGAGAGCGUCCCAU
GCGGGGCGAAGCGGCGGCGCAAGCCCGCCGUGGACCGCAUGGGAGUGAGAAUGCAGAC
AUGAGUAGCGAAAGACGGGUGAAGAUCCCGUCCGCUGGACGACCAAGGGUUCCAGGG
CCACGUUCAUCGUCCCUGGGUGAGUCGGGUCCUAAGGCGAGGCCGACAGGCGUAGUCG
AAUGGAUGAACGGGUCGAUAUUCCCGUACCGGCAUGCAACCGGCAAAACCGAAUCCGC
GAGUACUAACCUCCGGGUCCGGGCUUACGUCUCCUUCGGGAGCCGGAUGCCCUGGCCU
GUUGGGACCGUAACGGUAGUAGGUCAGCGCGGGAGUGACGCAGAAGGGUAGCCGGCC
GCGGAGGUGGUCUUCCGUGGUCAAGCACGCAGCCCGUCCCACAGGCAAAUCCGUGGGG
CGCGUGGGCGAGGUGCGAUGAUGGGCGCCGCAGGGCGCGAUAUCCGGUGAUCCCGGCU
GCCGAGAAAAGCUUCGGCGUGAGGCGGCGUGCCGCCCGUACCCCAAACCGACACUGGU
GGUCAGGUAGAGAAUACCAAAGCGAUCGAGCGAAUCCUGGUCAAGGAACUCGGCAAA
UCACUCCCGUUCCUUCGGUUUAAGGGAGACCCCCGAUGGUGAAGCGGCUUGCCCGCGG
AGCUUUUGGGGGUGGCACAGACCAGGGGGUAGCGACUGUUUACCAAAAACACAGGAG
CGUGCGAAGGCGCAAGCCGCUGUAUACGCUCUGACGCCUGCCCGGUGCCGGAAGGUUA
AGAGGAUCCGUCAGGCCUCGGCCGAAGCGGUGAAUUCAAGCCCCGGUAAACGGCGGUG
GUAACUAUAACCAUCCUAAGGUAGCGAAAUUCCUUGUCGGGUAAGUUCCGACCUGCA
CGAAUGGCGUAACGACUUCCCCACUGUCUCGACCAGGAGCUCGGCGAAAUUGCAGUAC
GAGUAAAGAUGCUCGUUAAGCGCAGAAGGACGAAAAGACCCCGGGACCUUUACUAUA
CCUUGGUAUUGGCAUUCGGUGUGGAUUGUGUAGCAUAGGCGGAAGGCUUCGAAGCCA
CGGCGCCAGCCGCGGUGGAGCCGCAAGGUGAAAUACCGCUCUGUUCGCAUUGGAUGUC
UAACCUCGACCAGUCAUCCUGGUCAGGGACAGUGCCUGGCGGGUAGUUUAACUGGGG
CGGUUGCCUCCCAAAGAGUAACGGAGGCGCUCAAAGGUUCCCUCAGCCCGGUUGGCAA
UCGGGUGGCGAGUGUAAGUGCACAAGGGAGCUUGACUGCGAGACUGACGGGUCGAGC
AGGGACGAAAGUCGGAACUAGUGAUCCGGUGCCGGUGUACGGACGCGGCAUCGCUCA
ACGGAUAAAAGGUACCCCGGGGAUAACAGGCUGAUCAUUCCCAAGAGUCCAUAUCGA
CGGGAUGGUUUGGCACCUCGAUGUCGGCUCGUCGCAUCCUGGGGCUGGAGCAGGUCCC
AAGGGUUCGGCUGUUCGCCGAUUAAAGCGGCACGCGAGCUGGGUUCAGAACGUCGUG
AGACAGUUUGGUCUCUAUCCUCUGCGCUCGUCGGAAUGUUGAGGAGGCCUGCCCAUA
GUACGAGAGGACCUGGGUGGACGAACCUCUGGUAUGCCGGUUGUCACGCCAGUGGCA
CGGCCGGUUGGCUACGUUCGGAAGGGAUAACCGCUGAAAGCAUCUAAGCGGGAAGCC
UGCUCCAAGAUAAGCAUUCCAUAGGACUACGGGUCCUUGAAGUCCCCAUGCAGAACAC
GUGGUUGAUAGGCCGGACGUGGAAGCCCCGCGAGGGGUGGAGCCGACCGGUACUAAC
GGACGAAAGGCAACAC
SEQ ID NO: 20, B. breve, Accession Number: CP021388.2282979.2286039
Bifidobacterium; Bifidobacterium breve. Sequence:
GUGUUGCCUGCAAGGGCGUAUGGUGGAUGCCUUGGCAGACAGGACCGAUGAAGGACG
UUUGAGGCUGCGAUAUGCCUCGGGGAGCCGCCAACAGGGCUUUGAUCCGAGGAUUUC
CGAAUGGGGGAACCCACCGGCCGUCAUGGGCCGGUACCGCCGUGUGCGGGGGGUACGC
AGGGAAGUGAAACAUCUCAGUACCUGCAGGAAAGGAUAUUCCGUGAGUAGUGGCGAG
CGAAAGCGGAUCAGGCCAAACCUUGCGCGUGUGAUACCCGUCGGGGGUUGCGCGCGGG
GUGUAGAGGGAUCGCGUUUGCCGGCUCCGACGGGCCGGCCGUCAGUUGCAUAAAACCA
UGUGCCAGGGGAACCGGGUUGAAUACCGGGCCGCAGAGGGUGAGGGCCCCGUACCCGA
ACGCGCAUGGUCUGGCGAUCGCGUCUCCCAAGUAGCACGGGCCCCGUGGAAUCCCGUG
CGAAUCUGUCCCGACCGUGGGAUAAGCCUAAAUAUUCCUGUCUGACCGAUAGCGAACG
AGUACCGUGAGGGAAAGGUGAAAAGUACCCCGGGAGGGGAGUGAAACAGUCUCUGAA
ACCAUGCGCCUACGAACCGUCGGGGCCCUCUUGUGGGGUGACGGCGUGCCUAUCGAAA
AAUGAGUCUGCGAGUCAGUGGCAUGUGGCGAGCAUAACCCGUGUGGGGUAUGCGUAG
CGAAGGCGAGUCUUAAAAGGCGUUUGAGUCGCGUGUCCUGGACCCGAAGCGGGAUGA
UCUAGCCCUGAGCAGGUUGAAGCGCGGGUAAGACCGUGUGGAGGACCGAACCCACCUG
GGUUGAAAACCGGGGGGAUGACUUGGGGCUAGGGGUGAAAGGCCAAUCAAAUUCCGU
GAUAGCUGGUUCUCUCCGAAAUGCAUUUAGGUGCAGCGUCGGAUCGAUUACAUCCGG
GGGGUAGAGCGACUGGAUGCUUGCGGGCCCAUAUCGGGUACCAACAGCAACCAAACUC
CGAAUACCCGUGAUGCGUAUCCCGGCAGUGAGUCGGCGGGGGAUAAGCUCCGUCGUCG
AAAGGGAAACAGCCCAGAUCGUCGUCUAAGGUCCCCAAGCGUGUGCUAAGUGGGAAA
GGAUGUGGAGUCGCAUAGACAGCCAGGAGGUUGGCUCAGAAGCAGCCACCCUUGAAA
GAGUGCGUAACAGCUCACUGGUCUAGUGGUUCCGCGCCGACAAUGUAGCGGGGCUCA
AGCACACCACCGAAGACGCGGCAGUAGCUUUGCUACUGGGUAGGAGAGCGUCCCAUGC
GGGGCGAAGCGGCGGCGCAAGCCCGCCGUGGACCGCAUGGGAGUGAGAAUGCAGACA
UGAGUAGCGAAAGACGGGUGAAGAUCCCGUCCGCUGGACGACCAAGGGUUCCAGGGC
CACGUUCAUCGUCCCUGGGUGAGUCGGGUCCUAAGGCGAGGCCGACAGGCGUAGUCGA
AUGGACGAACGGGUCGAUAUUCCCGUACCGGCAUGCAACCGGCAAAACCGAAUCCGCG
AGUACUAACCUCCGGGUCCGGGCUUACGUCUCCUUCGGGAGCCGGAUGCCCUGGCCUG
UUGGGACCGUAACGGUAGUAGGUCAGCGCGGGAGUGACGCAGAAGGGUAGCCGGCCG
CGGAGGUGGUCUUCCGUGGUCAAGCACGCAGCCCGUCCCACAGGCAAAUCCGUGGGGC
GCGUGGGCGAGGUGCGAUGAUGGGCGCCGCAGGGCGCGAUAUCCGGUGAUCCCGGCU
GCCGAGAAAAGCUUCGGCGUGAGGCGGCGUGCCGCCCGUACCCCAAACCGACACUGGU
GGUCAGGUAGAGAAUACCAAAGCGAUCGAGCGAAUCCUGGUCAAGGAACUCGGCAAA
UCACUCCCGUUCCUUCGGUUUAAGGGAGACCCCCGAUGGUGAACACACUUGCUGUGGG
AGCUUUUGGGGGUGGCACAGACCAGGGGGUAGCGACUGUUUACCAAAAACACAGGAG
CGUGCGAAGGCGCAAGCCGCUGUAUACGCUCUGACGCCUGCCCGGUGCCGGAAGGUUA
AGAGGAUCCGUCAGGCUUCGGCCGAAGCGGUGAAUUCAAGCCCCGGUAAACGGCGGU
GGUAACUAUAACCAUCCUAAGGUAGCGAAAUUCCUUGUCGGGUAAGUUCCGACCUGC
ACGAAUGGCGUAACGACUUCCCCACUGUCUCGACCAGGAGCUCGGCGAAAUUGCAGUA
CGAGUAAAGAUGCUCGUUAAGCGCAGAAGGACGAAAAGACCCCGGGACCUUUACUAU
ACCUUGGUAUUGGCAUUCGGUGUGGAUUGUGUAGCAUAGGCGGAAGGCUUCGAAGCC
ACGGCGCCAGCCGCGGUGGAGCCGCAAGGUGAAAUACCGCUCUGUUCGCAUUGGAUGU
CUAACCUCGACCAGUCAUCCUGGUCAGGGACAGUGCCUGGCGGGUAGUUUAACUGGG
GCGGUUGCCUCCCAAAGAGUAACGGAGGCGCUCAAAGGUUCCCUCAGCCCGGUUGGCA
AUCGGGUGGCGAGUGUAAGUGCACAAGGGAGCUUGACUGCGAGACUGACGGGUCGAG
CAGGGACGAAAGUCGGAACUAGUGAUCCGGUGCCGGUGUACGGACGCGGCAUCGCUC
AACGGAUAAAAGGUACCCCGGGGAUAACAGGCUGAUCAUUCCCAAGAGUCCAUAUCG
ACGGGAUGGUUUGGCACCUCGAUGUCGGCUCGUCGCAUCCUGGGGCUGGAGCAGGUCC
CAAGGGUUCGGCUGUUCGCCGAUUAAAGCGGCACGCGAGCUGGGUUCAGAACGUCGU
GAGACAGUUUGGUCUCUAUCCUCUGCGCUCGUCGGAAUGUUGAGGAGGCCUGCCCAU
AGUACGAGAGGACCUGGGUGGACGAACCUCUGGUAUGCCGGUUGUCACGCCAGUGGC
ACGGCCGGUUGGCUACGUUCGGAAGGGAUAACCGCUGAAAGCAUCUAAGCGGGAAGC
CUGCUCCAAGAUAAGCAUUCCAUAGGACUACGGGUCCUUGAAGUCCCCAUGCAGAACA
CGUGGUUGAUAGGCCGGACGUGGAAGCCCCGCGAGGGGUGGAGCCGACCGGUACUAA
CGGACGAAAGGCAACAC
Exemplary B. infantis sequences
SEQ ID NO: 21, B. infantis 16S sequence, 1526 nt (see e.g., NCBI Gene ID: 66505550)
TTTGTGGAGGGTTCGATTCTGGCTCAGGATGAACGCTGGCGGCGTGCTTAACACATGCAA
GTCGAACGGGATCCATCAGGCTTTGCTTGGTGGTGAGAGTGGCGAACGGGTGAGTAATG
CGTGACCGACCTGCCCCATACACCGGAATAGCTCCTGGAAACGGGTGGTAATGCCGGAT
GCTCCAGTTGATCGCATGGTCTTCTGGGAAAGCTTTCGCGGTATGGGATGGGGTCGCGTC
CTATCAGCTTGACGGCGGGGTAACGGCCCACCGTGGCTTCGACGGGTAGCCGGCCTGAG
AGGGCGACCGGCCACATTGGGACTGAGATACGGCCCAGACTCCTACGGGAGGCAGCAGT
GGGGAATATTGCACAATGGGCGCAAGCCTGATGCAGCGACGCCGCGTGAGGGATGGAG
GCCTTCGGGTTGTAAACCTCTTTTATCGGGGAGCAAGCGAGAGTGAGTTTACCCGTTGAA
TAAGCACCGGCTAACTACGTGCCAGCAGCCGCGGTAATACGTAGGGTGCAAGCGTTATC
CGGAATTATTGGGCGTAAAGGGCTCGTAGGCGGTTCGTCGCGTCCGGTGTGAAAGTCCAT
CGCTTAACGGTGGATCCGCGCCGGGTACGGGCGGGCTTGAGTGCGGTAGGGGAGACTGG
AATTCCCGGTGTAACGGTGGAATGTGTAGATATCGGGAAGAACACCAATGGCGAAGGCA
GGTCTCTGGGCCGTTACTGACGCTGAGGAGCGAAAGCGTGGGGAGCGAACAGGATTAGA
TACCCTGGTAGTCCACGCCGTAAACGGTGGATGCTGGATGTGGGGCCCGTTCCACGGGTT
CCGTGTCGGAGCTAACGCGTTAAGCATCCCGCCTGGGGAGTACGGCCGCAAGGCTAAAA
CTCAAAGAAATTGACGGGGGCCCGCACAAGCGGCGGAGCATGCGGATTAATTCGATGCA
ACGCGAAGAACCTTACCTGGGCTTGACATGTTCCCGACGGTCGTAGAGATACGGCTTCCC
TTCGGGGCGGGTTCACAGGTGGTGCATGGTCGTCGTCAGCTCGTGTCGTGAGATGTTGGG
TTAAGTCCCGCAACGAGCGCAACCCTCGCCCCGTGTTGCCAGCGGATTATGCCGGGAACT
CACGGGGGACCGCCGGGGTTAACTCGGAGGAAGGTGGGGATGACGTCAGATCATCATGC
CCCTTACGTCCAGGGCTTCACGCATGCTACAATGGCCGGTACAACGGGATGCGACGCGG
CGACGCGGAGCGGATCCCTGAAAACCGGTCTCAGTTCGGATCGCAGTCTGCAACTCGAC
TGCGTGAAGGCGGAGTCGCTAGTAATCGCGAATCAGCAACGTCGCGGTGAATGCGTTCC
CGGGCCTTGTACACACCGCCCGTCAAGTCATGAAAGTGGGCAGCACCCGAAGCCGGTGG
CCTAACCCCTTGTGGGATGGAGCCGTCTAAGGTGAGGCTCGTGATTGGGACTAAGTCGTA
ACAAGGTAGCCGTACCGGAAGGTGCGGCTGGATCACCTCCTTT
SEQ ID NO: 22, B. infantis, Accession Number: AP010889.2757707.2760771
Bifidobacterium; Bifidobacterium longum subsp. infantis. Sequence:
UGUGUUGCUUGCAAGGGCGUAUGGUGGAUGCCUUGGCAGACAGGACCGAUGAAGGAC
GUUUGAGGCUGCGAUAAGCCUCGGGGAGCCGCCUACAGGGCUUUGAUCCGAGGAUUU
CCGAAUGGGGGAACCCACCGGCCGUCAUGGGUCGGUACCGGCUUCGGCCGGGGGGUAC
GCAGGGAAGUGAAACAUCUCAGUACCUGCAGGAAAGGAUAUUCCGUGAGUAGUGGCG
AGCGAAAGCGGAUGAUGGCCAAACCUUGCGCGUGUGAUACCCGUCGGGGGUUGCGUG
UGGGGUGUUGCGGGAUCGCGUGUGCCGGCUCCGACGGGCCGGCCGGCAGUUGUAGAA
AACCAUGUGUCAGGGGAACCGGGUUGAAUACCGGGCCGCAGAGGGUGAGGGCCCCGU
ACCUGAACGCGCAUGGUCUGCCGAUCGCGUCUCCCAAGUAGCACGGGUCCCGUGGAAC
CCCGUGCGAAUCCGCCCGGACCGUCGGGUAAGCCUGAAUAUUCCUGUCUGACCGAUAG
CGAACGAGUACCGUGAGGGAAAGGUGAAAAGUACCCCGGGAGGGGAGUGAAACAGUC
UCUGAAACCGUGCGCCUACAAUCCGUCGGAGCCUCUUUGUGGGGUGACGGCGUGCCUA
UCGAAAAAUGAGUCUGCGAGUCAGUGGCAUGUGGCGAGCAUAACCCGUGUGGGGUAU
GCGUAGCGAAGGCGAGUCUUAAAAGGCGUUUAAAGUCGCGUGUCCUGGACCCGAAGC
GGGAUGAUCUAGCCCUGAGCAGGUUGAAGCGCGGGUAAGACCGUGUGGAGGACCGAA
CCCACCUGGGUUGAAAACCGGGGGGAUGACUUGGGGUUAGGGGUGAAAGGCCAAUCA
AAUUCCGUGAUAGCUGGUUCUCUCCGAAAUGCAUUUGGGUGCAGCGUCGGGUCAUUG
CGUCCCGGGGGUAGAGCUACUGGAUGCUUGCGGGCCCGUAUCGGGUACCAACAGCAAC
CAAACUCCGAAUACCGGUGACGUGUAUCCCGGCAGUGAGUCGGCGGGGGAUAAGCUU
CGUCGUCGAAAGGGAAACAGCCCAGACCGUCGUCUAAGGUCCCGAAGCGUGUGCUAAG
UGGGAAAGGAUGUGGAGUCGCAUAGACAGCCAGGAGGUUGGCUCAGAAGCAGCCAUC
CUUGAAAGAGUGCGUAACAGCUCACUGGUCUAGUGGUUCCGCGCCGACAAUGUAGCG
GGGCUCAAGCACACCACCGAAGACGCGGCAGUAUUUUGUACUGGGUAGGAGAGCGUC
CCAUGAGGGGCGAAGCGGCCGUGUGAACGCGCCGUGGACUUCAUGGGAGUGAGAAUG
CAGACAUGAGUAGCGAAAGACGGGUGAAGAUCCCGUCCGCUGGAUGACCAAGGGUUC
CAGGGCCACGUUCAUCGUCCCUGGGUGAGUCGGGUCCUAAGGCGAGGCCGACAGGCGU
AGUCGAAUGGAUGAACGGGUCGAUAUUCCCGUACCGGCUUUCAGCCGCCAGAACCGAG
GCUUGGAGUACUAACCUCCGGGUCCGGGCUUGCUUCCCCUUCGGGGGUGGGAUGCCUG
GGCCUGUUGGGACCGAUCCUUGUAGUAGGUCAGCGCAGGAGUGACGCAGAAGGGUAG
CCGGCCGCGGAGGUGGUUUUCCGUGGUCAAGCACGCAGCCCGUCCCGUAGGCAAAUCC
GCGGGGCGUGUGGGCGAGGUGCGAUGGUGGGGGCCUUGUGGCCCGAUAUCCGGUGAU
CCCGGCUGCCGAGAAAAGCUUCGGCGUGAGGCUCGAAGCCGCCCGUACCCCAAACCGA
CACUGGUGGUCAGGUAGAGAAUACCAAAGCGAUCGAGCGAAUCCUGGUCAAGGAACU
CGGCAAAUCACUCCCGUUCCUUCGGUUUAAGGGAGACCCCCGAUGGUGAGCCGCCUCG
CGCGGGGAGCUUUUGGGGGUGGCACAGACCAGGGGGUAGCGACUGUUUACCAAAAAC
ACAGGUGCAUGCGAAGGCGCAAGCCGCUGUAUAUGCACUGACGCCUGCCCGGUGCCGG
AAGGUUAAGAGGAUCCGUCAGCCUUCGGGUGAAGCGGUGAAUUCAAGCCCCGGUAAA
CGGCGGUGGUAACUAUAACCAUCCUAAGGUAGCGAAAUUCCUUGUCGGGUAAGUUCC
GACCUGCACGAAUGGCGUAACGACUUCCCCACUGUCUCGACCAGGAGCUCGGCGAAAU
UGCAGUACGAGUAAAGAUGCUCGUUAAGCGCAGAAGGACGAAAAGACCCCGGGACCU
UUACUAUACCUUGGUAUUGGCAUUCGGUGUGGAUUGUGUAGCAUAGGCGGGAGGCCG
AGAGGCAUGGUCGCCAGAUCGUGCGGAGCCGCAAGGUGAAAUACCGCUCUGUUCGCA
UUGGAUGUCUAACCUCGAACAGUGAUCCUGUUCAGGGACAGUGCCUGGCGGGUAGUU
UAACUGGGGCGGUUGCCUCCCAAAGAGUAACGGAGGCGCCCAAAGGUCCGCUCAGCCC
GGUUGGCAAUCGGGUGGCGAGUGCAAGUGCACAAGCGGGCUUGACUGCGAGGACGAC
GGUCCGAGCAGGGACGAAAGUCGGGACUAGUGAUCCGGUGCCGGUGUACGGACGCGG
CAUCGCUCAACGGAUAAAAGGUACCCCGGGGAUAACAGGCUGAUCAUUCCCAAGAGUC
CAUAUCGACGGGAUGGUUUGGCACCUCGAUGUCGGCUCGUCGCAUCCUGGGGCUGGA
GCAGGUCCCAAGGGUUCGGCUGUUCGCCGAUUAAAGCGGCACGCGAGCUGGGUUCAG
AACGUCGUGAGACAGUUUGGUCUCUAUCCUCUGCGCUCGUUGGAAUGUUGAGGAGGC
CUGCCCAUAGUACGAGAGGACCUGGGUGGACGAACCUCUGGUAUGCCGGUUGUCACGC
CAGUGGCACGGCCGGUUGGCUACGUUCGGAAGGGAUAACCGCUGAAAGCAUCUAAGC
GGGAAGCCUGCUCCAAGAUAAGCAUUCCGUGCAGCCUCGGGCUGCUGUAGUCCCCAUG
CAGAACACGUGGUCGAUAGGCCGGACGUGGAAGCCCCGCGAGGGGUGGAGCCGACCGG
UACUAACGGACGAAAGGCAACACUA
SEQ ID NO: 23, B. infantis, Accession Number: AP010889.2751543.2754607
Bifidobacterium; Bifidobacterium longum subsp. infantis. Sequence:
GCUUGCAAGGGCGUAUGGUGGAUGCCUUGGCAGACAGGACCGAUGAAGGACGUUUGA
GGCUGCGAUAUGCCUCGGGGAGCCGCCAACAGGGCUUUGAUCCGAGGAUUUCCGAAU
GGGGGAACCCACCGGCCGUCAUGGGUCGGUACCGGCUUCGGCCGGGGGGUACGCAGGG
AAGUGAAACAUCUCAGUACCUGCAGGAAAGGAUAUUCCGUGAGUAGUGGCGAGCGAA
AGCGGAUGAUGGCCAAACCUUGCGCGUGUGAUACCCGUCGGGGGUUGCGCGCGGGGU
GUUGCGGGAUCGCGUGUGCCGGCUCCGACGGGCCGGCCGGCAGUUGCAGAAAACCAUG
UGUGAGGGGAACCGGGUUGAAUACCGGGCCGCAGAGGGUGAGGGCCCCGUACCCGAA
CGCGCAUGGUCUGCCGAUCGCGUCUCCCAAGUAGCACGGGUCCCGUGGAACCCCGUGC
GAAUCCGCCCAGACCGUUGGGUAAGCCUGAAUAUUCCUGUCUGACCGAUAGCGAACGA
GUACCGUGAGGGAAAGGUGAAAAGUACCCCGGGAGGGGAGUGAAACAGUCUCUGAAA
CCGUGCGCCUACAAUCCGUCGGAGCCUUCUUUGUGGGGUGACGGCGUGCCUAUCGAAA
AAUGAGUCUGCGAGUCAGUGGCAUGUGGCGAGCAUAACCCGUGUGGGGUAUGCGUAG
CGAAGGCGAGUCUUAAAAGGCGUUUGAGUCGCGUGUCCUGGACCCGAAGCGGGAUGA
UCUAGCCCUGAGCAGGUUGAAGCGCGGGUAAGACCGCGUGGAGGACCGAACCCACCUG
GGUUGAAAACCGGGGGGAUGACUUGGGGCUAGGGGUGAAAGGCCAAUCAAAUUCCGU
GAUAGCUGGUUCUCUCCGAAAUGCAUUUGGGUGCAGCGUCGGGUCAUUACGUCCCGG
GGGUAGAGCUACUGGAUGCUUGCGGGCCCGUAUCGGGUACCAACAGCAACCAAACUCC
GAAUACCGGUGACGCGUAUCCCGGCAGUGAGUCGGCGGGGGAUAAGCUUCGUCGUCG
AAAGGGAAACAGCCCAGACCGUCGUCUAAGGUCCCGAAGCGUGUGCUAAGUGGGAAA
GGAUGUGGAGUCGCAUAGACAGCCAGGAGGUUGGCUCAGAAGCAGCCAUCCUUGAAA
GAGUGCGUAACAGCUCACUGGUCUAGUGGUUCCGCGCCGACAAUGUAGCGGGGCUCA
AGCACACCACCGAAGACGCGGCAGUAUCGUUUGGUACUGGGUAGGAGAGCGUCCCAU
GAGGGGCGAAGCGGGCGUGGAAGCGUCCGUGGACUUCAUGGGAGUGAGAAUGCAGAC
AUGAGUAGCGAAAGACGGGUGAAGAUCCCGUCCGCUGGAUGACCAAGGGUUCCAGGG
CCACGUUCAUCGUCCCUGGGUGAGUCGGGUCCUAAGGCGAGGCCGACAGGCGUAGUCG
AAUGGAUGAACGGGUCGAUAUUCCCGUACCGGCUUUCAGCCGCCAGAACCGAGGCUUG
GAGUACUAACCUCCGGGUCCGGGCUUACCGCUCCUUCGGGAGGGGGAUGCCCUGGCCU
GUUGGGACCGAUCCUUGUAGUAGGUCAGCGCAGGAGUGACGCAGAAGGGUAGCCGGC
CGCGGAGGUGGUUUUCCGUGGUCAAGCACGCAGCCCGUCCCGUAGGCAAAUCCGCGGG
GCGUGUGGGCGAGGUGCGAUGGUGGGGGCCUUAUGGCCCGAUAUCCGGUGAUCCCGG
CUGCCGAGAAAAGCUUCGGCGUCAGGCUCGAAGCCGCCCGUACCCCAAACCGACACUG
GUGGUCAGGUAGAGAAUACCAAAGCGAUCGAGCGAAUCCUGGUCAAGGAACUCGGCA
AAUCACUCCCGUUCCUUCGGUUUAAGGGAGACCCCCGAUGGUGAGCCGCCUCGCGCGG
GGAGCUUUUGGGGGUGGCACAGACCAGGGGGUAGCGACUGUUUACCAAAAACACAGG
UGCAUGCGAAGGCGCAAGCCGCUGUAUAUGCACUGACGCCUGCCCGGUGCCGGAAGGU
UAAGAGGAUCCGUCAGCCUUCGGGUGAAGCGGUGAAUUCAAGCCCCGGUAAACGGCG
GUGGUAACUAUAACCAUCCUAAGGUAGCGAAAUUCCUUGUCGGGUAAGUUCCGACCU
GCACGAAUGGCGUAACGACUUCCCCACUGUCUCGACCAGGAGCUCGGCGAAAUUGCAG
UACGAGUAAAGAUGCUCGUUAAGCGCAGAAGGACGAAAAGACCCCGGGACCUUUACU
AUACCUUGGUAUUGGCAUUCGGUGUGGAUUGUGUAGCAUAGGCGGGAGGCCGAGAGG
CAUGGUCGCCAGAUCGUGCGGAGCCGCAAGGUGAAAUACCGCUCUGUUCGCAUUGGA
UGUCUAACCUCGAACAGUCAUCCUGUUCAGGGACAGUGCCUGGCGGGUAGUUUAACU
GGGGCGGUUGCCUCCCAAAGAGUAACGGAGGCGCCCAAAGGUCCGCUCAGCCCGGUUG
GCAAUCGGGUGGCGAGUGCAAGUGCACAAGCGGGCUUGACUGCGAGGACGACGGUCC
GAGCAGGGACGAAAGUCGGGACUAGUGAUCCGGUGCCGGUGUACGGACGCGGCAUCG
CUCAACGGAUAAAAGGUACCCCGGGGAUAACAGGCUGAUCAUUCCCAAGAGUCCAUA
UCGACGGGAUGGUUUGGCACCUCGAUGUCGGCUCGUCGCAUCCUGGGGCUGGAGCAG
GUCCCAAGGGUUCGGCUGUUCGCCGAUUAAAGCGGCACGCGAGCUGGGUUCAGAACG
UCGUGAGACAGUUUGGUCUCUAUCCUCUGCGCUCGUUGGAAUGCUGAGGAGGCCUGC
CCAUAGUACGAGAGGACCUGGGUGGACGAACCUCUGGUAUGCCGGUUGUCACGCCAG
UGGCACGGCCGGUUGGCUACGUUCGGAAGGGAUAACCGCUGAAAGCAUCUAAGCGGG
AAGCCUGCUCCAAGAUAAGCAUUCCGUGCAGCCUCGAGCUGCUGUAGUCCCCAUGCAG
AACACGUGGUCGAUAGGCCGGACGUGGAAGCCCCGCGAGGGGUGGAGCCGACCGGUAC
UAACGGACGAAAGGCA
SEQ ID NO: 24, B. infantis, Accession Number: CP001095.2539820.2542677
Bifidobacterium; Bifidobacterium longum subsp. infantis ATCC 15697 = JCM 1222 =
DSM 20088.Sequence:
UGUGUUGCUUGCAAGGGCGUAUGGUGGAUGCCUUGGCAGACAGGACCGAUGAAGGAC
GUUUGAGGCUGCGAUAUGCCUCGGGGAGCCGCCAACAGGGCUUUGAUCCGAGGAUUU
CCGAAUGGGGGAACCCACCGACCGUCAUGGGUCGGUACCGGCUUCGGCCGGGGGGUAC
GCAGGGAAGUGAAACAUCUCAGUACCUGCAGGAAAGGAUAUUCCGUGAGUAGUGGCG
AGCGAAAGCGGAUGAUGGCCAAACCUUGCGCGUGUGAUACCCGUCGGGGGUUGCGCG
CGGGGUGUUGCGGGAUCGCGUGCGCCGGCUCCGACGGGCCGGCCGGCAGUUGCAGAAA
ACCAUGUGUCAGGGGAACCGGGUUGAAUACCGGGCCGCAGAGGGUGAGGGCCCCGUA
CCUGAACGCGCAUGGUCUGCCGAUCGCGUCUCCCAAGUAGCACGGGUCCCGUGGAACC
CCGUGCGAAUCCGCCCAGACCGUUGGGUAAGCCUGAAUAUUCCUGUCUGACCGAUAGC
GAACGAGUACCGUGAGGGAAAGGUGAAAAGUACCCCGGGAGGGGAGUGAAACAGUCU
CUGAAACCGUGCGCCUACAAUCCGUCGGAGCCCUUUUGUGGGGUGACGGCGUGCCUAU
CGAAAAAUGAGUCUGCGAGUCAGUGGCAUGUGGCGAGCAUAACCCGUGUGGGGUAUG
CGUAGCGAAGGCGAGUCUUAAAAGGCGUUUGAGUCGCGUGUCCUGGACCCGAAGCGG
GAUGAUCUAGCCCUGAGCAGGUUGAAGCGCGGGUAAGACCGCGUGGAGGACCGAACC
CACCUGGGUUGAAAACCGGGGGGAUGACUUGGGGCUAGGGGUGAAAGGCCAAUCAAA
UUCCGUGAUAGCUGGUUCUCUCCGAAAUGCAUUUGGGUGCAGCGUCGGGUCAUUACG
UCCCGGGGGUAGAGCUACUGGAUGCUUGCGGGCCCGUAUCGGGUACCAACAGCAACCA
AACUCCGAAUACCGGUGACGCGUAUCCCGGCAGUGAGUCGGCGGGGGAUAAGCUUCG
UCGUCGAAAGGGAAACAGCCCAGACCGUCGUCUAAGGUCCCGAAGCGUGUGCUAAGU
GGGAAAGGAUGUGGAGUCGCAUAGACAGCCAGGAGGUUGGCUCAGAAGCAGCCACCC
UUGAAAGAGUGCGUAACAGCUCACUGGUCUAGUGGUUCCGCGCCGACAAUGUAGCGG
GGCUCAAGCACACCACCGAAGACGCGGCAGUAUUUUGUACUGGGUAGGAGAGCGUCCC
AUGAGGGGCGAAGCGGCCGUGUGAACGCGCCGUGGACUUCAUGGGAGUGAGAAUGCA
GACAUGAGUAGCGAGAGACGGGUGAAGAUCCCGUCCGCUGGAUGACCAAGGGUUCCA
GGGCCACGUUCAUCGUCCCUGGGUGAGUCGGGUCCUAAGGCGAGGCCGACAGGCGUAG
UCGAAUGGAUGAACGGGUCGAUAUUCCCGUACCGGCUUUCAGCCGCCAAAACCGAGGC
UUGGAGUACUAACCUCCGGGUCCGGGCUUACUGCUCCUUCGGGGGCGGGAUGCCCUGG
CCUGUUGGGACCGAUCCUUGUAGUAGGUCAGCGCAGGAGUGACGCAGAAGGGUAGCC
GGCCGCGGAGGUGGUCUUCCGUGGUCAAGCACGCAGGGCGUGGGACAGGCAAAUCCG
UCCCGCAUGGGUCCGAGGUGCGAUGAUGGGGGCCUUAUGGCCCGAUAUCCGGUGAUCC
CGGCUGCCGAGAAAAGCUUCGGCGUCAGGCUCAAAGCCGCCCGUACCCCAAACCGACA
CUGGUGGUCAGGUAGAGAAUACCAAAGCGAUCGAGCGAAUCCUGGUCAAGGAACUCG
GCAAAUCACUCCCGUUCCUUCGGUUUAAGGGAGACCCCCGAUGGUGAACACACUUGCU
GUGGGAGCUUUUGGGGGUGGCACAGACCAGGGGGUAGCGACUGUUUACCAAAAACAC
AGGUGCAUGCGAAGGCGCAAGCCGCUGUAUAUGCACUGACGCCUGCCCGGUGCCGGAA
GGUUAAGAGGAUCCGUCAGCCUUCGGGUGAAGCGGUGAAUUCAAGCCCCGGUAAACG
GCGGUGGUAACUAUAACCAUCCUAAGGUAGCGAAAUUCCUUGUCGGGUAAGUUCCGA
CCUGCACGAAUGGCGUAACGACUUCCCCACUGUCUCGACCAGGAGCUCGGCGAAAUUG
CAGUACGAGUAAAGAUGCUCGUUAAGCGCAGAAGGACGAAAAGACCCCGGGACCUUU
ACUAUACCUUGGUAUUGGCAUUCGGUGUGGAUUGUGUAGCAUAGGCGGGAGGCCGAG
AGGCGCGGUCGCCAGAUCGCGCGGAGCCGUAAGGUGAAAUACCGCUCUGUUCGCAUUG
GAUGUCUAACCUCGAACAGUCAUCCUGUUCAGGGACAGUGCCUGGCGGGUAGUUUAA
CUGGGGCGGUUGCCUCCCAAAGAGUAACGGAGGCGCCCAAAGGUCCGCUCAGCCCGGU
UGGCAAUCGGGUGGCGAGUGCAAGUGCACAAGCGGGCUUGACUGCGAGGACGACGGU
CCGAGCAGGGACGAAAGUCGGGACUAGUGAUCCGGUGCCGGUGUACGGACGCGGCAU
CGCUCAACGGAUAAAAGGUACCCCGGGGAUAACAGGCUGAUCAUUCCCAAGAGUCCAU
AUCGACGGGAUGGUUUGGCACCUCGAUGUCGGCUCGUCGCAUCCUGGGGCUGGAGCA
GGUCCCAAGGGUUCGGCUGUUCGCCGAUUAAAGCGGCACGCGAGCUGGGUUCAGAAC
GUCGUGAGACAGUUUGGUCUCUAUCCUCUGCGCUCGUUGGAAUGCUGAGGAGGCCUG
CCCAUAGUACGAGAGGACCUGGGUGGACGAACCUCUGGUAUGCCGGUUGUCACGCCAG
UGGCACGGCCGGUUGGCUACGUUCGGAAGGGAUAACCGCUGAAAGCAUCUAAGCGGG
AAGCCUGCUCCAAGAUAAGCAUUCCGUGGAACCUUCGAGUUCCUGUAGUCCCCAUGCA
GAACACGUGGUCGAUAGGCCGGACGUGGAAGCCCCGCGAGGGGUGGAGCCGACCGGU
ACUAACGGACGAAGGGCAACACUAA
SEQ ID NO: 25, B. infantis, Accession Number: BCYG01000038.1.1181
Bifidobacterium; Bifidobacterium longum subsp. infantis. Sequence:
CCGCCUCGCGCGGGGAGCUUUUGGGGGUGGCACAGACCAGGGGGUAGCGACUGUUUA
CCAAAAACACAGGUGCAUGCGAAGGCGCAAGCCGCUGUAUAUGCACUGACGCCUGCCC
GGUGCCGGAAGGUUAAGAGGAUCCGUCAGCCUUCGGGUGAAGCGGUGAAUUCAAGCC
CCGGUAAACGGCGGUGGUAACUAUAACCAUCCUAAGGUAGCGAAAUUCCUUGUCGGG
UAAGUUCCGACCUGCACGAAUGGCGUAACGACUUCCCCACUGUCUCGACCAGGAGCUC
GGCGAAAUUGCAGUACGAGUAAAGAUGCUCGUUAAGCGCAGAAGGACGAAAAGACCC
CGGGACCUUUACUAUACCUUGGUAUUGGCAUUCGGUGUGGAUUGUGUAGCAUAGGCG
GGAGGCUUCGAAGCGUUGGCGCCAGCCAGUGCGGAGCCGUAAGGUGAAAUACCGCUC
UGUUCGCAUUGGAUGUCUAACCUCGAACAGUCAUCCUGUUCAGGGACAGUGCCUGGC
GGGUAGUUUAACUGGGGCGGUUGCCUCCCAAAGAGUAACGGAGGCGCCCAAAGGUCC
GCUCAGCCCGGUUGGCAAUCGGGUGGCGAGUGCAAGUGCACAAGCGGGCUUGACUGC
GAGGACGACGGUCCGAGCAGGGACGAAAGUCGGGACUAGUGAUCCGGUGCCGGUGUA
CGGACGCGGCAUCGCUCAACGGAUAAAAGGUACCCCGGGGAUAACAGGCUGAUCAUUC
CCAAGAGUCCAUAUCGACGGGAUGGUUUGGCACCUCGAUGUCGGCUCGUCGCAUCCUG
GGGCUGGAGCAGGUCCCAAGGGUUCGGCUGUUCGCCGAUUAAAGCGGCACGCGAGCU
GGGUUCAGAACGUCGUGAGACAGUUUGGUCUCUAUCCUCUGCGCUCGUUGGAAUGUU
GAGGAGGCCUGCCCAUAGUACGAGAGGACCUGGGUGGACGAACCUCUGGUAUGCCGG
UUGUCACGCCAGUGGCACGGCCGGUUGGCUACGUUCGGAAGGGAUAACCGCUGAAAG
CAUCUAAGCGGGAAGCCUGCUCCAAGAUAAGCAUUCCGUGCAGCCUCGGGCUGCUGUA
GUCCCCAUGCAGAACACGUGGUCGAUAGGCCGGACGUGGAAGCCCCGCGAGGGGUGGA
GCCGACCGGUACUAACGGACGAAAGGCAACACUCA
SEQ ID NO: 26, B. infantis, Accession Number: BCYF01000054.143.3206
Bifidobacterium; Bifidobacterium longum subsp. infantis. Sequence:
UGUUGCUUGCAAGGGCGUAUGGUGGAUGCCUUGGCAGACAGGACCGAUGAAGGACGU
UUGAGGCUGCGAUAUGCCUCGGGGAGCCGCCAACAGGGCUUUGAUCCGAGGAUUUCC
GAAUGGGGGAACCCACCGACCGUCAUGGGUCGGUACCGGCUUCGGCCGGGGGGUACGC
AGGGAAGUGAAACAUCUCAGUACCUGCAGGAAAGGAUAUUCCGUGAGUAGUGGCGAG
CGAAAGCGGAUGAUGGCCAAACCUUGCGCGUGUGAUACCCGUCGGGGGUUGCGCGCG
GGGUGUUGCGGGAUCGCGUGCGCCGGCUCCGACGGGCCGGCCGGCAGUUGCAGAAAAC
CAUGUGUCAGGGGAACCGGGUUGAAUACCGGGCCGCAGAGGGUGAGGGCCCCGUACC
UGAACGCGCAUGGUCUGCCGAUCGCGUCUCCCAAGUAGCACGGGUCCCGUGGAACCCC
GUGCGAAUCCGCCCAGACCGUUGGGUAAGCCUGAAUAUUCCUGUCUGACCGAUAGCGA
ACGAGUACCGUGAGGGAAAGGUGAAAAGUACCCCGGGAGGGGAGUGAAACAGUCUCU
GAAACCGUGCGCCUACAAUCCGUCGGAGCCCUUUUGUGGGGUGACGGCGUGCCUAUCG
AAAAAUGAGUCUGCGAGUCAGUGGCAUGUGGCGAGCAUAACCCGUGUGGGGUAUGCG
UAGCGAAGGCGAGUCUUAAAAGGCGUUUGAGUCGCGUGUCCUGGACCCGAAGCGGGA
UGAUCUAGCCCUGAGCAGGUUGAAGCGCGGGUAAGACCGCGUGGAGGACCGAACCCAC
CUGGGUUGAAAACCGGGGGGAUGACUUGGGGUUAGGGGUGAAAGGCCAAUCAAAUUC
CGUGAUAGCUGGUUCUCUCCGAAAUGCAUUUGGGUGCAGCGUCGGGUCAUUACGUCC
CGGGGGUAGAGCUACUGGAUGCUUGCGGGCCCGUAUCGGGUACCAACAGCAACCAAAC
UCCGAAUACCGGUGACGCGUAUCCCGGCAGUGAGUCGGCGGGGGAUAAGCUUCGUCG
UCGAAAGGGAAACAGCCCAGACCGUCGUCUAAGGUCCCGAAGCGUGUGCUAAGUGGG
AAAGGAUGUGGAGUCGCAUAGACAGCCAGGAGGUUGGCUCAGAAGCAGCCAUCCUUG
AAAGAGUGCGUAACAGCUCACUGGUCUAGUGGUUCCGCGCCGACAAUGUAGCGGGGC
UCAAGCACACCACCGAAGACGCGGCAGUAUUUUGUACUGGGUAGGAGAGCGUCCCAU
GAGGGGCGAAGCGGCCGUGUGAACGCGCCGUGGACUUCAUGGGAGUGAGAAUGCAGA
CAUGAGUAGCGAGAGACGGGUGAAGAUCCCGUCCGCUGGAUGACCAAGGGUUCCAGG
GCCACGUUCAUCGUCCCUGGGUGAGUCGGGUCCUAAGGCGAGGCCGACAGGCGUAGUC
GAAUGGAUGAACGGGUCGAUAUUCCCGUACCGGCUUUCAGCCGCCAAAACCGAGGCUU
GGAGUACUAACCUCCGGGUCCGGGCUUACUGCUCCUUCGGGGGCGGGAUGCCCUGGCC
UGUUGGGACCGAUCCUUGUAGUAGGUCAGCGCAGGAGUGACGCAGAAGGGUAGCCGG
CCGCGGAGGUGGUCUUCCGUGGUCAAGCACGCAGGGCGUGGGACAGGCAAAUCCGUCC
CGCAUGGGUCCGAGGUGCGAUGAUGGGGGCCUUAUGGCCCGAUAUCCGGUGAUCCCG
GCUGCCGAGAAAAGCUUCGGCGUCAGGCUCAAAGCCGCCCGUACCCCAAACCGACACU
GGUGGUCAGGUAGAGAAUACCAAAGCGAUCGAGCGAAUCCUGGUCAAGGAACUCGGC
AAAUCACUCCCGUUCCUUCGGUUUAAGGGAGACCCCCGAUGGUGAACCGCCUCGCGCG
GGGAGCUUUUGGGGGUGGCACAGACCAGGGGGUAGCGACUGUUUACCAAAAACACAG
GUGCAUGCGAAGGCGCAAGCCGCUGUAUAUGCACUGACGCCUGCCCGGUGCCGGAAGG
UUAAGAGGAUCCGUCAGCCUUCGGGUGAAGCGGUGAAUUCAAGCCCCGGUAAACGGC
GGUGGUAACUAUAACCAUCCUAAGGUAGCGAAAUUCCUUGUCGGGUAAGUUCCGACC
UGCACGAAUGGCGUAACGACUUCCCCACUGUCUCGACCAGGAGCUCGGCGAAAUUGCA
GUACGAGUAAAGAUGCUCGUUAAGCGCAGAAGGACGAAAAGACCCCGGGACCUUUAC
UAUACCUUGGUAUUGGCAUUCGGUGUGGAUUGUGUAGCAUAGGCGGGAGGCCGAGAG
GCGCGGUCGCCAGAUCGCGCGGAGCCGUAAGGUGAAAUACCGCUCUGUUCGCAUUGGA
UGUCUAACCUCGAACAGUCAUCCUGUUCAGGGACAGUGCCUGGCGGGUAGUUUAACU
GGGGCGGUUGCCUCCCAAAGAGUAACGGAGGCGCCCAAAGGUCCGCUCAGCCCGGUUG
GCAAUCGGGUGGCGAGUGCAAGUGCACAAGCGGGCUUGACUGCGAGGACGACGGUCC
GAGCAGGGACGAAAGUCGGGACUAGUGAUCCGGUGCCGGUGUACGGACGCGGCAUCG
CUCAACGGAUAAAAGGUACCCCGGGGAUAACAGGCUGAUCAUUCCCAAGAGUCCAUA
UCGACGGGAUGGUUUGGCACCUCGAUGUCGGCUCGUCGCAUCCUGGGGCUGGAGCAG
GUCCCAAGGGUUCGGCUGUUCGCCGAUUAAAGCGGCACGCGAGCUGGGUUCAGAACG
UCGUGAGACAGUUUGGUCUCUAUCCUCUGCGCUCGUUGGAAUGCUGAGGAGGCCUGC
CCAUAGUACGAGAGGACCUGGGUGGACGAACCUCUGGUAUGCCGGUUGUCACGCCAG
UGGCACGGCCGGUUGGCUACGUUCGGAAGGGAUAACCGCUGAAAGCAUCUAAGCGGG
AAGCCUGCUCCAAGAUAAGCAUUCCGUGGAACCUUCGAGUUCCUGUAGUCCCCAUGCA
GAACACGUGGUUGAUAGGCCGGACGUGGAAGCCCCGCGAGGGGUGGAGCCGACCGGU
ACUAACGGACGAAAGGCAACACUGA
Exemplary L. plantarum sequences
SEQ ID NO: 27, L. plantarum 16S sequence, 1474 nt (see e.g., Lactobacillus plantarum
strain NRRL B-14768 16S ribosomal RNA, partial sequence, NCBI Reference Sequence:
NR_042394.1)
GCTGGCGGCGTGCCTAATACATGCAAGTCGAACGAACTCTGGTATTGATTGGTGCTTGCA
TCATGATTTACATTTGAGTGAGTGGCGAACTGGTGAGTAACACGTGGGAAACCTGCCCA
GAAGCGGGGGATAACACCTGGAAACAGATGCTAATACCGCATAACAACTTGGACCGCAT
GGTCCGAGTTTGAAAGATGGCTTCGGCTATCACTTTTGGATGGTCCCGCGGCGTATTAGC
TAGATGGTGGGGTAACGGCTCACCATGGCAATGATACGTAGCCGACCTGAGAGGGTAAT
CGGCCACATTGGGACTGAGACACGGCCCAAACTCCTACGGGAGGCAGCAGTAGGGAATC
TTCCACAATGGACGAAAGTCTGATGGAGCAACGCCGCGTGAGTGAAGAAGGGTTTCGGC
TCGTAAAACTCTGTTGTTAAAGAAGAACATATCTGAGAGTAACTGTTCAGGTATTGACGG
TATTTAACCAGAAAGCCACGGCTAACTACGTGCCAGCAGCCGCGGTAATACGTAGGTGG
CAAGCGTTGTCCGGATTTATTGGGCGTAAAGCGAGCGCAGGCGGTTTTTTAAGTCTGATG
TGAAAGCCTTCGGCTCAACCGAAGAAGTGCATCGGAAACTGGGAAACTTGAGTGCAGAA
GAGGACAGTGGAACTCCATGTGTAGCGGTGAAATGCGTAGATATATGGAAGAACACCAG
TGGCGAAGGCGGCTGTCTGGTCTGTAACTGACGCTGAGGCTCGAAAGTATGGGTAGCAA
ACAGGATTAGATACCCTGGTAGTCCATACCGTAAACGATGAATGCTAAGTGTTGGAGGG
TTTCCGCCCTTCAGTGCTGCAGCTAACGCATTAAGCATTCCGCCTGGGGAGTACGGCCGC
AAGGCTGAAACTCAAAGGAATTGACGGGGGCCCGCACAAGCGGTGGAGCATGTGGTTTA
ATTCGAAGCTACGCGAAGAACCTTACCAGGTCTTGACATACTATGCAAATCTAAGAGATT
AGACGTTCCCTTCGGGGACATGGATACAGGTGGTGCATGGTTGTCGTCAGCTCGTGTCGT
GAGATGTTGGGTTAAGTCCCGCAACGAGCGCAACCCTTATTATCAGTTGCCAGCATTAAG
TTGGGCACTCTGGTGAGACTGCCGGTGACAAACCGGAGGAAGGTGGGGATGACGTCAAA
TCATCATGCCCCTTATGACCTGGGCTACACACGTGCTACAATGGATGGTACAACGAGTTG
CGAACTCGCGAGAGTAAGCTAATCTCTTAAAGCCATTCTCAGTTCGGATTGTAGGCTGCA
ACTCGCCTACATGAAGTCGGAATCGCTAGTAATCGCGGATCAGCATGCCGCGGTGAATA
CGTTCCCGGGCCTTGTACACACCGCCCGTCACACCATGAGAGTTTGTAACACCCAAAGTC
GGTGGGGTAACCTTTTAGGAACCAGCCGCCTAAGGTGGACAGATGAT
SEQ ID NO: 28, L. plantarum, Accession Number: CP026743.575947.578869
Lactiplantibacillus; Lactobacillus plantarum. Sequence:
AGGUUAAGUUAACAAGGGCGCAUGGUGAAUGCCUUGGCACUAGGAGCCGAUGAAGGA
CGGGACUAACACCGAUAUGCUUCGGGGAGCUGUACGUAAGCUAUGAUCCGGAGAUUU
CCGAAUGGGGCAACCCAGCAGUUUUAAUCAACUGUUACCACUAGAUGAAUUCAUAGU
CUAGUUGGAGGUAAACGCUGUGAACUGAAACAUCUCAUUAGCAGCAGGAAUAUAAAG
AAAUUUCGAUUCCCUAAGUAGCGGCGAGCGAACGGGGAACAGCCCAAACCAAAGUGC
UUGCACUUUGGGGUUGUAGGACUGAACAUUUGAGUUACCAAAGAACUUGAUAGUCGA
AGGAUUUGGGAAAAUCCGCCAUAGAUGGUGAUAGCCCAGUAGAUUAAAUCAAAUUCU
CUCAGUUCAGGAUCCUGAGUACGGCGGAACACGUGAAAUUCCGUCGGAAUCCGGGAG
GACCAUCUCCCAAGGCUAAAUACUACCUAGUGACCGAUAGUGAACCAGUACCGUGAGG
GAAAGGUGAAAAGCACCCCGGGAGGGGAGUGAAAUAGUUCCUGAAACCAUGUGCCUA
CAAUAAGUCAGAGCGCGUUAAUGCGUGAUGGCGUGCCUUUUGUAGAAUGAACCGGCG
AGUUAUGAUCCCGUGCAAGGUUAAGACUAAAAAGUCGGAGCCGUAGCGAAAGCGAGU
CUGAAAUGGGCGUUUUGAGUACGAGGUUAUAGACCCGAAACCAGGUGACCUAUCCAU
GUCCAGGUUGAAGGUGCGGUAAAACGCACUGGAGGACCGAACCCGUGUAAGUUGAAA
AUUGCUGGGAUGAGGUGUGGAUAGCGGUGAAAUUCCAAACGAACUUGGAGAUAGCUG
GUUCUCUCCGAAAUAGCUUUAGGGCUAGCCUCGGAAUUAGGAUCAUGGAGGUAGAGC
ACUAUUUGGACUAGGGGCCCGUCUUGGGUUACUGAAUUCAGAUAAACUCCGAAUGCC
AUUGAUUCAUAUCCGGGAGUCAGACGAUGAGUGAUAAGAUCCACCGUCGAAAGGGGA
ACAGCCCAGACCAUCAGUUAAGGUCCCUAAAUGUAUGCUAAGUGGAAAAGGAUGUGG
AGUUGCAUAGACAACUAGGAUGUUGGCUCAGAAGCAGCCACCAUUUAAAGAGUGCGU
AAUAGCUCACUAGUCGAGUGAUCCUGCGCCGAAAAUGUACCGGGGCUAAGCAUACUA
CCGAAACCAUGGAUACGACCAUUAGGUCGCGUGAUAGGAGAGCGUUCUAAGGGCGGU
GAAGCAAGAUCGUGAGGACUUGUGGAGCGCUUAGAAGUGAGAAUGCCGGUAUGAGUA
GCGAAAGAUAGGUGAGAAUCCUAUCCACCGAAUGACUAAGGUUUCCUGGGGAAGGCU
CGUCCUCCCAGGGUUAGUCGGGACCUAAGUCGAGGCCGAGAGGCGUAGACGAUGGAU
AACAGGUUGAGAUUCCUGUACUAGUUAAGUGCGUUUGAGCAAUGGAGGGACGCAGGA
GGCUAAGAUGUGCAUUCUGUUGGAUUAGAAUGUCCAAGCAGUAAGUCUGGUGAAGAG
UCAAAUGCUUUUCACUUUAAGGACAAGCUGUGAUGGGGAGCGAAAUUUAGUAGCGAA
GCGUCUGAUGUCACACUGCCGAGAAAAGCUUCUAGUGAGUACUUAACUACCCGUACCG
CAAACCGACACAGGUAGUCGAGGAGAGAAUCCUAAGGUGAGCGAGUGAACUCUCGUU
AAGGAACUCGGCAAAAUGACCCCGUAACUUCGGGAGAAGGGGUGCUGAUCGCAAGAU
CAGCCGCAGUGAAUAGGCCCAGGCGACUGUUUAUCAAAAACACAGGUCUCUGCAAAA
UCGUAAGAUGACGUAUAGGGGCUGACGCCUGCCCGGUGCUGGAAGGUUAAAAGGAUG
GGUUAGCUUCGGCGAAGCUCAGAAUUGAAGCCCCAGUAAACGGCGGCCGUAACUAUA
ACGGUCCUAAGGUAGCGAAAUUCCUUGUCGGGUAAGUUCCGACCCGCACGAAAGGCG
UAACGAUCUGGGCACUGUCUCAACGAGAGACUCGGUGAAAUUAUAUUGUCCGUGAAG
AUGCGGACUACCCGCGACAGGACGGAAAGACCCCAUGGAGCUUUACUGUAGUUUGAU
AUUGAGUGUUUGUACAGCUUGUACAGGAUAGGUAGGAGCCAUAGAAACCGGAACGCU
AGUUUCGGUGGAGGCGUUGGUGGGAUACUACCCUCGCUGUAUGACCACUCUAACCCGC
ACCACUAAUCGUGGUGGGAGACAGUGUCAGGUGGGCAGUUUGACUGGGGCGGUCGCC
UCCUAAAAAGUAACGGAGGCGCCCAAAGGUUCCCUCAGAAUGGUUGGAAAUCAUUCG
CAGAGUGUAAAGGCACAAGGGAGCUUGACUGCGAGACAGACAGGUCGAGCAGGGACG
AAAGUCGGGCUUAGUGAUCCGGUGGUACCGUAUGGAAGGGCCAUCGCUCAACGGAUA
AAAGCUACCCUGGGGAUAACAGGCUUAUCUCCCCCAAGAGUCCACAUCGACGGGGAGG
UUUGGCACCUCGAUGUCGGCUCAUCGCAUCCUGGGGCUGUAGUCGGUCCCAAGGGUUG
GGCUGUUCGCCCAUUAAAGCGGUACGCGAGCUGGGUUCAGAACGUCGUGAGACAGUU
CGGUCCCUAUCCGUCGCGGGCGUAGGAAAUUUGAGAGGACCUGUCCUUAGUACGAGA
GGACCGGGAUGGACAUACCUCUGGUGUACCAGUUGUGCCGCCAGGCGCAUCGCUGGGU
AGCUACGUAUGGAUGUGAUAAACGCUGAAAGCAUCUAAGUGUGAAACACACCUCGAG
AUGAGAUUUCCCAUUCCUUUAUGGAAGUAAGACCCCUGAAAGAUGAUCAGGUAGAUA
GGUUAGAAGUGGCAGUGCGGUGACGCAUGAAGCGGACUAAUACUAAUCGGUCGAGGA
CUUAACCAAG
SEQ ID NO: 29, L. plantarum, Accession Number: CP020816.489843.492761
Lactiplantibacillus; Lactobacillus plantarum. Sequence:
UUAAGUUAACAAGGGCGCAUGGUGAAUGCCUUGGCACUAGGAGCCGAUGAAGGACGG
GACUAACACCGAUAUGCUUCGGGGAGCUGUACGUAAGCUAUGAUCCGGAGAUUUCCG
AAUGGGGCAACCCAGCAGUUUUAAUCAACUGUUACCACUAGAUGAAUUCAUAGUCUA
GUUGGAGGUAAACGCUGUGAACUGAAACAUCUCAUUAGCAGCAGGAAUAUAAAGAAA
UUUCGAUUCCCUAAGUAGCGGCGAGCGAACGGGGAACAGCCCAAACCAAAGUGCUUGC
ACUUUGGGGUUGUAGGACUGAACAUUUGAGUUACCAAAGAACUUGAUAGUCGAAGGA
UUUGGGAAAAUCCGCCAUAGAUGGUGAUAGCCCAGUAGAUUAAAUCAAAUUCUCUCA
GUUCAGGAUCCUGAGUACGGCGGAACACGUGAAAUUCCGUCGGAAUCCGGGAGGACC
AUCUCCCAAGGCUAAAUACUACCUAGUGACCGAUAGUGAACCAGUACCGUGAGGGAA
AGGUGAAAAGCACCCCGGGAGGGGAGUGAAAUAGUUCCUGAAACCAUGUGCCUACAA
UAAGUCAGAGCGCGUUAAUGCGUGAUGGCGUGCCUUUUGUAGAAUGAACCGGCGAGU
UAUGAUCCCGUGCAAGGUUAAGACUAAAAAGUCGGAGCCGUAGCGAAAGCGAGUCUG
AAAUGGGCGUUUUGAGUACGAGGUUAUAGACCCGAAACCAGGUGACCUAUCCAUGUC
CAGGUUGAAGGUGCGGUAAAACGCACUGGAGGACCGAACCCGUGUAAGUUGAAAAUU
GCUGGGAUGAGGUGUGGAUAGCGGUGAAAUUCCAAACGAACUUGGAGAUAGCUGGUU
CUCUCCGAAAUAGCUUUAGGGCUAGCCUCGGAAUUAGGAUCAUGGAGGUAGAGCACU
AUUUGGACUAGGGGCCCGUCUUGGGUUACUGAAUUCAGAUAAACUCCGAAUGCCAUU
GAUUCAUAUCCGGGAGUCAGACGAUGAGUGAUAAGAUCCACCGUCGAAAGGGGAACA
GCCCAGACCAUCAGUUAAGGUCCCUAAAUGUAUGCUAAGUGGAAAAGGAUGUGGAGU
UGCAUAGACAACUAGGAUGUUGGCUCAGAAGCAGCCACCAUUUAAAGAGUGCGUAAU
AGCUCACUAGUCGAGUGAUCCUGCGCCGAAAAUGUACCGGGGCUAAGCAUACUACCGA
AACCAUGGAUGCGACCAUUAGGUCGCGUGAUAGGAGAGCGUUCUAAGGGCGGUGAAG
CAAGAUCGUGAGGACUUGUGGAGCGCUUAGAAGUGAGAAUGCCGGUAUGAGUAGCGA
AAGAUAGGUGAGAAUCCUAUCCACCGAAUGACUAAGGUUUCCUGGGGAAGGCUCGUC
CUCCCAGGGUUAGUCGGGACCUAAGUCGAGGCCGAGAGGCGUAGACGAUGGAUAACA
GGUUGAGAUUCCUGUACUAGUUAAGUGCGUUUGAGCAAUGGAGGGACGCAGGAGGCU
AAGAUGUGCAUUCUGUUGGAUUAGAAUGUCCAAGCAGUAAGUCUUGUGAAGAGUCAA
AUGCUUUUCACUUUAAGGACAAGCUGUGAUGGGGAGCGAAAUUUAGUAGCGAAGCGU
CUGAUGUCACACUGCCGAGAAAAGCUUCUAGUGAGUACUUAACUACCCGUACCGCAAA
CCGACACAGGUAGUCGAGGAGAGAAUCCUAAGGUGAGCGAGUGAACUCUCGUUAAGG
AACUCGGCAAAAUGACCCCGUAACUUCGGGAGAAGGGGUGCUGAUCGCAAGAUCAGC
CGCAGUGAAUAGGCCCAGGCGACUGUUUAUCAAAAACACAGGUCUCUGCAAAAUCGU
AAGAUGACGUAUAGGGGCUGACGCCUGCCCGGUGCUGGAAGGUUAAAAGGAUGGGUU
AGCUUCGGCGAAGCUCAGAAUUGAAGCCCCAGUAAACGGCGGCCGUAACUAUAACGG
UCCUAAGGUAGCGAAAUUCCUUGUCGGGUAAGUUCCGACCCGCACGAAAGGCGUAAC
GAUCUGGGCACUGUCUCAACGAGAGACUCGGUGAAAUUAUAUUGUCCGUGAAGAUGC
GGACUACCCGCGACAGGACGGAAAGACCCCAUGGAGCUUUACUGUAGCUUGAUAUUG
AGUGUUUGUACAGCUUGUACAGGAUAGGUAGGAGCCAUAGAAACCGGAACGCUAGUU
UCGGUGGAGGCGUUGGUGGGAUACUACCCUCGCUGUAUGACCACUCUAACCCGCACCA
CUAAUCGUGGUGGGAGACAGUGUCAGGUGGGCAGUUUGACUGGGGCGGUCGCCUCCU
AAAAAGUAACGGAGGCGCCCAAAGGUUCCCUCAGAAUGGUUGGAAAUCAUUCGCAGA
GUGUAAAGGCACAAGGGAGCUUGACUGCGAGACAGACAGGUCGAGCAGGGACGAAAG
UCGGGCUUAGUGAUCCGGUGGUACCGUAUGGAAGGGCCAUCGCUCAACGGAUAAAAG
CUACCCUGGGGAUAACAGGCUUAUCUCCCCCAAGAGUCCACAUCGACGGGGAGGUUUG
GCACCUCGAUGUCGGCUCAUCGCAUCCUGGGGCUGUAGUCGGUCCCAAGGGUUGGGCU
GUUCGCCCAUUAAAGCGGUACGCGAGCUGGGUUCAGAACGUCGUGAGACAGUUCGGU
CCCUAUCCGUCGCGGGCGUAGGAAAUUUGAGAGGACCUGUCCUUAGUACGAGAGGAC
CGGGAUGGACAUACCUCUGGUGUACCAGUUGUGCCGCCAGGCGCAUCGCUGGGUAGCU
ACGUAUGGAUGUGAUAAACGCUGAAAGCAUCUAAGUGUGAAACACACCUCGAGAUGA
GAUUUCCCAUUCCUUUAUGGAAGUAAGACCCCUGAAAGAUGAUCAGGUAGAUAGGUU
AGAAGUGGCAGUGCGGUGACGCAUGAAGCGGACUAAUACUAAUCGGUCGAGGACUUA
ACCAA
SEQ ID NO: 30, L. plantarum, Accession Number: CP025412.483014.485936
Lactiplantibacillus; Lactobacillus plantarum. Sequence:
AGGUUAAGUUAACAAGGGCGCAUGGUGAAUGCCUUGGCACUAGGAGCCGAUGAAGGA
CGGGACUAACACCGAUAUGCUUCGGGGAGCUGUACGUAAGCUAUGAUCCGGAGAUUU
CCGAAUGGGGCAACCCAGCAGUUUUAAUCAACUGUUACCACUAGAUGAAUUCAUAGU
CUAGUUGGAGGUAAACGCUGUGAACUGAAACAUCUCAUUAGCAGCAGGAAUAUAAAG
AAAUUUCGAUUCCCUAAGUAGCGGCGAGCGAACGGGGAACAGCCCAAACCAAAGUGC
UUGCACUUUGGGGUUGUAGGACUGAACAUUUGAGUUACCAAAGAACUUGAUAGUCGA
AGGAUUUGGGAAAAUCCGCCAUAGAUGGUGAUAGCCCAGUAGAUUAAAUCAAAUUCU
CUCAGUUCAGGAUCCUGAGUACGGCGGAACACGUGAAAUUCCGUCGGAAUCCGGGAG
GACCAUCUCCCAAGGCUAAAUACUACCUAGUGACCGAUAGUGAACCAGUACCGUGAGG
GAAAGGUGAAAAGCACCCCGGGAGGGGAGUGAAAUAGUUCCUGAAACCAUGUGCCUA
CAAUAAGUCAGAGCGCGUUAAUGCGUGAUGGCGUGCCUUUUGUAGAAUGAACCGGCG
AGUUAUGAUCCCGUGCAAGGUUAAGACUAAAAAGUCGGAGCCGUAGCGAAAGCGAGU
CUGAAAUGGGCGUUUUGAGUACGAGGUUAUAGACCCGAAACCAGGUGACCUAUCCAU
GUCCAGGUUGAAGGUGCGGUAAAACGCACUGGAGGACCGAACCCGUGUAAGUUGAAA
AUUGCUGGGAUGAGGUGUGGAUAGCGGUGAAAUUCCAAACGAACUUGGAGAUAGCUG
GUUCUCUCCGAAAUAGCUUUAGGGCUAGCCUCGGAAUUAGGAUCAUGGAGGUAGAGC
ACUAUUUGGACUAGGGGCCCGUCUUGGGUUACUGAAUUCAGAUAAACUCCGAAUGCC
AUUGAUUCAUAUCCGGGAGUCAGACGAUGAGUGAUAAGAUCCACCGUCGAAAGGGGA
ACAGCCCAGACCAUCAGUUAAGGUCCCUAAAUGUAUGCUAAGUGGAAAAGGAUGUGG
AGUUGCAUAGACAACUAGGAUGUUGGCUCAGAAGCAGCCACCAUUUAAAGAGUGCGU
AAUAGCUCACUAGUCGAGUGAUCCUGCGCCGAAAAUGUACCGGGGCUAAGCAUACUA
CCGAAACCAUGGAUGCGACCAUUAGGUCGCGUGAUAGGAGAGCGUUCUAAGGGCGGU
GAAGCAAGAUCGUGAGGACUUGUGGAGCGCUUAGAAGUGAGAAUGCCGGUAUGAGUA
GCGAAAGAUAGGUGAGAAUCCUAUCCACCGAAUGACUAAGGUUUCCUGGGGAAGGCU
CGUCCUCCCAGGGUUAGUCGGGACCUAAGUCGAGGCCGAGAGGCGUAGACGAUGGAU
AACAGGUUGAGAUUCCUGUACUAGUUAAGUGCGUUUGAGCAAUGGAGGGACGCAGGA
GGCUAAGAUGUGCAUUCUGUUGGAUUAGAAUGUCCAAGCAGUAAGUCUUGUGAAGAG
UCAAAUGCUUUUCACUUUAAGGACAAGCUGUGAUGGGGAGCGAAAUUUAGUAGCGAA
GCGUCUGAUGUCACACUGCCGAGAAAAGCUUCUAGUGAGUACUUAACUACCCGUACCG
CAAACCGACACAGGUAGUCGAGGAGAGAAUCCUAAGGUGAGCGAGUGAACUCUCGUU
AAGGAACUCGGCAAAAUGACCCCGUAACUUCGGGAGAAGGGGUGCUGAUCGCAAGAU
CAGCCGCAGUGAAUAGGCCCAGGCGACUGUUUAUCAAAAACACAGGUCUCUGCAAAA
UCGUAAGAUGACGUAUAGGGGCUGACGCCUGCCCGGUGCUGGAAGGUUAAAAGGAUG
GGUUAGCUUCGGCGAAGCUCAGAAUUGAAGCCCCAGUAAACGGCGGCCGUAACUAUA
ACGGUCCUAAGGUAGCGAAAUUCCUUGUCGGGUAAGUUCCGACCCGCACGAAAGGCG
UAACGAUCUGGGCACUGUCUCAACGAGAGACUCGGUGAAAUUAUAUUGUCCGUGAAG
AUGCGGACUACCCGCGACAGGACGGAAAGACCCCAUGGAGCUUUACUGUAGCUUGAU
AUUGAGUGUUUGUACAGCUUGUACAGGAUAGGUAGGAGCCAUAGAAACCGGAACGCU
AGUUUCGGUGGAGGCGUUGGUGGGAUACUACCCUCGCUGUAUGACCACUCUAACCCGC
ACCACUAAUCGUGGUGGGAGACAGUGUCAGGUGGGCAGUUUGACUGGGGCGGUCGCC
UCCUAAAAAGUAACGGAGGCGCCCAAAGGUUCCCUCAGAAUGGUUGGAAAUCAUUCG
CAGAGUGUAAAGGCACAAGGGAGCUUGACUGCGAGACAGACAGGUCGAGCAGGGACG
AAAGUCGGGCUUAGUGAUCCGGUGGUACCGUAUGGAAGGGCCAUCGCUCAACGGAUA
AAAGCUACCCUGGGGAUAACAGGCUUAUCUCCCCCAAGAGUCCACAUCGACGGGGAGG
UUUGGCACCUCGAUGUCGGCUCAUCGCAUCCUGGGGCUGUAGUCGGUCCCAAGGGUUG
GGCUGUUCGCCCAUUAAAGCGGUACGCGAGCUGGGUUCAGAACGUCGUGAGACAGUU
CGGUCCCUAUCCGUCGCGGGCGUAGGAAAUUUGAGAGGACCUGUCCUUAGUACGAGA
GGACCGGGAUGGACAUACCUCUGGUGUACCAGUUGUGCCGCCAGGCGCAUCGCUGGGU
AGCUACGUAUGGAUGUGAUAAACGCUGAAAGCAUCUAAGUGUGAAACACACCUCGAG
AUGAGAUUUCCCAUUCCUUUAUGGAAGUAAGACCCCUGAAAGAUGAUCAGGUAGAUA
GGUUAGAAGUGGCAGUGCGGUGACGCAUGAAGCGGACUAAUACUAAUCGGUCGAGGA
CUUAACCAAG
SEQ ID NO: 31, L. plantarum, Accession Number: CP029349.2063132.2066054
Lactiplantibacillus; Lactobacillus plantarum. Sequence:
AGGUUAAGUUAACAAGGGCGCAUGGUGAAUGCCUUGGCACUAGGAGCCGAUGAAGGA
CGGGACUAACACCGAUAUGCUUCGGGGAGCUGUACGUAAGCUAUGAUCCGGAGAUUU
CCGAAUGGGGCAACCCAGCAGUUUUAAUCAACUGUUACCACUAGAUGAAUUCAUAGU
CUAGUUGGAGGUAAACGCUGUGAACUGAAACAUCUCAUUAGCAGCAGGAAUAUAAAG
AAAUUUCGAUUCCCUAAGUAGCGGCGAGCGAACGGGGAACAGCCCAAACCAAAGUGC
UUGCACUUUGGGGUUGUAGGACUGAACAUUUGAGUUACCAAAGAACUUGAUAGUCGA
AGGAUUUGGGAAAAUCCGCCAUAGAUGGUGAUAGCCCAGUAGAUUAAAUCAAAUUCU
CUCAGUUCAGGAUCCUGAGUACGGCGGAACACGUGAAAUUCCGUCGGAAUCCGGGAG
GACCAUCUCCCAAGGCUAAAUACUACCUAGUGACCGAUAGUGAACCAGUACCGUGAGG
GAAAGGUGAAAAGCACCCCGGGAGGGGAGUGAAAUAGUUCCUGAAACCAUGUGCCUA
CAAUAAGUCAGAGCGCGUUAAUGCGUGAUGGCGUGCCUUUUGUAGAAUGAACCGGCG
AGUUAUGAUCCCGUGCAAGGUUAAGACUAAAAAGUCGGAGCCGUAGCGAAAGCGAGU
CUGAAAUGGGCGUUUUGAGUACGAGGUUAUAGACCCGAAACCAGGUGACCUAUCCAU
GUCCAGGUUGAAGGUGCGGUAAAACGCACUGGAGGACCGAACCCGUGUAAGUUGAAA
AUUGCUGGGAUGAGGUGUGGAUAGCGGUGAAAUUCCAAACGAACUUGGAGAUAGCUG
GUUCUCUCCGAAAUAGCUUUAGGGCUAGCCUCGGAAUUAGGAUCAUGGAGGUAGAGC
ACUAUUUGGACUAGGGGCCCGUCUUGGGUUACUGAAUUCAGAUAAACUCCGAAUGCC
AUUGAUUCAUAUCCGGGAGUCAGACGAUGAGUGAUAAGAUCCACCGUCGAAAGGGGA
ACAGCCCAGACCAUCAGUUAAGGUCCCUAAAUGUAUGCUAAGUGGAAAAGGAUGUGG
AGUUGCAUAGACAACUAGGAUGUUGGCUCAGAAGCAGCCACCAUUUAAAGAGUGCGU
AAUAGCUCACUAGUCGAGUGAUCCUGCGCCGAAAAUGUACCGGGGCUAAGCAUACUA
CCGAAACCAUGGAUGCGACCAUUAGGUCGCGUGAUAGGAGAGCGUUCUAAGGGCGGU
GAAGCAAGAUCGUGAGGACUUGUGGAGCGCUUAGAAGUGAGAAUGCCGGUAUGAGUA
GCGAAAGAUAGGUGAGAAUCCUAUCCACCGAAUGACUAAGGUUUCCUGGGGAAGGCU
CGUCCUCCCAGGGUUAGUCGGGACCUAAGUCGAGGCCGAGAGGCGUAGACGAUGGAU
AACAGGUUGAGAUUCCUGUACUAGUUAAGUGCGUUUGAGCAAUGGAGGGACGCAGGA
GGCUAAGAUGUGCAUUCUGUUGGAUUAGAAUGUCCAAGCAGUAAGUCUUGUGAAGAG
UCAAAUGCUUUUCACUUUAAGGACAAGCUGUGAUGGGGAGCGAAAUUUAGUAGCGAA
GCGUCUGAUGUCACACUGCCGAGAAAAGCUUCUAGUGAGUACUUAACUACCCGUACCG
CAAACCGACACAGGUAGUCGAGGAGAGAAUCCUAAGGUGAGCGAGUGAACUCUCGUU
AAGGAACUCGGCAAAAUGACCCCGUAACUUCGGGAGAAGGGGUGCUGAUCGCAAGAU
CAGCCGCAGUGAAUAGGCCCAGGCGACUGUUUAUCAAAAACACAGGUCUCUGCAAAA
UCGUAAGAUGACGUAUAGGGGCUGACGCCUGCCCGGUGCUGGAAGGUUAAAAGGAUG
GGUUAGCUUCGGCGAAGCUCAGAAUUGAAGCCCCAGUAAACGGCGGCCGUAACUAUA
ACGGUCCUAAGGUAGCGAAAUUCCUUGUCGGGUAAGUUCCGACCCGCACGAAAGGCG
UAACGAUCUGGGCACUGUCUCAACGAGAGACUCGGUGAAAUUAUAUUGUCCGUGAAG
AUGCGGACUACCCGCGACAGGACGGAAAGACCCCAUGGAGCUUUACUGUAGCUUGAU
AUUGAGUGUUUGUACAGCUUGUACAGGAUAGGUAGGAGCCAUAGAAACCGGAACGCU
AGUUUCGGUGGAGGCGUUGGUGGGAUACUACCCUCGCUGUAUGACCACUCUAACCCGC
ACCACUAAUCGUGGUGGGAGACAGUGUCAGGUGGGCAGUUUGACUGGGGCGGUCGCC
UCCUAAAAAGUAACGGAGGCGCCCAAAGGUUCCCUCAGAAUGGUUGGAAAUCAUUCG
CAGAGUGUAAAGGCACAAGGGAGCUUGACUGCGAGACAGACAGGUCGAGCAGGGACG
AAAGUCGGGCUUAGUGAUCCGGUGGUACCGUAUGGAAGGGCCAUCGCUCAACGGAUA
AAAGCUACCCUGGGGAUAACAGGCUUAUCUCCCCCAAGAGUCCACAUCGACGGGGAGG
UUUGGCACCUCGAUGUCGGCUCAUCGCAUCCUGGGGCUGUAGUCGGUCCCAAGGGUUG
GGCUGUUCGCCCAUUAAAGCGGUACGCGAGCUGGGUUCAGAACGUCGUGAGACAGUU
CGGUCCCUAUCCGUCGCGGGCGUAGGAAAUUUGAGAGGACCUGUCCUUAGUACGAGA
GGACCGGGAUGGACAUACCUCUGGUGUACCAGUUGUGCCGCCAGGCGCAUCGCUGGGU
AGCUACGUAUGGAUGUGAUAAACGCUGAAAGCAUCUAAGUGUGAAACACACCUCGAG
AUGAGAUUUCCCAUUCCUUUAUGGAAGUAAGACCCCUGAAAGAUGAUCAGGUAGAUA
GGUUAGAAGUGGCAGUGCGGUGACGCAUGAAGCGGACUAAUACUAAUCGGUCGAGGA
CUUAACCAAG
SEQ ID NO: 32, L. plantarum, Accession Number: LUXF01000016.189.3109
Lactiplantibacillus; Lactobacillus plantarum. Sequence:
GGUUAAGUUAACAAGGGCGCAUGGUGAAUGCCUUGGCACUAGGAGCCGAUGAAGGAC
GGGACUAACACCGAUAUGCUUCGGGGAGCUGUACGUAAGCUAUGAUCCGGAGAUUUC
CGAAUGGGGCAACCCAGCAGUUUUAAUCAACUGUUACCACUAGAUGAAUUCAUAGUC
UAGUUGGAGGUAAACGCUGUGAACUGAAACAUCUCAUUAGCAGCAGGAAUAUAAAGA
AAUUUCGAUUCCCUAAGUAGCGGCGAGCGAACGGGGAACAGCCCAAACCAAAGUGCU
UGCACUUUGGGGUUGUAGGACUGAACAUUUGAGUUACCAAAGAACUUGAUAGUCGAA
GGAUUUGGGAAAAUCCGCCAUAGAUGGUGAUAGCCCAGUAGAUUAAAUCAAAUUCUC
UCAGUUCAGGAUCCUGAGUACGGCGGAACACGUGAAAUUCCGUCGGAAUCCGGGAGG
ACCAUCUCCCAAGGCUAAAUACUACCUAGUGACCGAUAGUGAACCAGUACCGUGAGGG
AAAGGUGAAAAGCACCCCGGGAGGGGAGUGAAAUAGUUCCUGAAACCAUGUGCCUAC
AAUAAGUCAGAGCGCGUUAAUGCGUGAUGGCGUGCCUUUUGUAGAAUGAACCGGCGA
GUUAUGAUCCCGUGCAAGGUUAAGACUAAAAAGUCGGAGCCGUAGCGAAAGCGAGUC
UGAAAUGGGCGUUUUGAGUACGAGGUUAUAGACCCGAAACCAGGUGACCUAUCCAUG
UCCAGGUUGAAGGUGCGGUAAAACGCACUGGAGGACCGAACCCGUGUAAGUUGAAAA
UUGCUGGGAUGAGGUGUGGAUAGCGGUGAAAUUCCAAACGAACUUGGAGAUAGCUGG
UUCUCUCCGAAAUAGCUUUAGGGCUAGCCUCGGAAUUAGGAUCAUGGAGGUAGAGCA
CUAUUUGGACUAGGGGCCCGUCUUGGGUUACUGAAUUCAGAUAAACUCCGAAUGCCA
UUGAUUCAUAUCCGGGAGUCAGACGAUGAGUGAUAAGAUCCACCGUCGAAAGGGGAA
CAGCCCAGACCAUCAGUUAAGGUCCCUAAAUGUAUGCUAAGUGGAAAAGGAUGUGGA
GUUGCAUAGACAACUAGGAUGUUGGCUCAGAAGCAGCCACCAUUUAAAGAGUGCGUA
AUAGCUCACUAGUCGAGUGAUCCUGCGCCGAAAAUGUACCGGGGCUAAGCAUACUACC
GAAACCAUGGAUGCGACCAUUAGGUCGCGUGAUAGGAGAGCGUUCUAAGGGCGGUGA
AGCAAGAUCGUGAGGACUUGUGGAGCGCUUAGAAGUGAGAAUGCCGGUAUGAGUAGC
GAAAGAUAGGUGAGAAUCCUAUCCACCGAAUGACUAAGGUUUCCUGGGGAAGGCUCG
UCCUCCCAGGGUUAGUCGGGACCUAAGUCGAGGCCGAGAGGCGUAGACGAUGGAUAA
CAGGUUGAGAUUCCUGUACUAGUUAAGUGCGUUUGAGCAAUGGAGGGACGCAGGAGG
CUAAGAUGUGCAUUCUGUUGGAUUAGAAUGUCCAAGCAGUAAGUCUUGUGAAGAGUC
AAAUGCUUUUCACUUUAAGGACAAGCUGUGAUGGGGAGCGAAAUUUAGUAGCGAAGC
GUCUGAUGUCACACUGCCGAGAAAAGCUUCUAGUGAGUACUUAACUACCCGUACCGCA
AACCGACACAGGUAGUCGAGGAGAGAAUCCUAAGGUGAGCGAGUGAACUCUCGUUAA
GGAACUCGGCAAAAUGACCCCGUAACUUCGGGAGAAGGGGUGCUGAUCGCAAGAUCA
GCCGCAGUGAAUAGGCCCAGGCGACUGUUUAUCAAAAACACAGGUCUCUGCAAAAUC
GUAAGAUGACGUAUAGGGGCUGACGCCUGCCCGGUGCUGGAAGGUUAAAAGGAUGGG
UUAGCUUCGGCGAAGCUCAGAAUUGAAGCCCCAGUAAACGGCGGCCGUAACUAUAAC
GGUCCUAAGGUAGCGAAAUUCCUUGUCGGGUAAGUUCCGACCCGCACGAAAGGCGUA
ACGAUCUGGGCACUGUCUCAACGAGAGACUCGGUGAAAUUAUAUUGUCCGUGAAGAU
GCGGACUACCCGCGACAGGACGGAAAGACCCCAUGGAGCUUUACUGUAGCUUGAUAU
UGAGUGUUUGUACAGCUUGUACAGGAUAGGUAGGAGCCAUAGAAACCGGAACGCUAG
UUUCGGUGGAGGCGUUGGUGGGAUACUACCCUCGCUGUAUGACCACUCUAACCCGCAC
CACUAAUCGUGGUGGGAGACAGUGUCAGGUGGGCAGUUUGACUGGGGCGGUCGCCUC
CUAAAAAGUAACGGAGGCGCCCAAAGGUUCCCUCAGAAUGGUUGGAAAUCAUUCGCA
GAGUGUAAAGGCACAAGGGAGCUUGACUGCGAGACAGACAGGUCGAGCAGGGACGAA
AGUCGGGCUUAGUGAUCCGGUGGUACCGUAUGGAAGGGCCAUCGCUCAACGGAUAAA
AGCUACCCUGGGGAUAACAGGCUUAUCUCCCCCAAGAGUCCACAUCGACGGGGAGGUU
UGGCACCUCGAUGUCGGCUCAUCGCAUCCUGGGGCUGUAGUCGGUCCCAAGGGUUGGG
CUGUUCGCCCAUUAAAGCGGUACGCGAGCUGGGUUCAGAACGUCGUGAGACAGUUCG
GUCCCUAUCCGUCGCGGGCGUAGGAAAUUUGAGAGGACCUGUCCUUAGUACGAGAGG
ACCGGGAUGGACAUACCUCUGGUGUACCAGUUGUGCCGCCAGGCGCAUCGCUGGGUAG
CUACGUAUGGAUGUGAUAAACGCUGAAAGCAUCUAAGUGUGAAACACACCUCGAGAU
GAGAUUUCCCAUUCCUUUAUGGAAGUAAGACCCCUGAAAGAUGAUCAGGUAGAUAGG
UUAGAAGUGGCAGUGCGGUGACGCAUGAAGCGGACUAAUACUAAUCGGUCGAGGACU
UAACCAA
Exemplary L. acidophilus sequences
SEQ ID NO: 33, L. acidophilus 16S sequence, 1564 nt (see e.g., NCBI Reference
Number LA14_RS08025 16S ribosomal RNA Lactobacillus acidophilus La-14, NCBI Gene ID:
56943192)
AAAAACGAGAGTTTGATCCTGGCTCAGGACGAACGCTGGCGGCGTGCCTAATACATGCA
AGTCGAGCGAGCTGAACCAACAGATTCACTTCGGTGATGACGTTGGGAACGCGAGCGGC
GGATGGGTGAGTAACACGTGGGGAACCTGCCCCATAGTCTGGGATACCACTTGGAAACA
GGTGCTAATACCGGATAAGAAAGCAGATCGCATGATCAGCTTATAAAAGGCGGCGTAAG
CTGTCGCTATGGGATGGCCCCGCGGTGCATTAGCTAGTTGGTAGGGTAACGGCCTACCAA
GGCAATGATGCATAGCCGAGTTGAGAGACTGATCGGCCACATTGGGACTGAGACACGGC
CCAAACTCCTACGGGAGGCAGCAGTAGGGAATCTTCCACAATGGACGAAAGTCTGATGG
AGCAACGCCGCGTGAGTGAAGAAGGTTTTCGGATCGTAAAGCTCTGTTGTTGGTGAAGA
AGGATAGAGGTAGTAACTGGCCTTTATTTGACGGTAATCAACCAGAAAGTCACGGCTAA
CTACGTGCCAGCAGCCGCGGTAATACGTAGGTGGCAAGCGTTGTCCGGATTTATTGGGCG
TAAAGCGAGCGCAGGCGGAAGAATAAGTCTGATGTGAAAGCCCTCGGCTTAACCGAGGA
ACTGCATCGGAAACTGTTTTTCTTGAGTGCAGAAGAGGAGAGTGGAACTCCATGTGTAGC
GGTGGAATGCGTAGATATATGGAAGAACACCAGTGGCGAAGGCGGCTCTCTGGTCTGCA
ACTGACGCTGAGGCTCGAAAGCATGGGTAGCGAACAGGATTAGATACCCTGGTAGTCCA
TGCCGTAAACGATGAGTGCTAAGTGTTGGGAGGTTTCCGCCTCTCAGTGCTGCAGCTAAC
GCATTAAGCACTCCGCCTGGGGAGTACGACCGCAAGGTTGAAACTCAAAGGAATTGACG
GGGGCCCGCACAAGCGGTGGAGCATGTGGTTTAATTCGAAGCAACGCGAAGAACCTTAC
CAGGTCTTGACATCTAGTGCAATCCGTAGAGATACGGAGTTCCCTTCGGGGACACTAAGA
CAGGTGGTGCATGGCTGTCGTCAGCTCGTGTCGTGAGATGTTGGGTTAAGTCCCGCAACG
AGCGCAACCCTTGTCATTAGTTGCCAGCATTAAGTTGGGCACTCTAATGAGACTGCCGGT
GACAAACCGGAGGAAGGTGGGGATGACGTCAAGTCATCATGCCCCTTATGACCTGGGCT
ACACACGTGCTACAATGGACAGTACAACGAGGAGCAAGCCTGCGAAGGCAAGCGAATCT
CTTAAAGCTGTTCTCAGTTCGGACTGCAGTCTGCAACTCGACTGCACGAAGCTGGAATCG
CTAGTAATCGCGGATCAGCACGCCGCGGTGAATACGTTCCCGGGCCTTGTACACACCGCC
CGTCACACCATGGGAGTCTGCAATGCCCAAAGCCGGTGGCCTAACCTTCGGGAAGGAGC
CGTCTAAGGCAGGGCAGATGACTGGGGTGAAGTCGTAACAAGGTAGCCGTAGGAGAACC
TGCGGCTGGATCACCTCCTTT
SEQ ID NO: 34, L. acidophilus, Accession Number: CP000033.60957.64007
Lactobacillus; Lactobacillus acidophilus. Sequence:
GCAAAAAACCGAGACAAUCAAAGAGAACAGAUUGUAGAGCGACCGAGAAGAGAAUUC
UUGGGUAAGGUCAAGUAGAAAAGGGCGCACGGUGAAUGCCUAGGCACUAACAGCCGA
UGAAGGACGUGACGAACUACGAAAAGCUUCGGGGAGCGGUAAGUACGCAGUGAUCCG
GAGAUGUCCGAAUGGGGGAACCCAAUGCAGCGAUGCAUUAUUGGUUGAUGAAUAGAU
AGUCAAUCAAAGGAAGACGCAGUGAACUGAAACAUCUAAGUAGCUGCAGGAAGAGAA
AGAAAAAUCGAUUUCCUUAGUAGCGGCGAGCGAAGAGGAAAGAGCCCAAACCAAGUG
AUUUAUCAUUUGGGGUUGUAGGACUGCAAAGUGGUAGCGUAAGCGAUAGUUGAAUUA
UCUGGGAAGGUAAGCCAGAGAGGGUGAGAGCCCCGUAAGCGAAAUUGCGAGCGCGCC
UAGCAGAAUCCUGAGUAGGCCGGGACACGAGGAAUCCCGGUUGAAGCCGCGAGGACC
AUCUCGCAAGGCUAAAUACUAGUUAGUGACCGAUAGUGAACCAGUACCGUGAGGGAA
AGGUGAAAAGAACCCCGGAAGGGGAGUGAAAGAGAACCUGAAACCGUGUGUCUACAA
GUAGUCAAAGCACAUUAAAGUGCGAUGGCGUGCCUUUUGUAGAAUGAACCGGCGAGU
UACGUUAUCUAGCGAGGUUAAGUCAGAAAAGACGGAGCCGGAGCGAAAGCGAGUCUG
AAUAGGGCGAAGAGUUAGGUGACGUAGACCCGAAACCAAGUGACCUACCCAUGGCCA
GGCUGAAGGUGUGGUAAAACGCACUGGAGGGCCGAACCCACGUAAGUUAAAAAUUGC
GGGGAUGAGCUGUGGGUAGCGGUGAAAUUCCAAACGAACUUGGAGAUAGCUGGUUCU
CUCCGAAAUAGCUUUAGGGCUAGCCUCGUGGAGAGGAUAAUGGAGGUAGAGCUCUGU
UUGGACUAGGGGCCCGUCAGGGGUUACUGAAUCCAGAUAAACUGCGAAUUCCAUAUA
UCCAUACACGGGAGUCAGACUGCGAGUGAUAAGAUCCGUAGUCGAAAGGGAAACAGC
CCAGAUCACCAGUUAAGGUCCCCAAAUCUAUGCUAAGUGGAAAAGGAUGUGGAGUUG
CGUAGACAACUAGGACGUUGGCUCAGAAGCAGCCAUCAUUCAAAGAGUGCGUAAUAG
CUCACUAGUCGAGUGGCGCUGCGCCGAAAAUUUACCGGGGCUAAGCAUAGUACCGAA
ACUGUGGAUGCAUCGAAAGAUGCGUGGUAGGAGAGCGUUCUAAGUGCGGCGAAGGUU
AACCGAGAGGAUAAUUGGAGCGCUUAGAAGUGAGAAUGCCGGUAUGAGUAGCGAAAG
ACAGGUGAGAAUCCUGUCCGCCGAAAGACUAAGGUUUCCUGGGGCAGGCUCGUCCGCC
CAGGGUAAGUCGGGACCUAAGGCAAGGCCGAGAGGCGUAGUCGAUGGAUAACAGGUA
GAAAUUCCUGUACUGUGUUUAAUCGUUAUGAGCGAUGGAGGGACGCAGGAGGUGAAA
CACGCAUCGAGCUGGAUCGAUGUUCAAGCAACAAGUGCGGUUAAGAGUCAAAUGCUU
CUAACCAGCAACACGAGUUGUGAUGAGUAGCGAAGUAAUAGUAGCGAAGGUGAUGUA
AUCACACUGCCAAGAAAAGCUUCUAGCCAGAGAGGACAGACCCGUACCGCAAACCGAC
ACAGGUAGUCGAGUGGAGAACACUAAGGUGAGCGAGAGAACUCUCGUUAAGGAACUC
GGCAAAAUGACCCCGUAACUUCGGAAGAAGGGGUGCUGGCCACGAGAGUGGUUAGCC
GCAGUGAAUAGGCCCAAACAACUGUUUAUCAAAAACACAGGUCUCUGCAAAAUCGUA
AGAUGACGUAUAGAGGCUGACACCUGCCCGGUGCUGGAAGGUUAAGAGGAGAGCUUA
GCGAAAGCGGAGGUUCGAAUUGAAGCCCCAGUAAACGGCGGCCGUAACUAUAACGGU
CCUAAGGUAGCGAAAUUCCUUGUCGGGUAAGUUCCGACCUGCACGAAAGGUGUAAUG
AUUUGGGCACUGUCUCAACGAGAGACUCGGUGAAAUUAUAAUACCCGUGAAGAUGCG
GGUUACCCGCGACAGGACGGAAAGACCCCAUGGAGCUUCACUGUAGCUUGAUAUUGA
GUAUCUUUUAAACAUGUACAGGAUAGGUAGGAGCCAAGGAAGGCAGGACGCUAGUUU
UGCUGGAGGCAAUGUUGGGAUACUACCCUUGUUUGAAGGAUGCUCUAACCUCGACCU
GUAAGCCAGGUCAGGGACAGUGUCAGGUGGGCAGUUUGACUGGGGCGGUCGCCUCCU
AAAGUGUAACGGAGGCGCUCAAAGGUUCCCUCAGAAUGGUUGGAAAUCAUUCGCAGA
GUGUAAAGGUAUAAGGGAGCUUGACUGCGAGAGAAACAGCUCGAGCAGGGACGAAAG
UCGGACUUAGUGAUCUGGUGGUACCGUAUGGAAUGGCCAUCACUCAACGGAUAAAAG
CUACCCUGGGGAUAACAGGCUUAUCUCCCCCAAGAGUUCACAUCGACGGGGAGGUUUG
GCACCUCGAUGUCGGCUCGUCGCAUCCUGGGGCUGAAGUCGGUCCCAAGGGUUGGGCU
GUUCGCCCAUUAAAGCGGCACGCGAGCUGGGUUCAGAACGUCGUGAGACAGUUCGGU
CCCUAUCCGUCGUGGGCGUAGGAAAUUUGAGAGGAGCUGUCCUUAGUACGAGAGGAC
CGGGAUGGACGCACCGCUGGUGUACCAGUUGUUCCGCCAGGAGCAUCGCUGGGUAGCU
AUGUGCGGAAGGGAUAAGCGCUGAAAGCAUCUAAGUGCGAAGCCCCCCUCAAGAUGA
GAUUUCCUUUGCAAUAGCAGUAAGACACCUCAAAGACGAUGAGGUAGAUAGGCUGGG
AGUGGAAGUUCUGUGAAGAAUGGAGCGGACCAGUACUAAUCAGUCGAGGACUUGACC
AAAAGCGAAGCAAACUGUAAGGUUUUUUGCGAGAGAUUAUGUUUAGUUUUGAGCGUA
GUAGCUCAAAAGAGUACGGUGGCGA
SEQ ID NO: 35, L. acidophilus, Accession Number: CP010432.436310.439215
Lactobacillus; Lactobacillus acidophilus. Sequence:
GGUCAAGUAGAAAAGGGCGCACGGUGAAUGCCUAGGCACUAACAGCCGAUGAAGGAC
GUGACGAACUACGAAAAGCUUCGGGGAGCGGUAAGUACGCAGUGAUCCGGAGAUGUC
CGAAUGGGGGAACCCAAUGCAGCGAUGCAUUAUUGGUUGAUGAAUAGAUAGUCAAUC
AAAGGAAGACGCAGUGAACUGAAACAUCUAAGUAGCUGCAGGAAGAGAAAGAAAAAU
CGAUUUCCUUAGUAGCGGCGAGCGAAGAGGAAAGAGCCCAAACCAAGUGAUUUAUCA
UUUGGGGUUGUAGGACUGCAAAGUGGUAGCGUAAGCGAUAGUUGAAUUAUCUGGGAA
GGUAAGCCAGAGAGGGUGAGAGCCCCGUAAGCGAAAUUGCGAGCGCGCCUAGCAGAA
UCCUGAGUAGGCCGGGACACGAGGAAUCCCGGUUGAAGCCGCGAGGACCAUCUCGCAA
GGCUAAAUACUAGUUAGUGACCGAUAGUGAACCAGUACCGUGAGGGAAAGGUGAAAA
GAACCCCGGAAGGGGAGUGAAAGAGAACCUGAAACCGUGUGUCUACAAGUAGUCAAA
GCACAUUAAAGUGCGAUGGCGUGCCUUUUGUAGAAUGAACCGGCGAGUUACGUUAUC
UAGCGAGGUUAAGUCAGAAAAGACGGAGCCGGAGCGAAAGCGAGUCUGAAUAGGGCG
AAGAGUUAGGUGACGUAGACCCGAAACCAAGUGACCUACCCAUGGCCAGGCUGAAGG
UGUGGUAAAACGCACUGGAGGGCCGAACCCACGUAAGUUAAAAAUUGCGGGGAUGAG
CUGUGGGUAGCGGUGAAAUUCCAAACGAACUUGGAGAUAGCUGGUUCUCUCCGAAAU
AGCUUUAGGGCUAGCCUCGUGGAGAGGAUAAUGGAGGUAGAGCUCUGUUUGGACUAG
GGGCCCGUCAGGGGUUACUGAAUCCAGAUAAACUGCGAAUUCCAUAUAUCCAUACAC
GGGAGUCAGACUGCGAGUGAUAAGAUCCGUAGUCGAAAGGGAAACAGCCCAGAUCAC
CAGUUAAGGUCCCCAAAUCUAUGCUAAGUGGAAAAGGAUGUGGAGUUGCGUAGACAA
CUAGGACGUUGGCUCAGAAGCAGCCAUCAUUCAAAGAGUGCGUAAUAGCUCACUAGU
CGAGUGGCGCUGCGCCGAAAAUUUACCGGGGCUAAGCAUAGUACCGAAACUGUGGAU
GCAUCGAAAGAUGCGUGGUAGGAGAGCGUUCUAAGUGCGGCGAAGGUUAACCGAGAG
GAUAAUUGGAGCGCUUAGAAGUGAGAAUGCCGGUAUGAGUAGCGAAAGACAGGUGAG
AAUCCUGUCCGCCGAAAGACUAAGGUUUCCUGGGGCAGGCUCGUCCGCCCAGGGUAAG
UCGGGACCUAAGGCAAGGCCGAGAGGCGUAGUCGAUGGAUAACAGGUAGAAAUUCCU
GUACUGUGUUUAAUCGUUAUGAGCGAUGGAGGGACGCAGGAGGUGAAACACGCAUCG
AGCUGGAUCGAUGUUCAAGCAACAAGUGCGGUUAAGAGUCAAAUGCUUCUAACCAGC
AACACGAGUUGUGAUGAGUAGCGAAGUAAUAGUAGCGAAGGUGAUGUAAUCACACUG
CCAAGAAAAGCUUCUAGCCAGAGAGGACACACCCGUACCGCAAACCGACACAGGUAGU
CGAGUGGAGAACACUAAGGUGAGCGAGAGAACUCUCGUUAAGGAACUCGGCAAAAUG
ACCCCGUAACUUCGGAAGAAGGGGUGCUGGCCACGAGAGUGGUUAGCCGCAGUGAAU
AGGCCCAAACAACUGUUUAUCAAAAACACAGGUCUCUGCAAAAUCGUAAGAUGACGU
AUAGAGGCUGACACCUGCCCGGUGCUGGAAGGUUAAGAGGAGAGCUUAGCGAAAGCG
AAGGUUCGAAUUGAAGCCCCAGUAAACGGCGGCCGUAACUAUAACGGUCCUAAGGUA
GCGAAAUUCCUUGUCGGGUAAGUUCCGACCUGCACGAAAGGUGUAAUGAUUUGGGCA
CUGUCUCAACGAGAGACUCGGUGAAAUUAUAAUACCCGUGAAGAUGCGGGUUACCCG
CGACAGGACGGAAAGACCCCAUGGAGCUUCACUGUAGCUUGAUAUUGAGUAUCUUUU
AAACAUGUACAGGAUAGGUAGGAGCCAAGGAAGGCAGGACGCUAGUUUUGCUGGAGG
CAAUGUUGGGAUACUACCCUUGUUUGAAGGAUGCUCUAACCUCGACCUGUAAGCCAG
GUCAGGGACAGUGUCAGGUGGGCAGUUUGACUGGGGCGGUCGCCUCCUAAAGUGUAA
CGGAGGCGCUCAAAGGUUCCCUCAGAAUGGUUGGAAAUCAUUCGCAGAGUGUAAAGG
UAUAAGGGAGCUUGACUGCGAGAGAAACAGCUCGAGCAGGGACGAAAGUCGGACUUA
GUGAUCUGGUGGUACCGUAUGGAAGGGCCAUCACUCAACGGAUAAAAGCUACCCUGG
GGAUAACAGGCUUAUCUCCCCCAAGAGUUCACAUCGACGGGGAGGUUUGGCACCUCGA
UGUCGGCUCGUCGCAUCCUGGGGCUGAAGUCGGUCCCAAGGGUUGGGCUGUUCGCCCA
UUAAAGCGGCACGCGAGCUGGGUUCAGAACGUCGUGAGACAGUUCGGUCCCUAUCCG
UCGUGGGCGUAGGAAAUUUGAGAGGAGCUGUCCUUAGUACGAGAGGACCGGGAUGGA
CGCACCGCUGGUGUACCAGUUGUUCCGCCAGGAGCAUCGCUGGGUAGCUAUGUGCGGA
AGGGAUAAGCGCUGAAAGCAUCUAAGUGCGAAGCCCCCCUCAAGAUGAGAUUUCCUU
UGCAAUAGCAGUAAGACACCUCAAAGACGAUGAGGUAGAUAGGCUGGGAGUGGAAGU
UCUGUGAAGAAUGGAGCGGACCAGUACUAAUCAGUCGAGGACUUGACCAA
SEQ ID NO: 36, L. acidophilus, Accession Number: LWSH01000065.1.1873
Lactobacillus; Lactobacillus acidophilus. Sequence:
CAGACUGCGAGUGAUAAGAUCCGUAGUCGAAAGGGAAACAGCCCAGAUCACCAGUUA
AGGUCCCCAAAUCUAUGCUAAGUGGAAAAGGAUGUGGAGUUGCGUAGACAACUAGGA
CGUUGGCUCAGAAGCAGCCAUCAUUCAAAGAGUGCGUAAUAGCUCACUAGUCGAGUG
GCGCUGCGCCGAAAAUUUACCGGGGCUAAGCAUAGUACCGAAACUGUGGAUGCAUCG
AAAGAUGCGUGGUAGGAGAGCGUUCUAAGUGCGGCGAAGGUUAACCGAGAGGAUAAU
UGGAGCGCUUAGAAGUGAGAAUGCCGGUAUGAGUAGCGAAAGACAGGUGAGAAUCCU
GUCCGCCGAAAGACUAAGGUUUCCUGGGGCAGGCUCGUCCGCCCAGGGUAAGUCGGGA
CCUAAGGCAAGGCCGAGAGGCGUAGUCGAUGGAUAACAGGUAGAAAUUCCUGUACUG
UGUUUAAUCGUUAUGAGCGAUGGAGGGACGCAGGAGGUGAAACACGCAUCGAGCUGG
AUCGAUGUUCAAGCAACAAGUGCGGUUAAGAGUCAAAUGCUUCUAACCAGCAACACG
AGUUGUGAUGAGUAGCGAAGUAAUAGUAGCGAAGGUGAUGUAAUCACACUGCCAAGA
AAAGCUUCUAGCCAGAGAGGACACACCCGUACCGCAAACCGACACAGGUAGUCGAGUG
GAGAACACUAAGGUGAGCGAGAGAACUCUCGUUAAGGAACUCGGCAAAAUGACCCCG
UAACUUCGGAAGAAGGGGUGCUGGCCACGAGAGUGGUUAGCCGCAGUGAAUAGGCCC
AAACAACUGUUUAUCAAAAACACAGGUCUCUGCAAAAUCGUAAGAUGACGUAUAGAG
GCUGACACCUGCCCGGUGCUGGAAGGUUAAGAGGAGAGCUUAGCGAAAGCGAAGGUU
CGAAUUGAAGCCCCAGUAAACGGCGGCCGUAACUAUAACGGUCCUAAGGUAGCGAAA
UUCCUUGUCGGGUAAGUUCCGACCUGCACGAAAGGUGUAAUGAUUUGGGCACUGUCU
CAACGAGAGACUCGGUGAAAUUAUAAUACCCGUGAAGAUGCGGGUUACCCGCGACAG
GACGGAAAGACCCCAUGGAGCUUCACUGUAGCUUGAUAUUGAGUAUCUUUUAAACAU
GUACAGGAUAGGUAGGAGCCAAGGAAGGCAGGACGCUAGUUUUGCUGGAGGCAAUGU
UGGGAUACUACCCUUGUUUGAAGGAUGCUCUAACCUCGACCUGUAAGCCAGGUCAGG
GACAGUGUCAGGUGGGCAGUUUGACUGGGGCGGUCGCCUCCUAAAGUGUAACGGAGG
CGCUCAAAGGUUCCCUCAGAAUGGUUGGAAAUCAUUCGCAGAGUGUAAAGGUAUAAG
GGAGCUUGACUGCGAGAGAAACAGCUCGAGCAGGGACGAAAGUCGGACUUAGUGAUC
UGGUGGUACCGUAUGGAAGGGCCAUCACUCAACGGAUAAAAGCUACCCUGGGGAUAA
CAGGCUUAUCUCCCCCAAGAGUUCACAUCGACGGGGAGGUUUGGCACCUCGAUGUCGG
CUCGUCGCAUCCUGGGGCUGAAGUCGGUCCCAAGGGUUGGGCUGUUCGCCCAUUAAAG
CGGCACGCGAGCUGGGUUCAGAACGUCGUGAGACAGUUCGGUCCCUAUCCGUCGUGGG
CGUAGGAAAUUUGAGAGGAGCUGUCCUUAGUACGAGAGGACCGGGAUGGACGCACCG
CUGGUGUACCAGUUGUUCCGCCAGGAGCAUCGCUGGGUAGCUAUGUGCGGAAGGGAU
AAGCGCUGAAAGCAUCUAAGUGCGAAGCCCCCCUCAAGAUGAGAUUUCCUUUGCAAU
AGCAGUAAGACACCUCAAAGACGAUGAGGUAGAUAGGCUGGGAG
SEQ ID NO: 37, L. acidophilus, Accession Number: CBLR010000038.101.3004
Lactobacillus; Lactobacillus acidophilus. Sequence:
UCAAGUAGAAAAGGGCGCACGGUGAAUGCCUAGGCACUAACAGCCGAUGAAGGACGU
GACGAACUACGAAAAGCUUCGGGGAGCGGUAAGUACGCAGUGAUCCGGAGAUGUCCG
AAUGGGGGAACCCAAUGCAGCGAUGCAUUAUUGGUUGAUGAAUAGAUAGUCAAUCAA
AGGAAGACGCAGUGAACUGAAACAUCUAAGUAGCUGCAGGAAGAGAAAGAAAAAUCG
AUUUCCUUAGUAGCGGCGAGCGAAGAGGAAAGAGCCCAAACCAAGUGAUUUAUCAUU
UGGGGUUGUAGGACUGCAAAGUGGUAGCGUAAGCGAUAGUUGAAUUAUCUGGGAAGG
UAAGCCAGAGAGGGUGAGAGCCCCGUAAGCGAAAUUGCGAGCGCGCCUAGCAGAAUC
CUGAGUAGGCCGGGACACGAGGAAUCCCGGUUGAAGCCGCGAGGACCAUCUCGCAAGG
CUAAAUACUAGUUAGUGACCGAUAGUGAACCAGUACCGUGAGGGAAAGGUGAAAAGA
ACCCCGGAAGGGGAGUGAAAGAGAACCUGAAACCGUGUGUCUACAAGUAGUCAAAGC
ACAUUAAAGUGCGAUGGCGUGCCUUUUGUAGAAUGAACCGGCGAGUUACGUUAUCUA
GCGAGGUUAAGUCAGAAAAGACGGAGCCGGAGCGAAAGCGAGUCUGAAUAGGGCGAA
GAGUUAGGUGACGUAGACCCGAAACCAAGUGACCUACCCAUGGCCAGGCUGAAGGUG
UGGUAAAACGCACUGGAGGGCCGAACCCACGUAAGUUAAAAAUUGCGGGGAUGAGCU
GUGGGUAGCGGUGAAAUUCCAAACGAACUUGGAGAUAGCUGGUUCUCUCCGAAAUAG
CUUUAGGGCUAGCCUCGUGGAGAGGAUAAUGGAGGUAGAGCUCUGUUUGGACUAGGG
GCCCGUCAGGGGUUACUGAAUCCAGAUAAACUGCGAAUUCCAUAUAUCCAUACACGG
GAGUCAGACUGCGAGUGAUAAGAUCCGUAGUCGAAAGGGAAACAGCCCAGAUCACCA
GUUAAGGUCCCCAAAUCUAUGCUAAGUGGAAAAGGAUGUGGAGUUGCGUAGACAACU
AGGACGUUGGCUCAGAAGCAGCCAUCAUUCAAAGAGUGCGUAAUAGCUCACUAGUCG
AGUGGCGCUGCGCCGAAAAUUUACCGGGGCUAAGCAUAGUACCGAAACUGUGGAUGC
AUCGAAAGAUGCGUGGUAGGAGAGCGUUCUAAGUGCGGCGAAGGUUAACCGAGAGGA
UAAUUGGAGCGCUUAGAAGUGAGAAUGCCGGUAUGAGUAGCGAAAGACAGGUGAGAA
UCCUGUCCGCCGAAAGACUAAGGUUUCCUGGGGCAGGCUCGUCCGCCCAGGGUAAGUC
GGGACCUAAGGCAAGGCCGAGAGGCGUAGUCGAUGGAUAACAGGUAGAAAUUCCUGU
ACUGUGUUUAAUCGUUAUGAGCGAUGGAGGGACGCAGGAGGUGAAACACGCAUCGAG
CUGGAUCGAUGUUCAAGCAACAAGUGCGGUUAAGAGUCAAAUGCUUCUAACCAGCAA
CACGAGUUGUGAUGAGUAGCGAAGUAAUAGUAGCGAAGGUGAUGUAAUCACACUGCC
AAGAAAAGCUUCUAGCCAGAGAGGACACACCCGUACCGCAAACCGACACAGGUAGUCG
AGUGGAGAACACUAAGGUGAGCGAGAGAACUCUCGUUAAGGAACUCGGCAAAAUGAC
CCCGUAACUUCGGAAGAAGGGGUGCUGGCCACGAGAGUGGUUAGCCGCAGUGAAUAG
GCCCAAACAACUGUUUAUCAAAAACACAGGUCUCUGCAAAAUCGUAAGAUGACGUAU
AGAGGCUGACACCUGCCCGGUGCUGGAAGGUUAAGAGGAGAGCUUAGCGAAAGCGAA
GGUUCGAAUUGAAGCCCCAGUAAACGGCGGCCGUAACUAUAACGGUCCUAAGGUAGC
GAAAUUCCUUGUCGGGUAAGUUCCGACCUGCACGAAAGGUGUAAUGAUUUGGGCACU
GUCUCAACGAGAGACUCGGUGAAAUUAUAAUACCCGUGAAGAUGCGGGUUACCCGCG
ACAGGACGGAAAGACCCCAUGGAGCUUCACUGUAGCUUGAUAUUGAGUAUCUUUUAA
ACAUGUACAGGAUAGGUAGGAGCCAAGGAAGGCAGGACGCUAGUUUUGCUGGAGGCA
AUGUUGGGAUACUACCCUUGUUUGAAGGAUGCUCUAACCUCGACCUGUAAGCCAGGU
CAGGGACAGUGUCAGGUGGGCAGUUUGACUGGGGCGGUCGCCUCCUAAAGUGUAACG
GAGGCGCUCAAAGGUUCCCUCAGAAUGGUUGGAAAUCAUUCGCAGAGUGUAAAGGUA
UAAGGGAGCUUGACUGCGAGAGAAACAGCUCGAGCAGGGACGAAAGUCGGACUUAGU
GAUCUGGUGGUACCGUAUGGAAGGGCCAUCACUCAACGGAUAAAAGCUACCCUGGGG
AUAACAGGCUUAUCUCCCCCAAGAGUUCACAUCGACGGGGAGGUUUGGCACCUCGAUG
UCGGCUCGUCGCAUCCUGGGGCUGAAGUCGGUCCCAAGGGUUGGGCUGUUCGCCCAUU
AAAGCGGCACGCGAGCUGGGUUCAGAACGUCGUGAGACAGUUCGGUCCCUAUCCGUCG
UGGGCGUAGGAAAUUUGAGAGGAGCUGUCCUUAGUACGAGAGGACCGGGAUGGACGC
ACCGCUGGUGUACCAGUUGUUCCGCCAGGAGCAUCGCUGGGUAGCUAUGUGCGGAAG
GGAUAAGCGCUGAAAGCAUCUAAGUGCGAAGCCCCCCUCAAGAUGAGAUUUCCUUUG
CAAUAGCAGUAAGACACCUCAAAGACGAUGAGGUAGAUAGGCUGGGAGUGGAAGUUC
UGUGAAGAAUGGAGCGGACCAGUACUAAUCAGUCGAGGACUUGACCAA
SEQ ID NO: 38, L. acidophilus, Accession Number: KC161284.1.671 Lactobacillus;
Lactobacillus acidophilus. Sequence:
CCCGUCACACCAUGGGAGUCUGCAAUGCCCAAAGCCGGCGUUCUAACCUUCGGGAAGG
AGCCGUCUAAGGCAGGGCAGAUGACUGGGGUGAAGUCGUAACAAGGUAGCCGUAGGA
GAACCUGCGGCUGGAUCACCUCCUUUCUAAGGAAGCGAAGGAUAUGGAGAGUAGAAA
UACUAAGAGAAGUAUCCAGAGCAAGCGGAAGCACACUAAGAAACUUUGUUUAGUUUU
GAGGGUAGUACCUCAAAAGAGUUAGUACAUUGAAAACUGAAUAUAAUCCAAGCAAAA
AACCGAGACAAUCAAAGAGAACAGAUUGUAGAGCGACCGAGAAGAGAAUUCUUGGGU
AAGGUCAAGUAGAAAAGGGCGCACGGUGAAUGCCUAGGCACUAACAGCCGAUGAAGG
ACGUGACGAACUACGAAAAGCUUCGGGGAGCGGUAAGUACGCAGUGAUCCGGAGAUG
UCCGAAUGGGGGAACCCAAUGCAGCGAUGCAUUAUUGGUUGAUGAAUAGAUAGUCAA
UCAAAGGAAGACGCAGUGAACUGAAACAUCUAAGUAGCUGCAGGAAGAGAAAGAAAA
AUCGAUUUCCUUAGUAGCGGCGAGCGAAGAGGAAAGAGCCCAAACCAAGUGAUUUAU
CAUUUGGGGUUGUAGGACUGCAAAGUGGUAGCGUAAGCGAUAG
SEQ ID NO: 39, L. acidophilus, Accession Number: NXEY01000033.151.1480
Salmonella; Lactobacillus acidophilus. Sequence:
GGGUUGUGAGGUUAAGCGACUAAGCGUACACGGUGGAUGCCCUGGCAGUCAGAGGCG
AUGAAGGGCGUGCUAAUCUGCGAUAAGCGCCGGUAAGGUGAUAUGAACCGUUAUAAC
CGGCGAUACCCGAAUGGGGAAACCCAGUGUGACUCGUCACACUAUCAUUAACUGAAUC
CAUAGGUUAAUGAGGCGAACCGGGGGAACUGAAACAUCUAAGUACCCCGAGGAAAAG
AAAUCAACCGAGAUUCCCCCAGUAGCGGCGAGCGAACGGGGAGGAGCCCAGAGCCUGA
AUCAGCAUGUGUGUUAGUGGAAGCGUCUGGAAAGGCGCGCGAUACAGGGUGACAGCC
CCGUACACAAAAGCGCAUGUGCUGUGAGCUCGAUGAGUAGGGCGGGACACGUGGUAU
CCUGUCUGAAUAUGGGGGGACCAUCCUCCAAGGCUAAAUACUCCUGACUGACCGAUAG
UGAACCAGUACCGUGAGGGAAAGGCGAAAAGAACCCCGGCGAGGGGAGUGAAAAAGA
ACCUGAAACCGUGUACGUACAAGCAGUGGGAGCACCCUUUGGGUGUGACUGCGUACC
UUUUGUAUAAUGGGUCAGCGACUUAUAUUCUGUAGCAAGGUUAACCGAAUAGGGGAG
CCGGAGGGAAACCGAGUCUUAACCGGGCGUUAAGUUGCAGGGUAUAGACCCGAAACC
CGGUGAUCUAGCCAUGGGCAGGUUGAAGGUUGGGUAACACUAACUGGAGGACCGAAC
CGACUAAUGUUGAAAAAUUAGCGGAUGACCUGUGGCUGGGGGUGAAAGGCCAAUCAA
ACCGGGAGAUAGCUGGUUCUCCCCGAAAGCUAUUUAGGUAGCGCCUCGUGAAUUCAU
CUCCGGGGGUAGAGCACUGUUUCGGCUAGGGGGCCAUCCCGGCUUACCAACCCGAUGC
AAACUGCGAAUACCGGAGAAUGUUAUCACGGGAGACACACGKCGGGUGCUAACGUCC
GGYGUSAAGAGGGAAACAACCCAGACCGCCAGCUAAGGUCCCUAAAUAUUGCUAAGUG
GGAAACGAAGUGGGAAGGCUAAAACAGUCAGGAGGUUGGCUUAGAAGCAGCCACCCU
UUAAAGAAAGCGUAAUAGCUCACUGAUCGAGUCGUCCUGCGCGGAAGAUGUAACGGG
GCUAAGCAAUAUACCGAAGCUGCGGAUGCACGUAAGUGCAUGGUAGGAGAGCGUUCU
GUAAGCCUGUGAAGGUGUCUUGUAAAGGAUGCUGGAGGUAUCAGAAGUGCGAAUGCU
GACAUGAGUAGCGAUAAAGGGGGUGAAAGGCCCCCUCGCCGUAAGCCCAAGGUUUCC
UACGCAACGUUCAU
Exemplary L. rhamnosus sequences
SEQ ID NO: 40, L. rhamnosus 16S sequence, 1521 nt (see e.g., Lactobacillus rhamnosus
strain JCM 1136 16S ribosomal RNA, partial sequence NCBI Reference Sequence:
NR_043408.1)
GRTSAACGCTSGCGGCGTGCCTAATACATGCAAGTCGAACGAGTTCTGATTATTGAAAGG
TGCTTGCATCTTGATTTAATTTTGAACGAGTGGCGGACGGGTGAGTAACACGTGGGTAAC
CTGCCCTTAAGTGGGGGATAACATTTGGAAACAGATGCTAATACCGCATAAATCCAAGA
ACCGCATGGTTCTTGGCTGAAAGATGGCGTAAGCTATCGCTTTTGGATGGACCCGCGGCG
TATTAGCTAGTTGGTGAGGTAACGGCTCACCAAGGCAATGATACGTAGCCGAACTGAGA
GGTTGATCGGCCACATTGGGACTGAGACACGGCCCAAACTCTACGGGAGGCAGCAGTAG
GGAATCTTCCACAATGGACGCAAGTCTGATGGAGCAACGCCGCGTGAGTNAAGAAGGCT
TTCGGGTCGTAAAACTCTGTTGTTGGAGAAGAATGGTCGGCAGAGTAACTGTTGTCGGCG
TGACGGTATCCAACCAGAAAGCCACGGCTAACTACGTGCCAGCAGCCGCGGTAATACGT
AGGTGGCAAGCGTTATCCGGATTTATTGGGCGTAAAGCGAGCGCAGGCGGTTTTTTAAGT
CTGATGTGAAAGCCCTCGGCTTAACCGAGGAAGTGCATCGGAAACTGGGAAACTTGAGT
NCAGAAGAGGACAGTGGAACTCCATGTGTAGCGGTGAAATGCGTAGATATATGGAAGAA
CACCAGTGGCGAAGGCGGCTGTCTGGTCTGTAACTGACGCTGAGGCTCGAAAGCATGGG
TAGCGAACAGGATTAGATACCCTGGTAGTCCATGCCGTAAACGATGAATGCTAGGTGTT
GGAGGGTTTCCGCCCTTCAGTGCCGCAGCTAACGCATTAAGCATTCCGCCTGGGGAGTAC
GACCGCAAGGTTGAAACTCAAAGGAATTGACGGGGGCCCGCACAAGCGGTGGAGCATGT
GGTTTAATTCGAAGCAACGCGAAGAACCTTACCAGGTCTTGACATCTTTTGATCACCTGA
GAGATCAGGTTTCCCCTTCGGGGGCAAAATGACAGGTGGTGCATGGTTGTCGTCAGCTCG
TGTCGTGAGATGTTGGGTTAAGTCCCGCAACGAGCGCAACCCTTATGACTAGTTGCCAGC
ATTTAGTTGGGCACTCTAGTAAGACTGCCGGTGACAAACCGGAGGAAGGTGGGGATGAC
GTCAAATCATCATGCCCCTTATGACCTGGGCTACACACGTGCTACAATGGATGGTACAAC
GAGTTGCGAGACCGCGAGGTCAAGCTAATCTCTTAAAGCCATTCTCAGTTCGGACTGTAG
GCTGCAACTCGCCTACACGAAGTCGGAATCGCTAGTAATCGCGGATCAGCACGCCGCGG
TGAATACGTTCCCGGGCCTTGTACACACCGCCCGTCACACCATGAGAGTTTGTAACACCC
GAAGCCGGTGGCGTAACCCTTTTAGGGAGCGAGCCGTCTAAGGTGGGNCAAATGATTAG
GGTGAAGTCGTAACAAGGTAGCCGTAGGAGAACC
SEQ ID NO: 41, L. rhamnosus, Accession Number: JUPX01000234.110.2909
Lacticaseibacillus; Lactobacillus rhamnosus. Sequence:
AGGGCGCACGGUGGAUGCCUUGGCACUAGGAGCCGAUGAAGGACGGAACUAAUACCG
AUAUGCUUCGGGGAGCUAUAAGUAAGCUUUGAUCCGGAGAUUUCCGAAUGGGGGAAC
CCAGUACACAUCAGUGUAUUGCCUGCAAGUGAAUACAUAGCUUGUUGGCGGCAGACG
CGGGGAACUGAAACAUCUCAGUACCCGCAGGAAGAGAAAGAAAACUCGAUUCCCAUA
GUAGCGGCGAGCGAAGUGGGAAGAGCCCAAACCGAGAAGCUUGCUUCUCGGGGUUGU
AGGACUGGACAUUGGAGUUACCAAAGUUCGACGUAGUCGAAGUCAGCUGGAAAGCUG
CGCCAUAGAAGGUGAAAGCCCUGUAAACGAAACGGCGGACUCUCCGUCCAGGAUCCUG
AGUACGGCGGAACACGUGAAAUUCCGUCGGAAUCCGGGAGGACCAUCUCCCAAGGCUA
AAUACUCCCUAGUGACCGAUAGUGAACCAGUACCGUGAGGGAAAGGUGAAAAGCACC
CCGGAAGGGGAGUGAAACAGUUCCUGAAACCGUGUGCCUACAAUUAGUCAAAGCCCG
UUAACGGGUAAUGGCGUGCCUUUUGUAGAAUGAACCGGCGAGUUACGUUUGCCUGCG
AGGUUAAGAUGAAAAGUCGGAGCCGGAGCGAAAGCGAGUCUGAACAGGGCGCUUCAG
UAGGUAGAUGUAGACCCGAAACCAAGUGACCUACCCAUGACCAGGUUGAAGGUGUGG
UGAAACACACUGGAGGACCGAACCCAUGUAUGUUGAAAAAUGCUGGGAUGAGUUGUG
GGUAGCGGUGAAAUUCCAAACGAACUUGGAGAUAGCUGGUUCUCUCCGAAAUAGCUU
UAGGGUUAGCCUCGGAGGAUGGAUCAUGGAGGUAGAGCACUGUUUGAACUAGGGGCC
CGUCAAGGGUUACUGAAUUCAGAUAAACUCCGAAUACCAUUGAUCUUACUCCGGGAG
UCAGACAGUGAGCGAUAAGGUCCAUUGUCGAAAGGGGAACAGCCCAGAUCACCAGUU
AAGGUCCCUAAAUUUAUGCUAAGUGGAAAAGGAUGUGGCGUUGCACAGACAACUAGG
AUGUUGGCUCAGAAGCAGCCACCAUUUAAAGAGUGCGUAAUAGCUCACUAGUCGAGU
GGCACUGCGCCGAAAAUAUACCGGGGCUAAGCAUAAUACCGAAACUGUGGGUGCACCC
GUCAGGGUGCGCGGUAGGAGAGCGUUCUAAGGGCGUUGAAGGUCGAUCGUGAGGACG
GCUGGAGCGCUUAGAAGUGAGAAUGCCGGCAUGAGUAGCGAAAGAUCAGUGAGAAUC
UGAUCCACCGUAUGACUAAGGUUUCCUGGGGAAGGCUCGUCCUCCCAGGGUUAGUCG
GGAUCUAAGGCGAGGCCGCAAGGCGUAGUCGAUGACAAGCAGGUUGAGAUUCCUGCA
CUAGUUUAUUUUGUUUAAGCGAUGGAGGGACGCAGGAGGCUAAGGAAAGCGCACGGC
UGGAAAAGUGCGUCCAAGCAGUAAGUCCGGUAGCGAGUGAAAUGCUUGCCGCCUUAA
GGACAAGCUGUGAUGGGGAGCGAAAUUAAAGUAGCGAAGUUCCUGAUGUCACACUGC
CAAGAAAAGCUUCUAGUGAGAAAUAAACUACCCGUACCGCAAACCGACACAGGUAGU
CGAGGAGAGUAUCCUCAGGUGAGCGAGCGAACUCUCGUUAAGGAACUCGGCAAAAUG
ACCCCGUAACUUCGGAAGAAGGGGUGCUGACCGCAAGGUCAGCCGCAGUGAAUAGGCC
CAAACAACUGUUUAUCAAAAACACAGGUCUCUGCUAAAUCGUAAGAUGAUGUAUAGG
GGCUGACGCCUGCCCGGUGCUGGAAGGUUAAGAGGAUGAGUUAGCGCAAGCGAAGCC
CAGAAUUGAAGCCCCAGUAAACGGCGGCCGUAACUAUAACGGUCCUAAGGUAGCGAA
AUUCCUUGUCGGGUAAGUUCCGACCCGCACGAAAGGCGUAAUGAUUUGGGCACUGUC
UCAACGAGAGACUCGGUGAAAUUAUAGUACCCGUGAAGAUGCGGGUUACCCGCGACA
GGACGGAAAGACCCCAUGGAGCUUUACUGUAGCUUGAUAUUGAGUGUUGGUACCGCU
UGUACAGGAUAGGUAGGAGCCGUAGAGAUCGGAACGCUAGUUUCGAUGGAGGCAUUG
GUGGGAUACUACCCUAGCUGUAUGAACACUCUAACCCGCGCCACUGAUCGUGGCGGGA
GACAGUGUCAGGUAGGCAGUUUGACUGGGGCGGUCGCCUCCUAAAAUGUAACGGAGG
CGCCCAAAGGUUCCCUCAGAAUGGUUGGAAAUCAUUCGCAGAGUGUAAAGGUAGAAG
GGAGCUUGACUGCGAGACUGACAAGUCGAGCAGGGACGAAAGUCGGGCUUAGUGAUC
CGGUGGUUCCGUAUGGAAGGGCCAUCGCUCAACGGAUAAAAGCUACCCUGGGGAUAA
CAGGCUUAUCUCCCCCAAGAGUCCACAUCGACGGGGAGGUUUGGCACCUCGAUGUCGG
CUCAUCGCAUCCUGGGGCUGUAGUCGGUCCCAAGGGUUGGGCUGUUCGCCCAUUAAAG
CGGUACGCGAGCUGGGUUCAGAACGUCGUGAGACAGUUCGGUCCCUAUCCGUCGCGGG
CGCAGGAAAUUUGAGAGGAGCUGUCCUUAGUACGAGAGGACCGGGAUGGACGUUCCG
CUGGUGUACCAGUUGUGCCGCCAGGCGCAUCGCUGGGUAGCUAUGAACGGAAGGGAU
AAACGCUGAAAGCAUCUAAGUGUGAAGCCCCCCUCGAGAUGAGAUUUCCCAUUCCU
SEQ ID NO: 42, L. rhamnosus, Accession Number: JTIN01000086.137.3056
Lacticaseibacillus; Lactobacillus rhamnosus. Sequence:
AGGUUAAGUUACAAAGGGCGCACGGUGGAUGCCUUGGCACUAGGAGCCGAUGAAGGA
CGGAACUAAUACCGAUAUGCUUCGGGGAGCUAUAAGUAAGCUUUGAUCCGGAGAUUU
CCGAAUGGGGGAACCCAGUACACAUCAGUGUAUUGCCUGCAAGUGAAUACAUAGCUU
GUUGGCGGCAGACGCGGGGAACUGAAACAUCUCAGUACCCGCAGGAAGAGAAAGAAA
ACUCGAUUCCCAUAGUAGCGGCGAGCGAAGUGGGAAGAGCCCAAACCGAGAAGCUUG
CUUCUCGGGGUUGUAGGACUGGACAUUGGAGUUACCAAAGUUCGACGUAGUCGAAGU
CAGCUGGAAAGCUGCGCCAUAGAAGGUGAAAGCCCUGUAAACGAAACGGCGGACUCU
CCGUCCAGGAUCCUGAGUACGGCGGAACACGUGAAAUUCCGUCGGAAUCCGGGAGGAC
CAUCUCCCAAGGCUAAAUACUCCCUAGUGACCGAUAGUGAACCAGUACCGUGAGGGAA
AGGUGAAAAGCACCCCGGAAGGGGAGUGAAACAGUUCCUGAAACCGUGUGCCUACAA
UUAGUCAAAGCCCGUUAACGGGUAAUGGCGUGCCUUUUGUAGAAUGAACCGGCGAGU
UACGUUUGCCUGCGAGGUUAAGAUGAAAAGUCGGAGCCGGAGCGAAAGCGAGUCUGA
ACAGGGCGCUUCAGUAGGUAGAUGUAGACCCGAAACCAAGUGACCUACCCAUGACCAG
GUUGAAGGUGUGGUGAAACACACUGGAGGACCGAACCCAUGUAUGUUGAAAAAUGCU
GGGAUGAGUUGUGGGUAGCGGUGAAAUUCCAAACGAACUUGGAGAUAGCUGGUUCUC
UCCGAAAUAGCUUUAGGGUUAGCCUCGGAGGAUGGAUCAUGGAGGUAGAGCACUGUU
UGAACUAGGGGCCCGUCAAGGGUUACUGAAUUCAGAUAAACUCCGAAUACCAUUGAU
CUUACUCCGGGAGUCAGACAGUGAGCGAUAAGGUCCAUUGUCGAAAGGGGAACAGCC
CAGAUCACCAGUUAAGGUCCCUAAAUUUAUGCUAAGUGGAAAAGGAUGUGGCGUUGC
ACAGACAACUAGGAUGUUGGCUCAGAAGCAGCCACCAUUUAAAGAGUGCGUAAUAGC
UCACUAGUCGAGUGGCACUGCGCCGAAAAUAUACCGGGGCUAAGCAUAAUACCGAAA
CUGUGGGUGCACCCGUCAGGGUGCGCGGUAGGAGAGCGUUCUAAGGGCGUUGAAGGU
CGAUCGUGAGGACGGCUGGAGCGCUUAGAAGUGAGAAUGCCGGCAUGAGUAGCGAAA
GAUCAGUGAGAAUCUGAUCCACCGUAUGACUAAGGUUUCCUGGGGAAGGCUCGUCCU
CCCAGGGUUAGUCGGGAUCUAAGGCGAGGCCGCAAGGCGUAGUCGAUGACAAGCAGG
UUGAGAUUCCUGCACUAGUUUAUUUUGUUUAAGCGAUGGAGGGACGCAGGAGGCUAA
GGAAAGCGCACGGCUGGAAAAGUGCGUCCAAGCAGUAAGUCCGGUAGCGAGUGAAAU
GCUUGCCGCCUUAAGGACAAGCUGUGAUGGGGAGCGAAAUUAAAGUAGCGAAGUUCC
UGAUGUCACACUGCCAAGAAAAGCUUCUAGUGAGAAAUAAACUACCCGUACCGCAAA
CCGACACAGGUAGUCGAGGAGAGUAUCCUCAGGUGAGCGAGCGAACUCUCGUUAAGG
AACUCGGCAAAAUGACCCCGUAACUUCGGAAGAAGGGGUGCUGACCGCAAGGUCAGCC
GCAGUGAAUAGGCCCAAACAACUGUUUAUCAAAAACACAGGUCUCUGCUAAAUCGUA
AGAUGAUGUAUAGGGGCUGACGCCUGCCCGGUGCUGGAAGGUUAAGAGGAUGAGUUA
GCGCAAGCGAAGCCCAGAAUUGAAGCCCCAGUAAACGGCGGCCGUAACUAUAACGGUC
CUAAGGUAGCGAAAUUCCUUGUCGGGUAAGUUCCGACCCGCACGAAAGGCGUAAUGA
UUUGGGCACUGUCUCAACGAGAGACUCGGUGAAAUUAUAGUACCCGUGAAGAUGCGG
GUUACCCGCGACAGGACGGAAAGACCCCAUGGAGCUUUACUGUAGCUUGAUAUUGAG
UGUUGGUACCGCUUGUACAGGAUAGGUAGGAGCCGUAGAGAUCGGAACGCUAGUUUC
GAUGGAGGCAUUGGUGGGAUACUACCCUAGCUGUAUGAACACUCUAACCCGCGCCACU
AAUCGUGGCGGGAGACAGUGUCAGGUAGGCAGUUUGACUGGGGCGGUCGCCUCCUAA
AAUGUAACGGAGGCGCCCAAAGGUUCCCUCAGAAUGGUUGGAAAUCAUUCGCAGAGU
GUAAAGGUAGAAGGGAGCUUGACUGCGAGACUGACAAGUCGAGCAGGGACGAAAGUC
GGGCUUAGUGAUCCGGUGGUUCCGUAUGGAAGGGCCAUCGCUCAACGGAUAAAAGCU
ACCCUGGGGAUAACAGGCUUAUCUCCCCCAAGAGUCCACAUCGACGGGGAGGUUUGGC
ACCUCGAUGUCGGCUCAUCGCAUCCUGGGGCUGUAGUCGGUCCCAAGGGUUGGGCUGU
UCGCCCAUUAAAGCGGUACGCGAGCUGGGUUCAGAACGUCGUGAGACAGUUCGGUCCC
UAUCCGUCGCGGGCGCAGGAAAUUUGAGAGGAGCUGUCCUUAGUACGAGAGGACCGG
GAUGGACGUUCCGCUGGUGUACCAGUUGUGCCGCCAGGCGCAUCGCUGGGUAGCUAU
GAACGGAAGGGAUAAACGCUGAAAGCAUCUAAGUGUGAAGCCCCCCUCGAGAUGAGA
UUUCCCAUUCCUAUAUGGAAGUAAGACCCCUGAAAGAUGAUCAGGUAGAUAGGCUGG
AAGUGGAAGUGCAGCGAUGCAUGGAGCGGACCAGUACUAAUCGGUCGAGGACUUAAC
CAAG
SEQ ID NO: 43, L. rhamnosus, Accession Number: CP014201.1702655.1705574
Lacticaseibacillus; Lactobacillus rhamnosus. Sequence:
AGGUUAAGUUACAAAGGGCGCACGGUGGAUGCCUUGGCACUAGGAGCCGAUGAAGGA
CGGAACUAAUACCGAUAUGCUUCGGGGAGCUAUAAGUAAGCUUUGAUCCGGAGAUUU
CCGAAUGGGGGAACCCAGUACACAUCAGUGUAUUGCCUGCAAGUGAAUACAUAGCUU
GUUGGCGGCAGACGCGGGGAACUGAAACAUCUCAGUACCCGCAGGAAGAGAAAGAAA
ACUCGAUUCCCAUAGUAGCGGCGAGCGAAGUGGGAAGAGCCCAAACCGAGAAGCUUG
CUUCUCGGGGUUGUAGGACUGGACAUUGGAGUUACCAAAGUUCGACGUAGUCGAAGU
CAGCUGGAAAGCUGCGCCAUAGAAGGUGAAAGCCCUGUAAACGAAACGGCGGACUCU
CCGUCCAGGAUCCUGAGUACGGCGGAACACGUGAAAUUCCGUCGGAAUCCGGGAGGAC
CAUCUCCCAAGGCUAAAUACUCCCUAGUGACCGAUAGUGAACCAGUACCGUGAGGGAA
AGGUGAAAAGCACCCCGGAAGGGGAGUGAAACAGUUCCUGAAACCGUGUGCCUACAA
UUAGUCAAAGCCCGUUAACGGGUAAUGGCGUGCCUUUUGUAGAAUGAACCGGCGAGU
UACGUUUGCCUGCGAGGUUAAGAUGAAAAGUCGGAGCCGGAGCGAAAGCGAGUCUGA
ACAGGGCGCUUCAGUAGGUAGAUGUAGACCCGAAACCAAGUGACCUACCCAUGACCAG
GUUGAAGGUGUGGUGAAACACACUGGAGGACCGAACCCAUGUAUGUUGAAAAAUGCU
GGGAUGAGUUGUGGGUAGCGGUGAAAUUCCAAACGAACUUGGAGAUAGCUGGUUCUC
UCCGAAAUAGCUUUAGGGUUAGCCUCGGAGGAUGGAUCAUGGAGGUAGAGCACUGUU
UGAACUAGGGGCCCGUCAAGGGUUACUGAAUUCAGAUAAACUCCGAAUACCAUUGAU
CUUACUCCGGGAGUCAGACAGUGAGCGAUAAGGUCCAUUGUCGAAGGGGGAACAGCC
CAGAUCACCAGUUAAGGUCCCUAAAUUUAUGCUAAGUGGAAAAGGAUGUGGCGUUGC
ACAGACAACUAGGAUGUUGGCUCAGAAGCAGCCACCAUUUAAAGAGUGCGUAAUAGC
UCACUAGUCGAGUGGCACUGCGCCGAAAAUAUACCGGGGCUAAGCAUAAUACCGAAA
CUGUGGGUGCACCCGUCAGGGUGCGCGGUAGGAGAGCGUUCUAAGGGCGUUGAAGGU
CGAUCGUGAGGACGGCUGGAGCGCUUAGAAGUGAGAAUGCCGGCAUGAGUAGCGAAA
GAUCAGUGAGAAUCUGAUCCACCGUAUGACUAAGGUUUCCUGGGGAAGGCUCGUCCU
CCCAGGGUUAGUCGGGAUCUAAGGCGAGGCCGCAAGGCGUAGUCGAUGACAAGCAGG
UUGAGAUUCCUGCACUAGUUUAUUUUGUUUAAGCGAUGGAGGGACGCAGGAGGCUAA
GGAAAGCGCACGGCUGGAAAAGUGCGUCCAAGCAGUAAGUCCGGUAGCGAGUGAAAU
GCUUGCCGCCUUAAGGACAAGCUGUGAUGGGGAGCGAAAUUAAAGUAGCGAAGUUCC
UGAUGUCACACUGCCAAGAAAAGCUUCUAGUGAGAAAUAAACUACCCGUACCGCAAA
CCGACACAGGUAGUCGAGGAGAGUAUCCUCAGGUGAGCGAGCGAACUCUCGUUAAGG
AACUCGGCAAAAUGACCCCGUAACUUCGGAAGAAGGGGUGCUGACCGCAAGGUCAGCC
GCAGUGAAUAGGCCCAAACAACUGUUUAUCAAAAACACAGGUCUCUGCUAAAUCGUA
AGAUGAUGUAUAGGGGCUGACGCCUGCCCGGUGCUGGAAGGUUAAGAGGAUGAGUUA
GCGCAAGCGAAGCCCAGAAUUGAAGCCCCAGUAAACGGCGGCCGUAACUAUAACGGUC
CUAAGGUAGCGAAAUUCCUUGUCGGGUAAGUUCCGACCCGCACGAAAGGCGUAAUGA
UUUGGGCACUGUCUCAACGAGAGACUCGGUGAAAUUAUAGUACCCGUGAAGAUGCGG
GUUACCCGCGACAGGACGGAAAGACCCCAUGGAGCUUUACUGUAGCUUGAUAUUGAG
UGUUGGUACCGCUUGUACAGGAUAGGUAGGAGCCGUAGAGAUCGGAACGCUAGUUUC
GAUGGAGGCAUUGGUGGGAUACUACCCUAGCUGUAUGAACACUCUAACCCGCGCCACU
GAUCGUGGCGGGAGACAGUGUCAGGUAGGCAGUUUGACUGGGGCGGUCGCCUCCUAA
AAUGUAACGGAGGCGCCCAAAGGUUCCCUCAGAAUGGUUGGAAAUCAUUCGCAGAGU
GUAAAGGUAGAAGGGAGCUUGACUGCGAGACUGACAAGUCGAGCAGGGACGAAAGUC
GGGCUUAGUGAUCCGGUGGUUCCGUAUGGAAGGGCCAUCGCUCAACGGAUAAAAGCU
ACCCUGGGGAUAACAGGCUUAUCUCCCCCAAGAGUCCACAUCGACGGGGAGGUUUGGC
ACCUCGAUGUCGGCUCAUCGCAUCCUGGGGCUGUAGUCGGUCCCAAGGGUUGGGCUGU
UCGCCCAUUAAAGCGGUACGCGAGCUGGGUUCAGAACGUCGUGAGACAGUUCGGUCCC
UAUCCGUCGCGGGCGCAGGAAAUUUGAGAGGAGCUGUCCUUAGUACGAGAGGACCGG
GAUGGACGUUCCGCUGGUGUACCAGUUGUGCCGCCAGGCGCAUCGCUGGGUAGCUAU
GAACGGAAGGGAUAAACGCUGAAAGCAUCUAAGUGUGAAGCCCCCCUCGAGAUGAGA
UUUCCCAUUCCUAUAUGGAAGUAAGACCCCUGAAAGAUGAUCAGGUAGAUAGGCUGG
AAGUGGAAGUGCAGCGAUGCAUGGAGCGGACCAGUACUAAUCGGUCGAGGACUUAAC
CAAG
SEQ ID NO: 44, L. rhamnosus, Accession Number: AFYD01000005.110.1639
Lacticaseibacillus; Lactobacillus rhamnosus. Sequence:
GAAGUGAGAAUGCCGGCAUGAGUAGCGAAAGAUCAGUGAGAAUCUGAUCCACCGUAU
GACUAAGGUUUCCUGGGGAAGGCUCGUCCUCCCAGGGUUAGUCGGGAUCUAAGGCGA
GGCCGCAAGGCGUAGUCGAUGACAAGCAGGUUGAGAUUCCUGCACUAGUUUAUUUUG
UUUAAGCGAUGGAGGGACGCAGGAGGCUAAGGAAAGCGCACGGCUGGAAAAGUGCGU
CCAAGCAGUAAGUCCGGUAGCGAGUGAAAUGCUUGCCGCCUUAAGGACAAGCUGUGA
UGGGGAGCGAAAUUAAAGUAGCGAAGUUCCUGAUGUCACACUGCCAAGAAAAGCUUC
UAGUGAGAAAUAAACUACCCGUACCGCAAACCGACACAGGUAGUCGAGGAGAGUAUC
CUCAGGUGAGCGAGCGAACUCUCGUUAAGGAACUCGGCAAAAUGACCCCGUAACUUCG
GAAGAAGGGGUGCUGACCGCAAGGUCAGCCGCAGUGAAUAGGCCCAAACAACUGUUU
AUCAAAAACACAGGUCUCUGCUAAAUCGUAAGAUGAUGUAUAGGGGCUGACGCCUGC
CCGGUGCUGGAAGGUUAAGAGGAUGAGUUAGCGCAAGCGAAGCCCAGAAUUGAAGCC
CCAGUAAACGGCGGCCGUAACUAUAACGGUCCUAAGGUAGCGAAAUUCCUUGUCGGG
UAAGUUCCGACCCGCACGAAAGGCGUAAUGAUUUGGGCACUGUCUCAACGAGAGACU
CGGUGAAAUUAUAGUACCCGUGAAGAUGCGGGUUACCCGCGACAGGACGGAAAGACC
CCAUGGAGCUUUACUGUAGCUUGAUAUUGAGUGUUGGUACCGCUUGUACAGGAUAGG
UAGGAGCCGUAGAGAUCGGAACGCUAGUUUCGAUGGAGGCAUUGGUGGGAUACUACC
CUAGCUGUAUGAACACUCUAACCCGCGCCACUAAUCGUGGCGGGAGACAGUGUCAGGU
AGGCAGUUUGACUGGGGCGGUCGCCUCCUAAAAUGUAACGGAGGCGCCCAAAGGUUC
CCUCAGAAUGGUUGGAAAUCAUUCGCAGAGUGUAAAGGUAGAAGGGAGCUUGACUGC
GAGACUGACAAGUCGAGCAGGGACGAAAGUCGGGCUUAGUGAUCCGGUGGUUCCGUA
UGGAAGGGCCAUCGCUCAACGGAUAAAAGCUACCCUGGGGAUAACAGGCUUAUCUCCC
CCAAGAGUCCACAUCGACGGGGAGGUUUGGCACCUCGAUGUCGGCUCAUCGCAUCCUG
GGGCUGUAGUCGGUCCCAAGGGUUGGGCUGUUCGCCCAUUAAAGCGGUACGCGAGCU
GGGUUCAGAACGUCGUGAGACAGUUCGGUCCCUAUCCGUCGCGGGCGCAGGAAAUUU
GAGAGGAGCUGUCCUUAGUACGAGAGGACCGGGAUGGACGUUCCGCUGGUGUACCAG
UUGUGCCGCCAGGCGCAUCGCUGGGUAGCUAUGAACGGAAGGGAUAAACGCUGAAAG
CAUCUAAGUGUGAAGCCCCCCUCGAGAUGAGAUUUCCCAUUCCU
Exemplary L. paracasei sequences
SEQ ID NO: 45, L. paracasei 16S sequence, 1522 nt (see e.g., Lactobacillus paracasei
strain R094 16S ribosomal RNA gene, partial sequence, NCBI Reference Sequence:
NR_025880.1
GATGAACGCTGGCGGCGTGCCTAATACATGCAAGTCGAACGAGTTCTCGTTGATGATCG
GTGCTTGCACCGAGATTCAACATGGAACGAGTGGCGGACGGGTGAGTAACACGTGGGTA
ACCTGCCCTTAAGTGGGGGATAACATTTGGAAACAGATGCTAATACCGCATAGATCCAA
GAACCGCATGGTTCTTGGCTGAAAGATGGCGTAAGCTATCGCTTTTGGATGGACCCGCGG
CGTATTAGCTAGTTGGTGAGGTAATGGCTCACCAAGGCGATGATACGTAGCCGAACTGA
GAGGTTGATCGGCCACATTGGGACTGAGACACGGCCCAAACTCCTACGGGAGGCAGCAG
TAGGGAATCTTCCACAATGGACGCAAGTCTGATGGAGCAACGCCGCGTGAGTGAAGAAG
GCTTTCGGGTCGTAAAACTCTGTTGTTGGAGAAGAATGGTCGGCAGAGTAACTGTTGTCG
GCGTGACGGTATCCAACCAGAAAGCCACGGCTAACTACGTGCCAGCAGCCGCGGTAATA
CGTAGGTGGCAAGCGTTATCCGGATTTATTGGGCGTAAAGCGAGCGCAGGCGGTTTTTTA
AGTCTGATGTGAAAGCCCTCGGCTTAACCGAGGAAGCGCATCGGAAACTGGGAAACTTG
AGTGCAGAAGAGGACAGTGGAACTCCATGTGTAGCGGTGAAATGCGTAGATATATGGAA
GAACACCAGTGGCGAAGGCGGCTGTCTGGTCTGTAACTGACGCTGAGGCTCGAAAGCAT
GGGTAGCGAACAGGATTAGATACCCTGGTAGTCCATGCCGTAAACGATGAATGCTAGGT
GTTGGAGGGTTTCCGCCCTTCAGTGCCGCAGCTAACGCATTAAGCATTCCGCCTGGGGAG
TACGACCGCAAGGTTGAAACTCAAAGGAATTGACGGGGGCCCGCACAAGCGGTGGAGC
ATGTGGTTTAATTCGAAGCAACGCGAAGAACCTTACCAGGTCTTGACATCTTTTGATCAC
CTGAGAGATCAGGTTTCCCCTTCGGGGGCAAAATGACAGGTGGTGCATGGTTGTCGTCAG
CTCGTGTCGTGAGATGTTGGGTTAAGTCCCGCAACGAGCGCAACCCTTATGACTAGTTGC
CAGCATTTAGTTGGGCACTCTAGTAAGACTGCCGGTGACAAACCGGAGGAAGGTGGGGA
TGACGTCAAATCATCATGCCCCTTATGACCTGGGCTACACACGTGCTACAATGGATGGTA
CAACGAGTTGCGAGACCGCGAGGTCAAGCTAATCTCTTAAAGCCATTCTCAGTTCGGACT
GTAGGCTGCAACTCGCCTACACGAAGTCGGAATCGCTAGTAATCGCGGATCAGCACGCC
GCGGTGAATACGTTCCCGGGCCTTGTACACACCGCCCGTCACACCATGAGAGTTTGTAAC
ACCCGAAGCCGGTGGCGTAACCCTTTTAGGGAGCGAGCCGTCTAAGGTGGGACAAATGA
TTAGGGTGAAGTCGTAACAAGGTAGCCGTAGGAGAACC
SEQ ID NO: 46, L. paracasei, Accession Number: CP002391.1862810.1865725
Lacticaseibacillus; Lactobacillus paracasei. Sequence:
UUAAGUUACAAAGGGCGCACGGUGGAUGCCUUGGCACUAGGAGCCGAUGAAGGACGG
AACUAAUACCGAUAUGCUUCGGGGAGCUAUAAGUAAGCUUUGAUCCGGAGAUUUCCG
AAUGGGGGAACCCAGUACACAUCAGUGUAUUGCUUGUCAGUGAAUACAUAGCUGGCC
GGCGGCAGACGCGGGGAACUGAAACAUCUCAGUACCCGCAGGAAGAGAAAGAAAACU
CGAUUCCCAUAGUAGCGGCGAGCGAAGUGGGAAGAGCCCAAACCGAGAAGCUUGCUU
CUCGGGGUUGUAGGACUGGACAUUGGAGUUACCAAAGUCCGACGUAGUCGAAGUCAG
CUGGAAAGCUGCGCCACAGAAGGUGAAAGCCCUGUAAGCGAAACGUCGAACCCUCCGU
CCAGGAUCCUGAGUACGGCGGAACACGUGAAAUUCCGUCGGAAUCCGGGAGGACCAUC
UCCCAAGGCUAAAUACUCCCUAGUGACCGAUAGUGAACCAGUACCGUGAGGGAAAGG
UGAAAAGCACCCCGGAAGGGGAGUGAAACAGUUCCUGAAACCGUGUGCCUACAAUUA
GUCAAAGCCCGUUAAUGGGUAAUGGCGUGCCUUUUGUAGAAUGAACCGGCGAGUUAC
GUUUGCUUGCGAGGUUAAGAUGAAAAGUCGGAGCCGUAGCGAAAGCGAGUCUGAACA
GGGCGAAUGAGUAAGCAGAUGUAGACCCGAAACCAAGUGACCUACCCAUGACCAGGU
UGAAGGUGUGGUGAAACACACUGGAGGACCGAACCCAUGUAUGUUGAAAAAUGCUGG
GAUGAGUUGUGGGUAGCGGUGAAAUUCCAAACGAACUUGGAGAUAGCUGGUUCUCUC
CGAAAUAGCUUUAGGGUUAGCCUCGGAGGAUGGAUCAUGGAGGUAGAGCACUGUUUG
AACUAGGGGCCCAUCAAGGGUUACUGAAUUCAGAUAAACUCCGAAUACCAUCGAUCU
UACUCCGGGAGUCAGACAGUGAGCGAUAAGGUCCAUUGUCGAAAGGGGAACAGCCCA
GAUCACCAGUUAAGGUCCCUAAAUUUAUGCUAAGUGGAAAAGGAUGUGGCGUUGCAC
AGACAACUAGGAUGUUGGCUCAGAAGCAGCCACCAUUUAAAGAGUGCGUAAUAGCUC
ACUAGUCGAGUGGCACUGCGCCGAAAAUAUACCGGGGCUAAGCAUAAUACCGAAACU
GUGGGUGCACCCGUAAGGGUGCGCGGUAGGAGAGCGUUCUAAGGGCGUUGAAGGUCG
AUCGUGAGGACGGCUGGAGCGCUUAGAAGUGAGAAUGCCGGCAUGAGUAGCGAAAGA
UCAGUGAGAAUCUGAUCCACCGUAUGACUAAGGUUUCCUGGGGAAGGCUCGUCCUCCC
AGGGUUAGUCGGGAUCUAAGGCGAGGCCGAGAGGCGUAGUCGAUGACAAGCAGGUUG
AGAUUCCUGCACUAGUUUAUUUUGUUUAAGCGAUGGAGGGACGCAGGAGGCUAAGGA
AAGCGCACGGCUGGAAAUGUGCGCCCAAGCAGCAAGUCUGACAGCGAGUGAAAUGCU
UGCAGUCUUAAGGACAAGCUGUGAUGGGGAGCGAAAUUAUAGUAGCGAAGUUCCUGA
UGUCACACUGCCAAGAAAAGCUUCUAGUGAGAAAUAAACUACCCGUACCGCAAACCGA
CACAGGUAGUCGAGGAGAGUAUCCUCAGGUGAGCGAGCGAACUCUCGUUAAGGAACU
CGGCAAAAUGACCCCGUAACUUCGGAAGAAGGGGUGCUGACCGCAAGGUCAGCCGCAG
UGAAUAGGCCCAAACAACUGUUUAUCAAAAACACAGGUCUCUGCUAAAUCGUAAGAU
GAUGUAUAGGGGCUGACGCCUGCCCGGUGCUGGAAGGUUAAGAGGAUGAGUUAGCGC
AAGCGAAGCCCAGAAUUGAAGCCCCAGUAAACGGCGGCCGUAACUAUAACGGUCCUAA
GGUAGCGAAAUUCCUUGUCGGGUAAGUUCCGACCCGCACGAAAGGCGUAAUGAUUUG
GGCACUGUCUCAACGAGAGACUCGGUGAAAUUAUAGUACCCGUGAAGAUGCGGGUUA
CCCGCGACAGGACGGAAAGACCCCAUGGAGCUUUACUGUAGCUUGAUAUUGAGUGUU
UGUACCGCUUGUACAGGAUAGGUAGGAGCCGUAGAGAUCGGAACGCUAGUUUCGAUG
GAGGCGUUGGUGGGAUACUACCCUAGCUGUAUGAACACUCUAACCCGCGCCACUAAUC
GUGGCGGGAGACAGUGUCAGGUAGGCAGUUUGACUGGGGCGGUCGCCUCCUAAAAUG
UAACGGAGGCGCCCAAAGGUUCCCUCAGAAUGGUUGGAAAUCAUUCGCAGAGUGUAA
AGGUAUAAGGGAGCUUGACUGCGAGACUGACAAGUCGAGCAGGGACGAAAGUCGGGC
UUAGUGAUCCGGUGGUUCCGUAUGGAAGGGCCAUCGCUCAACGGAUAAAAGCUACCC
UGGGGAUAACAGGCUUAUCUCCCCCAAGAGUCCACAUCGACGGGGAGGUUUGGCACCU
CGAUGUCGGCUCAUCGCAUCCUGGGGCUGUAGUCGGUCCCAAGGGUUGGGCUGUUCGC
CCAUUAAAGCGGUACGCGAGCUGGGUUCAGAACGUCGUGAGACAGUUCGGUCCCUAU
CCGUCGCGGGCGCAGGAAAUUUGAGAGGAGCUGUCCUUAGUACGAGAGGACCGGGAU
GGACGUUCCGCUGGUGUACCAGUUGUGCCGCCAGGCGCAUCGCUGGGUAGCUAUGAAC
GGCAGGGAUAAACGCUGAAAGCAUCUAAGUGUGAAGCCCCCCUCGAGAUGAGAUUUC
CCAUUCCUAUAUGGAAGUAAGACCCCUGAGAGAUGAUCAGGUAGAUAGGCUGGAAGU
GGAAGUGCAGCGAUGCAUGGAGCGGACCAGUACUAAUCGGUCGAGGACUUAACCAA
SEQ ID NO: 47, L. paracasei, Accession Number: CP016355.2612922.2615840
Lacticaseibacillus; Lactobacillus paracasei. Sequence:
AGGUUAAGUUACAAAGGGCGCACGGUGGAUGCCUUGGCACUAGGAGCCGAUGAAGGA
CGGAACUAAUACCGAUAUGCUUCGGGGAGCUAUAAGUAAGCUUUGAUCCGGAGAUUU
CCGAAUGGGGGAACCCAGUACACAUCAGUGUAUUGCUUGUCAGUGAAUACAUAGCUG
GCCGGCGGCAGACGCGGGGAACUGAAACAUCUCAGUACCCGCAGGAAGAGAAAGAAA
ACUCGAUUCCCAUAGUAGCGGCGAGCGAAGUGGGAAGAGCCCAAACCGAGAAGCUUG
CUUCUCGGGGUUGUAGGACUGGACAUUGGAGUUACCAAAGUCCGACGUAGUCGAAGU
CAGCUGGAAAGCUGCGCCACAGAAGGUGAAAGCCCUGUAAGCGAAACGUCGAACCCUC
CGUUCAGGAUCCUGAGUACGGCGGAACACGUGAAAUUCCGUCGGAAUCCGGGAGGAC
CAUCUCCCAAGGCUAAAUACUCCCUAGUGACCGAUAGUGAACCAGUACCGUGAGGGAA
AGGUGAAAAGCACCCCGGAAGGGGAGUGAAACAGUUCCUGAAACCGUGUGCCUACAA
UUAGUCAAAGCCCGUUAAUGGGUAAUGGCGUGCCUUUUGUAGAAUGAACCGGCGAGU
UACGUUUGCUUGCGAGGUUAAGAUGAAAAGUCGGAGCCGUAGCGAAAGCGAGUCUGA
ACAGGGCGAAUGAGUAAGCAGAUGUAGACCCGAAACCAAGUGACCUACCCAUGACCA
GGUUGAAGGUGUGGUGAAACACACUGGAGGACCGAACCCAUGUAUGUUGAAAAAUGC
UGGGAUGAGUUGUGGGUAGCGGUGAAAUUCCAAACGAACUUGGAGAUAGCUGGUUCU
CUCCGAAAUAGCUUUAGGGUUAGCCUCGGAGGAUGGAUCAUGGAGGUAGAGCACUGU
UUGAACUAGGGGCCCAUCAAGGGUUACUGAAUUCAGAUAAACUCCGAAUACCAUCGA
UCUUACUCCGGGAGUCAGACAGUGAGCGAUAAGGUCCAUUGUCGAAAGGGGAACAGC
CCAGAUCACCAGUUAAGGUCCCUAAAUUUAUGCUAAGUGGAAAAGGAUGUGGCGUUG
CACAGACAACUAGGAUGUUGGCUCAGAAGCAGCCACCAUUUAAAGAGUGCGUAAUAG
CUCACUAGUCGAGUGGCACUGCGCCGAAAAUAUACCGGGGCUAAGCAUAAUACCGAA
ACUGUGGGUGCACCCGUAAGGGUGCGCGGUAGGAGAGCGUUCUAAGGGCGUUGAAGG
UCGAUCGUGAGGACGGCUGGAGCGCUUAGAAGUGAGAAUGCCGGCAUGAGUAGCGAA
AGAUCAGUGAGAAUCUGAUCCACCGUAUGACUAAGGUUUCCUGGGGAAGGCUCGUCC
UCCCAGGGUUAGUCGGGAUCUAAGGCGAGGCCGAGAGGCGUAGUCGAUGACAAGCAG
GUUGAGAUUCCUGCACUAGUUUAUUUUGUUUAAGCGAUGGAGGGACGCAGGAGGCUA
AGGAAAGCGCACGGCUGGAAAUGUGCGCCCAAGCAGCAAGUCUGACAGCGAGUGAAA
UGCUUGCAGUCUUAAGGACAAGCUGUGAUGGGGAGCGAAAUUAUAGUAGCGAAGUUC
CUGAUGUCACACUGCCAAGAAAAGCUUCUAGUGAGAAAUAAACUACCCGUACCGCAA
ACCGACACAGGUAGUCGAGGAGAGUAUCCUCAGGUGAGCGAGCGAACUCUCGUUAAG
GAACUCGGCAAAAUGACCCCGUAACUUCGGAAGAAGGGGUGCUGACCGCAAGGUCAG
CCGCAGUGAAUAGGCCCAAACAACUGUUUAUCAAAAACACAGGUCUCUGCUAAAUCG
UAAGAUGAUGUAUAGGGGCUGACGCCUGCCCGGUGCUGGAAGGUUAAGAGGAUGAGU
UAGCGCAAGCGAAGCCCAGAAUUGAAGCCCCAGUAAACGGCGGCCGUAACUAUAACGG
UCCUAAGGUAGCGAAAUUCCUUGUCGGGUAAGUUCCGACCCGCACGAAAGGCGUAAU
GAUUUGGGCACUGUCUCAACGAGAGACUCGGUGAAAUUAUAGUACCCGUGAAGAUGC
GGGUUACCCGCGACAGGACGGAAAGACCCCAUGGAGCUUUACUGUAGCUUGAUAUUG
AGUGUUUGUACCGCUUGUACAGGAUAGGUAGGAGCCGUAGAGAUCGGAACGCUAGUU
UCGAUGGAGGCGUUGGUGGGAUACUACCCUAGCUGUAUGAACACUCUAACCCGCGCCA
CUAAUCGUGGCGGGAGACAGUGUCAGGUAGGCAGUUUGACUGGGGCGGUCGCCUCCU
AAAAUGUAACGGAGGCGCCCAAAGGUUCCCUCAGAAUGGUUGGAAAUCAUUCGCAGA
GUGUAAAGGUAUAAGGGAGCUUGACUGCGAGACUGACAAGUCGAGCAGGGACGAAAG
UCGGGCUUAGUGAUCCGGUGGUUCCGUAUGGAAGGGCCAUCGCUCAACGGAUAAAAG
CUACCCUGGGGAUAACAGGCUUAUCUCCCCAAGAGUCCACAUCGACGGGGAGGUUUGG
CACCUCGAUGUCGGCUCAUCGCAUCCUGGGGCUGUAGUCGGUCCCAAGGGUUGGGCUG
UUCGCCCAUUAAAGCGGUACGCGAGCUGGGUUCAGAACGUCGUGAGACAGUUCGGUC
CCUAUCCGUCGCGGGCGCAGGAAAUUUGAGAGGAGCUGUCCUUAGUACGAGAGGACC
GGGAUGGACGUUCCGCUGGUGUACCAGUUGUGCCGCCAGGCGCAUCGCUGGGUAGCU
AUGAACGGCAGGGAUAAACGCUGAAAGCAUCUAAGUGUGAAGCCCCCCUCGAGAUGA
GAUUUCCCAUUCCUAUAUGGAAGUAAGACCCCUGAGAGAUGAUCAGGUAGAUAGGCU
GGAAGUGGAAGUGCAGCGAUGCAUGGAGCGGACCAGUACUAAUCGGUCGAGGACUUA
ACCAAG
SEQ ID NO: 48, L. paracasei, Accession Number: ANKB01000105.125.3044
Lacticaseibacillus; Lactobacillus paracasei. Sequence:
AGGUUAAGUUACAAAGGGCGCACGGUGGAUGCCUUGGCACUAGGAGCCGAUGAAGGA
CGGAACUAAUACCGAUAUGCUUCGGGGAGCUAUAAGUAAGCUUUGAUCCGGAGAUUU
CCGAAUGGGGGAACCCAGUACACAUCAGUGUAUUGCUUGUCAGUGAAUACAUAGCUG
GCCGGCGGCAGACGCGGGGAACUGAAACAUCUCAGUACCCGCAGGAAGAGAAAGAAA
ACUCGAUUCCCAUAGUAGCGGCGAGCGAAGUGGGAAGAGCCCAAACCGAGAAGCUUG
CUUCUCGGGGUUGUAGGACUGGACAUUGGAGUUACCAAAGUCCGACGUAGUCGAAGU
CAGCUGGAAAGCUGCGCCACAGAAGGUGAAAGCCCUGUAAGCGAAACGUCGAACCCUC
CGUCCAGGAUCCUGAGUACGGCGGAACACGUGAAAUUCCGUCGGAAUCCGGGAGGACC
AUCUCCCAAGGCUAAAUACUCCCUAGUGACCGAUAGUGAACCAGUACCGUGAGGGAA
AGGUGAAAAGCACCCCGGAAGGGGAGUGAAACAGUUCCUGAAACCGUGUGCCUACAA
UUAGUCAAAGCCCGUUAAUGGGUAAUGGCGUGCCUUUUGUAGAAUGAACCGGCGAGU
UACGUUUGCUUGCGAGGUUAAGAUGAAAAGUCGGAGCCGUAGCGAAAGCGAGUCUGA
ACAGGGCGAAUGAGUAAGCAGAUGUAGACCCGAAACCAAGUGACCUACCCAUGACCA
GGUUGAAGGUGUGGUGAAACACACUGGAGGACCGAACCCAUGUAUGUUGAAAAAUGC
UGGGAUGAGUUGUGGGUAGCGGUGAAAUUCCAAACGAACUUGGAGAUAGCUGGUUCU
CUCCGAAAUAGCUUUAGGGUUAGCCUCGGAGGAUGGAUCAUGGAGGUAGAGCACUGU
UUGAACUAGGGGCCCAUCAAGGGUUACUGAAUUCAGAUAAACUCCGAAUACCAUCGA
UCUUACUCCGGGAGUCAGACAGUGAGCGAUAAGGUCCAUUGUCGAAAGGGGAACAGC
CCAGAUCACCAGUUAAGGUCCCUAAAUUUAUGCUAAGUGGAAAAGGAUGUGGCGUUG
CACAGACAACUAGGAUGUUGGCUCAGAAGCAGCCACCAUUUAAAGAGUGCGUAAUAG
CUCACUAGUCGAGUGGCACUGCGCCGAAAAUAUACCGGGGCUAAGCAUAAUACCGAA
ACUGUGGGUGCACCCGUAAGGGUGCGCGGUAGGAGAGCGUUCUAAGGGCGUUGAAGG
UCGAUCGUGAGGACGGCUGGAGCGCUUAGAAGUGAGAAUGCCGGCAUGAGUAGCGAA
AGAUCAGUGAGAAUCUGAUCCACCGUAUGACUAAGGUUUCCUGGGGAAGGCUCGUCC
UCCCAGGGUUAGUCGGGAUCUAAGGCGAGGCCGAGAGGCGUAGUCGAUGACAAGCAG
GUUGAGAUUCCUGCACUAGUUUAUUUUGUUUAAGCGAUGGAGGGACGCAGGAGGCUA
AGGAAAGCGCACGGCUGGAAAUGUGCGCCCAAGCAGCAAGUCUGACAGCGAGUGAAA
UGCUUGCAGUCUUAAGGACAAGCUGUGAUGGGGAGCGAAAUUAUAGUAGCGAAGUUC
CUGAUGUCACACUGCCAAGAAAAGCUUCUAGUGAGAAAUAAACUACCCGUACCGCAA
ACCGACACAGGUAGUCGAGGAGAGUAUCCUCAGGUGAGCGAGCGAACUCUCGUUAAG
GAACUCGGCAAAAUGACCCCGUAACUUCGGAAGAAGGGGUGCUGACCGCAAGGUCAG
CCGCAGUGAAUAGGCCCAAACAACUGUUUAUCAAAAACACAGGUCUCUGCUAAAUCG
UAAGAUGAUGUAUAGGGGCUGACGCCUGCCCGGUGCUGGAAGGUUAAGAGGAUGAGU
UAGCGCAAGCGAAGCCCAGAAUUGAAGCCCCAGUAAACGGCGGCCGUAACUAUAACGG
UCCUAAGGUAGCGAAAUUCCUUGUCGGGUAAGUUCCGACCCGCACGAAAGGCGUAAU
GAUUUGGGCACUGUCUCAACGAGAGACUCGGUGAAAUUAUAGUACCCGUGAAGAUGC
GGGUUACCCGCGACAGGACGGAAAGACCCCAUGGAGCUUUACUGUAGCUUGAUAUUG
AGUGUUUGUACCGCUUGUACAGGAUAGGUAGGAGCCGUAGAGAUCGGAACGCUAGUU
UCGAUGGAGGCGUUGGUGGGAUACUACCCUAGCUGUAUGAACACUCUAACCCGCGCCA
CUAAUCGUGGCGGGAGACAGUGUCAGGUAGGCAGUUUGACUGGGGCGGUCGCCUCCU
AAAAUGUAACGGAGGCGCCCAAAGGUUCCCUCAGAAUGGUUGGAAAUCAUUCGCAGA
GUGUAAAGGUAUAAGGGAGCUUGACUGCGAGACUGACAAGUCGAGCAGGGACGAAAG
UCGGGCUUAGUGAUCCGGUGGUUCCGUAUGGAAGGGCCAUCGCUCAACGGAUAAAAG
CUACCCUGGGGAUAACAGGCUUAUCUCCCCCAAGAGUCCACAUCGACGGGGAGGUUUG
GCACCUCGAUGUCGGCUCAUCGCAUCCUGGGGCUGUAGUCGGUCCCAAGGGUUGGGCU
GUUCGCCCAUUAAAGCGGUACGCGAGCUGGGUUCAGAACGUCGUGAGACAGUUCGGU
CCCUAUCCGUCGCGGGCGCAGGAAAUUUGAGAGGAGCUGUCCUUAGUACGAGAGGAC
CGGGAUGGACGUUCCGCUGGUGUACCAGUUGUGCCGCCAGGCGCAUCGCUGGGUAGCU
AUGAACGGCAGGGAUAAACGCUGAAAGCAUCUAAGUGUGAAGCCCCCCUCGAGAUGA
GAUUUCCCAUUCCUAUAUGGAAGUAAGACCCCUGAGAGAUGAUCAGGUAGAUAGGCU
GGAAGUGGAAGUGCAGCGAUGCAUGGAGCGGACCAGUACUAAUCGGUCGAGGACUUA
ACCAAG
Exemplary L. casei sequences
SEQ ID NO: 49, L. casei 16S, Lacticaseibacillus casei DSM 20011 = JCM 1134 =
ATCC 393 16S ribosomal RNA, partial sequence, 1517 nucleotides (nt)
TCCTGGCTCAGGATGAACGCTGGCGGCGTGCCTAATACATGCAAGTCGAACGAGTTTTGG
TCGATGAACGGTGCTTGCACTGAGATTCGACTTAAAACGAGTGGCGGACGGGTGAGTAA
CACGTGGGTAACCTGCCCTTAAGTGGGGGATAACATTTGGAAACAGATGCTAATACCGC
ATAAATCCAAGAACCGCATGGTTCTTGGCTGAAAGATGGCGTCAAGCTATCGCTTTTGGA
TGGACCCGCGGCGTATTAGCTAGTTGGTGAGGTAACGGCTCACCAAGGCGATGATACGT
AGCCGAACTGAGAGGTTGATCGGCCACATTGGGACTGAGACACGGCCCAAACTCCTACG
GGAGGCAGCAGTAGGGAATCTTCCACAATGGACGCAAGTCTGATGGAGCAACGCCGCGT
GAGTGAAGAAGGCTTTCGGGTCGTAAAACTCTGTTGTTGGAGAAGAATGGTCGGCAGAG
TAACTGTTGTCGGCGTGACGGTATCCAACCAGAAAGCCACGGCTAACTACGTGCCAGCA
GCCGCGGTAATACGTAGGTGGCAAGCGTTATCCGGATTTATTGGGCGTAAAGCGAGCGC
AGGCGGTTTTTTAAGTCTGATGTGAAAGCCCTCGGCTTAACCGAGGAAGCGCATCGGAA
ACTGGGAAACTTGAGTGCAGAAGAGGACAGTGGAACTCCATGTGTAGCGGTGAAATGCG
TAGATATATGGAAGAACACCAGTGGCGAAGGCGGCTGTCTGGTCTGTAACTGACGCTGA
GGCTCGAAAGCATGGGTAGCGAACAGGATTAGATACCCTGGTAGTCCATGCCGTAAACG
ATGAATGCTAGGTGTTGGAGGGTTTCCGCCCTTCAGTGCCGCAGCTAACGCATTAAGCAT
TCCGCCTGGGGAGTACGACCGCAAGGTTGAAACTCAAAGGAATTGACGGGGGCCCGCAC
AAGCGGTGGAGCATGTGGTTTAATTCGAAGCAACGCGAAGAACCTTACCAGGTCTTGAC
ATCTTTTGATCACCTGAGAGATCAGGTTTCCCCTTCGGGGGCAAAATGACAGGTGGTGCA
TGGTTGTCGTCAGCTCGTGTCGTGAGATGTTGGGTTAAGTCCCGCAACGAGCGCAACCCT
TATGACTAGTTGCCAGCATTGAGTTGGGCACTCTAGTAAGACTGCCGGTGACAAACCGG
AGGAAGGTGGGGATGACGTCAAATCATCATGCCCCTTATGACCTGGGCTACACACGTGC
TACAATGGATGGTACAACGAGTTGCGAGACCGCGAGGTCAAGCTAATCTCTTAAAGCCA
TTCTCAGTTCGGACTGTAGGCTGCAACTCGCCTACACGAAGTCGGAATCGCTAGTAATCG
CGGATCAGCACGCCGCGGTGAATACGTTCCCGGGCCTTGTACACACCGCCCGTCACACCA
TGAGAGTTTGTAACACCCGAAGCCGGTGGCGTAACCCTTTTAGGGAGCGAGCCGTCTAA
GGTGGGACAAATGATTAGGGTGAAGTCGTAACAAG
SEQ ID NO: 50, L. casei, Accession Number: AB092640.223.705 Lacticaseibacillus;
Lactobacillus casei. Sequence:
GGUUAAGUUACAAAGGGCGCACGGUGGAUGCCUUGGCACUAGGAGCCGAUGAAGGAC
GGAACUAAUACCGAUAUGCUUCGGGGAGCUAUAAGUAAGCUUUGAUCCGGAGAUUUC
CGAAUGGGGGAACCCAGUACACAUCAGUGUAUUGCCUGCAAGUGAAUACAUAGCUUG
UUGGCGGCAGACGCGGGGAACUGAAACAUCUCAGUACCCGCAGGAAGAGAAAGAAAA
CUCGAUUCCCAUAGUAGCGGCGAGCGAAGUGGGAAGAGCCCAAACCGAGAAGCUUGC
UUCUCGGGGUUGUAGGACUGGACAUUGGAGUUACCAAAGUUCGACGUAGUCGAAGUC
AGCUGGAAAGCUGCGCCAUAGAAGGUGAAAGCCCUGUAAACGAAACGGCGGACUCUC
CGUCCAGGAUCCUGAGUACGGCGGAACACGUGAAAUUCCGUCGGAAUCCGGGAGGACC
AUCUCCCAAGGCUAAAUACUCCCUAG
SEQ ID NO: 51, L. casei, Accession Number: AY221478.1.556 Lacticaseibacillus;
Lactobacillus casei. Sequence:
UAGGAGCGAUGAAGGACGGAACUAAUACCGAUAUGCUUCGGGGAGCUAUAAGUAAGC
UUUGAUCCGGAGAUUUCCGAAUGGGGGAACCCAGUACACAUCAGUGUAUUGCUUGUC
AGUGAAUACAUAGCUGGCCGGCGGCAGACGCGGGGAACUGAAACAUCUCAGUACCCGC
AGGAAGAGAAAGAAAACUCGAUUCCCAUAGUAGCGGCGAGCGAAGUGGGAAGAGCCC
AAACCGAGAAGCUUGCUUCUCGGGGUUGUAGGACUGGACAUUGGAGUUACCAAAGUC
CGACGUAGUCGAAGUCAGCUGGAAAGCUGCGCCACAGAAGGUGAAAGCCCUGUAAGC
GAAACGUCGAACCCUCCGUCCAGGAUCCUGAGUACGGCGGAACACGUGAAAUUCCGUC
GGAAUCCGGGAGGACCAUCUCCCAAGGCUAAAUACUCCCUAGUGACCGAUAGUGAACC
AGUACCGUGAGGGAAAGGUGAAAAGCACCCCGGAAGGGGAGUGAAACAGUUCCUGAA
ACCGUGUGCCUACAAUUAGUCAAAGCCCGUUAAUGGUAAU
SEQ ID NO: 52, L. casei, Accession Number: FM177140.909841.912756
Lacticaseibacillus; Lactobacillus casei. Sequence:
UUAAGUUACAAAGGGCGCACGGUGGAUGCCUUGGCACUAGGAGCCGAUGAAGGACGG
AACUAAUACCGAUAUGCUUCGGGGAGCUAUAAGUAAGCUUUGAUCCGGAGAUUUCCG
AAUGGGGGAACCCAGUACACAUCAGUGUAUUGCUUGUCAGUGAAUACAUAGCUGGCC
GGCGGCAGACGCGGGGAACUGAAACAUCUCAGUACCCGCAGGAAGAGAAAGAAAACU
CGAUUCCCAUAGUAGCGGCGAGCGAAGUGGGAAGAGCCCAAACCGAGAAGCUUGCUU
CUCGGGGUUGUAGGACUGGACAUUGGAGUUACCAAAGUCCGACGUAGUCGAAGUCAG
CUGGAAAGCUGCGCCACAGAAGGUGAAAGCCCUGUAAGCGAAACGUCGAACCCUCCGU
CCAGGAUCCUGAGUACGGCGGAACACGUGAAAUUCCGUCGGAAUCCGGGAGGACCAUC
UCCCAAGGCUAAAUACUCCCUAGUGACCGAUAGUGAACCAGUACCGUGAGGGAAAGG
UGAAAAGCACCCCGGAAGGGGAGUGAAACAGUUCCUGAAACCGUGUGCCUACAAUUA
GUCAAAGCCCGUUAAUGGGUAAUGGCGUGCCUUUUGUAGAAUGAACCGGCGAGUUAC
GUUUGCUUGCGAGGUUAAGAUGAAAAGUCGGAGCCGUAGCGAAAGCGAGUCUGAACA
GGGCGAAUGAGUAAGCAGAUGUAGACCCGAAACCAAGUGACCUACCCAUGACCAGGU
UGAAGGUGUGGUGAAACACACUGGAGGACCGAACCCAUGUAUGUUGAAAAAUGCUGG
GAUGAGUUGUGGGUAGCGGUGAAAUUCCAAACGAACUUGGAGAUAGCUGGUUCUCUC
CGAAAUAGCUUUAGGGUUAGCCUCGGAGGAUGGAUCAUGGAGGUAGAGCACUGUUUG
AACUAGGGGCCCAUCAAGGGUUACUGAAUUCAGAUAAACUCCGAAUACCAUCGAUCU
UACUCCGGGAGUCAGACAGUGAGCGAUAAGGUCCAUUGUCGAAAGGGGAACAGCCCA
GAUCACCAGUUAAGGUCCCUAAAUUUAUGCUAAGUGGAAAAGGAUGUGGCGUUGCAC
AGACAACUAGGAUGUUGGCUCAGAAGCAGCCACCAUUUAAAGAGUGCGUAAUAGCUC
ACUAGUCGAGUGGCACUGCGCCGAAAAUAUACCGGGGCUAAGCAUAAUACCGAAACU
GUGGGUGCACCCGUAAGGGUGCGCGGUAGGAGAGCGUUCUAAGGGCGUUGAAGGUCG
AUCGUGAGGACGGCUGGAGCGCUUAGAAGUGAGAAUGCCGGCAUGAGUAGCGAAAGA
UCAGUGAGAAUCUGAUCCACCGUAUGACUAAGGUUUCCUGGGGAAGGCUCGUCCUCCC
AGGGUUAGUCGGGAUCUAAGGCGAGGCCGAGAGGCGUAGUCGAUGACAAGCAGGUUG
AGAUUCCUGCACUAGUUUAUUUUGUUUAAGCGAUGGAGGGACGCAGGAGGCUAAGGA
AAGCGCACGGCUGGAAAUGUGCGCCCAAGCAGCAAGUCUGACAGCGAGUGAAAUGCU
UGCAGUCUUAAGGACAAGCUGUGAUGGGGAACGAAAUUAUAGUAGCGAAGUUCCUGA
UGUCACACUGCCAAGAAAAGCUUCUAGUGAGAAAUAAACUACCCGUACCGCAAACCGA
CACAGGUAGUCGAGGAGAGUAUCCUCAGGUGAGCGAGCGAACUCUCGUUAAGGAACU
CGGCAAAAUGACCCCGUAACUUCGGAAGAAGGGGUGCUGACCGCAAGGUCAGCCGCAG
UGAAUAGGCCCAAACAACUGUUUAUCAAAAACACAGGUCUCUGCUAAAUCGUAAGAU
GAUGUAUAGGGGCUGACGCCUGCCCGGUGCUGGAAGGUUAAGAGGAUGAGUUAGCGC
AAGCGAAGCCCAGAAUUGAAGCCCCAGUAAACGGCGGCCGUAACUAUAACGGUCCUAA
GGUAGCGAAAUUCCUUGUCGGGUAAGUUCCGACCCGCACGAAAGGCGUAAUGAUUUG
GGCACUGUCUCAACGAGAGACUCGGUGAAAUUAUAGUACCCGUGAAGAUGCGGGUUA
CCCGCGACAGGACGGAAAGACCCCAUGGAGCUUUACUGUAGCUUGAUAUUGAGUGUU
UGUACCGCUUGUACAGGAUAGGUAGGAGCCGUAGAGAUCGGAACGCUAGUUUCGAUG
GAGGCGUUGGUGGGAUACUACCCUAGCUGUAUGAACACUCUAACCCGCGCCACUAAUC
GUGGCGGGAGACAGUGUCAGGUAGGCAGUUUGACUGGGGCGGUCGCCUCCUAAAAUG
UAACGGAGGCGCCCAAAGGUUCCCUCAGAAUGGUUGGAAAUCAUUCGCAGAGUGUAA
AGGUAUAAGGGAGCUUGACUGCGAGACUGACAAGUCGAGCAGGGACGAAAGUCGGGC
UUAGUGAUCCGGUGGUUCCGUAUGGAAGGGCCAUCGCUCAACGGAUAAAAGCUACCC
UGGGGAUAACAGGCUUAUCUCCCCCAAGAGUCCACAUCGACGGGGAGGUUUGGCACCU
CGAUGUCGGCUCAUCGCAUCCUGGGGCUGUAGUCGGUCCCAAGGGUUGGGCUGUUCGC
CCAUUAAAGCGGUACGCGAGCUGGGUUCAGAACGUCGUGAGACAGUUCGGUCCCUAU
CCGUCGCGGGCGCAGGAAAUUUGAGAGGAGCUGUCCUUAGUACGAGAGGACCGGGAU
GGACGUUCCGCUGGUGUACCAGUUGUGCCGCCAGGCGCAUCGCUGGGUAGCUAUGAAC
GGCAGGGAUAAACGCUGAAAGCAUCUAAGUGUGAAGCCCCCCUCGAGAUGAGAUUUC
CCAUUCCUAUAUGGAAGUAAGACCCCUGAGAGAUGAUCAGGUAGAUAGGCUGGAAGU
GGAAGUGCAGCGAUGCAUGGAGCGGACCAGUACUAAUCGGUCGAGGACUUAACCAA

It has been surprisingly discovered that postbiotic compositions prepared according to the presently disclosed methods have unique metabolite profiles and advantageous properties. In at least some instances, the presently disclosed postbiotic compositions have a metabolite profile exhibiting elevated levels or one or more advantageous metabolites selected from the group consisting of 3-hydroxybutyric acid, quercetin, phloionolic acid, wedelolactone, luteolin, N-[(2S)-2-hydroxypropanoyl]-L-leucine, organic acids including citric acid, succinic acid, lactic acid, glycerol and acetic acid, as well as indole organic acids, vitamin C, and any combination thereof.

According to another aspect of the present disclosure, oral postbiotic formulations comprising the presently disclosed postbiotic compositions are provided. The oral postbiotic formulations can be formulated as a tablet, pill, capsule, or microcapsule. In other instances, the oral postbiotic formulation can be formulated for buccal, sublabial, or sublingual administration. In still other instances, the oral postbiotic formulation can be a liquid suspension. The oral postbiotic formulations can be useful for the treatment or prevention of disruption of gut microbiota associated with an antibiotic treatment in a subject. The oral postbiotic formulations can also be useful for the treatment or prevention of dysbiosis or dysbacteriosis associated with an antibiotic treatment in a subject.

According to another aspect of the present disclosure, a method of treating or preventing the disruption of gut microbiota associated with an antibiotic treatment in a subject is provided. The method includes administering to the subject a pharmaceutically effective amount of any one of the presently disclosed postbiotic compositions or postbiotic formulations prepared according to the presently disclosed method and processes. The administering to the subject can include oral administration in the form of a tablet, pill, capsule, or microcapsule. Alternatively, the administering to the subject can include buccal, sublabial, or sublingual administration or oral administration in the form of a liquid suspension.

According to another aspect of the present disclosure, a method of treating or preventing dysbiosis or dysbacteriosis associated with an antibiotic treatment in a subject is provided. The method includes administering to the subject a pharmaceutically effective amount of any one of the presently disclosed postbiotic compositions or postbiotic formulations prepared according to the presently disclosed method and processes. The administering to the subject can include oral administration in the form of a tablet, pill, capsule, or microcapsule. Alternatively, the administering to the subject can include buccal, sublabial, or sublingual administration or oral administration in the form of a liquid suspension.

According to another aspect of the present disclosure, a method of mitigating or preventing antibiotic resistance in a subject receiving an antibiotic treatment is provided. The method includes administering to the subject a pharmaceutically effective amount of any one of the presently disclosed postbiotic compositions or postbiotic formulations prepared according to the presently disclosed method and processes. The administering to the subject can include oral administration in the form of a tablet, pill, capsule, or microcapsule. Alternatively, the administering to the subject can include buccal, sublabial, or sublingual administration or oral administration in the form of a liquid suspension.

According to another aspect of the present disclosure, a combination therapy for administering an antibiotic to a subject in need thereof while reducing or preventing antimicrobial resistance in the subject is provided. The combination therapy can include administration of a therapeutically effective amount of an antibiotic to the subject and administration of a therapeutically effective amount of one of the presently disclosed postbiotic compositions or postbiotic formulations, prepared according to the presently disclosed methods and processes. The administering to the subject can include oral administration in the form of a tablet, pill, capsule, or microcapsule. Alternatively, the administering to the subject can include buccal, sublabial, or sublingual administration or oral administration in the form of a liquid suspension. Cancer

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

In some embodiments of any of the aspects, the cancer is any cancer that can be treated with chemotherapy or immunotherapy. Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

In some embodiments of any of the aspects, the methods described herein can further comprise administering a cancer therapy, e.g., as part of a combinatorial therapy. Non-limiting examples of a cancer therapy can be selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g., see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

In some embodiments of any of the aspects, the methods described herein can further comprise administering an immune checkpoint inhibitor, e.g., as part of a combinatorial therapy. Non-limiting examples of immune checkpoint inhibitors (ICIs) include: pembrolizumab (Keytruda®), nivolumab (Opdivo®), cemiplimab (Libtayo®), spartalizumab, camrelizumab (AiRuiKa™), sintilimab (TYVYT®), tislelizumab, toripalimab (Tuoyi™), dostarlimab (JEMPERLI), INCMGA00012, AMP-224, AMP-514 (MEDI0608), atezolizumab (Tecentriq®), avelumab (Bavencio®), envafolimab (KN035), cosibelimab (CK-301), AUNP12, CA-170, BMS-986189, BMS-936559 (MDX-1105), durvalumab (IMFINZI®), tremelimumab, and ipilimumab (Yervoy®). See e.g., U.S. Pat. Nos. 5,811,097, 5,855,887, 6,051,227, 6,682,736, 6,984,720, 7,595,048, 7,605,238, 7,943,743, 8,008,449, 8,217,149, 8,354,509, 8,383,796, 8,728,474, 8,735,553, 8,779,105, 8,779,108, 8,907,053, 8,900,587, 8,952,136, 9,067,999, 9,073,994, 9,683,048, 9,987,500, U.S. Ser. No. 10/160,736, U.S. Ser. No. 10/316,089, U.S. Ser. No. 10/441,655, U.S. Ser. No. 10/590,199, U.S. Ser. No. 11/225,522, US Patent Publication US2014341917; Storz et al., MAbs. 2016 January; 8(1): 10-26; the contents of each of which are incorporated herein by reference in their entireties.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used herein, the term “dysbiosis” (also referred to as “dysbacteriosis” or “disruption of gut microbiota”) refers to a disruption to the microbiota homeostasis caused by an imbalance in the microflora, changes in their functional composition and metabolic activities, or a shift in their local distribution. It is a term for a microbial imbalance or maladaptation on or inside the body, such as an impaired microbiota. For example, a part of the human microbiota, such as the skin flora, gut flora, or vaginal flora, can become deranged, with normally dominating species underrepresented and normally outcompeted or contained species increasing to fill the void. Dysbiosis is most commonly reported as a condition in the gastrointestinal tract, but applies to other niches as well. As non-limiting examples, dysbiosis can be caused by antibiotic treatment, chemotherapy treatment, or administration of a dysbiosis-causing medication or medical treatment.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of dysbiosis (e.g., following antibiotic treatment, chemotherapy treatment, or administration of a dysbiosis-causing medication or medical treatment). A subject can be male or female.

A subject can be one who has been previously diagnosed with or identified as suffering from or having a condition in need of treatment (e.g. dysbiosis) or one or more complications related to such a condition, and optionally, have already undergone treatment for dysbiosis or the one or more complications related to dysbiosis. Alternatively, a subject can also be one who has not been previously diagnosed as having dysbiosis or one or more complications related to dysbiosis. For example, a subject can be one who exhibits one or more risk factors for dysbiosis or one or more complications related to dysbiosis or a subject who does not exhibit risk factors. In some embodiments of any of the aspects, the subject is immunocompromised, e.g., due to a medical treatment such as chemotherapy or cancer immunotherapy.

A “subject in need” of treatment for a particular condition can be a subject having that condition, diagnosed as having that condition, or at risk of developing that condition.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

In some embodiments, sequencing comprises 16S rRNA gene sequencing, which can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes with a next-generation sequencing technology as described herein (see also e.g., U.S. Pat. Nos. 5,654,418; 6,344,316; and 8,889,358; and US Patent Application Numbers US 2013/0157265 and US 2018/0195111, which are incorporated by reference in their entireties).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. dysbiosis. The term “treating” includes reducing or alleviating at least one adverse effect or symptom of a condition, disease or disorder associated with dysbiosis. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

In some embodiments of any of the aspects, the cells (e.g., bacterial cells) can be maintained in culture. As used herein, “maintaining” refers to continuing the viability of a cell or population of cells. A maintained population of cells will have at least a subpopulation of metabolically active cells.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADAM Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:

1. A fermentation substrate composition for use in a fermentation process to prepare a postbiotic composition, the fermentation substrate comprising herbal material comprising at least one of an herb of the Astragalus family, an herb of the Solanaceae or nightshade family, a berry of the Sambucus L. genus, and a legume of the Lens orientalis or Lens culinaris family, in combination with liquid water sufficient to suspend or submerge the herbal material.
2. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with liquid water sufficient to suspend or submerge the herbal material.
   3. A fermentation substrate composition for use in a fermentation process to prepare a postbiotic composition, the fermentation substrate comprising
herbal material comprising ashwagandha root and elderberry,
in combination with liquid water sufficient to suspend or submerge the herbal material.
   4. A fermentation substrate composition for use in a fermentation process to prepare a postbiotic composition, the fermentation substrate comprising
herbal material comprising an herb of the Solanaceae or nightshade family and a berry of thez Sambucus L. genus,
in combination with at least one Bifidobacterium species and at least one Lactobacillus species, and
liquid water sufficient to suspend or submerge the herbal material.
   5. A fermentation substrate composition for use in a fermentation process to prepare a postbiotic composition, the fermentation substrate comprising
herbal material comprising ashwagandha root and elderberry,
in combination with at least one Bifidobacterium species and at least one Lactobacillus species, and
liquid water sufficient to suspend or submerge the herbal material.
   6. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with at least two of the following: B. lactis, B. infantis, B. breve, L. paracasei, L. rhamnosus, and/or L. casei, and
liquid water sufficient to suspend or submerge the herbal material.
   7. A fermentation substrate composition for use in a fermentation process to prepare a postbiotic composition, the fermentation substrate comprising
herbal material comprising ashwagandha root and elderberry,
in combination with at least two of the following: B. lactis, B. infantis, B. breve, L. paracasei, L. rhamnosus, and/or L. casei, and
liquid water sufficient to suspend or submerge the herbal material.
   8. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with B. lactis, B. infantis, B. breve, L. paracasei, L. rhamnosus, and L. casei, and
liquid water sufficient to suspend or submerge the herbal material.
   9. A fermentation substrate composition for use in a fermentation process to prepare a postbiotic composition, the fermentation substrate comprising
herbal material comprising ashwagandha root and elderberry,
in combination with B. lactis, B. infantis, B. breve, L. paracasei, L. rhamnosus, and L. casei, and
liquid water sufficient to suspend or submerge the herbal material.
   10. The fermentation substrate of any one of the preceding paragraphs, further comprising one or more of glucose, sucrose, fructose, honey, and molasses.
   11. The fermentation substrate of any one of the preceding paragraphs, wherein the herbal material is provided as a dried powder prior to combination with water.
   12. The fermentation substrate of any one of the preceding paragraphs, which comprises an herb of the Astragalus family and an herb of the Solanaceae or nightshade family.
   13. The fermentation substrate of any one of the preceding paragraphs, which comprises an herb of the Astragalus family and a berry of the Sambucus L. genus.
   14. The fermentation substrate of any one of the preceding paragraphs, which comprises an herb of the Astragalus family and a legume of the Lens orientalis or Lens culinaris family.
   15. The fermentation substrate of any one of the preceding paragraphs, which comprises an herb of the Astragalus family, an herb of the Solanaceae or nightshade family and a berry of the Sambucus L. genus.
   16. The fermentation substrate of any one of the preceding paragraphs, which comprises an herb of the Astragalus family, an herb of the Solanaceae or nightshade family and a legume of the Lens orientalis or Lens culinaris family.
   17. The fermentation substrate of any one of the preceding paragraphs, which comprises an herb of the Astragalus family, a berry of the Sambucus L. genus and a legume of the Lens orientalis or Lens culinaris family.
   18. The fermentation substrate of any one of the preceding paragraphs, which comprises an herb of the Solanaceae or nightshade family and a berry of the Sambucus L. genus.
   19. The fermentation substrate of any one of the preceding paragraphs, which comprises an herb of the Solanaceae or nightshade family and a legume of the Lens orientalis or Lens culinaris family.
   20. The fermentation substrate of any one of the preceding paragraphs, which comprises an herb of the Solanaceae or nightshade family, a berry of the Sambucus L. genus and a legume of the Lens orientalis or Lens culinaris family.
   21. The fermentation substrate of any one of the preceding paragraphs, which comprises a berry of the Sambucus L. genus and a legume of the Lens orientalis or Lens culinaris family.
   22. The fermentation substrate of any one of the preceding paragraphs, which comprises an herb of the Astragalus family, an herb of the Solanaceae or nightshade family, a berry of the Sambucus L. genus and a legume of the Lens orientalis or Lens culinaris family.
   23. The fermentation substrate of any one of the preceding paragraphs, wherein the herb of the Astragalus family is Astragalus membranaceus root.
   24. The fermentation substrate of any one of the preceding paragraphs, wherein the herb of the Solanaceae or nightshade family is ashwagandha root.
   25. The fermentation substrate of any one of the preceding paragraphs, wherein the berry of the Sambucus L. genus is elderberry.
   26. The fermentation substrate of any one of the preceding paragraphs, wherein the legume of the Lens orientalis or Lens culinaris family is a lentil.
   27. The fermentation substrate of any one of the preceding paragraphs, wherein the legume is a red lentil.
   28. A fermentation substrate composition for use in a fermentation process to prepare a postbiotic composition, the fermentation substrate comprising at least one herbal material selected from Astragalus membranaceus root, ashwagandha, elderberry and red lentil, in combination with liquid water sufficient to suspend or submerge the herbal material.
   29. The fermentation substrate of any one of the preceding paragraphs, further comprising one or more of glucose, sucrose, fructose, honey, and molasses.
   30. The fermentation substrate of any one of the preceding paragraphs, wherein the herbal material is provided as a dried powder prior to combination with water.
   31. The fermentation substrate of any one of the preceding paragraphs, wherein the fermentation substrate comprises Astragalus membranaceus root and Ashwagandha.
   32. The fermentation substrate of any one of the preceding paragraphs, wherein the fermentation substrate comprises Astragalus membranaceus root and elderberry.
   33. The fermentation substrate of any one of the preceding paragraphs, wherein the fermentation substrate comprises Astragalus membranaceus root and red lentil.
   34. The fermentation substrate of any one of the preceding paragraphs, wherein the fermentation substrate comprises ashwagandha and elderberry.
   35. The fermentation substrate of any one of the preceding paragraphs, wherein the fermentation substrate comprises elderberry and red lentil.
   36. The fermentation substrate of any one of the preceding paragraphs, wherein the fermentation substrate comprises Astragalus membranaceus root, ashwagandha and elderberry.
   37. The fermentation substrate of any one of the preceding paragraphs, wherein the fermentation substrate comprises Astragalus membranaceus root, ashwagandha and red lentil.
   38. The fermentation substrate of any one of the preceding paragraphs, wherein the fermentation substrate comprises Astragalus membranaceus root, elderberry and red lentil.
   39. The fermentation substrate of any one of the preceding paragraphs, wherein the fermentation substrate comprises Astragalus membranaceus root, ashwagandha, elderberry and red lentil.
   40. The fermentation substrate of any one of the preceding paragraphs, wherein the fermentation substrate comprises about 2% by weight to about 10% by weight Astragalus membranaceus root.
   41. The fermentation substrate of any one of the preceding paragraphs, wherein the fermentation substrate comprises about 2% by weight to about 10% by weight ashwagandha.
   42. The fermentation substrate of any one of the preceding paragraphs, wherein the fermentation substrate comprises about 2% by weight to about 10% by weight elderberry.
   43. The fermentation substrate of any one of the preceding paragraphs, wherein the fermentation substrate comprises about 0.5% by weight to about 3% by weight red lentil.
   44. The fermentation substrate of any one of the preceding paragraphs, wherein the fermentation substrate comprises about 2% by weight to about 10% by weight glucose, sucrose, fructose, honey, or molasses.
   45. The fermentation substrate of any one of the preceding paragraphs, wherein the fermentation substrate comprises about 70% by weight to about 95% by weight water.
   46. The fermentation substrate of any one of the preceding paragraphs, wherein the fermentation substrate comprises about 2.5% by weight to about 5% by weight Astragalus, about 2.5% by weight to about 5% by weight ashwagandha, about 2.5% by weight to about 5% by weight elderberry, about 2.5% by weight to about 5% by weight sucrose or molasses and about 80% by weight to about 90% by weight water.
   47. The fermentation substrate of any one of the preceding paragraphs, wherein the fermentation substrate comprises about 2.5% by weight to about 5% by weight Astragalus, about 2.5% by weight to about 5% by weight ashwagandha, about 2.5% by weight to about 5% by weight elderberry, about 0.5% by weight to about 1.5% by weight red lentil, about 2.5% by weight to about 5% by weight sucrose or molasses and about 80% by weight to about 90% by weight water.
   48. The fermentation substrate of any one of the preceding paragraphs, which further comprises at least one Bifidobacterium species and at least one Lactobacillus species.
   49. The fermentation substrate of any one of the preceding paragraphs, wherein the Bifidobacterium is selected from the group consisting of B. lactis, B. breve, B. infantis, and any combination thereof.
   50. The fermentation substrate of any one of the preceding paragraphs, wherein the Lactobacillus is selected from the group consisting of L. plantarum, L. acidophilus, L. rhamnosus, L. paracasei, L. casei, and any combination thereof.
   51. The fermentation substrate of any one of the preceding paragraphs, which comprises at least two of the following: B. lactis, B. infantis, B. breve, L. paracasei, L. rhamnosus, and/or L. casei.
   52. A postbiotic composition prepared according to a process comprising: (a) preparing a culture of microorganisms; (b) preparing a fermentation substrate composition of any one of the preceding paragraphs; (c) inoculating the fermentation substrate composition with a culture of microorganisms to generate an inoculate composition; (d) fermenting the inoculate composition for a predetermined amount of time to generate a fermented inoculate composition and; (e) lyophilizing or spray-drying the fermented inoculate composition to obtain the postbiotic composition.
   53. The postbiotic composition of any one of the preceding paragraphs, further comprising a carrier.
   54. The postbiotic composition of any one of the preceding paragraphs, wherein the carrier is resistant starch or maltodextrin.
   55. The postbiotic composition of any one of the preceding paragraphs, wherein the predetermined amount of time for fermentation is about 24 hours to about 10 days.
   56. The postbiotic composition of any one of the preceding paragraphs, wherein the culture of microorganisms comprises at least one Bifidobacterium species and at least one Lactobacillus species.
   57. The postbiotic composition of any one of the preceding paragraphs, wherein the Bifidobacterium is selected from the group consisting of B. lactis, B. breve, B. infantis, and any combination thereof.
   58. The postbiotic composition of any one of the preceding paragraphs, wherein the Lactobacillus is selected from the group consisting of L. plantarum, L. acidophilus, L. rhamnosus, L. paracasei, L. casei, and any combination thereof.
   59. The postbiotic composition of any one of the preceding paragraphs, wherein the culture of microorganisms comprises a microorganism concentration from about 1.0×10⁸ CFU/mL to about 1×10¹² CFU/mL.
   60. The postbiotic composition of any one of the preceding paragraphs, wherein fermenting the inoculate composition comprises sealing the inoculate composition in a fermentation vat under substantially anaerobic conditions.
   61. The postbiotic composition of any one of the preceding paragraphs, wherein fermenting the inoculate composition further comprises incubating the inoculate composition at a temperature from about 33° C. to about 40° C.
   62. The postbiotic composition of any one of the preceding paragraphs, wherein fermenting the inoculate composition further comprises purging the fermentation vat with nitrogen gas such that the percentage of oxygen in the fermentation vat is maintained <1.5%.
   63. The postbiotic composition of any one of the preceding paragraphs, wherein the inoculate composition is maintained at a pH from about 5.0 to about 8.0. 64. The postbiotic composition of any one of the preceding paragraphs, wherein the final bacteria content post fermentation is about 1.0×10⁸ to about 1×10¹¹ cfu/ml.
   65. The postbiotic composition of any one of the preceding paragraphs, wherein the bacterial content after drying is about 0 cfu/g to about 10×10¹⁰ cfu/g.
   66. The postbiotic composition of any one of the preceding paragraphs, wherein the bacterial content after spray drying is about 0 cfu/g.
   67. The postbiotic composition of any one of the preceding paragraphs, wherein the bacterial content after freeze drying is at most about 10¹⁰ cfu/g.
   68. The postbiotic composition of any one of the preceding paragraphs, which comprises at least one metabolite selected from Table 2.
   69. The postbiotic composition of any one of the preceding paragraphs, which comprises at least one metabolite selected from the group consisting of 3-hydroxybutyric acid, quercetin, phloionolic acid, wedelolactone, luteolin, N-[(2S)-2-hydroxypropanoyl]-L-leucine, an indole organic acid or any combination thereof.
   70. The postbiotic composition of any one of the preceding paragraphs, which comprises each of 3-hydroxybutyric acid, quercetin, phloionolic acid, wedelolactone, luteolin and N-[(2S)-2-hydroxypropanoyl]-L-leucine and an indole organic acid.
   71. The postbiotic composition of any one of the preceding paragraphs, which comprises one or more organic acids produced by the fermentation, selected from citric acid, succinic acid, lactic acid, glycerol and acetic acid.
   72. The postbiotic composition of any one of the preceding paragraphs, which comprises each of citric acid, succinic acid, lactic acid, glycerol and acetic acid.
   73. The postbiotic composition of any one of the preceding paragraphs, which comprises nucleic acid including sequences selected from: B. lactis, B. breve, B. infantis, L. plantarum, L. acidophilus, L. rhamnosus, L. casei, and/or L. paracasei.
   74. The postbiotic composition of any one of the preceding paragraphs, which comprises nucleic acid molecules that can hybridize with primer sequences selected from:

B. lactis:
(SEQ ID NO: 1)
TGGAGGGTTCGATTCTGGCTCAGGATGAACGCTG,
B. breve:
(SEQ ID NO: 2)
CCGGATGCTCCATCACAC,
B. breve:
(SEQ ID NO: 3)
ACAAAGTGCCTTGCTCCCT,
B. infantis:
(SEQ ID NO: 4)
TTCCAGTTGATCGCATGGTC,
B. infantis:
(SEQ ID NO: 5)
GGAAACCCCATCTCTGGGAT,
L. plantarum:
(SEQ ID NO: 6)
GCTGGCAATGCCATCGTGCT,
L. plantarum:
(SEQ ID NO: 7)
TCTCAACGGTTGCTGTATCG,
L. acidophilus:
(SEQ ID NO: 8)
CCTTTCTAAGGAAGCGAAGGAT,
L. acidophilus:
(SEQ ID NO: 9)
ACGCTTGGTATTCCAAATCGC,
L. rhamnosus:
(SEQ ID NO: 10)
GCCGATCGTTGACGTTAGTTGG,
L. rhamnosus:
(SEQ ID NO: 11)
CAGCGGTTATGCGATGCGAAT,
L. paracasei:
(SEQ ID NO: 12)
CAATGCCGTGGTTGTTGGAA,
or
L. paracasei:
(SEQ ID NO: 13)
GCCAATCACCGCATTAATCG.

75. A postbiotic composition comprising 3-hydroxybutyric acid, quercetin, phloionolic acid, wedelolactone, luteolin, N-[(2S)-2-hydroxypropanoyl]-L-leucine and an indole organic acid.
76. The postbiotic composition of any one of the preceding paragraphs, further comprising bacteria of the genera Bifidobacterium and Lactobacillus.
77. The postbiotic composition of any one of the preceding paragraphs, further comprising a 16S RNA having a nucleic acid sequence at least 90% identical to one of SEQ ID NO: 14-16 (B. lactis), SEQ ID NO: 17-20 (B. breve), SEQ ID NO: 21-26 (B. infantis), SEQ ID NO: 27-32 (L. plantarum), SEQ ID NO: 33-39 (L. acidophilus), SEQ ID NO: 40-44 (L. rhamnosus), SEQ ID NO: 45-48 (L. paracasei), or SEQ ID NO: 49-52 (L. casei).
78. The postbiotic composition of any one of the preceding paragraphs, further comprising one or more organic acids selected from citric acid, succinic acid, lactic acid, glycerol and acetic acid.
79. The postbiotic composition of any one of the preceding paragraphs, which comprises each of citric acid, succinic acid, lactic acid, glycerol and acetic acid.
80. A composition for oral delivery, the composition comprising a postbiotic composition of any one of the preceding paragraphs, formulated for oral delivery.
81. The composition of any one of the preceding paragraphs, wherein the composition is formulated as a tablet, pill, capsule, or microcapsule.
82. The composition of any one of the preceding paragraphs, wherein the formulation comprises a liquid suspension.
83. A pharmaceutical composition comprising a composition of any one of the preceding paragraphs and a pharmaceutically acceptable carrier.
84. A method of treating or preventing disruption of gut microbiota associated with an antibiotic treatment, chemotherapy treatment, or administration of a dysbiosis-causing medication or medical treatment in a subject, the method comprising administering to the subject an amount of a composition of any one of the preceding paragraphs effective to treat or prevent the disruption.
85. The method of any one of the preceding paragraphs, wherein the medical treatment comprises a cancer immunotherapy.
86. The method of any one of the preceding paragraphs, wherein the cancer immunotherapy comprises immune checkpoint modulator/inhibitor therapy, hematopoietic cell transplantation therapy, CAR-T therapy, a dendritic cell vaccine, or any other approach that facilitates or activates an immune cell response against a cancer.
87. The method of any one of the preceding paragraphs, wherein the medical treatment comprises vaccination.
88. The method of any one of the preceding paragraphs, wherein the medical treatment comprises treatment with a dysbiosis-causing drug.
89. The method of any one of the preceding paragraphs, wherein the dysbiosis-causing drug is selected from the group consisting of acid-blocking medications, proton-pump inhibitors (PPIs), H2 blockers, birth control, steroids, antipsychotics, opioids, metformin, SSRIs, nonsteroidal anti-inflammatory drugs (NSAIDs), and any combination thereof.
90. A method of treating cancer, the method comprising administering a cancer immunotherapy and administering a composition of any one of the preceding paragraphs to a subject in need thereof, wherein the administering is effective to treat the cancer.
91. A method of treating cancer, the method comprising administering a CAR-T therapy and administering a composition of any one of the preceding paragraphs to a subject in need thereof, wherein the administering is effective to treat the cancer.
92. A method of treating cancer, the method comprising administering chemotherapy and administering a composition of any one of the preceding paragraphs to a subject in need thereof, wherein the administering is effective to treat the cancer.
93. A method of treating an infection, the method comprising administering at least one antibiotic and administering a composition of any one of the preceding paragraphs to a subject in need thereof, wherein the administering is effective to treat the infection.
94. A method of increasing neutrophil engraftment, the method comprising administering an effective amount of a composition of any one of the preceding paragraphs to a subject in need thereof.
95. A method of treating or preventing intestinal mucositis associated with chemotherapy, the method comprising administering chemotherapy and administering a composition of any one of the preceding paragraphs to a subject in need thereof, wherein the administering is effective to treat the intestinal mucositis.
96. The method of any one of the preceding paragraphs, wherein administering the composition is associated with at least one of the following outcomes, as compared to a negative control such as a subject not receiving the composition or the treated subject prior to being administered the composition: decreased incidence of cancer relapse (e.g., relapse-free); increased cancer survival; decreased time to neutrophil engraftment; increased peripheral blood mononuclear cell recovery trajectories (e.g., higher peripheral blood mononuclear cell counts); decreased incidence of febrile neutropenia; decreased blood stream infection incidence; and/or decreased 30-day readmission events.
97. The method of any one of the preceding paragraphs, wherein administering the composition in combination with chemotherapy is associated with an improvement in intestinal mucositis associated with the chemotherapy, as compared to a negative control such as a subject not receiving the composition or the treated subject prior to being administered the composition.
98. A method of preparing a postbiotic composition, the method comprising: (a) preparing a culture of microorganisms; (b) preparing a fermentation substrate composition of any one of the preceding paragraphs; (c) inoculating the fermentation substrate composition with the culture of microorganisms to generate an inoculate composition; and (d) fermenting the inoculate composition for a predetermined amount of time to generate a fermented inoculate composition.
99. The method of any one of the preceding paragraphs, wherein the predetermined amount of time is about 24 hours to about 10 days.
100. The method of any one of the preceding paragraphs, wherein the culture of microorganisms comprises at least one Bifidobacterium species and at least one Lactobacillus species.
101. The method of any one of the preceding paragraphs, wherein the Bifidobacterium is selected from the group consisting of B. lactis, B. breve, B. infantis, and any combination thereof.
102. The method of any one of the preceding paragraphs, wherein the Lactobacillus is selected from the group consisting of L. plantarum, L. acidophilus, L. rhamnosus, L. paracasei, L. casei, and any combination thereof.
103. The method of any one of the preceding paragraphs, wherein the culture of microorganisms comprises a microorganism concentration from about 1.0×10⁸ CFU/mL to about 1×10¹² CFU/mL.
104. The method of any one of the preceding paragraphs, wherein fermenting the inoculate composition comprises sealing the inoculate composition in a fermentation vat under substantially anaerobic conditions.
105. The method of any one of the preceding paragraphs, wherein fermenting the inoculate composition comprises purging the fermentation vat with nitrogen gas such that the percentage of oxygen in the fermentation vat is maintained ≤1.5%.
106. The method of any one of the preceding paragraphs, wherein fermenting the inoculate composition comprises incubating the inoculate composition at a temperature from about 33° C. to about 40° C.
107. The method of any one of the preceding paragraphs, wherein fermenting the inoculate composition comprises maintaining the pH of the fermentation in the range of about 5.0 to about 7.5.
108. The method of any one of the preceding paragraphs, further comprising lyophilizing or spray-drying the fermented inoculate composition to obtain the postbiotic composition.
109. The method of any one of the preceding paragraphs, further comprising formulating the postbiotic composition for oral delivery.
110. The method of any one of the preceding paragraphs, further comprising formulating the postbiotic composition as a tablet, pill, capsule, or microcapsule.
111. The method of any one of the preceding paragraphs, further comprising formulating the postbiotic composition in a liquid suspension.
112. A pharmaceutical composition comprising a composition prepared by the method of any one of the preceding paragraphs, and a pharmaceutically acceptable carrier.
113. A method of treating cancer, the method comprising administering at least one cancer treatment and administering a postbiotic composition to a subject in need thereof, wherein the administering is effective to treat the cancer, wherein the postbiotic composition is prepared according to a process comprising: (a) preparing a culture of microorganisms comprising B. lactis, B. infantis, B. breve, L. paracasei, L. rhamnosus, and/or L. casei; (b) preparing a fermentation substrate composition comprising herbal material comprising ashwagandha root and elderberry in combination with liquid water sufficient to suspend or submerge the herbal material; (c) inoculating the fermentation substrate composition with the culture of microorganisms to generate an inoculate composition; (d) fermenting the inoculate composition for a predetermined amount of time to generate a fermented inoculate composition and; (e) lyophilizing or spray-drying the fermented inoculate composition to obtain the postbiotic composition.

The technology described herein is further illustrated by the following examples which in no way should be construed as being further limiting.

### EXAMPLES

### Example 1—Preparation of Bifidobacterium Cultures

A 1 L flask was washed with soap and rinsed with hot water and sanitized. 500 mL of MRS broth was prepared and stirred without autoclaving. The 500 mL aliquot of MRS broth was inoculated with Bifidobacterium culture powder comprising B. lactis, B. breve, and B. infantis such that a target concentration of 1.00E+10 CFU/mL was achieved.

### Example 2—Preparation of Lactobacillus Cultures

A 1 L flask was washed with soap and rinsed with hot water and sanitized. 500 mL of MRS broth was prepared and stirred without autoclaving. The 500 mL aliquot of MRS broth was inoculated with Lactobacillus culture powder comprising L. plantarum, L. acidophilus, L. rhamnosus, and L. paracasei such that a target concentration of 1.00E+10 CFU/mL was achieved.

### Example 3—Preparation of Herbal Substrate Composition

A blender was washed with soap, rinsed with hot water, and sanitized. The herbal substrate components listed in Table 4 were introduced into a blender and blended until smooth. The pH of the of herbal substrate composition was adjusted to 6.5 with 1M NaOH.

TABLE 4
Herbal Substrate Components

| Component | Weight (kg) | Weight (% by wt) |
|---|---|---|
| Astragalus (powder) | 5.83 | 3.5% |
| Ashwagandha (powder) | 5.83 | 3.5% |
| Elderberry (powder) | 5.83 | 3.5% |
| Molasses | 5.83 | 3.5% |
| Red Lentil | 1.67 | 1.0% |
| Filtered Water | 142 | 85.0% |
| Total | 167 | 100.0% |
| Total Dry Weight | 25 | |
| Total Wet Weight | 167 | |

An alternative formulation can comprise the following: ashwagandha powder, 3.5% by weight; elderberry powder, 3.5% by weight; sucrose, 3.5% by weight, admixed with water. The term “Withania somnifera Extract” can be used interchangeably with “ashwagandha” or “ashwagandha powder.” In some embodiments of any of the aspects, the elderberry powder comprises Elderberry Juice Extract. In some embodiments, the formulation can comprise the following: Withania somnifera Extract, 3.5% by weight; Elderberry Juice Extract, 3.5% by weight; sucrose, 3.5% by weight, admixed with water.

### Example 4—Fermentation Process to Prepare Postbiotic Composition

One example of fermentation to produce a postbiotic composition is described below. A fermentation vat was washed with soap, rinsed with hot water, and sanitized. The herbal substrate composition was transferred to the fermentation vat. The vat containing the herbal substrate composition was inoculated by pipette with the Bifidobacterium cultures from Example 1 (the starter concentration was 3.00E+10 CFU/mL for Bifidobacterium) and with the Lactobacillus culture from Example 2 (the starter concentration was 4.00E+10 CFU/mL for Lactobacillus). The target concentration was 2.00E+06 CFU/mL. During fermentation, the fermentation vat was sealed and purged with nitrogen gas (99.99% purity) so that an oxygen concentration of <1% was achieved. The fermentation vat was incubated at 37° C. The pH of the fermentation volume was adjusted to 6.5 with 1M NaOH, as needed. On day 5, the fermented composition was lyophilized to produce the postbiotic composition.

### Example 5—Clinical Efficacy of the Postbiotic Compositions

Thirty-two patients were enrolled in a randomized, placebo-controlled trial. The patients were treated in a clinic with oral antibiotics for conditions not related to the gut (e.g., an ear infection). After providing informed consent, each patient received a conventional probiotic composition to take alongside their antibiotic and either: (1) a placebo control or (2) a postbiotic composition as described herein. Three stool samples were collected for each patient during the final days of the antibiotic course to assess the primary endpoint of the trial, and two more at later time points to obtain additional data. 16S rRNA gene sequencing data of the gut microbiota was analyzed with a priori defined analyses and statistics.

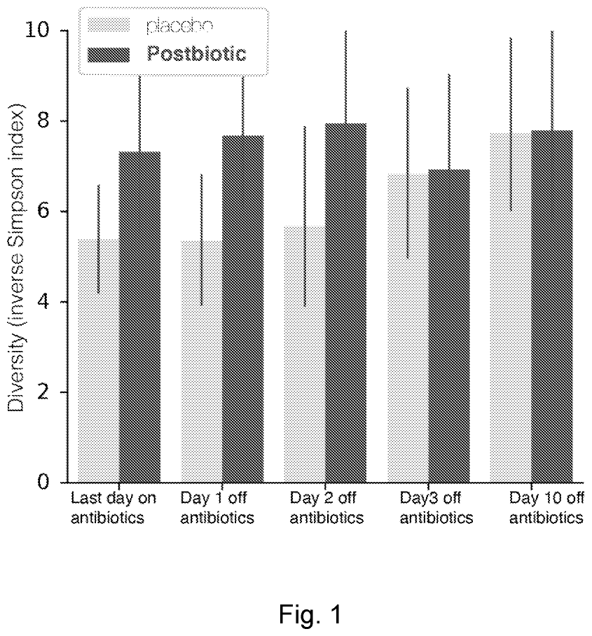

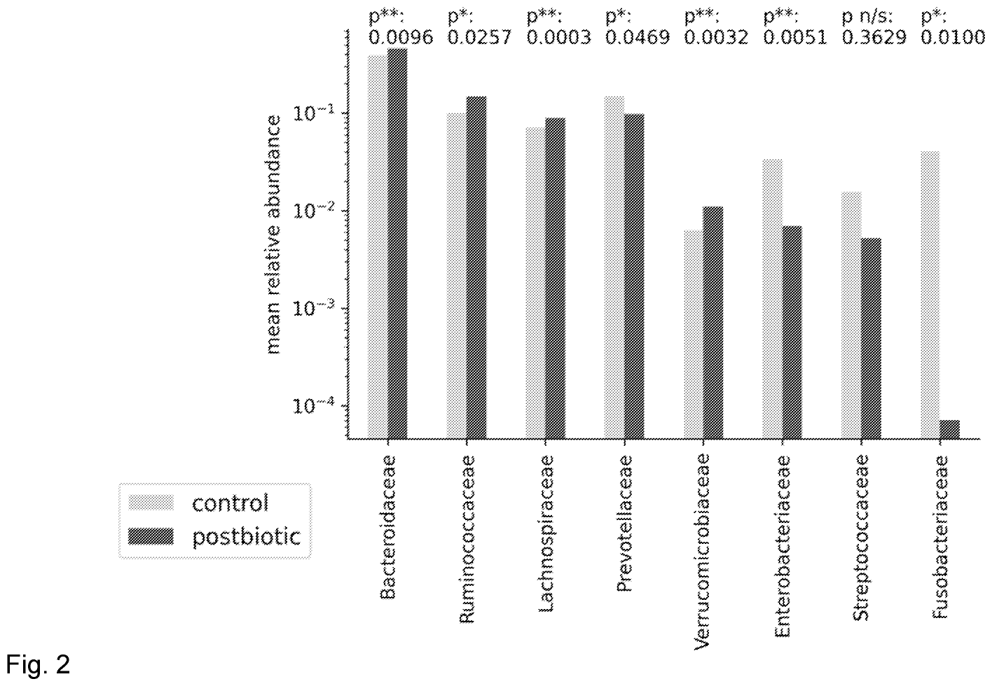

The results of the randomized placebo controlled trial showed that the microbial diversity in the gut microbiota of patients who received the presently disclosed compositions was significantly higher after antibiotic treatment than in the placebo control, as shown in FIG. 1. In particular, higher relative abundances of health-associated microbial families was observed as well as a reduction, relative to the control arm, of potentially pathogenic, dysbiotic microbial families, as shown in FIG. 2. The patients' microbiomes were measured during the end and after their antibiotic course as this is when the antibiotic induced microbiota damage is expected to be maximal. On the final day of the antibiotic courses, diversity was protected by administration of the presently disclosed compositions. Furthermore, in the absence of administration of the presently disclosed compositions, the control arm remained worse until 10 days after antibiotic administration.

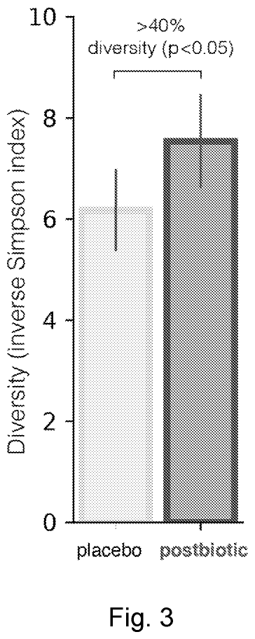

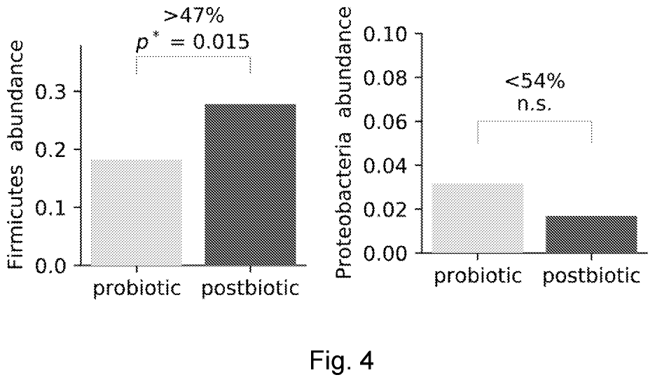

As shown in FIG. 3, administration of the presently disclosed probiotic compositions prevented damage to microbiota diversity after the antibiotic course resulting in significantly higher mean diversity (>40% diversity) than the conventional probiotic composition. As shown in FIG. 4, administration of the presently disclosed postbiotic compositions also demonstrated a greater retention of healthy bacteria, such as Firmicutes, and prevented an increase of bacteria that could be pathogenic, such as Proteobacteria. As a result, the presently disclosed postbiotic compositions can be effective in protecting gut microbiota during administration of antibiotics and can prevent collateral antibiotic-induced gut microbiota dysbiosis. Therefore, the presently disclosed postbiotic compositions and methods can be useful to reduce antibiotic resistance genes, protect against common side effects of antibiotic treatments (including diarrhea), and protect against secondary infections, especially in vulnerable populations.

### Example 6—Exemplary Protocol for Preparing and Testing a Postbiotic Composition for Use in Cancer Therapy

A postbiotic composition can be prepared according to Examples 1-4. For example, the bacteria B. lactis, B. infantis, B. breve, L. paracasei, L. rhamnosus, and L. casei can be used to ferment a fermentation substrate composition comprising ashwagandha root and elderberry (e.g., elderberry juice). In some embodiments, the formulation can comprise the following: Withania somnifera Extract, 3.5% by weight; Elderberry Juice Extract, 3.5% by weight; sucrose, 3.5% by weight, admixed with water.

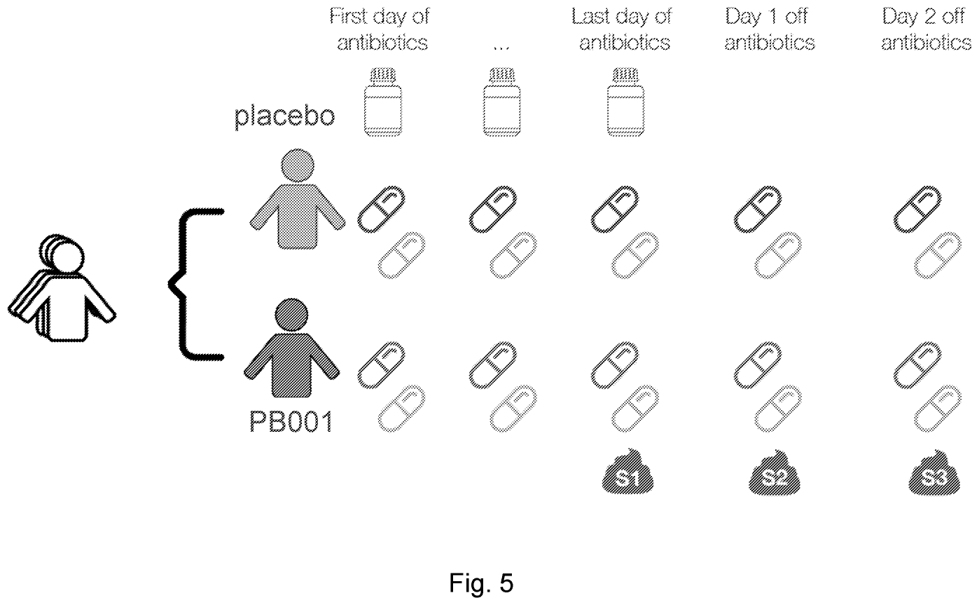

The postbiotic composition prepared from the above exemplary fermentation or a placebo can be administered to cancer patients, as shown in FIG. 5. In place of or in addition to the antibiotic treatments shown in FIG. 5, the cancer patients can be administered at least one cancer treatment, such as at least one cancer immunotherapy (e.g., immune checkpoint modulator/inhibitor therapy, hematopoietic cell transplantation therapy, CAR-T therapy, a dendritic cell vaccine, or any other approach that facilitates or activates an immune cell response against a cancer) or at least one chemotherapy. It is contemplated herein that a patient can be administered both at least one cancer treatment (e.g., chemotherapy, cancer immunotherapy) and at least one antibiotic. An ordinarily skilled clinician or oncologist can select and administer cancer therapy or therapies appropriate for a given patient's cancer. A range of such cancer therapies tends to negatively influence the gut microbiome and/or gut barrier function, and patients receiving those therapies can therefore benefit from postbiotic treatment as described herein, which helps maintain and/or restore microbiome and barrier function. An ordinarily skilled clinician or oncologist will know whether a given cancer therapeutic approach negatively influences the gut microbiome, its function or gut barrier function.

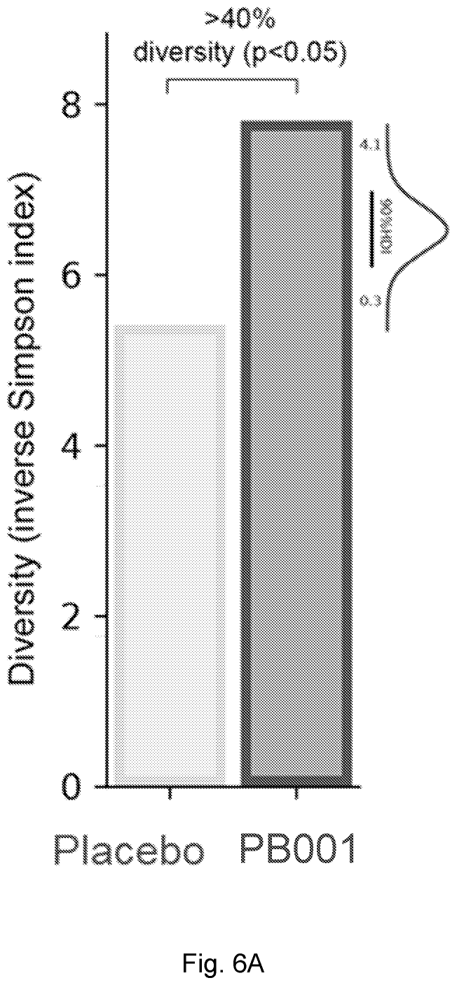

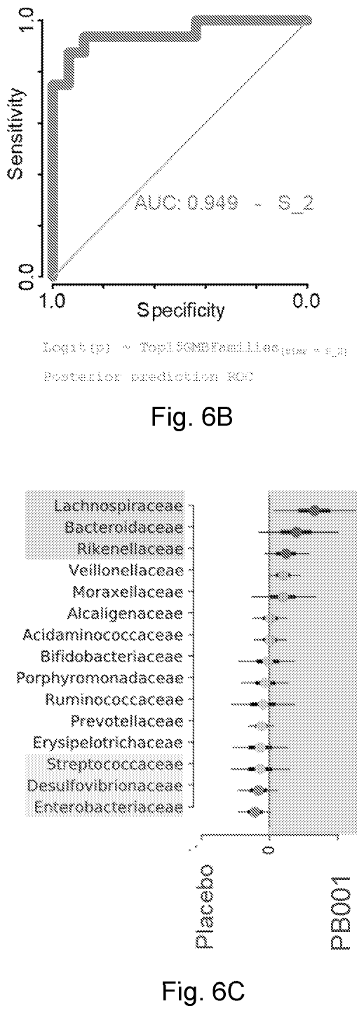

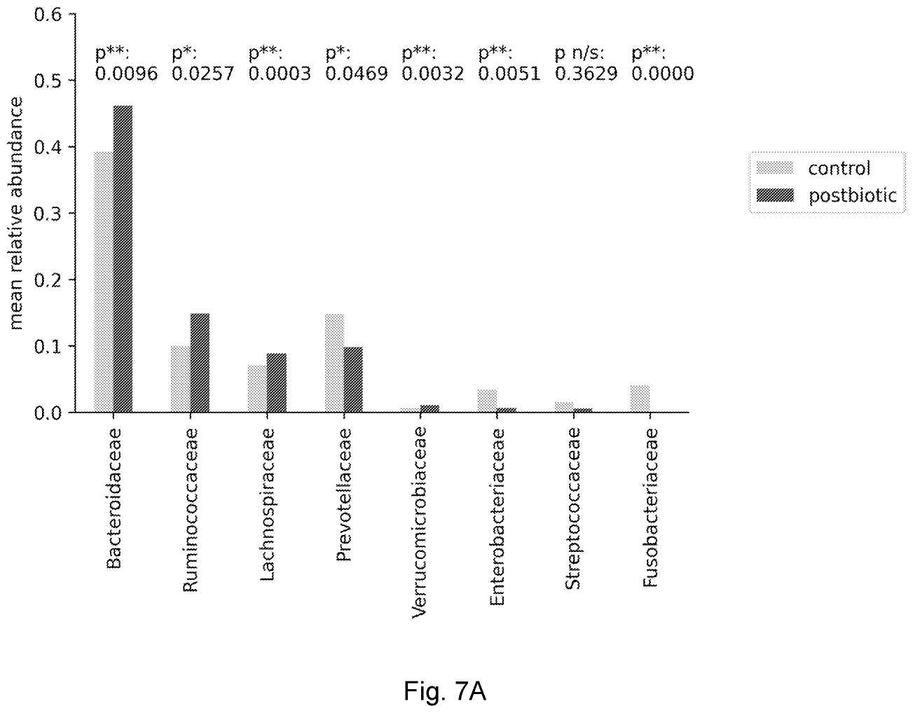

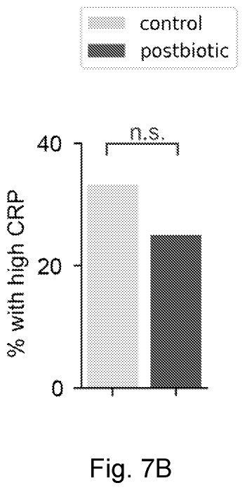

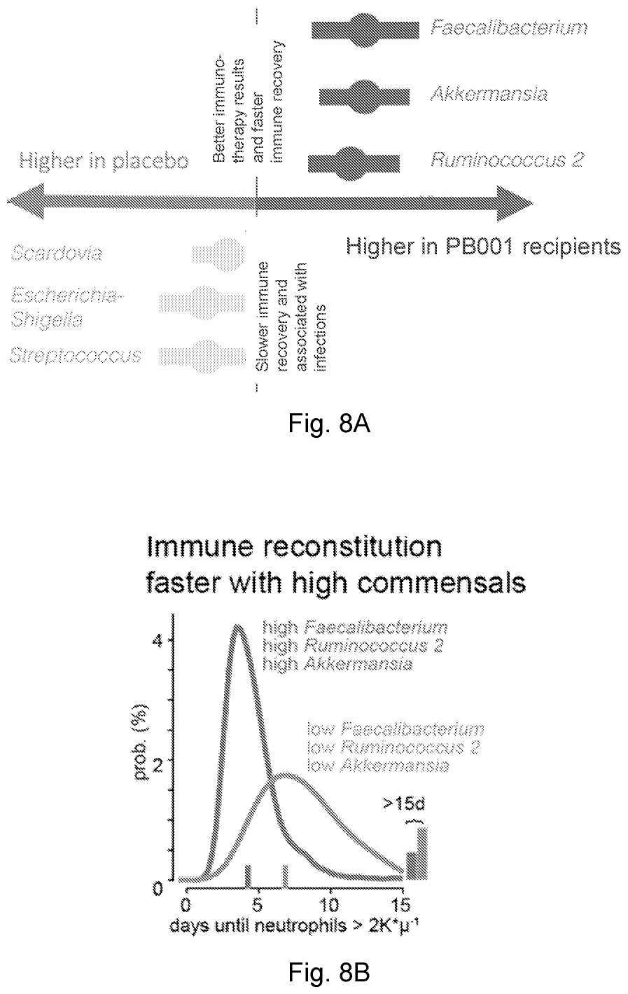

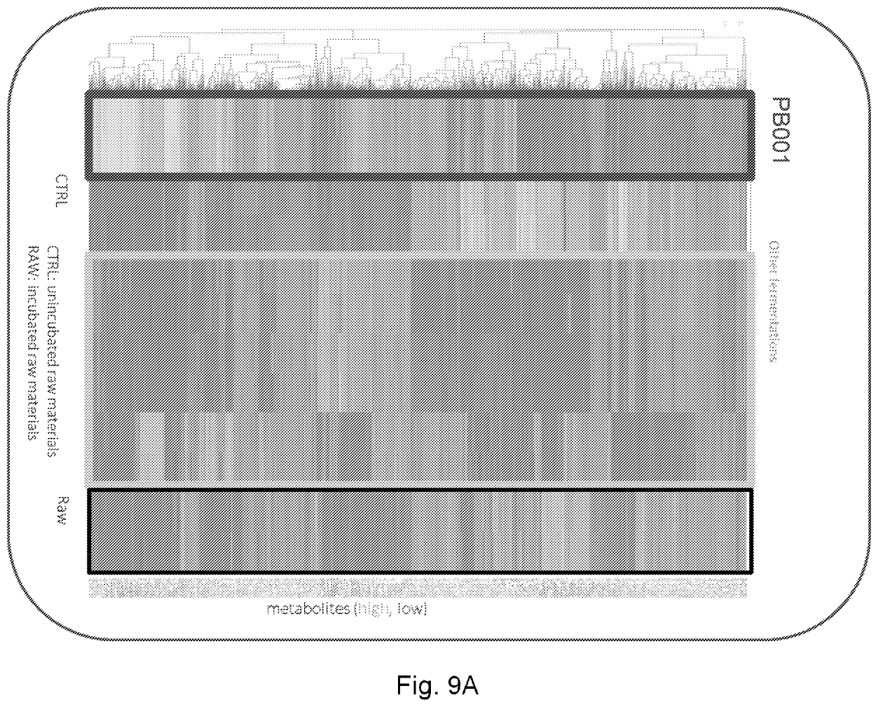

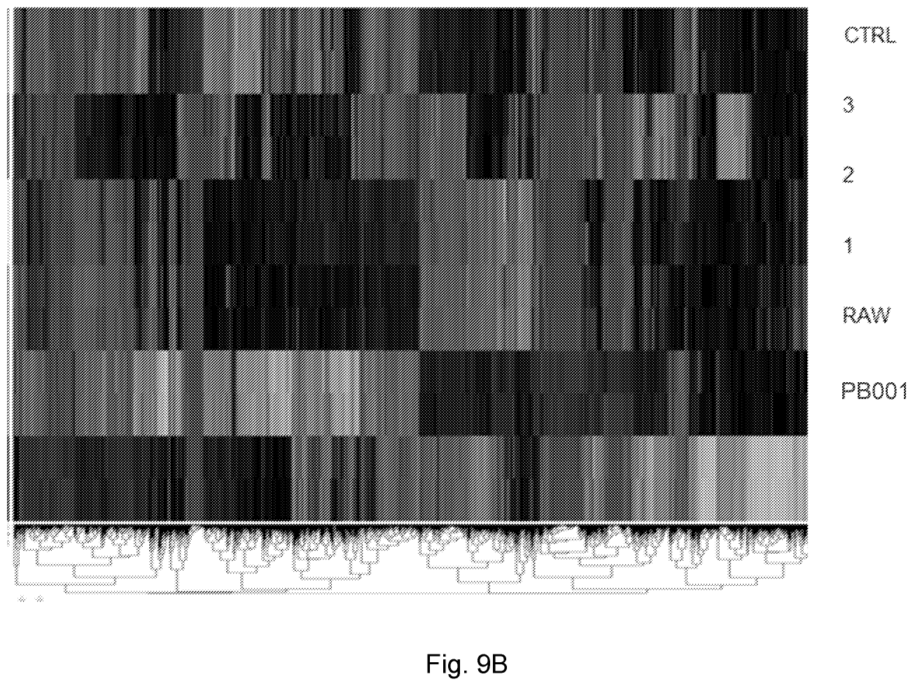

Postbiotic compositions described herein have been shown to increase health-associated bacterial families in the microbiome. Without wishing to be bound by theory, it is contemplated herein that administration of the postbiotic in combination with a cancer therapy to cancer patients will result in increased health-associated bacterial families in the microbiome, better immunotherapy results, and faster immune recovery compared to placebo and cancer treatment, e.g., with results similar to those shown in FIG. 1-4 and/or FIG. 6-9.

It is contemplated herein that administration of the postbiotic composition in combination with a cancer treatment will be associated with at least one of the following outcomes, as compared to a negative control such as a subject receiving a placebo, a subject not receiving the composition or the treated subject prior to being administered the postbiotic composition: decreased incidence of cancer relapse (e.g., relapse-free); increased cancer survival; decreased time to neutrophil engraftment; increased peripheral blood mononuclear cell recovery trajectories (e.g., higher peripheral blood mononuclear cell counts); decreased incidence of febrile neutropenia; decreased blood stream infection incidence; and/or decreased 30-day readmission events.

In some embodiments of any of the aspects, administration of the postbiotic composition in combination with chemotherapy will be associated with an improvement in intestinal mucositis associated with chemotherapy (e.g., decreased intestinal mucositis), as compared to a negative control such as a subject receiving a placebo, a subject not receiving the composition or the treated subject prior to being administered the postbiotic composition.

It is contemplated herein that administration of the postbiotic composition in combination with a cancer treatment will be associated with an increased efficacy of the cancer treatment compared to a patient receiving a placebo and the cancer treatment.

What is claimed is:

1. A fermentation substrate composition for use in a fermentation process to prepare a postbiotic composition, the fermentation substrate comprising herbal material comprising at least one of an herb of the Astragalus family, an herb of the Solanaceae or nightshade family, a berry of the Sambucus L. genus, and a legume of the Lens orientalis or Lens culinaris family, in combination with liquid water sufficient to suspend or submerge the herbal material.

2. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with liquid water sufficient to suspend or submerge the herbal material.

3. A fermentation substrate composition for use in a fermentation process to prepare a postbiotic composition, the fermentation substrate comprising
herbal material comprising ashwagandha root and elderberry,
in combination with liquid water sufficient to suspend or submerge the herbal material.

4. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with at least one Bifidobacterium species and at least one Lactobacillus species, and
liquid water sufficient to suspend or submerge the herbal material.

5. A fermentation substrate composition for use in a fermentation process to prepare a postbiotic composition, the fermentation substrate comprising
herbal material comprising ashwagandha root and elderberry,
in combination with at least one Bifidobacterium species and at least one Lactobacillus species, and
liquid water sufficient to suspend or submerge the herbal material.

6. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with at least two of the following: B. lactis, B. infantis, B. breve, L. paracasei, L. rhamnosus, and/or L. casei, and
liquid water sufficient to suspend or submerge the herbal material.

7. A fermentation substrate composition for use in a fermentation process to prepare a postbiotic composition, the fermentation substrate comprising
herbal material comprising ashwagandha root and elderberry,
in combination with at least two of the following: B. lactis, B. infantis, B. breve, L. paracasei, L. rhamnosus, and/or L. casei, and
liquid water sufficient to suspend or submerge the herbal material.

8. A fermentation substrate composition for use in a fermentation process to prepare a postbiotic composition, the fermentation substrate comprising
herbal material comprising an herb of the Solanaceae or nightshade family and a berry of the Sambucus L. genus,
in combination with B. lactis, B. infantis, B. breve, L. paracasei, L. rhamnosus, and L. casei, and
liquid water sufficient to suspend or submerge the herbal material.

9. A fermentation substrate composition for use in a fermentation process to prepare a postbiotic composition, the fermentation substrate comprising
herbal material comprising ashwagandha root and elderberry,
in combination with B. lactis, B. infantis, B. breve, L. paracasei, L. rhamnosus, and L. casei, and
liquid water sufficient to suspend or submerge the herbal material.

10. The fermentation substrate of any one of claims 1-9, further comprising one or more of glucose, sucrose, fructose, honey, and molasses.

11. The fermentation substrate of any one of claims 1-9, wherein the herbal material is provided as a dried powder prior to combination with water.

12. The fermentation substrate of any one of claims 1-9, which comprises an herb of the Astragalus family and an herb of the Solanaceae or nightshade family.

13. The fermentation substrate of any one of claims 1-9, which comprises an herb of the Astragalus family and a berry of the Sambucus L. genus.

14. The fermentation substrate of any one of claims 1-9, which comprises an herb of the Astragalus family and a legume of the Lens orientalis or Lens culinaris family.

15. The fermentation substrate of any one of claims 1-9, which comprises an herb of the Astragalus family, an herb of the Solanaceae or nightshade family and a berry of the Sambucus L. genus.

16. The fermentation substrate of any one of claims 1-9, which comprises an herb of the Astragalus family, an herb of the Solanaceae or nightshade family and a legume of the Lens orientalis or Lens culinaris family.

17. The fermentation substrate of any one of claims 1-9, which comprises an herb of the Astragalus family, a berry of the Sambucus L. genus and a legume of the Lens orientalis or Lens culinaris family.

18. The fermentation substrate of any one of claims 1-9, which comprises an herb of the Solanaceae or nightshade family and a berry of the Sambucus L. genus.

19. The fermentation substrate of any one of claims 1-9, which comprises an herb of the Solanaceae or nightshade family and a legume of the Lens orientalis or Lens culinaris family.

20. The fermentation substrate of any one of claims 1-9, which comprises an herb of the Solanaceae or nightshade family, a berry of the Sambucus L. genus and a legume of the Lens orientalis or Lens culinaris family.

21. The fermentation substrate of any one of claims 1-9, which comprises a berry of the Sambucus L. genus and a legume of the Lens orientalis or Lens culinaris family.

22. The fermentation substrate of any one of claims 1-9, which comprises an herb of the Astragalus family, an herb of the Solanaceae or nightshade family, a berry of the Sambucus L. genus and a legume of the Lens orientalis or Lens culinaris family.

23. The fermentation substrate of any one of claims 1-9, wherein the herb of the Astragalus family is Astragalus membranaceus root.

24. The fermentation substrate of any one of claims 1-9, wherein the herb of the Solanaceae or nightshade family is ashwagandha root.

25. The fermentation substrate of any one of claims 1-9, wherein the berry of the Sambucus L. genus is elderberry.

26. The fermentation substrate of any one of claims 1-9, wherein the legume of the Lens orientalis or Lens culinaris family is a lentil.

27. The fermentation substrate of claim 1, wherein the legume is a red lentil.

28. A fermentation substrate composition for use in a fermentation process to prepare a postbiotic composition, the fermentation substrate comprising at least one herbal material selected from Astragalus membranaceus root, ashwagandha, elderberry and red lentil, in combination with liquid water sufficient to suspend or submerge the herbal material.

29. The fermentation substrate of claim 28, further comprising one or more of glucose, sucrose, fructose, honey, and molasses.

30. The fermentation substrate of claim 28, wherein the herbal material is provided as a dried powder prior to combination with water.

31. The fermentation substrate of any one of claims 28-30, wherein the fermentation substrate comprises Astragalus membranaceus root and Ashwagandha.

32. The fermentation substrate of any one of claims 28-30, wherein the fermentation substrate comprises Astragalus membranaceus root and elderberry.

33. The fermentation substrate of any one of claims 28-30, wherein the fermentation substrate comprises Astragalus membranaceus root and red lentil.

34. The fermentation substrate of any one of claims 28-30, wherein the fermentation substrate comprises ashwagandha and elderberry.

35. The fermentation substrate of any one of claims 28-30, wherein the fermentation substrate comprises elderberry and red lentil.

36. The fermentation substrate of any one of claims 28-30, wherein the fermentation substrate comprises Astragalus membranaceus root, ashwagandha and elderberry.

37. The fermentation substrate of any one of claims 28-30, wherein the fermentation substrate comprises Astragalus membranaceus root, ashwagandha and red lentil.

38. The fermentation substrate of any one of claims 28-30, wherein the fermentation substrate comprises Astragalus membranaceus root, elderberry and red lentil.

39. The fermentation substrate of any one of claims 28-30, wherein the fermentation substrate comprises Astragalus membranaceus root, ashwagandha, elderberry and red lentil.

40. The fermentation substrate of any one of claims 1-9 or 28, wherein the fermentation substrate comprises about 2% by weight to about 10% by weight Astragalus membranaceus root.

41. The fermentation substrate of any one of claims 1-9 or 28, wherein the fermentation substrate comprises about 2% by weight to about 10% by weight ashwagandha.

42. The fermentation substrate of any one of claims 1-9 or 28, wherein the fermentation substrate comprises about 2% by weight to about 10% by weight elderberry.

43. The fermentation substrate of any one of claims 1-9 or 28, wherein the fermentation substrate comprises about 0.5% by weight to about 3% by weight red lentil.

44. The fermentation substrate of any one of claims 1-9 or 28, wherein the fermentation substrate comprises about 2% by weight to about 10% by weight glucose, sucrose, fructose, honey, or molasses.

45. The fermentation substrate of any one of claims 1-9 or 28, wherein the fermentation substrate comprises about 70% by weight to about 95% by weight water.

46. The fermentation substrate of any one of claims 1-9 or 28, wherein the fermentation substrate comprises about 2.5% by weight to about 5% by weight Astragalus, about 2.5% by weight to about 5% by weight ashwagandha, about 2.5% by weight to about 5% by weight elderberry, about 2.5% by weight to about 5% by weight sucrose or molasses and about 80% by weight to about 90% by weight water.

47. The fermentation substrate of any one of claims 1-9 or 28, wherein the fermentation substrate comprises about 2.5% by weight to about 5% by weight Astragalus, about 2.5% by weight to about 5% by weight ashwagandha, about 2.5% by weight to about 5% by weight elderberry, about 0.5% by weight to about 1.5% by weight red lentil, about 2.5% by weight to about 5% by weight sucrose or molasses and about 80% by weight to about 90% by weight water.

48. The fermentation substrate of any one of claims 1-9 or 28, which further comprises at least one Bifidobacterium species and at least one Lactobacillus species.

49. The fermentation substrate of claim 48, wherein the Bifidobacterium is selected from the group consisting of B. lactis, B. breve, B. infantis, and any combination thereof.

50. The fermentation substrate of claim 48, wherein the Lactobacillus is selected from the group consisting of L. plantarum, L. acidophilus, L. rhamnosus, L. paracasei, L. casei, and any combination thereof.

51. The fermentation substrate of any one of claims 1-9 or 28, which comprises at least two of the following: B. lactis, B. infantis, B. breve, L. paracasei, L. rhamnosus, and/or L. casei.

52. A postbiotic composition prepared according to a process comprising: (a) preparing a culture of microorganisms; (b) preparing a fermentation substrate composition of any one of claims 1-9 or 28; (c) inoculating the fermentation substrate composition with a culture of microorganisms to generate an inoculate composition; (d) fermenting the inoculate composition for a predetermined amount of time to generate a fermented inoculate composition and; (e) lyophilizing or spray-drying the fermented inoculate composition to obtain the postbiotic composition.

53. The postbiotic composition of claim 52, further comprising a carrier.

54. The postbiotic composition of claim 53, wherein the carrier is resistant starch or maltodextrin.

55. The postbiotic composition of claim 52, wherein the predetermined amount of time for fermentation is about 24 hours to about 10 days.

56. The postbiotic composition of claim 52, wherein the culture of microorganisms comprises at least one Bifidobacterium species and at least one Lactobacillus species.

57. The postbiotic composition of claim 56, wherein the Bifidobacterium is selected from the group consisting of B. lactis, B. breve, B. infantis, and any combination thereof.

58. The postbiotic composition of claim 56, wherein the Lactobacillus is selected from the group consisting of L. plantarum, L. acidophilus, L. rhamnosus, L. paracasei, L. casei, and any combination thereof.

59. The postbiotic composition of claim 52, wherein the culture of microorganisms comprises a microorganism concentration from about 1.0×10⁸ CFU/mL to about 1×10¹² CFU/mL.

60. The postbiotic composition of claim 52, wherein fermenting the inoculate composition comprises sealing the inoculate composition in a fermentation vat under substantially anaerobic conditions.

61. The postbiotic composition of claim 52, wherein fermenting the inoculate composition further comprises incubating the inoculate composition at a temperature from about 33° C. to about 40° C.

62. The postbiotic composition of claim 52, wherein fermenting the inoculate composition further comprises purging the fermentation vat with nitrogen gas such that the percentage of oxygen in the fermentation vat is maintained ≤1.5%.

63. The postbiotic composition of claim 52, wherein the inoculate composition is maintained at a pH from about 5.0 to about 8.0.

64. The postbiotic composition of claim 52, wherein the final bacteria content post fermentation is about 1.0×10⁸ to about 1×10¹¹ cfu/ml.

65. The postbiotic composition of claim 52, wherein the bacterial content after drying is about 0 cfu/g to about 10×10¹⁰ cfu/g.

66. The postbiotic composition of claim 52, wherein the bacterial content after spray drying is about 0 cfu/g.

67. The postbiotic composition of claim 52, wherein the bacterial content after freeze drying is at most about 10¹⁰ cfu/g.

68. The postbiotic composition of claim 52, which comprises at least one metabolite selected from Table 2.

69. The postbiotic composition of claim 52, which comprises at least one metabolite selected from the group consisting of 3-hydroxybutyric acid, quercetin, phloionolic acid, wedelolactone, luteolin, N-[(2S)-2-hydroxypropanoyl]-L-leucine, an indole organic acid or any combination thereof.

70. The postbiotic composition of claim 52, which comprises each of 3-hydroxybutyric acid, quercetin, phloionolic acid, wedelolactone, luteolin and N-[(2S)-2-hydroxypropanoyl]-L-leucine and an indole organic acid.

71. The postbiotic composition of claim 52, which comprises one or more organic acids produced by the fermentation, selected from citric acid, succinic acid, lactic acid, glycerol and acetic acid.

72. The postbiotic composition of claim 52, which comprises each of citric acid, succinic acid, lactic acid, glycerol and acetic acid.

73. The postbiotic composition of claim 52, which comprises nucleic acid including sequences selected from: B. lactis, B. breve, B. infantis, L. plantarum, L. acidophilus, L. rhamnosus, L. casei, and/or L. paracasei.

74. The postbiotic composition of claim 52, which comprises nucleic acid molecules that can hybridize with primer sequences selected from: B. lactis: TGGAGGGTTCGATTCTGGCTCAGGATGAACGCTG (SEQ ID NO: 1), B. breve: CCGGATGCTCCATCACAC (SEQ ID NO: 2), B. breve: ACAAAGTGCCTTGCTCCCT (SEQ ID NO: 3), B. infantis: TTCCAGTTGATCGCATGGTC (SEQ ID NO: 4), B. infantis: GGAAACCCCATCTCTGGGAT (SEQ ID NO: 5), L. plantarum: GCTGGCAATGCCATCGTGCT (SEQ ID NO: 6), L. plantarum: TCTCAACGGTTGCTGTATCG (SEQ ID NO: 7), L. acidophilus: CCTTTCTAAGGAAGCGAAGGAT (SEQ ID NO: 8), L. acidophilus: ACGCTTGGTATTCCAAATCGC (SEQ ID NO: 9), L. rhamnosus: GCCGATCGTTGACGTTAGTTGG (SEQ ID NO: 10), L. rhamnosus: CAGCGGTTATGCGATGCGAAT (SEQ ID NO: 11), L. paracasei: CAATGCCGTGGTTGTTGGAA (SEQ ID NO: 12), or L. paracasei: GCCAATCACCGCATTAATCG (SEQ ID NO: 13).

75. A postbiotic composition comprising 3-hydroxybutyric acid, quercetin, phloionolic acid, wedelolactone, luteolin, N-[(2S)-2-hydroxypropanoyl]-L-leucine and an indole organic acid.

76. The postbiotic composition of claim 75, further comprising bacteria of the genera Bifidobacterium and Lactobacillus.

77. The postbiotic composition of claim 75 or 76, further comprising a 16S RNA having a nucleic acid sequence at least 90% identical to one of SEQ ID NO: 14-16 (B. lactis), SEQ ID NO: 17-20 (B. breve), SEQ ID NO: 21-26 (B. infantis), SEQ ID NO: 27-32 (L. plantarum), SEQ ID NO: 33-39 (L. acidophilus), SEQ ID NO: 40-44 (L. rhamnosus), SEQ ID NO: 45-48 (L. paracasei), or SEQ ID NO: 49-52 (L. casei).

78. The postbiotic composition of claim 75 or 76, further comprising one or more organic acids selected from citric acid, succinic acid, lactic acid, glycerol and acetic acid.

79. The postbiotic composition of claim 78, which comprises each of citric acid, succinic acid, lactic acid, glycerol and acetic acid.

80. A composition for oral delivery, the composition comprising a postbiotic composition of claim 52, formulated for oral delivery.

81. The composition of claim 80, wherein the composition is formulated as a tablet, pill, capsule, or microcapsule.

82. The composition of claim 80, wherein the formulation comprises a liquid suspension.

83. A pharmaceutical composition comprising a composition of any one of claims 43-69 and a pharmaceutically acceptable carrier.

84. A method of treating or preventing disruption of gut microbiota associated with an antibiotic treatment, chemotherapy treatment, or administration of a dysbiosis-causing medication or medical treatment in a subject, the method comprising administering to the subject an amount of a composition of claim 52 effective to treat or prevent the disruption.

85. The method of claim 84, wherein the medical treatment comprises a cancer immunotherapy.

86. The method of claim 85, wherein the cancer immunotherapy comprises immune checkpoint modulator/inhibitor therapy, hematopoietic cell transplantation therapy, CAR-T therapy, a dendritic cell vaccine, or any other approach that facilitates or activates an immune cell response against a cancer.

87. The method of claim 84, wherein the medical treatment comprises vaccination.

88. The method of claim 84, wherein the medical treatment comprises treatment with a dysbiosis-causing drug.

89. The method of claim 88, wherein the dysbiosis-causing drug is selected from the group consisting of acid-blocking medications, proton-pump inhibitors (PPIs), H2 blockers, birth control, steroids, antipsychotics, opioids, metformin, SSRIs, nonsteroidal anti-inflammatory drugs (NSAIDs), and any combination thereof.

90. A method of treating cancer, the method comprising administering a cancer immunotherapy and administering a composition of claim 52 to a subject in need thereof, wherein the administering is effective to treat the cancer.

91. A method of treating cancer, the method comprising administering a CAR-T therapy and administering a composition of claim 52 to a subject in need thereof, wherein the administering is effective to treat the cancer.

92. A method of treating cancer, the method comprising administering chemotherapy and administering a composition of claim 52 to a subject in need thereof, wherein the administering is effective to treat the cancer.

93. A method of treating an infection, the method comprising administering at least one antibiotic and administering a composition of claim 52 to a subject in need thereof, wherein the administering is effective to treat the infection.

94. A method of increasing neutrophil engraftment, the method comprising administering an effective amount of a composition of claim 52 to a subject in need thereof.

95. A method of treating or preventing intestinal mucositis associated with chemotherapy, the method comprising administering chemotherapy and administering a composition of claim 52 to a subject in need thereof, wherein the administering is effective to treat the intestinal mucositis.

96. The method of claim 84, wherein administering the composition is associated with at least one of the following outcomes, as compared to a negative control such as a subject not receiving the composition or the treated subject prior to being administered the composition: decreased incidence of cancer relapse (e.g., relapse-free); increased cancer survival; decreased time to neutrophil engraftment; increased peripheral blood mononuclear cell recovery trajectories (e.g., higher peripheral blood mononuclear cell counts); decreased incidence of febrile neutropenia; decreased blood stream infection incidence; and/or decreased 30-day readmission events.

97. The method of claim 84, wherein administering the composition in combination with chemotherapy is associated with an improvement in intestinal mucositis associated with the chemotherapy, as compared to a negative control such as a subject not receiving the composition or the treated subject prior to being administered the composition.

98. A method of preparing a postbiotic composition, the method comprising: (a) preparing a culture of microorganisms; (b) preparing a fermentation substrate composition of any one of claims 1-9 or 28; (c) inoculating the fermentation substrate composition with the culture of microorganisms to generate an inoculate composition; and (d) fermenting the inoculate composition for a predetermined amount of time to generate a fermented inoculate composition.

99. The method of claim 98, wherein the predetermined amount of time is about 24 hours to about 10 days.

100. The method of claim 98, wherein the culture of microorganisms comprises at least one Bifidobacterium species and at least one Lactobacillus species.

101. The method of claim 100, wherein the Bifidobacterium is selected from the group consisting of B. lactis, B. breve, B. infantis, and any combination thereof.

102. The method of claim 100, wherein the Lactobacillus is selected from the group consisting of L. plantarum, L. acidophilus, L. rhamnosus, L. paracasei, L. casei, and any combination thereof.

103. The method of claim 98, wherein the culture of microorganisms comprises a microorganism concentration from about 1.0×10⁸ CFU/mL to about 1×10¹² CFU/mL.

104. The method of claim 98, wherein fermenting the inoculate composition comprises sealing the inoculate composition in a fermentation vat under substantially anaerobic conditions.

105. The method of claim 98, wherein fermenting the inoculate composition comprises purging the fermentation vat with nitrogen gas such that the percentage of oxygen in the fermentation vat is maintained ≤1.5%.

106. The method of claim 98, wherein fermenting the inoculate composition comprises incubating the inoculate composition at a temperature from about 33° C. to about 40° C.

107. The method of claim 98, wherein fermenting the inoculate composition comprises maintaining the pH of the fermentation in the range of about 5.0 to about 7.5.

108. The method of claim 98, further comprising lyophilizing or spray-drying the fermented inoculate composition to obtain the postbiotic composition.

109. The method of claim 98, further comprising formulating the postbiotic composition for oral delivery.

110. The method of claim 98, further comprising formulating the postbiotic composition as a tablet, pill, capsule, or microcapsule.

111. The method of claim 98, further comprising formulating the postbiotic composition in a liquid suspension.

112. A pharmaceutical composition comprising a composition prepared by the method of claim 98, and a pharmaceutically acceptable carrier.

113. A method of treating cancer, the method comprising administering at least one cancer treatment and administering a postbiotic composition to a subject in need thereof, wherein the administering is effective to treat the cancer, wherein the postbiotic composition is prepared according to a process comprising: (a) preparing a culture of microorganisms comprising B. lactis, B. infantis, B. breve, L. paracasei, L. rhamnosus, and/or L. casei; (b) preparing a fermentation substrate composition comprising herbal material comprising ashwagandha root and elderberry in combination with liquid water sufficient to suspend or submerge the herbal material; (c) inoculating the fermentation substrate composition with the culture of microorganisms to generate an inoculate composition; (d) fermenting the inoculate composition for a predetermined amount of time to generate a fermented inoculate composition and; (e) lyophilizing or spray-drying the fermented inoculate composition to obtain the postbiotic composition.
